(19) United States
(12) Patent Application Publication
(10) Pub. No.: US 2024/0190831 A1
(43) Pub. Date: Jun. 13, 2024

(54) TRANSTHYRETIN STABILIZING COMPOUNDS

(71) Applicant: Protego Biopharma, Inc., San Diego, CA (US)

(72) Inventors: Richard F. Labaudiniere, Charleston, SC (US); Bradley Dean Tait, North Andover, MA (US); Hank Michael James Petrassi, La Jolla, CA (US)

(21) Appl. No.: 18/419,081

(22) Filed: Jan. 22, 2024

Related U.S. Application Data
(63) Continuation of application No. PCT/US2022/038460, filed on Jul. 27, 2022.
(60) Provisional application No. 63/203,691, filed on Jul. 28, 2021.

(51) Int. Cl. C07D 263/57; C07D 413/06; C07D 413/12; C07D 413/14; C07D 471/04; C07D 487/04

(52) U.S. Cl. CPC C07D 263/57; C07D 413/06; C07D 413/12; C07D 413/14; C07D 471/04; C07D 487/04

(57) ABSTRACT

Provided herein are compounds having activity against TTR related conditions, and pharmaceutically accepted salts and solvates thereof. Also provided are methods of using the compounds for inhibiting and preventing TTR aggregation and/or amyloid formation in the peripheral nerves, kidney, cardiac tissue, eye and CNS, and of treating a subject with peripheral TTR amyloidosis.

### RELATED APPLICATIONS

This application is a continuation under 35 USC § 120 of International Application No. PCT/US2022/038460, filed Jul. 27, 2022, which claims priority to U.S. provisional application No. 63/203,691, filed Jul. 28, 2021. The contents of the above-referenced applications are incorporated by reference herein in their entirety.

### BACKGROUND

Transthyretin (TTR) amyloidosis is a severely debilitating, and ultimately fatal, systemic condition induced by the accumulation of TTR amyloid within tissues in amounts sufficient to impair normal function. The transthyretin (TTR) amyloidosis (ATTR) are fatal progressive sporadic (WT TTR aggregates) or autosomal dominant degenerative diseases (mutant and WT TTR aggregates). The ATTR's are caused by dissociation of tetramer TTR subunits, followed by monomer misfolding, and misassembly into a spectrum of TTR aggregate structures, including amyloid fibrils. TTR is synthesized and secreted primarily by the liver (which is not a site of aggregate deposition) into the blood, by retinal pigment and ciliary pigment epithelial cells into the eye, and by the choroid plexus into the central nervous system (CNS). The clinical expression is variable among different mutations and different populations, and even the same population with the same mutation can present significant variability. The age of onset varies between the 20s and the 90s. The TTR amyloidosis present with a diversity of symptoms and phenotypes, including peripheral polyneuropathy, autonomic neuropathy, cardiomyopathy, carpal tunnel syndrome, ocular amyloid angiopathy and leptomeningeal amyloid angiopathy, reflecting the different sources of TTR synthesis and the susceptibilities of various tissues to discrete toxic aggregate structures comprised of different TTR sequences. The peripheral nerves and the heart are the organs most frequently affected by TTR amyloid deposition, leading to ATTR-familial amyloid polyneuropathy (ATTR-FAP) and ATTR-familial amyloid cardiomyopathy (ATTR-FAC), respectively. Wild-type TTR can also be deposited as amyloid, particularly in the heart leading to wild-type transthyretin amyloid, also known as senile systemic amyloidosis (SSA). The main feature of ATTR-FAP is progressive, length-dependent degenerative sensorimotor and autonomic neuropathy. Cardiac involvement in ATTR can range from asymptomatic atrioventricular block to severe and rapidly progressive cardiomyopathy and heart failure and include arrhythmias and conduction disturbances, and cardiac infiltration with ventricular wall thickness progressing to heart failure. Average life expectancy in symptomatic FAP without treatment is 10 years, in FAC and SSA it is perhaps half that or less. Deposition of TTR amyloid in the eye and brain are associated with oculoleptomeningeal amyloidosis (ATTR-OLMA), a rare form of TTR amyloidosis with an average life expectancy of 4 to 12 years after onset. The sources of misfolded TTR in the brain and eye are the choroid plexus, the retinal pigment epithelium and ciliary pigment epithelium, respectively. TTR oculopathy is characterized, initially by dry eyes, then by progressive TTR amyloid deposition in the iris and anterior capsule of the lens. Conjunctival amyloid vasculopathy, scalloped pupils, glaucoma, vitreous opacities and finally retinal amyloid angiopathy complete the ocular pathological cascade. Vitreous opacity is treated by vitrectomy and intraocular lens implantation, however recurrent vitreous opacities occur in 14% of the treated eyes. Glaucoma is a major ocular manifestation in ATTR patients and the leading cause of irreversible blindness in these patients. Occurrence of glaucoma in this patient population is significantly increased in eyes with amyloid deposition (vitreous opacity, amyloid deposition on the pupils, fringed pupils and scalloped pupils). Trabeculectomy with mitomycin C is a standard eye surgical treatment in moderate and advanced glaucoma patients. The surgical probability of success of trabeculectomy, at 5 years, is very low (<20%) in ATTR patients, compared to 70% in non-TTR glaucoma patients. Post-surgery complications of ocular decompression retinopathy and neovascular glaucoma, caused by amyloid angiopathy are significantly increased in ATTR patient population. In addition, TTR amyloid deposition in the meninges and vessels of the brain and spinal cord is manifested clinically by transient focal neurological episodes (TFNE) most common 10-15 years after disease onset. TFNEs include transient ischemic attack-like episodes, stroke, aura-like episodes and epileptic seizures—with symptoms lasting several min to several hours to days. TFNEs frequency, duration of symptoms and cerebral TTR amyloid deposition increase with time. The phenotype-genotype relationships in ATTR are not completely understood. More than 100 TTR mutations have been associated with ATTR. Historically, several one-point mutations have been associated with one major phenotype: V30M for ATTR-PN, V1221 and wt for ATTR-FAC, D18G and Y114C for oculoleptomeningeal amyloidosis. In fact, most of the TTR variants are associated with mixed phenotypes. As ATTR is a systemic disease, other organs can become involved as the disease progresses. Recent evidence suggests that ocular and CNS amyloid depositions occur in a large proportion of ATTR-FAP patients and can become manifest 5-15 years post polyneuropathy onset and in those patients with long-standing disease and with extended survival after effective treatment targeting peripheral symptoms. Cerebral imaging by 11C-PiB PET-scan and brain biopsies indicates that cerebral TTR amyloid deposition exists prior to any overt CNS manifestations (10 years before FNE onset). Amyloid deposition is found in conjunctival vessel walls in 89% of V30M TTR-FAP patients prior to vitreous opacity. Depositions of amyloid on iris and anterior capsule of the lens are present in 40% of V30M TTR-FAP patients at 15 yrs post disease onset, in 70% at 20 years and above 80% at 25 yrs. Since 1993, liver transplantation (LT), in which the liver producing the amyloidogenic mutant TTR protein is replaced by one producing wild-type TTR, a crude form of gene therapy, was the only treatment option for ATTR-FAP. The 10-year patient survival is 79% in patients with the V30M TTR variant after LT. Clinical improvement of sensory neuropathy has been observed in 42% of subjects during the first 6 months after LT. However, LT does not prevent locally synthesized mutant-TTR amyloid deposition in the eye and brain. Variant TTR amyloid deposition has been found in vitreous humor and brains of LT ATTR-FAP patients. With or without LT treatment, prevalence of all ocular manifestations increases with disease duration. Glaucoma and vitreous opacity prevalence is up to 25% at 25 yrs. In fact, a significantly higher prevalence of amyloid deposition on the iris, on the anterior capsule of the lens and in the vitreous, and of scalloped iris is observed in liver transplanted patients versus non-transplanted patients. Furthermore, up to 31% of post-LT V30M ATTR-FAP patients will develop focal CNS manifestations 10 to 15 years post disease onset. The frequency of both cerebral amyloid deposition and FNE's increase with disease duration post LT. Tafamidis, a small molecule TTR stabilizer that inhibits TTR dissociation, misfolding and aggregation has been approved for the treatment of ATTR-FAP and ATTR-FAC in the US, EU, Japan and Brazil and in 37 additional countries. The drug is well tolerated and treatment is associated with a significant delay in the progression of peripheral neurological impairment. Tafamidis treatment significantly increase survival when compared to the natural course of the disease. In a survey conducted examining clinical data from 11 sites (in 6 countries), V30M ATTR patients treated with tafamidis or LT continue to develop ocular symptoms, vitreous opacity and glaucoma. Moreover, tafamidis failed to halt progression of oculoleptomeningeal amyloidosis in a Ala36Pro TTR patient. Tafamidis brain and eye penetrance is not sufficient to stop TTR aggregation in the eye and CNS. Despite the much lower TTR concentration in CSF and the eye compared to that in plasma (0.4-2.8 mg/dL in CSF, 0.6 mg/dL in eye versus 16-35 mg/dL in plasma), tafamidis levels in CSF and vitreous of currently tafamidis-treated FAP patients are only 2% and 0.5%, respectively, of that in plasma, leading to low tafamidis/TTR stoichiometric ratio: ≤1 in vitreous and CSF versus 2.4 in plasma. Similarly, while promising in the treatment of peripheral disease, siRNA (Alnylam) and ASO's (Ions) directed against TTR, as currently formulated, are unable to penetrate the eye or the brain, rendering them ineffective in treating the cerebral and ocular components of the TTR amyloidosis. Thus, even with the considerable progress made in therapeutic management of ATTR-FAP and ATTR-FAC, the ocular and CNS manifestations of ATTR represent a significant unmet medical need, especially when considering the prospect of prolonged survival of such patients with current treatments or those under development that effectively halt peripheral disease progression. It is probable that, with prolonged survival, serious eye disease and CNS manifestations may occur in a large proportion of ATTR patients.

### SUMMARY

Provided herein are compounds, compositions and methods for stabilizing transthyretin misfolding. In one embodiment, the compounds for use in the compositions and methods provided herein have Formula I. In another embodiment, the compounds for use in the compositions and methods provided herein have Formula II.

Also provided herein are methods of treatment of diseases and disorders resulting from transthyretin misfolding by administering a compound or composition provided herein. Further provided are methods of treatment of diseases or disorders resulting from transthyretin amyloidosis by administering a compound or composition provided herein. In other embodiments, provided herein is a method of inhibiting and preventing transthyretin aggregation and/or amyloid formation in the eye or CNS by administering a compound or composition provided herein. In another embodiment, provided herein is a method of treatment of peripheral transthyretin amyloidosis or ocular or cerebral amyloid angiopathy by administering a compound or a composition provided herein. In other embodiments, provided herein is a method of treatment of familial amyloid polyneuropathy, familial amyloid cardiomyopathy, TTR oculoleptomeningeal amyloidosis or senile systemic amyloidosis by administering a compound or a composition provided herein.

### DETAILED DESCRIPTION

### I. Definitions

The abbreviations used herein have their conventional meaning within the chemical and biological arts.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e. C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e. C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e. C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having eight or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and a heteroatom selected from the group consisting of O, N, P, Si and S, and wherein the nitrogen and sulfur atoms may optionally be oxidized and the nitrogen heteroatom may have an alkyl substituent to fulfill valency and/or may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein.

The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, deuterium, —OR′, ═O, ═NR′, ═N—OR′, —NR′R″, —SR′, halo, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″″′)═NR″″, —NR—C(NR′R″)═NR″″′, —S(O)R′, —S(O)₂R′, —S(O)₂NR′R″, —NRSO₂R′, —CN and —NO₂ in a number ranging from zero to the number of hydrogen atoms in such radical.

In one embodiment, substituent moieties for cycloalkyl, heterocycloalkyl, cycloalkenyl, and heterocycloalkenyl groups also include substituted and unsubstituted alkyl, substituted and unsubstituted alkenyl, and substituted and unsubstituted alkynyl. R′, R″, R″′ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from deuterium, halo, substituted and unsubstituted alkyl, substituted and unsubstituted alkenyl, and substituted and unsubstituted alkynyl, —OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R″′ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

When a compound provided herein includes more than one R group, for example, each of the R groups is independently selected as are each R′, R″, R′″ and R″″ groups when more than one of these groups is present.

Two of the substituent moieties on adjacent atoms of an aryl or heteroaryl ring may optionally form a ring of the formula -Q′—C(O)—(CRR′)_{q}-Q″-, wherein Q′ and Q″ are independently —NR—, —O—, —CRR′— or a single bond, and q is an integer of from 0 to 3.

Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R″′ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The term “pharmaceutically acceptable salts” refers to salts of the compounds provided herein which are prepared with relatively nontoxic acids or bases known to those of skill in the art, depending on the particular substituent moieties found on the compounds provided herein. When compounds provided herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds provided herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds provided herein contain both basic and acidic functionalities that allow the compounds to be converted into either base or acid addition salts.

The neutral forms of the compounds provided herein are in one embodiment regenerated by contacting the salt with a base or acid and isolating the parent compound in a conventional manner known to those of skill in the art.

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

In some embodiments, each substituted aryl and/or heterocycloalkyl is substituted with a substituent group, a size limited substituent group, or a lower substituent group. A “substituent group,” as used herein, means a group selected from the following moieties:
—OH, —NH₂, —SH, —CN, —CF₃, oxo, halo, unsubstituted alkyl, unsubstituted heteroalkyl, unsubstituted cycloalkyl, unsubstituted heterocycloalkyl, unsubstituted aryl, unsubstituted heteroaryl, and
alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “treating” refers to any indicia of success in the therapy or amelioration of one or more symptoms of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The therapy or amelioration of symptoms can be based on objective or subjective parameters; including the results of a physical examination, neuropsychiatric exams, and/or a psychiatric evaluation. For example, in one embodiment, the methods provided herein successfully treat a patient's delirium by decreasing the incidence of disturbances in consciousness or cognition.

Solid and dashed wedge bonds indicate stereochemistry as customary in the art. A “squiggle” bond (i.e., “ [symbol] ” indicates either R- or S-stereochemistry.

### II. Compounds and Compositions

In one embodiment, provided herein is a compound for use in the compositions and methods provided herein having Formula I:

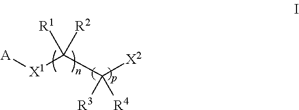

or a pharmaceutically acceptable salt or solvate thereof, wherein:
A [symbol] is

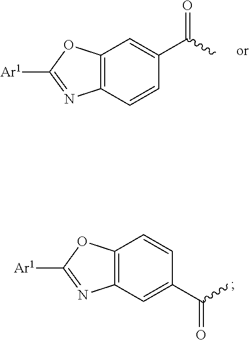

X¹ is O or NR⁵;
X² is H, halo, heteroaryl, CN, OR⁶ or NR⁷R⁸;
n is an integer from 1-2;
p is an integer from 0-3;
Ar¹ is aryl or heteroaryl, optionally substituted with halo, OR⁹, CN, COOH, CONR⁷R⁸, alkyl, haloalkyl, —(CR¹⁰R¹¹)_{q}OR⁹, —(CR¹⁰R¹¹)_{q}NR⁷R⁸ or —(CR¹⁰R¹¹)_{q}SH;
q is an integer from 0-6;
R¹-R⁸ are selected from (i)-(viii):
   (i) R¹, R², R³ and R⁴ are each independently H, halo, haloalkyl, alkyl, aryl, heteroaryl, aralkyl or heteroaralkyl (all optionally substituted); and R⁵, R⁶, R⁷ and R⁸ are each independently H, haloalkyl, alkyl, aryl, heteroaryl, aralkyl, heteroaralkyl (all optionally substituted) or —(CH₂)ₘOR⁹; where m is an integer from 1-3; or
   (ii) R¹ and R³ together with the atoms to which they are attached form a 3-6 membered ring, and R² and R⁴-R⁸ are selected as above; or
   (iii) R¹ and R⁵ together with the atoms to which they are attached form a 3-6 membered ring, and R²-R⁴ and R⁶-R⁸ are selected as above; or
   (iv) R¹ and R⁷ together with the atoms to which they are attached form a 3-6 membered ring, and R²-R⁶ and R⁸ are selected as above; or
   (v) R¹ and R⁶ together with the atoms to which they are attached form a 3-6 membered ring, and R²-R⁶ and R⁷-R⁸ are selected as above; or
   (vi) R³ and R⁵ together with the atoms to which they are attached form a 3-6 membered ring, and R¹-R², R⁴ and R⁶-R⁸ are selected as above; or
   (vii) R³ and R⁸ together with the atoms to which they are attached form a 3-6 membered ring, and R¹-R² and R⁴-R⁷ are selected as above; or
   (viii) R⁵ and R⁷ together with the atoms to which they are attached form a 3-6 membered ring, and R¹-R⁴, R⁶ and R⁸ are selected as above;
R⁹ is H, haloalkyl, alkyl, aryl, heteroaryl, aralkyl or heteroaralkyl (all optionally substituted); and
R¹⁰ and R¹¹ are each independently H, halogen, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted) or OR⁹.

In another embodiment, the compounds of Formula I provided herein for use in the compositions and methods are those wherein:
A [symbol] is

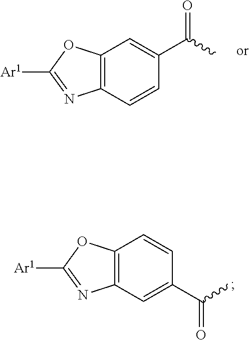

X¹ is O or NR⁵;
X² is H, halo, heteroaryl, CN, OR⁶ or NR⁷R⁸;
n is 1;
p is an integer from 0-3;
Ar¹ is aryl or heteroaryl, optionally substituted with halo, OR⁹, CN, COOH, CONR⁷R⁸, alkyl, haloalkyl, —(CR¹⁰R¹¹)_{q}OR⁹, —(CR¹⁰R¹¹)_{q}NR⁷R⁸ or —(CR¹⁰R¹¹)_{q}SH;
q is an integer from 0-6;
R¹-R⁸ are selected from (i)-(vi):
   (i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
   (ii) R¹ and R³ together with the atoms to which they are attached form a 3-6 membered ring, and R² and R⁴-R⁸ are selected as above; or
   (iii) R¹ and R⁷ together with the atoms to which they are attached form a 3-6 membered ring, and R²-R⁶ and R⁸ are selected as above; or
   (iv) R¹ and R⁶ together with the atoms to which they are attached form a 3-6 membered ring, and R²-R⁵ and R⁷-R⁸ are selected as above; or
   (v) R³ and R⁸ together with the atoms to which they are attached form a 3-6 membered ring, and R¹-R² and R⁴-R⁷ are selected as above; or
   (vi) R⁵ and R⁷ together with the atoms to which they are attached form a 3-6 membered ring, and R¹-R⁴, R⁶ and R⁸ are selected as above;
R⁹ is H or alkyl; and
R¹⁰ and R¹¹ are each independently H, halogen, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted) or OR⁹.

In another embodiment, the compounds of Formula I provided herein for use in the compositions and methods are those wherein:
A [symbol] is

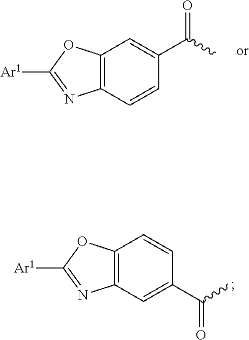

X¹ is O or NR⁵;
X² is H, halo, heteroaryl, CN, OR⁶ or NR⁷R⁸;
n is 1;
p is an integer from 0-3;
Ar¹ is aryl, optionally substituted with halo, OR⁹, CN, COOH, CONR⁷R¹ or haloalkyl;
R¹-R⁸ are selected from (i)-(vi):
   (i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
   (ii) R¹ and R³ together with the atoms to which they are attached form a 3-6 membered ring, and R² and R⁴-R⁸ are selected as above; or
   (iii) R¹ and R⁷ together with the atoms to which they are attached form a 3-6 membered ring, and R²-R⁶ and R⁸ are selected as above; or
   (iv) R¹ and R⁶ together with the atoms to which they are attached form a 3-6 membered ring, and R²-R⁵ and R⁷-R⁸ are selected as above; or
   (v) R³ and R⁸ together with the atoms to which they are attached form a 3-6 membered ring, and R¹-R² and R⁴-R⁷ are selected as above; or
   (vi) R⁵ and R⁷ together with the atoms to which they are attached form a 3-6 membered ring, and R¹-R⁴, R⁶ and R⁸ are selected as above; and
R⁹ is H or alkyl.

In another embodiment, the compounds of Formula I provided herein for use in the compositions and methods are those wherein:
A [symbol] is

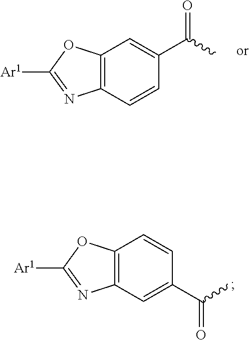

X¹ is O or NR⁵;
X² is H, halo, heteroaryl, CN, OR⁶ or NR⁷R⁸;
n is 1;
p is an integer from 0-3;
Ar¹ is aryl, optionally substituted with halo;
R¹-R⁸ are selected from (i)-(vi):
   (i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
   (ii) R¹ and R³ together with the atoms to which they are attached form a 3-6 membered ring, and R² and R⁴-R⁸ are selected as above; or
   (iii) R¹ and R⁷ together with the atoms to which they are attached form a 3-6 membered ring, and R²-R⁶ and R⁸ are selected as above; or
   (iv) R¹ and R⁶ together with the atoms to which they are attached form a 3-6 membered ring, and R²-R⁵ and R⁷-R⁸ are selected as above; or
   (v) R³ and R¹ together with the atoms to which they are attached form a 3-6 membered ring, and R¹-R² and R⁴-R⁷ are selected as above; or
   (vi) R⁵ and R⁷ together with the atoms to which they are attached form a 3-6 membered ring, and R¹-R⁴, R⁶ and R⁸ are selected as above; and
R⁹ is H or alkyl.

In another embodiment, the compounds of Formula I provided herein for use in the compositions and methods are those wherein:
A [symbol] is

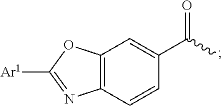

X¹ is O or NR⁵;

X² is H, halo, heteroaryl, CN, OR⁶ or NR⁷R⁸;

n is 1;

p is an integer from 0-3;

Ar¹ is aryl, optionally substituted with halo;

R¹-R⁸ are selected from (i)-(vi):
(i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
   (ii) R¹ and R³ together with the atoms to which they are attached form a 3-6 membered carbocyclic ring, and R² and R⁴-R⁸ are selected as above; or
   (iii) R¹ and R⁷ together with the atoms to which they are attached form a 3-6 membered heterocyclic ring, and R²-R⁶ and R⁸ are selected as above; or
   (iv) R¹ and R⁶ together with the atoms to which they are attached form a 3-6 membered heterocyclic ring, and R²-R⁵ and R⁷-R⁸ are selected as above; or
   (v) R³ and R⁸ together with the atoms to which they are attached form a 3-6 membered heterocyclic ring, and R¹-R² and R⁴-R⁷ are selected as above; or
   (vi) R⁵ and R⁷ together with the atoms to which they are attached form a 3-6 membered heterocyclic ring, and R¹-R⁴, R⁶ and R⁸ are selected as above; and
R⁹ is H or alkyl.

In another embodiment, X¹ in Formula I is O. In another embodiment, X¹ in Formula I is NR⁵.

In another embodiment, X² in Formula I is H. In another embodiment, X² in Formula I is halo. In another embodiment, X² in Formula I is F. In another embodiment, X² in Formula I is heteroaryl. In another embodiment, X² in Formula I is imidazolyl, pyridinyl, pyrimidinyl, pyrazinyl or pyridazinyl. In another embodiment, X² in Formula I is 2- or 4-imidazolyl, 3- or 4-pyridinyl, 2- or 4-pyrimidinyl, 2-pyrazinyl or 3-pyridazinyl. In another embodiment, X² in Formula I is CN. In another embodiment, X² in Formula I is OR⁶. In another embodiment, X² in Formula I is OH or OCH₃. In another embodiment, X² in Formula I is NR⁷R⁸. In another embodiment, X² in Formula I is NHMe or NMe₂.

In another embodiment, p in Formula I is 0. In another embodiment, p in Formula I is 1. In another embodiment, p in Formula I is 2. In another embodiment, p in Formula I is 3.

In another embodiment, Ar¹ in Formula I is phenyl optionally substituted with halo.

In another embodiment, Ar¹ in Formula I is phenyl substituted with 1-2 halo. In another embodiment, Ar¹ in Formula I is dichlorophenyl. In another embodiment, Ar¹ in Formula I is 3,5-dichlorophenyl.

In another embodiment, R¹ in Formula I is H or alkyl. In another embodiment, R¹ in Formula I is H, methyl or ethyl. In another embodiment, R¹ in Formula I is H

In another embodiment, R² in Formula I is H.

In another embodiment, R³ in Formula I is H, halo or alkyl. In another embodiment, R³ in Formula I is H, F or methyl. In another embodiment, R³ in Formula I is H.

In another embodiment, R⁴ in Formula I is H, halo or alkyl. In another embodiment, R⁴ in Formula I is H, F or methyl. In another embodiment, R⁴ in Formula I is H.

In another embodiment, R⁵ in Formula I is H or alkyl. In another embodiment, R⁵ in Formula I is H or methyl. In another embodiment, R⁵ in Formula I is H.

In another embodiment, R⁶ in Formula I is H or alkyl. In another embodiment, R⁶ in Formula I is H or methyl. In another embodiment, R⁶ in Formula I is H.

In another embodiment, R⁷ in Formula I is H or alkyl. In another embodiment, R⁷ in Formula I is H or methyl. In another embodiment, R⁷ in Formula I is H. In another embodiment, R⁷ in Formula I is methyl.

In another embodiment, R⁸ in Formula I is H or alkyl. In another embodiment, R⁸ in Formula I is H or methyl. In another embodiment, R⁸ in Formula I is H. In another embodiment, R⁸ in Formula I is methyl.

In another embodiment, R⁹ in Formula I is H or alkyl. In another embodiment, R⁹ in Formula I is H or methyl. In another embodiment, R⁹ in Formula I is H. In another embodiment, R⁹ in Formula I is methyl.

In another embodiment, R¹⁰ and R¹¹ in Formula I are each independently H, alkyl or OR⁹. In another embodiment, R¹⁰ and R¹¹ in Formula I are each independently H, methyl or OH. In another embodiment, R¹⁰ and R¹¹ in Formula I are each independently H or methyl.

In another embodiment, R¹ and R³ in Formula I together with the atoms to which they are attached form a 3-6 membered carbocyclic ring. In another embodiment, R¹ and R³ in Formula I together form methylene, ethylene or propylene.

In another embodiment, R¹ and R⁵ in Formula I together with the atoms to which they are attached form a 3-6 membered heterocyclic ring. In another embodiment, R¹ and R⁵ in Formula I together form optionally substituted ethylene, propylene, butylene or pentylene.

In another embodiment, R¹ and R⁵ in Formula I together form ethylene, propylene, butylene or pentylene, each optionally substituted with OH, halo, hydroxyalkyl, alkyl, perfluoroalkyl, spirocycloalkyl or fused cycloalkyl. In another embodiment, R¹ and R⁵ in Formula I together form propylene or butylene, each optionally substituted with OH, F, CH₂OH, methyl, CF₃, spirocyclopentyl, spirocyclobutyl or fused cyclopropyl. In another embodiment, R¹ and R⁵ in Formula I together form propylene or butylene. In another embodiment, R¹ and R⁵ in Formula I together form butylene.

In another embodiment, R¹ and R⁷ in Formula I together with the atoms to which they are attached form a 3-6 membered heterocyclic ring. In another embodiment, R¹ and R⁷ in Formula I together form optionally substituted methylene, ethylene or propylene. In another embodiment, R¹ and R⁷ in Formula I together form methylene, ethylene, propylene, —CH₂C(O)— or —CH₂CF₂—. In another embodiment, R¹ and R⁷ in Formula I together form methylene, ethylene or propylene. In another embodiment, R¹ and R⁷ in Formula I together form ethylene or propylene. In another embodiment, R¹ and R⁷ in Formula I together form ethylene.

In another embodiment, R¹ and R⁶ in Formula I together with the atoms to which they are attached form a 3-6 membered heterocyclic ring. In another embodiment, R¹ and R⁶ in Formula I together form methylene or ethylene.

In another embodiment, R³ and R⁸ in Formula I together with the atoms to which they are attached form a 3-6 membered heterocyclic ring. In another embodiment, R³ and R⁸ in Formula I together form propylene or butylene.

In another embodiment, R⁵ and R⁷ in Formula I together with the atoms to which they are attached form a 3-6 membered heterocyclic ring. In another embodiment, R⁵ and R⁷ in Formula I together form ethylene.

In another embodiment, the compounds of Formula I are those where X¹ is O; X² is NR⁷R⁸; R¹ and R⁷ together with the atoms to which they are attached form a 3-6 membered heterocyclic ring; R², R³ and R⁴ are each independently H, halo, haloalkyl, alkyl, aryl, heteroaryl, aralkyl or heteroaralkyl (all optionally substituted); R⁸ is independently H, haloalkyl, alkyl, aryl, heteroaryl, aralkyl, heteroaralkyl (all optionally substituted) or —(CH₂)ₘOR⁹ where m is an integer from 1-3; n is 1 and p is 2. In another embodiment, the compounds of Formula I are those where X¹ is O, X² is NR⁷R⁸; R¹ and R⁷ together form optionally substituted ethylene or propylene; R², R³ and R⁴ are each independently H, halo or alkyl; R⁸ is independently H or alkyl; n is 1 and p is 2. In another embodiment, the compounds of Formula I are those where X¹ is O, X² is NR⁷R⁸; R¹ and R⁷ together form ethylene or propylene; R² is H or methyl; R³ and R⁴ are each independently H, halo or alkyl; R⁸ is H, alkyl or haloalkyl; n is 1 and p is 2. In another embodiment, the compounds of Formula I are those where X¹ is O, X² is NR⁷R⁸; R¹ and R⁷ together form ethylene or propylene; R² is methyl; R³ and R⁴ are each independently H; R⁸ is ethyl, 2,2,2-trifluoroethyl, 2-fluoro-1-ethyl or 2,2-difluoro-1-ethyl; n is 1 and p is 2.

In another embodiment, the compounds of Formula I are those where X¹ is NR⁵; X² is H; R¹ and R⁵ together with the atoms to which they are attached form a 3-6 membered heterocyclic ring; R² is independently H, halo, haloalkyl, alkyl, aryl, heteroaryl, aralkyl or heteroaralkyl (all optionally substituted); n is 1 and p is 0. In another embodiment, the compounds of Formula I are those where X¹ is NR⁵; X² is H; R¹ and R⁵ together form optionally substituted ethylene, propylene, butylene or pentylene; R² is independently H, halo or alkyl; n is 1 and p is 0. In another embodiment, the compounds of Formula I are those where X¹ is NR⁵; X² is H; R¹ and R⁵ together form propylene or butylene, each optionally substituted with OH, F, CH₂OH, methyl, CF₃, spirocyclopentyl, spirocyclobutyl or fused cyclopropyl; R² is H; n is 1 and p is 0.

In another embodiment, the compounds of Formula I are those where X¹ is NR⁵; X² is H; R¹ and R³ together with the atoms to which they are attached form a 3-6 membered heterocyclic ring; R² and R⁴ are each independently H, halo, haloalkyl, alkyl, aryl, heteroaryl, aralkyl or heteroaralkyl (all optionally substituted); R⁵ is H or alkyl; n is 1 and p is 1. In another embodiment, the compounds of Formula I are those where X¹ is NR⁵; X² is H; R¹ and R³ together form methylene, ethylene or propylene; R² and R⁴ are each independently H, halo or alkyl; R⁵ is H or alkyl; n is 1 and p is 1. In another embodiment, the compounds of Formula I are those where X¹ is NR⁵; X² is H; R¹ and R³ together form propylene optionally substituted with OH; R² and R⁴ are each independently H or methyl; R⁵ is H; n is 1 and p is 1. In another embodiment, the compounds of Formula I are those where X¹ is NR⁵; X² is H; R¹ and R³ together form propylene optionally substituted with OH; R², R⁴ and R⁵ are each H; n is 1 and p is 1.

In another embodiment, the compounds provided herein for use in the compositions and methods provided herein have Formula II:

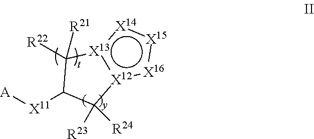

or a pharmaceutically acceptable salt or solvate thereof, wherein:
A [symbol] is

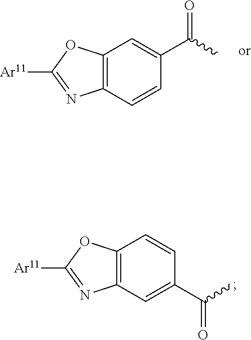

X¹¹ is O or NR²⁶.
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl or heteroaryl, optionally substituted with halo, OR³⁰, CN, COOH, CONR³¹R³², alkyl, haloalkyl, —(CR³³R³⁴)_{q}OR³⁰, —(CR³³R³⁴)_{q}NR³¹R³² or —(CR³³R³⁴)_{q}SH or CF₃;
q is an integer from 0-6;
R²¹, R²², R²³, R²⁴, R²⁵, R²⁶, R²⁷, R²⁸, R²⁹, R³⁰, R³¹, R³², R³⁵ and R³⁶ are each independently H, alkyl, aryl, aralkyl, heteroaryl or heteroaralkyl (all optionally substituted); and
R³³ and R³⁴ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR³⁰.

In another embodiment, the compounds provided herein for use in the compositions and methods provided herein have Formula II, wherein:
A [symbol] is

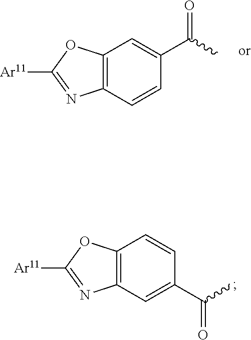

X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo, OR³⁰, CN, COOH, CONR³¹R³² or haloalkyl;
R²¹, R²², R²³, R²⁴, R²⁵, R²⁶, R²⁷, R²⁸, R²⁹, R³⁰, R³¹, R³², R³⁵ and R³⁶ are each independently H, alkyl, aryl, aralkyl, heteroaryl or heteroaralkyl (all optionally substituted).

In another embodiment, the compounds provided herein for use in the compositions and methods provided herein have Formula II, wherein:
A [symbol] is

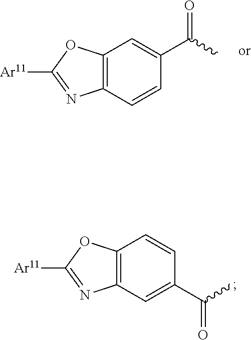

X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo; and
R²¹, R²², R²³, R²⁴, R²⁵, R²⁶, R²⁷, R²⁸, R²⁹, R³⁵ and R³⁶ are each independently H, alkyl, aryl, aralkyl, heteroaryl or heteroaralkyl (all optionally substituted).

In another embodiment, the compounds provided herein for use in the compositions and methods provided herein have Formula II, wherein:
A [symbol] is

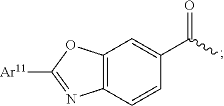

X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo; and
R²¹, R²², R²³, R²⁴, R²⁵, R²⁶, R²⁷, R²⁸, R²⁹, R³⁵ and R³⁶ are each independently H, alkyl, aryl, aralkyl, heteroaryl or heteroaralkyl (all optionally substituted).

In another embodiment, X¹¹ in Formula II is O. In another embodiment, X¹¹ in Formula II is NR²⁶. In another embodiment, X¹¹ in Formula II is NH.

In another embodiment, the ring formed by X¹²—X¹⁶ is one of the following:

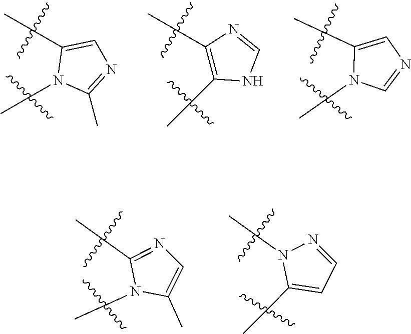

In another embodiment, X¹² in Formula II is N. In another embodiment, X¹² in Formula II is C.

In another embodiment, X¹³ in Formula II is C or N. In another embodiment, X¹³ in Formula II is C. In another embodiment, X¹³ in Formula II is N.

In another embodiment, X¹⁴ in Formula II is CR²⁹ or N. In another embodiment, X¹⁴ in Formula II is CH or N. In another embodiment, X¹⁴ in Formula II is CR²⁹. In another embodiment, X¹⁴ in Formula II is CH. In another embodiment, X¹⁴ in Formula II is N.

In another embodiment, X¹⁵ in Formula II is CR²⁸. In another embodiment, X¹⁵ in Formula II is CH or N. In another embodiment, X¹⁵ in Formula II is CH. In another embodiment, X¹⁵ in Formula II is N.

In another embodiment, X¹⁶ in Formula II is CR²⁵. In another embodiment, X¹⁶ in Formula II is CH, CMe or NH. In another embodiment, X¹⁶ in Formula II is CH. In another embodiment, X¹⁶ in Formula II is CMe. In another embodiment, X¹⁶ in Formula II is NR²⁶. In another embodiment, X¹⁶ in Formula II is NH.

In another embodiment, R³⁵ and R³⁶ in Formula II are independently H or optionally substituted alkyl. In another embodiment, R³⁵ and R³⁶ in Formula II are independently H or unsubstituted alkyl. In another embodiment, R³⁵ and R³⁶ in Formula II are H. In another embodiment, R³⁵ and R³⁶ in Formula II are independently H or methyl.

In another embodiment, t in Formula II is 2 or 3 and y in Formula II is 0 or 1. In another embodiment, t in Formula II is 1 and y in Formula II is 1. In another embodiment, t in Formula II is 2 and y in Formula II is 0 or 1. In another embodiment, t in Formula II is 2 and y in Formula II is 1. In another embodiment, t in Formula II is 2 and y in Formula II is 0.

In another embodiment, Ar¹¹ in Formula II is phenyl, optionally substituted with halo. In another embodiment, Ar¹¹ in Formula II is phenyl, optionally substituted with chloro. In another embodiment, Ar¹¹ in Formula II is dichlorophenyl. In another embodiment, Ar¹¹ in Formula II is 3,5-dichlorophenyl.

In another embodiment, R²¹, R²², R²³, R²⁴, R²⁵, R²⁶, R²⁷, R²⁸, R²⁹, R³⁰, R³¹, R³², R³⁵ and R³⁶ in Formula II are each independently H or optionally substituted alkyl. In another embodiment, R²¹, R²², R²³, R²⁴, R²⁵, R²⁶, R²⁷ and R²⁸ in Formula II are each independently H or unsubstituted alkyl. In another embodiment, R²¹, R²², R²³, R²⁴, R²⁵, R²⁶, R²⁷ and R²⁸ in Formula II are each independently H or methyl. In another embodiment, R²¹, R²², R²³ and R²⁴ in Formula II are each independently H or unsubstituted alkyl. In another embodiment, R²¹, R²², R²³ and R²⁴ in Formula II are each H. In another embodiment, R²⁵, R²⁸, R²⁹, R³⁵ and R³⁶ in Formula II are independently H or unsubstituted alkyl. In another embodiment, R²⁵, R²⁸, R²⁹, R³⁵ and R³⁶ in Formula II are independently H or methyl. In another embodiment, R²⁶ and R²⁷ in Formula II are each H. In another embodiment, R³⁰ is H or unsubstituted alkyl. In another embodiment, R³⁰ is H or methyl. In another embodiment, R³¹ and R³² are independently H or unsubstituted alkyl. In another embodiment, R³¹ and R³² are independently H or methyl.

In some embodiments, the compound of Formula I or II has the structure:

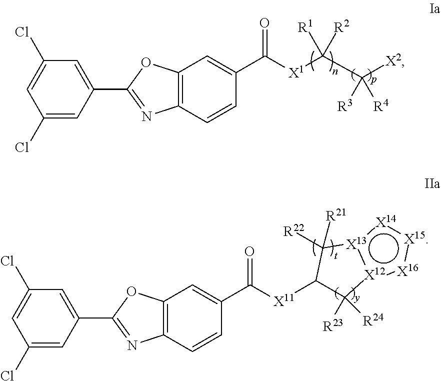

In some embodiments, the compound provided herein for use in the compositions and methods provided herein is selected from the compounds in Table 1.

TABLE 1

| Compound # | Structure |
|---|---|
| 1 | 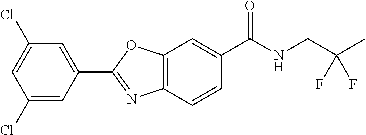 [SMILES: CC(F)(F)CNC(=O)c1ccc2nc(-c3cc(Cl)cc(Cl)c3)oc2c1] |
| 2 | 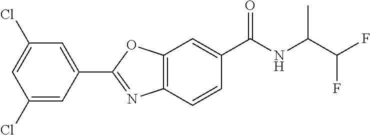 [SMILES: CC(NC(=O)c1ccc2nc(-c3cc(Cl)cc(Cl)c3)oc2c1)C(F)F] |
| 3 | 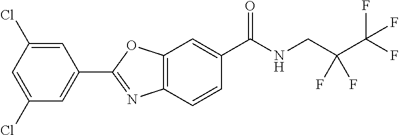 [SMILES: O=C(NCC(F)(F)C(F)(F)F)c1ccc2nc(-c3cc(Cl)cc(Cl)c3)oc2c1] |
| 4 | 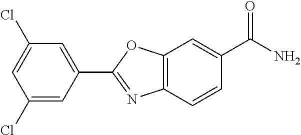 [SMILES: NC(=O)c1ccc2nc(-c3cc(Cl)cc(Cl)c3)oc2c1] |
| 5 | 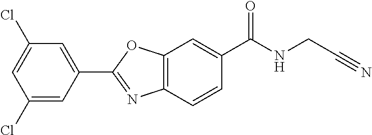 [SMILES: N#CCNC(=O)c1ccc2nc(-c3cc(Cl)cc(Cl)c3)oc2c1] |
| 6 | 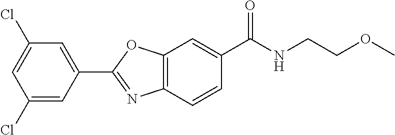 [SMILES: COCCNC(=O)c1ccc2nc(-c3cc(Cl)cc(Cl)c3)oc2c1] |
| 7 | 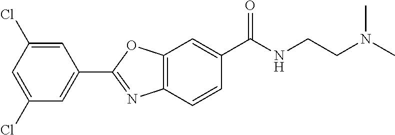 [SMILES: CN(C)CCNC(=O)c1ccc2nc(-c3cc(Cl)cc(Cl)c3)oc2c1] |
| 8 | 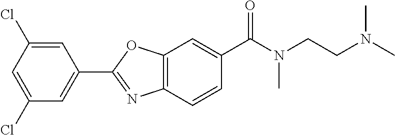 [SMILES: CN(C)CCN(C)C(=O)c1ccc2nc(-c3cc(Cl)cc(Cl)c3)oc2c1] |
| 9 | 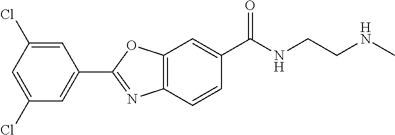 [SMILES: CNCCNC(=O)c1ccc2nc(-c3cc(Cl)cc(Cl)c3)oc2c1] |
| 10 | 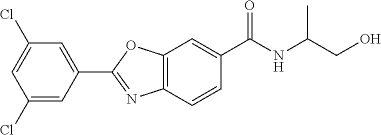 [SMILES: CC(CO)NC(=O)c1ccc2nc(-c3cc(Cl)cc(Cl)c3)oc2c1] |
| 11 | 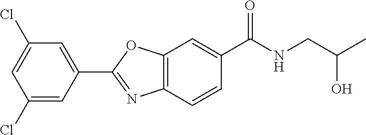 [SMILES: CC(O)CNC(=O)c1ccc2nc(-c3cc(Cl)cc(Cl)c3)oc2c1] |
| 12 | 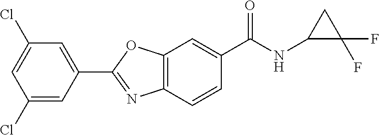 [SMILES: O=C(NC1CC1(F)F)c1ccc2nc(-c3cc(Cl)cc(Cl)c3)oc2c1] |
| 13 | 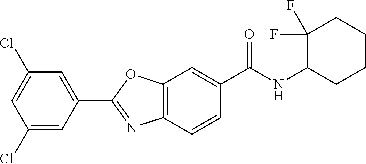 [SMILES: O=C(NC1CCCCC1(F)F)c1ccc2nc(-c3cc(Cl)cc(Cl)c3)oc2c1] |
| 14 | 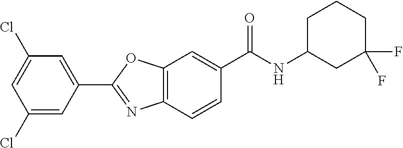 [SMILES: O=C(NC1CCCC(F)(F)C1)c1ccc2nc(-c3cc(Cl)cc(Cl)c3)oc2c1] |
| 15 | 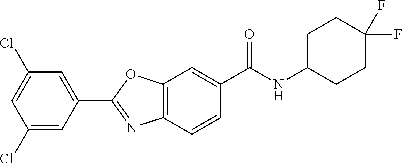 [SMILES: O=C(NC1CCC(F)(F)CC1)c1ccc2nc(-c3cc(Cl)cc(Cl)c3)oc2c1] |
| 16 | 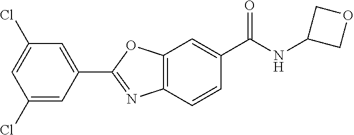 [SMILES: O=C(NC1COC1)c1ccc2nc(-c3cc(Cl)cc(Cl)c3)oc2c1] |
| 17 | 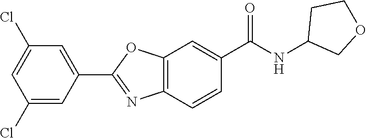 [SMILES: O=C(NC1CCOC1)c1ccc2nc(-c3cc(Cl)cc(Cl)c3)oc2c1] |
| 18 | 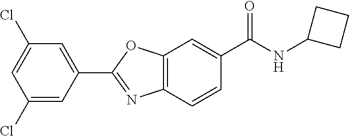 [SMILES: O=C(NC1CCC1)c1ccc2nc(-c3cc(Cl)cc(Cl)c3)oc2c1] |
| 19 | 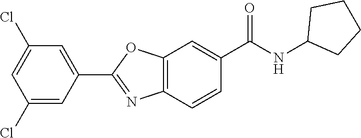 [SMILES: O=C(NC1CCCC1)c1ccc2nc(-c3cc(Cl)cc(Cl)c3)oc2c1] |
| 20 | 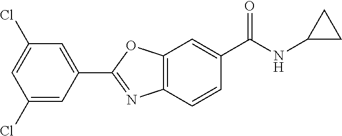 [SMILES: O=C(NC1CC1)c1ccc2nc(-c3cc(Cl)cc(Cl)c3)oc2c1] |
| 21 | 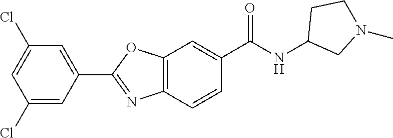 [SMILES: CN1CCC(NC(=O)c2ccc3nc(-c4cc(Cl)cc(Cl)c4)oc3c2)C1] |
| 22 | 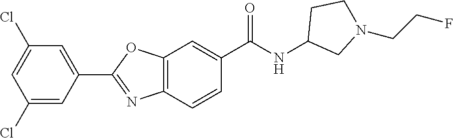 [SMILES: O=C(NC1CCN(CCF)C1)c1ccc2nc(-c3cc(Cl)cc(Cl)c3)oc2c1] |
| 23 | 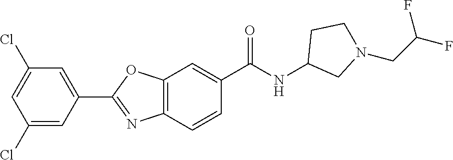 [SMILES: O=C(NC1CCN(CC(F)F)C1)c1ccc2nc(-c3cc(Cl)cc(Cl)c3)oc2c1] |
| 24 | 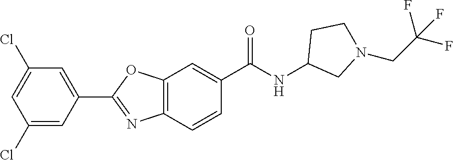 [SMILES: O=C(NC1CCN(CC(F)(F)F)C1)c1ccc2nc(-c3cc(Cl)cc(Cl)c3)oc2c1] |
| 25 | 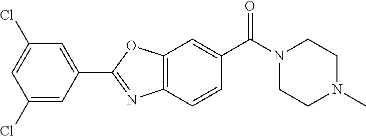 [SMILES: CN1CCN(C(=O)c2ccc3nc(-c4cc(Cl)cc(Cl)c4)oc3c2)CC1] |
| 26 | 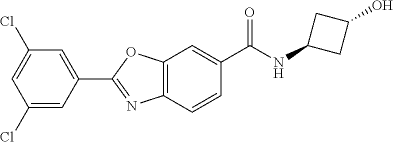 [SMILES: O=C(N[C@H]1C[C@H](O)C1)c1ccc2nc(-c3cc(Cl)cc(Cl)c3)oc2c1] |
| 27 | 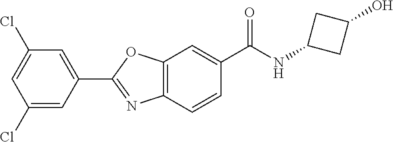 [SMILES: O=C(N[C@H]1C[C@@H](O)C1)c1ccc2nc(-c3cc(Cl)cc(Cl)c3)oc2c1] |
| 28 | 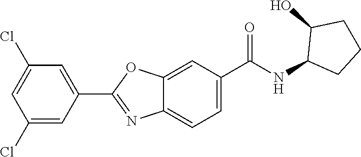 [SMILES: O=C(N[C@@H]1CCC[C@@H]1O)c1ccc2nc(-c3cc(Cl)cc(Cl)c3)oc2c1] |
| 29 | 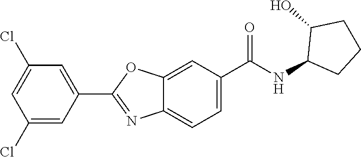 [SMILES: O=C(N[C@@H]1CCC[C@H]1O)c1ccc2nc(-c3cc(Cl)cc(Cl)c3)oc2c1] |
| 30 | 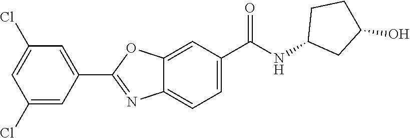 [SMILES: O=C(N[C@@H]1CC[C@H](O)C1)c1ccc2nc(-c3cc(Cl)cc(Cl)c3)oc2c1] |
| 31 | 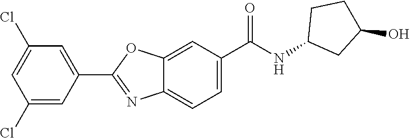 [SMILES: O=C(N[C@@H]1CC[C@@H](O)C1)c1ccc2nc(-c3cc(Cl)cc(Cl)c3)oc2c1] |
| 32 | 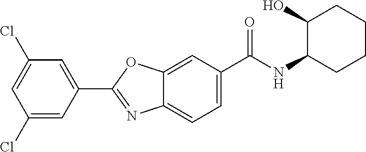 [SMILES: O=C(N[C@@H]1CCCC[C@@H]1O)c1ccc2nc(-c3cc(Cl)cc(Cl)c3)oc2c1] |
| 33 | 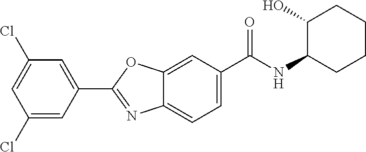 [SMILES: O=C(N[C@@H]1CCCC[C@H]1O)c1ccc2nc(-c3cc(Cl)cc(Cl)c3)oc2c1] |
| 34 | 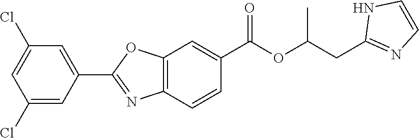 [SMILES: CC(Cc1ncc[nH]1)OC(=O)c1ccc2nc(-c3cc(Cl)cc(Cl)c3)oc2c1] |
| 35 | 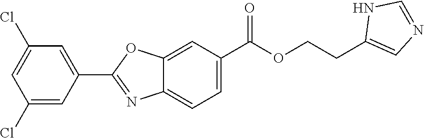 [SMILES: O=C(OCCc1cnc[nH]1)c1ccc2nc(-c3cc(Cl)cc(Cl)c3)oc2c1] |
| 36 | 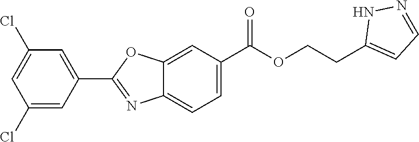 [SMILES: O=C(OCCc1ccn[nH]1)c1ccc2nc(-c3cc(Cl)cc(Cl)c3)oc2c1] |
| 37 | 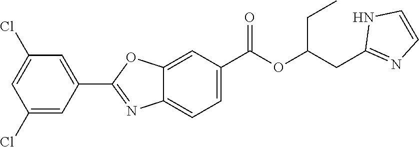 [SMILES: CCC(Cc1ncc[nH]1)OC(=O)c1ccc2nc(-c3cc(Cl)cc(Cl)c3)oc2c1] |
| 38 | 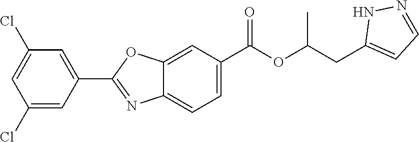 [SMILES: CC(Cc1ccn[nH]1)OC(=O)c1ccc2nc(-c3cc(Cl)cc(Cl)c3)oc2c1] |
| 39 | 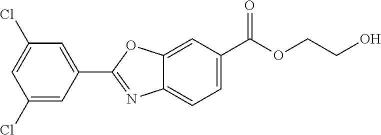 [SMILES: O=C(OCCO)c1ccc2nc(-c3cc(Cl)cc(Cl)c3)oc2c1] |
| 40 | 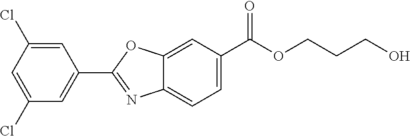 [SMILES: O=C(OCCCO)c1ccc2nc(-c3cc(Cl)cc(Cl)c3)oc2c1] |
| 41 | 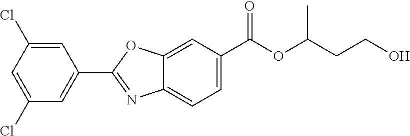 [SMILES: CC(CCO)OC(=O)c1ccc2nc(-c3cc(Cl)cc(Cl)c3)oc2c1] |
| 42 | 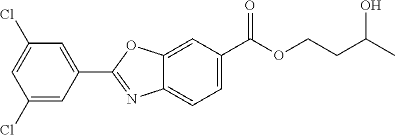 [SMILES: CC(O)CCOC(=O)c1ccc2nc(-c3cc(Cl)cc(Cl)c3)oc2c1] |
| 43 | 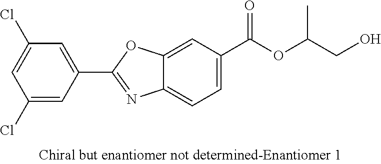 [SMILES: CC(CO)OC(=O)c1ccc2nc(-c3cc(Cl)cc(Cl)c3)oc2c1] |
| 44 | 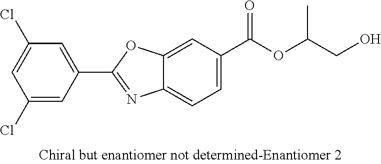 [SMILES: CC(CO)OC(=O)c1ccc2nc(-c3cc(Cl)cc(Cl)c3)oc2c1] |
| 45 | 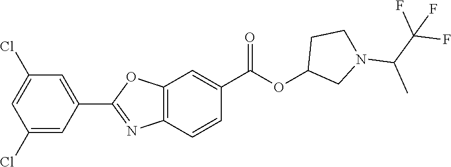 [SMILES: CC(N1CCC(OC(=O)c2ccc3nc(-c4cc(Cl)cc(Cl)c4)oc3c2)C1)C(F)(F)F] |
| 46 | 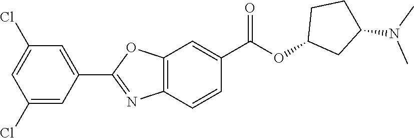 [SMILES: CN(C)[C@H]1CC[C@@H](OC(=O)c2ccc3nc(-c4cc(Cl)cc(Cl)c4)oc3c2)C1] |
| 47 | 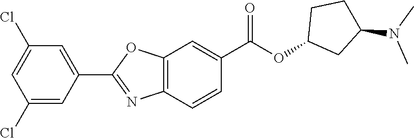 [SMILES: CN(C)[C@@H]1CC[C@@H](OC(=O)c2ccc3nc(-c4cc(Cl)cc(Cl)c4)oc3c2)C1] |
| 48 | 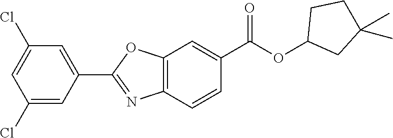 [SMILES: CC1(C)CCC(OC(=O)c2ccc3nc(-c4cc(Cl)cc(Cl)c4)oc3c2)C1] |
| 49 | 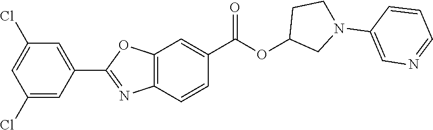 [SMILES: O=C(OC1CCN(c2cccnc2)C1)c1ccc2nc(-c3cc(Cl)cc(Cl)c3)oc2c1] |
| 50 | 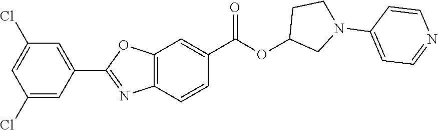 [SMILES: O=C(OC1CCN(c2ccncc2)C1)c1ccc2nc(-c3cc(Cl)cc(Cl)c3)oc2c1] |
| 51 | 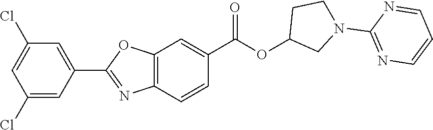 [SMILES: O=C(OC1CCN(c2ncccn2)C1)c1ccc2nc(-c3cc(Cl)cc(Cl)c3)oc2c1] |
| 52 | 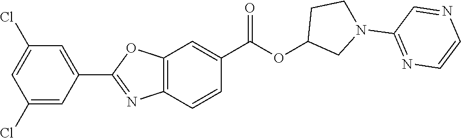 [SMILES: O=C(OC1CCN(c2cnccn2)C1)c1ccc2nc(-c3cc(Cl)cc(Cl)c3)oc2c1] |
| 53 | 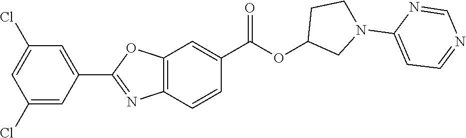 [SMILES: O=C(OC1CCN(c2ccncn2)C1)c1ccc2nc(-c3cc(Cl)cc(Cl)c3)oc2c1] |
| 54 | 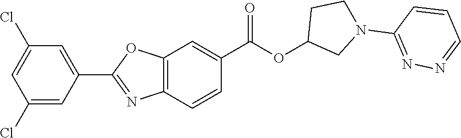 [SMILES: O=C(OC1CCN(c2cccnn2)C1)c1ccc2nc(-c3cc(Cl)cc(Cl)c3)oc2c1] |
| 55 | 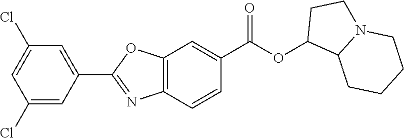 [SMILES: O=C(OC1CCN2CCCCC12)c1ccc2nc(-c3cc(Cl)cc(Cl)c3)oc2c1] |
| 56 | 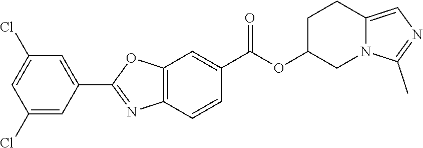 [SMILES: Cc1ncc2n1CC(OC(=O)c1ccc3nc(-c4cc(Cl)cc(Cl)c4)oc3c1)CC2] |
| 57 | 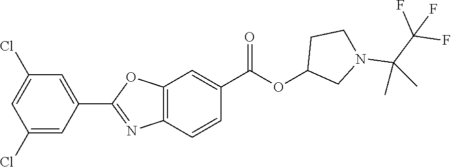 [SMILES: CC(C)(N1CCC(OC(=O)c2ccc3nc(-c4cc(Cl)cc(Cl)c4)oc3c2)C1)C(F)(F)F] |
| 58 | 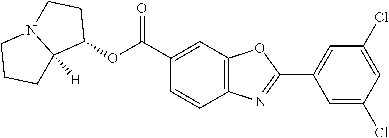 [SMILES: O=C(O[C@H]1CCN2CCC[C@H]12)c1ccc2nc(-c3cc(Cl)cc(Cl)c3)oc2c1] |
| 59 | 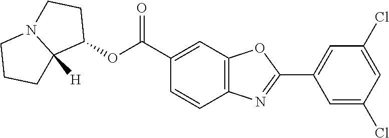 [SMILES: O=C(O[C@H]1CCN2CCC[C@@H]12)c1ccc2nc(-c3cc(Cl)cc(Cl)c3)oc2c1] |
| 60 | 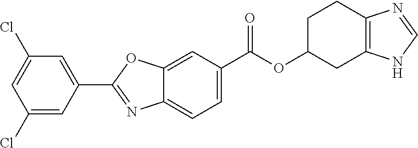 [SMILES: O=C(OC1CCc2nc[nH]c2C1)c1ccc2nc(-c3cc(Cl)cc(Cl)c3)oc2c1] |
| 61 | 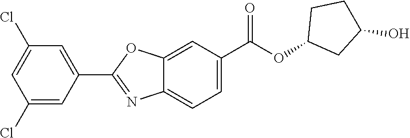 [SMILES: O=C(O[C@@H]1CC[C@H](O)C1)c1ccc2nc(-c3cc(Cl)cc(Cl)c3)oc2c1] |
| 62 | 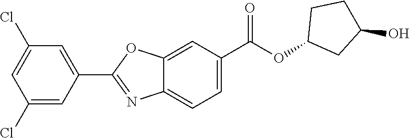 [SMILES: O=C(O[C@@H]1CC[C@@H](O)C1)c1ccc2nc(-c3cc(Cl)cc(Cl)c3)oc2c1] |
| 63 | 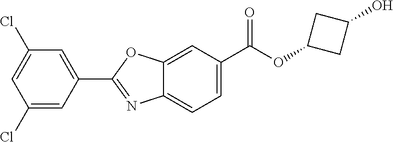 [SMILES: O=C(O[C@H]1C[C@@H](O)C1)c1ccc2nc(-c3cc(Cl)cc(Cl)c3)oc2c1] |
| 64 | 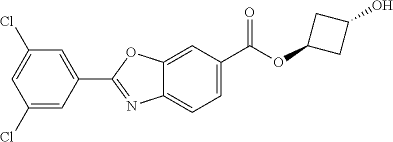 [SMILES: O=C(O[C@H]1C[C@H](O)C1)c1ccc2nc(-c3cc(Cl)cc(Cl)c3)oc2c1] |
| 65 | 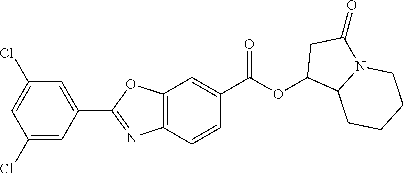 [SMILES: O=C(OC1CC(=O)N2CCCCC12)c1ccc2nc(-c3cc(Cl)cc(Cl)c3)oc2c1] |
| 66 | 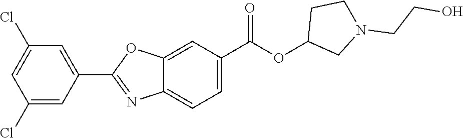 [SMILES: O=C(OC1CCN(CCO)C1)c1ccc2nc(-c3cc(Cl)cc(Cl)c3)oc2c1] |
| 67 | 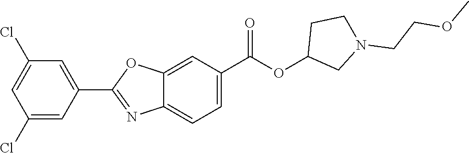 [SMILES: COCCN1CCC(OC(=O)c2ccc3nc(-c4cc(Cl)cc(Cl)c4)oc3c2)C1] |
| 68 | 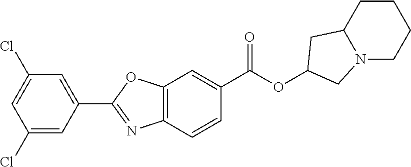 [SMILES: O=C(OC1CC2CCCCN2C1)c1ccc2nc(-c3cc(Cl)cc(Cl)c3)oc2c1] |
| 69 | 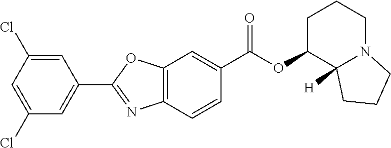 [SMILES: O=C(O[C@H]1CCCN2CCC[C@H]12)c1ccc2nc(-c3cc(Cl)cc(Cl)c3)oc2c1] |
| 70 | 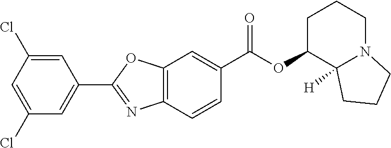 [SMILES: O=C(O[C@H]1CCCN2CCC[C@@H]12)c1ccc2nc(-c3cc(Cl)cc(Cl)c3)oc2c1] |
| 71 | 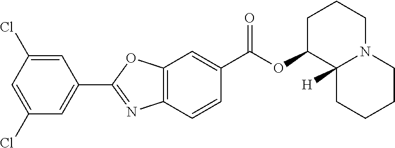 [SMILES: O=C(O[C@H]1CCCN2CCCC[C@H]12)c1ccc2nc(-c3cc(Cl)cc(Cl)c3)oc2c1] |
| 72 | 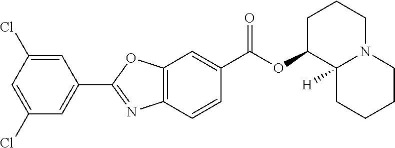 [SMILES: O=C(O[C@H]1CCCN2CCCC[C@@H]12)c1ccc2nc(-c3cc(Cl)cc(Cl)c3)oc2c1] |
| 73 | 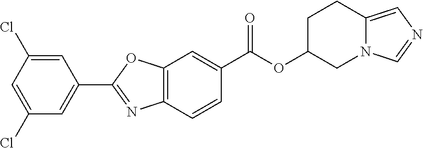 [SMILES: O=C(OC1CCc2cncn2C1)c1ccc2nc(-c3cc(Cl)cc(Cl)c3)oc2c1] |
| 74 | 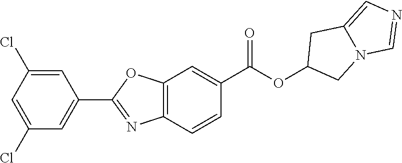 [SMILES: O=C(OC1Cc2cncn2C1)c1ccc2nc(-c3cc(Cl)cc(Cl)c3)oc2c1] |
| 75 | 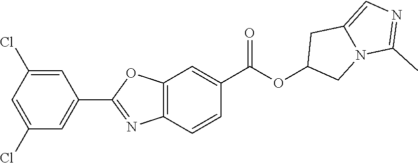 [SMILES: Cc1ncc2n1CC(OC(=O)c1ccc3nc(-c4cc(Cl)cc(Cl)c4)oc3c1)C2] |
| 76 | 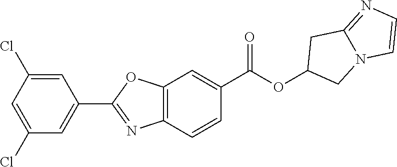 [SMILES: O=C(OC1Cc2nccn2C1)c1ccc2nc(-c3cc(Cl)cc(Cl)c3)oc2c1] |
| 77 | 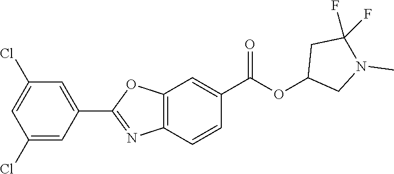 [SMILES: CN1CC(OC(=O)c2ccc3nc(-c4cc(Cl)cc(Cl)c4)oc3c2)CC1(F)F] |
| 78 | 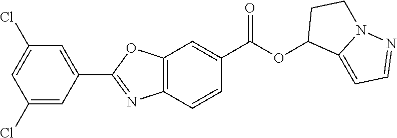 [SMILES: O=C(OC1CCn2nccc21)c1ccc2nc(-c3cc(Cl)cc(Cl)c3)oc2c1] |
| 79 | 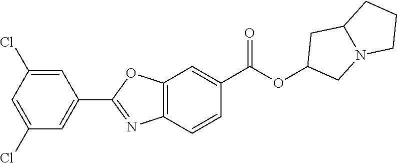 [SMILES: O=C(OC1CC2CCCN2C1)c1ccc2nc(-c3cc(Cl)cc(Cl)c3)oc2c1] |
| 80 | 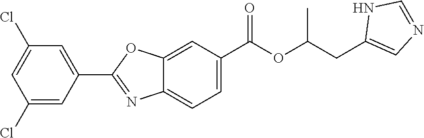 [SMILES: CC(Cc1cnc[nH]1)OC(=O)c1ccc2nc(-c3cc(Cl)cc(Cl)c3)oc2c1] |
| 81 | 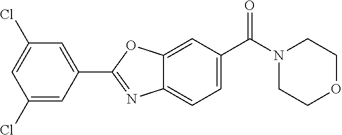 [SMILES: O=C(c1ccc2nc(-c3cc(Cl)cc(Cl)c3)oc2c1)N1CCOCC1] |
| 82 | 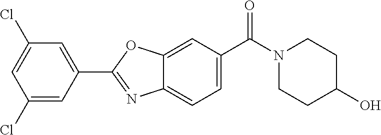 [SMILES: O=C(c1ccc2nc(-c3cc(Cl)cc(Cl)c3)oc2c1)N1CCC(O)CC1] |
| 83 | 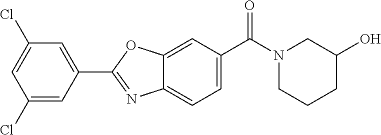 [SMILES: O=C(c1ccc2nc(-c3cc(Cl)cc(Cl)c3)oc2c1)N1CCCC(O)C1] |
| 84 | 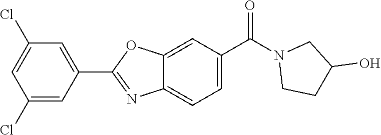 [SMILES: O=C(c1ccc2nc(-c3cc(Cl)cc(Cl)c3)oc2c1)N1CCC(O)C1] |
| 85 | 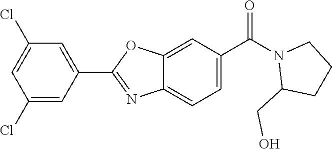 [SMILES: O=C(c1ccc2nc(-c3cc(Cl)cc(Cl)c3)oc2c1)N1CCCC1CO] |
| 86 | 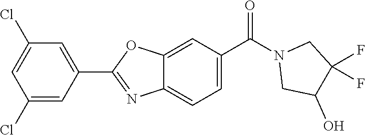 [SMILES: O=C(c1ccc2nc(-c3cc(Cl)cc(Cl)c3)oc2c1)N1CC(O)C(F)(F)C1] |
| 87 | 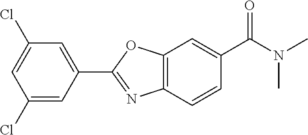 [SMILES: CN(C)C(=O)c1ccc2nc(-c3cc(Cl)cc(Cl)c3)oc2c1] |
| 88 | 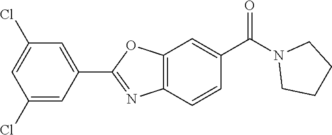 [SMILES: O=C(c1ccc2nc(-c3cc(Cl)cc(Cl)c3)oc2c1)N1CCCC1] |
| 89 | 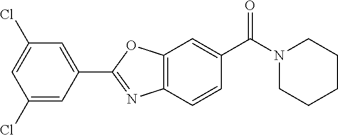 [SMILES: O=C(c1ccc2nc(-c3cc(Cl)cc(Cl)c3)oc2c1)N1CCCCC1] |
| 90 | 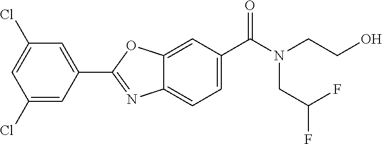 [SMILES: O=C(c1ccc2nc(-c3cc(Cl)cc(Cl)c3)oc2c1)N(CCO)CC(F)F] |
| 91 | 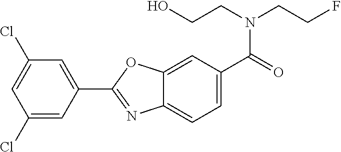 [SMILES: O=C(c1ccc2nc(-c3cc(Cl)cc(Cl)c3)oc2c1)N(CCO)CCF] |
| 92 | 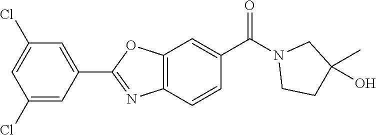 [SMILES: CC1(O)CCN(C(=O)c2ccc3nc(-c4cc(Cl)cc(Cl)c4)oc3c2)C1] |
| 93 | 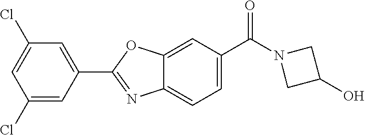 [SMILES: O=C(c1ccc2nc(-c3cc(Cl)cc(Cl)c3)oc2c1)N1CC(O)C1] |
| 94 | 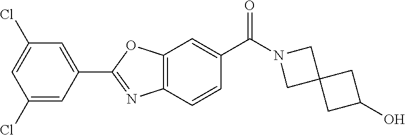 [SMILES: O=C(c1ccc2nc(-c3cc(Cl)cc(Cl)c3)oc2c1)N1CC2(CC(O)C2)C1] |
| 95 | 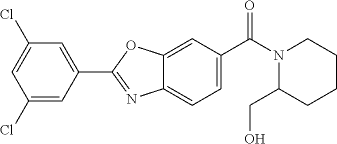 [SMILES: O=C(c1ccc2nc(-c3cc(Cl)cc(Cl)c3)oc2c1)N1CCCCC1CO] |
| 96 | 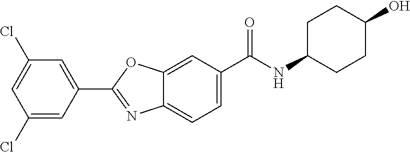 [SMILES: O=C(N[C@H]1CC[C@@H](O)CC1)c1ccc2nc(-c3cc(Cl)cc(Cl)c3)oc2c1] |
| 97 | 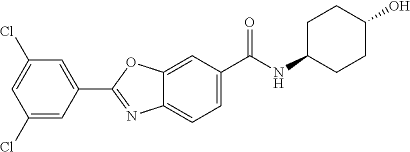 [SMILES: O=C(N[C@H]1CC[C@H](O)CC1)c1ccc2nc(-c3cc(Cl)cc(Cl)c3)oc2c1] |
| 98 | 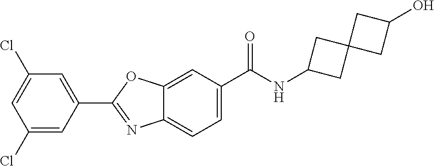 [SMILES: O=C(NC1CC2(CC(O)C2)C1)c1ccc2nc(-c3cc(Cl)cc(Cl)c3)oc2c1] |
| 99 | 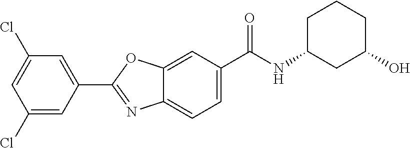 [SMILES: O=C(N[C@@H]1CCC[C@H](O)C1)c1ccc2nc(-c3cc(Cl)cc(Cl)c3)oc2c1] |
| 100 | 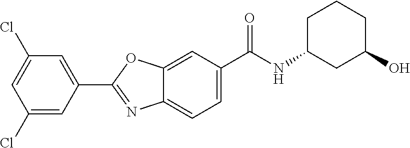 [SMILES: O=C(N[C@@H]1CCC[C@@H](O)C1)c1ccc2nc(-c3cc(Cl)cc(Cl)c3)oc2c1] |
| 101 | 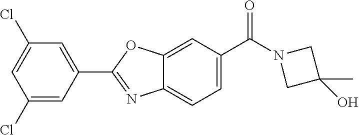 [SMILES: CC1(O)CN(C(=O)c2ccc3nc(-c4cc(Cl)cc(Cl)c4)oc3c2)C1] |
| 102 | 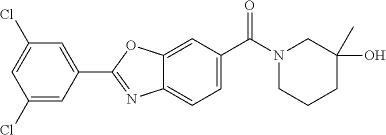 [SMILES: CC1(O)CCCN(C(=O)c2ccc3nc(-c4cc(Cl)cc(Cl)c4)oc3c2)C1] |
| 103 | 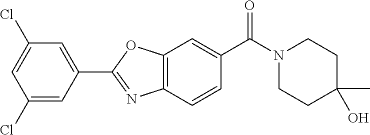 [SMILES: CC1(O)CCN(C(=O)c2ccc3nc(-c4cc(Cl)cc(Cl)c4)oc3c2)CC1] |
| 104 | 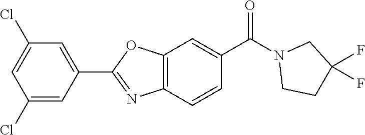 [SMILES: O=C(c1ccc2nc(-c3cc(Cl)cc(Cl)c3)oc2c1)N1CCC(F)(F)C1] |
| 105 | 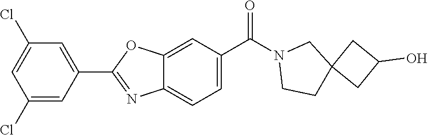 [SMILES: O=C(c1ccc2nc(-c3cc(Cl)cc(Cl)c3)oc2c1)N1CCC2(CC(O)C2)C1] |
| 106 | 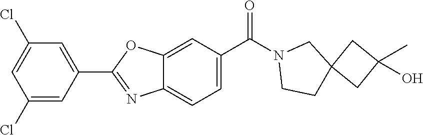 [SMILES: CC1(O)CC2(CCN(C(=O)c3ccc4nc(-c5cc(Cl)cc(Cl)c5)oc4c3)C2)C1] |
| 107 | 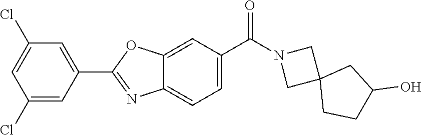 [SMILES: O=C(c1ccc2nc(-c3cc(Cl)cc(Cl)c3)oc2c1)N1CC2(CCC(O)C2)C1] |
| 108 | 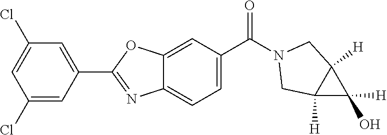 [SMILES: O=C(c1ccc2nc(-c3cc(Cl)cc(Cl)c3)oc2c1)N1C[C@@H]2[C@H](O)[C@@H]2C1] |
| 109 | 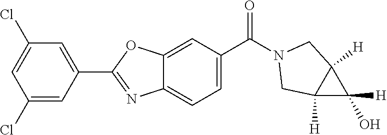 [SMILES: O=C(c1ccc2nc(-c3cc(Cl)cc(Cl)c3)oc2c1)N1C[C@@H]2[C@@H](O)[C@@H]2C1] |
| 110 | 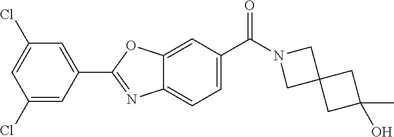 [SMILES: CC1(O)CC2(CN(C(=O)c3ccc4nc(-c5cc(Cl)cc(Cl)c5)oc4c3)C2)C1] |
| 111 | 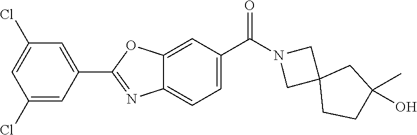 [SMILES: CC1(O)CCC2(CN(C(=O)c3ccc4nc(-c5cc(Cl)cc(Cl)c5)oc4c3)C2)C1] |
| 112 | 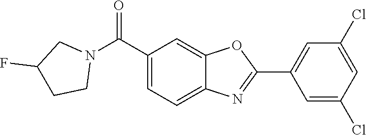 [SMILES: O=C(c1ccc2nc(-c3cc(Cl)cc(Cl)c3)oc2c1)N1CCC(F)C1] |
| 113 | 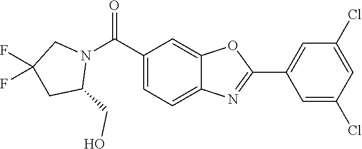 [SMILES: O=C(c1ccc2nc(-c3cc(Cl)cc(Cl)c3)oc2c1)N1CC(F)(F)C[C@H]1CO] |
| 114 | 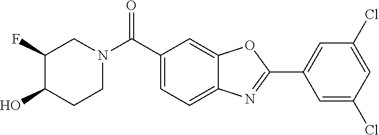 [SMILES: O=C(c1ccc2nc(-c3cc(Cl)cc(Cl)c3)oc2c1)N1CC[C@@H](O)[C@@H](F)C1] |
| 115 | 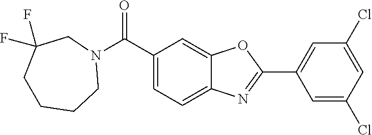 [SMILES: O=C(c1ccc2nc(-c3cc(Cl)cc(Cl)c3)oc2c1)N1CCCCC(F)(F)C1] |
| 116 | 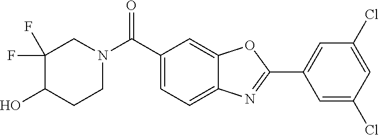 [SMILES: O=C(c1ccc2nc(-c3cc(Cl)cc(Cl)c3)oc2c1)N1CCC(O)C(F)(F)C1] |
| 117 | 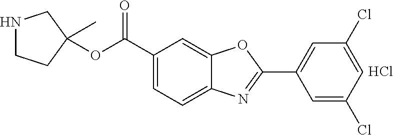 [SMILES: CC1(OC(=O)c2ccc3nc(-c4cc(Cl)cc(Cl)c4)oc3c2)CCNC1 ; Cl] |
| 118 | 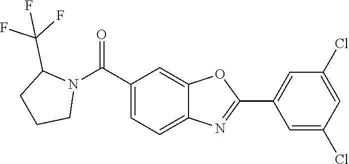 [SMILES: O=C(c1ccc2nc(-c3cc(Cl)cc(Cl)c3)oc2c1)N1CCCC1C(F)(F)F] |
| 119 | 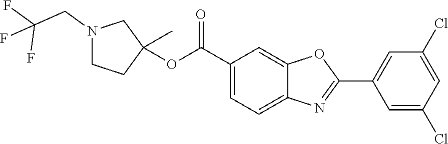 [SMILES: CC1(OC(=O)c2ccc3nc(-c4cc(Cl)cc(Cl)c4)oc3c2)CCN(CC(F)(F)F)C1] |
| 120 | 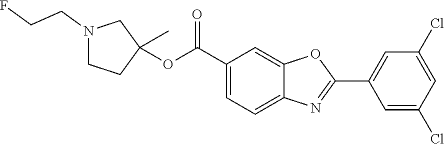 [SMILES: CC1(OC(=O)c2ccc3nc(-c4cc(Cl)cc(Cl)c4)oc3c2)CCN(CCF)C1] |
| 121 | 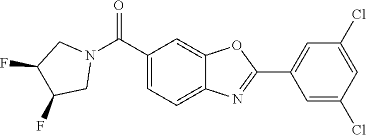 [SMILES: O=C(c1ccc2nc(-c3cc(Cl)cc(Cl)c3)oc2c1)N1C[C@@H](F)[C@@H](F)C1] |
| 122 | 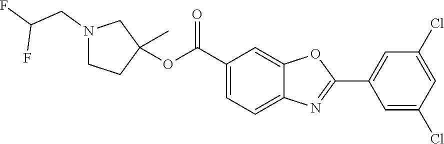 [SMILES: CC1(OC(=O)c2ccc3nc(-c4cc(Cl)cc(Cl)c4)oc3c2)CCN(CC(F)F)C1] |
| 123 | 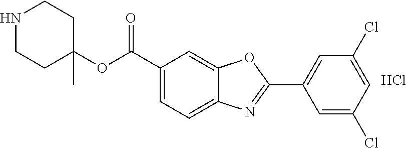 [SMILES: CC1(OC(=O)c2ccc3nc(-c4cc(Cl)cc(Cl)c4)oc3c2)CCNCC1 ; Cl] |
| 124 | 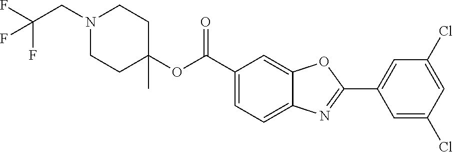 [SMILES: CC1(OC(=O)c2ccc3nc(-c4cc(Cl)cc(Cl)c4)oc3c2)CCN(CC(F)(F)F)CC1] |
| 125 | 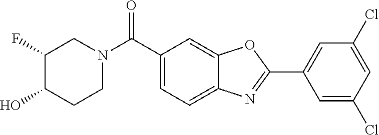 [SMILES: O=C(c1ccc2nc(-c3cc(Cl)cc(Cl)c3)oc2c1)N1CC[C@H](O)[C@H](F)C1] |
| 126 | 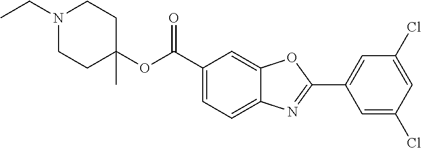 [SMILES: CCN1CCC(C)(OC(=O)c2ccc3nc(-c4cc(Cl)cc(Cl)c4)oc3c2)CC1] |

### III. Syntheses of the Compounds

The compounds provided herein may be prepared using standard techniques well known to those of skill in the art. For example, the compounds may be prepared by standard coupling methods (e.g., DCC, DMAP) well known to those of skill in the art:

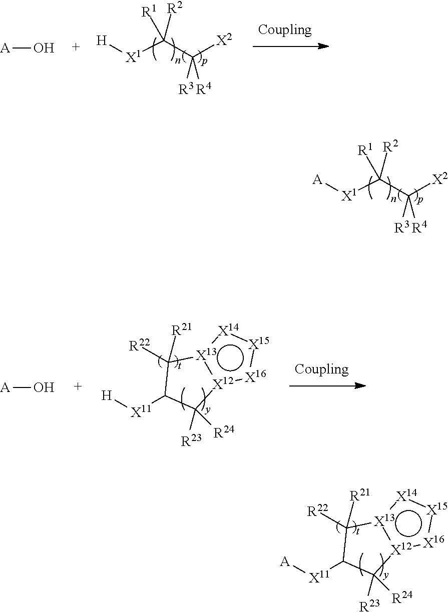

Alternatively, the compounds may be made by reacting an acyl chloride with an alcohol or amine under standard conditions (e.g., DIPEA, DMAP) well known to those of skill in the art:

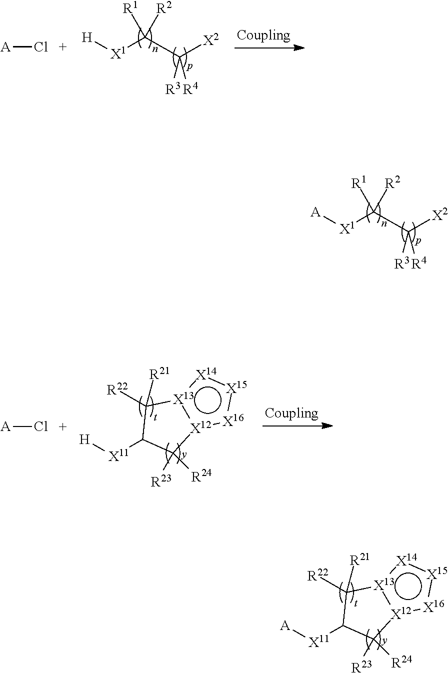

The compounds provided herein may also be made by cyclization of the corresponding α-hydroxy amide under standard conditions well known to those of skill in the art:

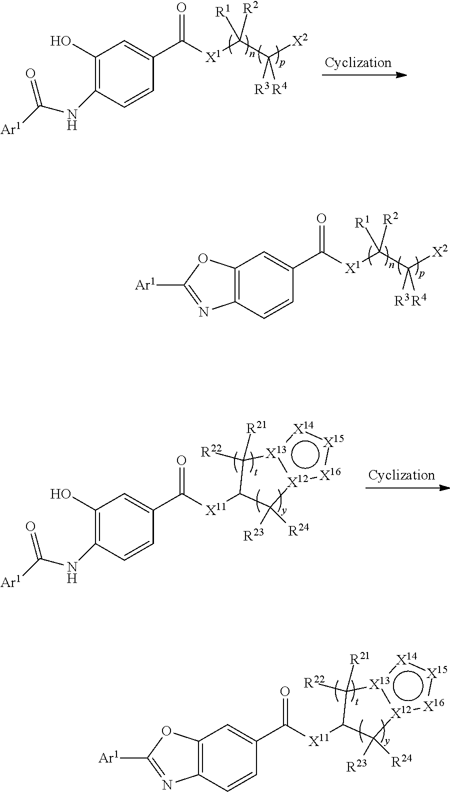

### IV. Methods of Use

The compounds provided herein are useful in treating transthyretin amyloid disease. Without being bound by any theory, the compounds act by inhibiting and preventing TTR aggregation and/or amyloid formation by stabilizing native tetrameric TTR structure therefore preventing dissociation of the tetramer TTR and the deposition of TTR amyloid fibrils in all relevant tissues for TTR amyloid diseases. The transthyretin amyloid disease can be, for example, familial amyloid polyneuropathy (ATTR-FAP), familial amyloid cardiomyopathy (ATTR-FAC), senile systemic amyloidosis and TTR oculoleptomeningeal amyloidosis (ATTR-OLMA).

Prodrugs of TTR stabilizers with good brain and eye penetration should fulfill the current unmet medical need (ocular and cerebral amyloid angiopathies) as an oral drug, by parenteral, intravenous or other injectable delivery, or by local delivery (such as topical eye or intranasal delivery). Tafamidis and diflunisal, two TTR stabilizers with demonstrated clinical efficacy to treat peripheral TTR amyloidosis, are very poor brain and eye penetrating drugs. Compounds provided herein have improved brain penetration by systemic administration and deliver increased levels of TTR stabilizer in the brain. Because the Blood brain barrier (BBB), the blood CSF barrier (BCSFB) and the blood-ocular barrier (BOB) share similarities in microscopic structure, it is recognized in the art that one site may serve as a pharmacokinetic surrogate for the others. Therefore, one of skill in the art would expect a brain penetrating compound to penetrate the eye as well.

Compounds described herein can also be delivered locally to the eye or by intranasal delivery.

Compounds described herein may be useful for treating human patients with TTR oculoleptomeningeal amyloidosis in ATTR patients, including but not restricted to ATTR-OLMA and ATTR-FAP patients.

Combination therapy may include, but is not limited to liver transplantation, TTR stabilizer such as tafamidis, knock-down therapies such as anti-TTR siRNA and antisense (patisiran and inotersen).

### V. Methods of Treating Disease

In another embodiment, a method of treating a subject with peripheral TTR amyloidosis is provided. The method includes administering to a subject having peripheral TTR amyloidosis an effective amount of a compound of Formula I or II. Diseases contemplated in the practice of the methods disclosed herein include familial amyloid polyneuropathy (ATTR-FAP), familial amyloid cardiomyopathy (ATTR-FAC), senile systemic amyloidosis and diseases related to TTR oculoleptomeningeal amyloidosis in ATTR patients, including but not restricted to ATTR-OLMA and ATTR-FAP patients.

### VI. Pharmaceutical Compositions

In another embodiment, provided herein are pharmaceutical compositions. The pharmaceutical composition includes a pharmaceutically acceptable excipient and a compound provided herein (e.g., Formula I or II).

The pharmaceutical compositions provided herein are typically used to treat a disorder or condition using TTR stabilizer therapies.

In an exemplary embodiment, the pharmaceutical composition includes from 1 μg to 2000 mg of a compound disclosed herein, e.g., 1 μg to 1 mg, 1 mg to 10 mg, 1 mg to 100 mg, 1 mg to 1000 mg, 1 mg to 1500 mg, or even 1 mg to 2000 mg.

A. Formulations

The compounds provided herein can be formulated and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds provided herein can also be administered by injection, that is, intravenously, intramuscularly, intracutaneously, subcutaneously, intraduodenally, or intraperitoneally. Also, the compounds provided herein can be administered by inhalation, for example, intranasally. Additionally, the compounds provided herein can be administered transdermally. The compounds provided herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the pharmaceutical compositions provided herein may be adapted for oral administration. In some embodiments, the pharmaceutical composition is in the form of a tablet. Moreover, provided herein are pharmaceutical compositions including a pharmaceutically acceptable carrier or excipient and either a compound provided herein, or a pharmaceutically acceptable salt of a compound provided herein.

For preparing pharmaceutical compositions from the compounds provided herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of REMINGTON'S PHARMACEUTICAL SCIENCES, Maack Publishing Co, Easton PA (“Remington's”).

In powders, the carrier is a finely divided solid, which is in a mixture with the finely divided compound provided herein. In tablets, the compound provided herein is mixed with the carrier having the necessary binding properties in suitable proportions and compacted in the shape and size desired.

The powders and tablets preferably contain from 5% or 10% to 70% of the compound provided herein. Suitable carriers are magnesium carbonate, magnesium stearate, talc, sugar, lactose, pectin, dextrin, starch, gelatin, tragacanth, methylcellulose, sodium carboxymethyl-cellulose, a low melting wax, cocoa butter, and the like. The term “preparation” is intended to include the formulation of the compound provided herein with encapsulating material as a carrier providing a capsule in which the compound provided herein with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethylcellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of compound provided herein (i.e., dosage). Pharmaceutical preparations provided herein can also be used orally using, for example, push-fit capsules made of gelatin, as well as soft, sealed capsules made of gelatin and a coating such as glycerol or sorbitol. Push-fit capsules can contain compounds of Formula I or II mixed with a filler or binders such as lactose or starches, lubricants such as talc or magnesium stearate, and, optionally, stabilizers. In soft capsules, the compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound provided herein in water and adding suitable colorants, flavors, stabilizers, and thickening agents as desired. Aqueous suspensions suitable for oral use can be made by dispersing the finely divided compound provided herein in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the compound provided herein, colorants, flavors, stabilizers, buffers, artificial and natural sweeteners, dispersants, thickeners, solubilizing agents, and the like.

Oil suspensions can be formulated by suspending a compound provided herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds provided herein can be delivered by transdermally, by a topical route, formulated as applicator sticks, solutions, suspensions, emulsions, gels, creams, ointments, pastes, jellies, paints, powders, and aerosols.

The compounds provided herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The compounds provided herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compounds provided herein are useful for parenteral administration, such as intravenous (IV) administration or administration into a body cavity or lumen of an organ. The formulations for administration will commonly comprise a solution of the compound provided herein dissolved in a pharmaceutically acceptable carrier.

Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compound provided herein in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the compound provided herein can be delivered by the use of liposomes which fuse with the cellular membrane or are endocytosed, i.e., by employing ligands attached to the liposome, or attached directly to the compound provided herein, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the compound provided herein. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of compound provided herein in a unit dose preparation may be varied or adjusted from 0.1 mg to 10000 mg, more typically 1.0 mg to 1000 mg, most typically 10 mg to 500 mg, according to the particular application and the potency of the compound provided herein. The composition can, if desired, also contain other compatible therapeutic agents.

In another embodiment, the compound provided herein can be delivered using ocular topical administration of nanoparticles, emulsions, nanostructured lipid carriers, liposomes or nanosuspensions. In another embodiment, the compound provided herein can be delivered by intravitreal administration using biodegradable implants, non-biodegradable implants, biodegradable microspheres, nanoparticles, dendrimers, hydrogels of chronic or microelectromechanical systems (see, e.g., Varela-Fernandez et al. Pharmaceutics 2020, 12(3):269). In another embodiment, the compound provided herein may be administered by periocular, subconjunctival, suprachoroidal or subretinal injection.

Compounds provided herein may be metabolized by cells and then converted to the active TTR stabilizer.

B. Effective Dosages

Pharmaceutical compositions provided herein include compositions wherein the compound provided herein is contained in a therapeutically effective amount, i.e., in an amount effective to achieve its intended purpose. The actual amount effective for a particular application will depend on the condition being treated. For example, when administered in methods to treat TTR related conditions, such compositions will contain an amount of compound provided herein effective to achieve the desired result.

The dosage and frequency (single or multiple doses) of compound provided herein administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds provided herein.

For any compound provided herein, the therapeutically effective amount can be initially determined from cell culture assays. Target concentrations will be those concentrations of compound provided herein that are capable of decreasing viral activity as measured, for example, using the methods provided herein.

Therapeutically effective amounts for use in humans may be determined from animal models. For example, a dose for humans can be formulated to achieve a concentration that has been found to be effective in animals. The dosage in humans can be adjusted by monitoring viral inhibition and adjusting the dosage upwards or downwards, as described above.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound provided herein. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound provided herein effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of compound provided herein by considering factors such as compound potency, relative bioavailability, patient body weight, presence and severity of adverse side effects, mode of administration, and the toxicity profile of the selected agent.

### VII. EXAMPLES

The examples below are meant to illustrate certain embodiments provided herein, and not to limit the scope of this disclosure.

Abbreviations: CDI—carbonyldiimidazole; DCM—Dichloromethane; DMAP—4-dimethylaminopyridine; DMF—dimethylformamide; h—hour; hrs—hours; RT—room temperature; TEA—Triethylamine; TBAF—Tetra-n-butylammonium fluoride; THF—tetrahydrofuran; TLC—thin layer chromatography

The following references provide synthetic and analytical procedures that would be useful to those of skill in the art in preparing and analyzing the compounds provided here.

Each reference disclosed herein is incorporated by reference in its entirety for all purposes.
1. Polish Journal of Chemistry 1985, 59(5-6), 613-620
2. Eur. Pat. Appl., 1229027.
3. WO 2013/119916 A2.
4. WO 2017/148964.
5. Tetrahedron Letters 1989, 39, 11, 1283-1286.
6. Journal of Organic Chemistry 2009, 74, 2, 925-928.
7. WO 2017/48528.
8. European Journal of Medicinal Chemistry, 2012, 52, 159-172.
9. US 2019/233440.

¹H NMR Conditions: Instrument Type: AVANCE III 400 or AVANCE III 400 HD or AVANCE NEO; Probe Type: 5 mm PABBO BB or 5 mm CPP BBO; Frequency (MHz): 400.1300; Temperature (Degree ° C.): 27.

LCMS Methods:

Method 1: Instrument: SHIMADZU LCMS-2020; Column: Kinetex EVO C18 2.1×30 mm, 5 μm; Mobile Phase: A: 0.0375% TFA in water (v/v), B: 0.01875% TFA in Acetonitrile (v/v); Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

Method 2: Instrument: SHIMADZU LCMS-2020; Column: Kinetex EVO C18 2.1×30 mm, 5 μm; Mobile Phase: A: 0.025% NH₃·H₂O in water (v/v), B: Acetonitrile; Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

HPLC Methods

Method 1: Instrument: SHIMADZU LC-20AB; Column: Kinetex C18 LC Column 4.6×50 mm, 5 μm; Mobile Phase: A: 0.0375% TFA in water (v/v), B: 0.01875% TFA in Acetonitrile (v/v); Gradient: 0.0 min 10% B→2.40 min 80% B→3.70 min 80% B→3.71 min 10% B→4.00 min 10% B; Flow: 1.5 mL/min; Column Temp: 50° C.; Detector: PDA (220 nm & 215 nm & 254 nm).

Method 2: Instrument: SHIMADZU LC-20AB; Column: XBridge C18, 2.1×50 mm, 5 μm; Mobile Phase: A: 0.025% NH₃·H₂O in water (v/v), B: Acetonitrile; Gradient: 0.0 min 10% B→4.20 min 80% B→5.30 min 80% B→5.31 min 10% B→6.00 min 10% B; Flow: 0.8 mL/min; Column Temp: 40° C.; Detector: PDA (220 nm & 215 nm & 254 nm).

Method 3: Instrument: SHIMADZU LC-20AB; Column: XBridge C18, 2.1×50 mm, 3.5 μm; Mobile Phase: A: 0.025% NH₃·H₂O in water (v/v), B: Acetonitrile; Gradient: 0.0 min 30% B→3.00 min 90% B→3.50 min 90% B→3.51 min 30% B→4.00 min 30% B; Flow: 1.2 mL/min; Column Temp: 50° C.; Detector: PDA (220 nm & 215 nm & 254 nm).

### Example 1

### Compound 1: 2-(3,5-dichlorophenyl)-N-(2,2-difluoropropyl)benzo[d]oxazole-6-carboxamide

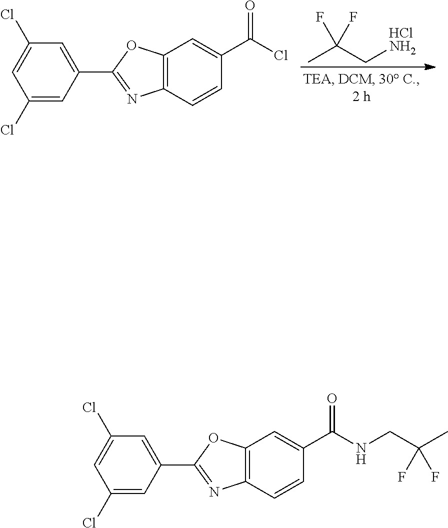

2-(3,5-dichlorophenyl)benzo[d]oxazole-6-carbonyl chloride (100 mg, 0.31 mmol) was added to the mixture of 2,2-difluoropropan-1-amine hydrochloride (40 mg, 0.31 mmol) and TEA (155 mg, 1.53 mmol) in DCM (2 mL) and stirred for 2 hrs at 30° C. The mixture was quenched with water (30 mL) and extracted with EtOAc (30 mL×3). The organic layer was washed with brine (50 mL) and concentrated under reduced pressure. The crude product was purified by column chromatography (SiO₂, Petroleum ether/Ethyl acetate=10/1 to 1/1) to afford the title compound (68 mg, 55.34% yield, 96% purity) as a white solid.

LCMS: m/z 385.0 [M+H]⁺

¹H NMR (400 MHz, CDCl₃) δ=8.17 (d, J=1.2 Hz, 2H), 8.12 (s, 1H), 7.86-7.77 (m, 2H), 7.56 (s, 1H), 6.47 (s, 1H), 3.93 (dt, J=6.4, 13.6 Hz, 2H), 1.72 (t, J=18.8 Hz, 3H).

### Example 2

### Compound 2: 2-(3,5-dichlorophenyl)-N-(1,1-difluoropropan-2-yl)benzo[d]oxazole-6-carboxamide

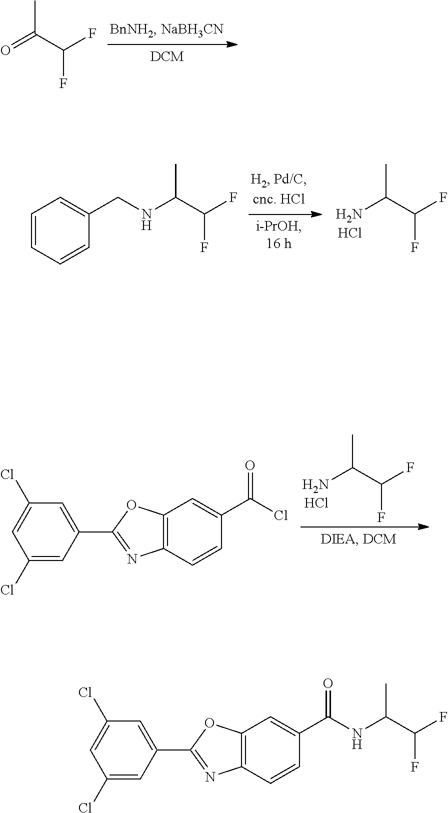

### Step 1: N-benzyl-1,1-difluoropropan-2-amine

To a solution of 1,1-difluoropropan-2-one (1 g, 10.63 mmol) in DCM (40 mL) was added BnNH₂ (1.14 g, 10.63 mmol) and NaBH(OAc)₃ (6.76 g, 31.89 mmol). The reaction mixture was stirred for 12 hrs at 25° C. The reaction mixture was quenched with saturated NaHCO₃ solution (30 mL) and extracted with EtOAc (60 mL). The organic layer was separated and washed with brine (30 mL), dried over Na₂SO₄, and concentrated under reduced pressure. The residue was purified by silica gel column (petroleum ether:EtOAc=30:1 to 20:1) to give the title compound (1.0 g, 51% yield) as a colorless oil.

¹H NMR (400 MHz, CDCl₃) δ=7.29-7.17 (m, 5H), 5.73-5.44 (m, 1H), 3.81 (q, J=9.2 Hz, 2H), 2.96-2.86 (m, 1H), 1.32 (bs, 1H), 1.10 (d, J=6.4 Hz, 3H).

### Step 2: 1,1-difluoropropan-2-amine hydrochloride

To a solution of N-benzyl-1,1-difluoro-propan-2-amine (200 mg, 1.08 mmol) in i-PrOH (5 mL) was added Pd/C (20 mg, 10% purity) and HCl (0.2 mL, 36% purity). The reaction mixture was degassed under reduced pressure and purged with H₂ for three times. The reaction mixture was stirred for 12 hrs under H₂ balloon (15 Psi) at 25° C. The reaction mixture was filtered through a pad of celite and washed with methanol (10 mL×3). The filtrate was concentrated under reduced pressure to give the title compound (120 mg, 84% yield) as yellow solid, which was used for next step without purification.

¹H NMR (400 MHz, DMSO-d₆) δ=8.69 (bs, 3H), 6.39-6.12 (m, 1H), 3.80-3.74 (m, 1H), 1.25 (d, J=6.8 Hz, 3H).

### Step 3: 2-(3,5-dichlorophenyl)-N-(1,1-difluoropropan-2-yl)benzo[d]oxazole-6-carboxamide

To a solution of 1,1-difluoropropan-2-amine (90 mg, 0.69 mmol, HCl salt) in DCM (6 mL) was DIEA (178 mg, 1.38 mmol, 3 eq) and 2-(3,5-dichlorophenyl)benzo[d]oxazole-6-carbonyl chloride (150 mg, 0.46 mmol). The reaction mixture was stirred for 12 hrs at 25° C. The reaction mixture was filtered and washed with DCM (5 mL×3). The cake was collected. The cake was dried over under reduced pressure without purification to afford the title compound (137.15 mg, 77% yield, 99.6% purity) as a white solid.

LCMS: m/z 418.9 [M+H]⁺

¹H NMR (400 MHz, CDCl₃) δ=8.17 (d, J=2.0 Hz, 2H), 8.11 (s, 1H), 7.85-7.77 (m, 2H), 7.56 (t, J=1.6 Hz, 1H), 6.15-6.13 (m, 1H), 6.10-5.82 (m, 1H), 4.70-4.55 (m, 1H), 1.39 (d, J=6.8 Hz, 3H).

### Example 3

### Compound 3: 2-(3,5-dichlorophenyl)-N-(2,2,3,3,3-pentafluoropropyl)benzo[d]oxazole-6-carboxamide

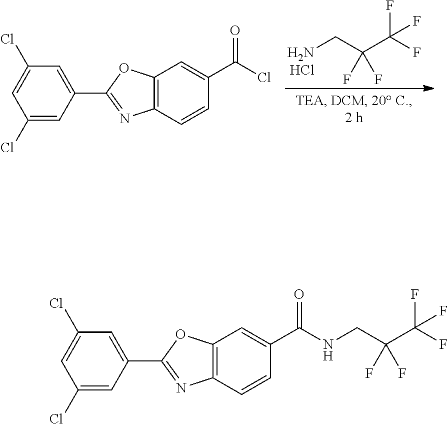 [SMILES: O=C(Cl)c1ccc2nc(-c3cc(Cl)cc(Cl)c3)oc2c1 ; Cl ; NCC(F)(F)C(F)(F)F ; O=C(NCC(F)(F)C(F)(F)F)c1ccc2nc(-c3cc(Cl)cc(Cl)c3)oc2c1]

2-(3,5-dichlorophenyl)benzo[d]oxazole-6-carbonyl chloride (200 mg, 0.61 mmol) was added to the mixture of 2,2,3,3,3-pentafluoropropan-1-amine hydrochloride (114 mg, 0.61 mmol) and TEA (310 mg, 3.06 mmol) in DCM (3 mL) and stirred for 2 hrs at 20° C. The mixture was quenched with water (30 mL) and extracted with EtOAc (30 mL×3). The organic layer was washed with brine (50 mL) and concentrated under reduced pressure. The crude product was purified by column chromatography (SiO₂, Petroleum ether/Ethyl acetate=5/1 to 1/1) to afford the title compound (157.49 mg, 54.46% yield, 96% purity) as a white solid.

LCMS: m/z 438.9 [M+H]⁺

¹H NMR (400 MHz, CDCl₃) δ=8.18 (d, J=1.2 Hz, 2H), 8.13 (s, 1H), 7.90-7.76 (m, 2H), 7.57 (s, 1H), 6.38 (s, 1H), 4.25 (dt, J=6.4, 14.8 Hz, 2H).

### Example 4

### Compound 4: 2-(3,5-dichlorophenyl)benzo[d]oxazole-6-carboxamide

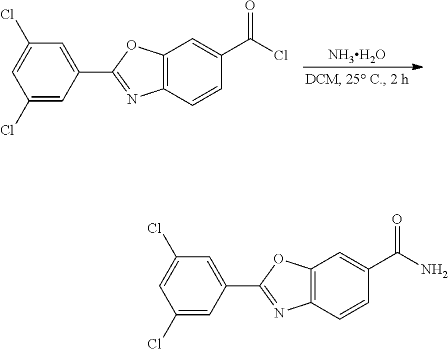 [SMILES: O=C(Cl)c1ccc2nc(-c3cc(Cl)cc(Cl)c3)oc2c1 ; "NH3·H2O" ; NC(=O)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the solution of NH₃·H₂O (162.60 mg, 1.53 mmol) in DCM (2 mL) and stirred for 2 h at 25° C. The reaction mixture was filtered and washed with DCM (5 mL×3). The solid was collected and dried under reduced pressure to afford the title compound (86.98 mg, 92.30% yield, 99.8% purity) as a white solid.

LCMS: m/z 306.8 [M+H]⁺.

¹H NMR (400 MHz, DMSO-d₆) δ=8.32-8.28 (m, 1H), 8.18 (s, 3H), 8.00 (d, J=8.8 Hz, 1H), 7.96 (s, 1H), 7.91 (d, J=8.0 Hz, 1H), 7.55 (s, 1H)

### Example 5

### Compound 5: N-(cyanomethyl)-2-(3,5-dichlorophenyl)benzo[d]oxazole-6-carboxamide

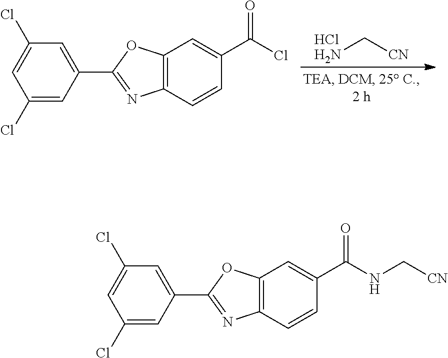

2-(3,5-dichlorophenyl)benzo[d]oxazole-6-carbonyl chloride (100 mg, 0.31 mmol) was added to the mixture of 2-aminoacetonitrile (28.33 mg, 0.31 umol HCl salt) and TEA (155 mg, 1.53 mmol) in DCM (2 mL) and stirred for 2 h at 25° C. The mixture was quenched with water (30 mL) and filtered. the cake was washed with DCM (5 mL×3). The cake was collected and dried under reduced pressure to afford the title compound (49.45 mg, 45.27% yield, 97.0% purity) as a white solid.

LCMS: m/z 346.0 [M+H]⁺.

¹H NMR (400 MHz, DMSO-d₆) δ=9.39 (t, J=4.8 Hz, 1H), 8.29 (s, 1H), 8.18 (d, J=2.0 Hz, 2H), 8.04-7.90 (m, 3H), 4.38 (d, J=5.2 Hz, 2H).

### Example 6

### Compound 6: 2-(3,5-dichlorophenyl)-N-(2-methoxyethyl)benzo[d]oxazole-6-carboxamide

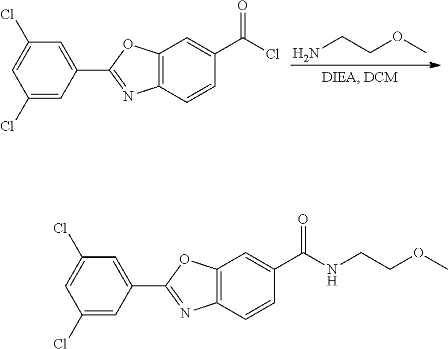

To a solution of 2-methoxyethanamine (41 mg, 0.55 mmol) in DCM (6 mL) was added DIEA (178 mg, 1.38 mmol) and 2-(3,5-dichlorophenyl)benzo[d]oxazole-6-carbonyl chloride (150 mg, 0.46 mmol). The reaction mixture was stirred for 12 h at 25° C. The reaction mixture was concentrated under reduced pressure. The residue was recrystallized from DCM (5 mL) to afford the title compound (95.62 mg, 56% yield, 98.5% purity) as white solid.

LCMS: m/z 364.8 [M+H]⁺.

¹H NMR (400 MHz, DMSO-d₆) δ=8.74-8.722 (m, 1H), 8.26 (s, 1H), 8.17 (d, J=2.0 Hz, 2H), 7.99-7.90 (m, 3H), 3.51-3.46 (m, 4H), 3.29 (s, 3H).

### Example 7

### Compound 7: 2-(3,5-dichlorophenyl)-N-(2-(dimethylamino)ethyl)benzo[d]oxazole-6-carboxamide

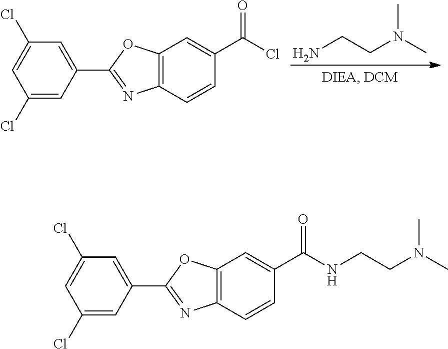

To a solution of N′,N′-dimethylethane-1,2-diamine (49 mg, 0.55 mmol) in DCM (6 mL) was added DIEA (178 mg, 1.38 mmol) and 2-(3,5-dichlorophenyl)benzo[d]oxazole-6-carbonyl chloride (150 mg, 459 mmol). The reaction mixture was stirred for 12 h at 25° C. The reaction mixture was concentrated under reduced pressure. The residue was recrystallized from DCM (5 mL) to afford the title compound (69.23 mg, 39% yield, 98.2% purity) as white solid.

LCMS: m/z 377.8 [M+H]⁺.

¹H NMR (400 MHz, CDCl₃) δ=8.16 (d, J=2.0 Hz, 3H), 7.87-7.80 (m, 2H), 7.54 (t, J=1.6 Hz, 1H), 7.21 (bs, 1H), 3.61 (q, J=5.6 Hz, 2H), 2.65 (t, J=5.6 Hz, 2H), 2.37 (s, 6H).

### Example 8

### Compound 8: 2-(3,5-dichlorophenyl)-N-(2-(dimethylamino)ethyl)-N-methylbenzo[d]oxazole-6-carboxamide

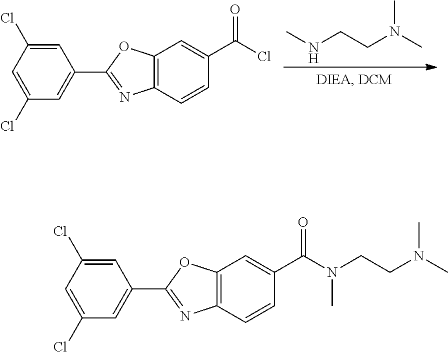

To a solution of N¹,N¹,N²-trimethylethane-1,2-diamine (70 mg, 0.69 mmol) in DCM (5 mL) was added DIEA (178 mg, 1.38 mmol) and 2-(3,5-dichlorophenyl)benzo[d]oxazole-6-carbonyl chloride (150 mg, 0.46 mmol) at 25° C. The reaction mixture was stirred for 12 h at 25° C. The mixture was concentrated under reduced pressure. The crude was purified by prep-HPLC (column: Welch Ultimate XB—NH₂ 250×50×10 μm; mobile phase: [Hexane-EtOH, neutral]; B %: 2%-25%, 10 min) to afford the title compound (86.34 mg, 48% yield, 99.2% purity) as white solid.

LCMS: m/z 392.1 [M+H]⁺.

¹H NMR (400 MHz, CDCl₃) δ=8.15 (d, J=1.6 Hz, 2H), 7.79 (d, J=8.4 Hz, 1H), 7.72 (s, 1H), 7.54 (t, J=1.6 Hz, 1H), 7.47 (d, J=8.0 Hz, 1H), 3.75 (bs, 1H), 3.41-3.37 (m, 1H), 3.08 (bs, 4H), 2.73 (bs, 1H), 2.42 (bs, 6H).

### Example 9

### Compound 9: 2-(3,5-dichlorophenyl)-N-(2-(methylamino)ethyl)benzo[d]oxazole-6-carboxamide hydrochloride

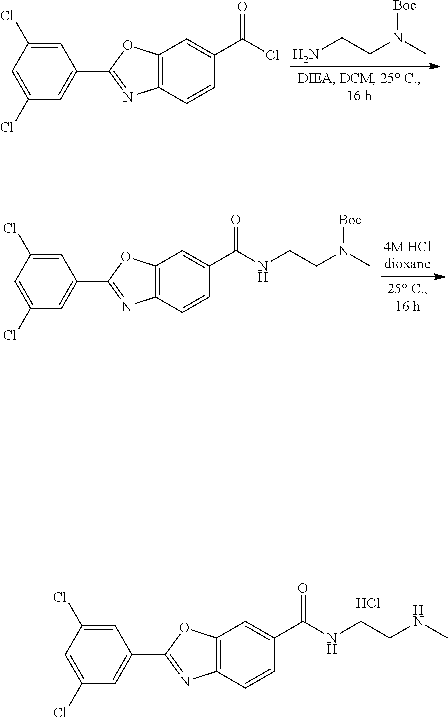

### Step 1: tert-butyl (2-(2-(3,5-dichlorophenyl)benzo[d]oxazole-6-carboxamido)ethyl)(methyl)carbamate

To a solution of tert-butyl N-(2-aminoethyl)-N-methyl-carbamate (120 mg, 0.69 mmol) in DCM (10 mL) was added DIEA (178 mg, 1.38 mmol) and 2-(3,5-dichlorophenyl)benzo[d]oxazole-6-carbonyl chloride (150 mg, 0.46 mmol) at 25° C. The reaction mixture was stirred for 16 h at 25° C. The mixture was concentrated under reduced pressure. The residue was purified by flash silica gel chromatography (20 g SepaFlash® Silica Flash Column, Eluent of 0-90% EA/Pet. ether gradient @ 20 mL/min) to afford the title compound (200 mg, 93% yield, 99.7% purity) as white solid.

### Step 2: 2-(3,5-dichlorophenyl)-N-(2-(methylamino)ethyl)benzo[d]oxazole-6-carboxamide hydrochloride

A solution of tert-butyl N-[2-[[2-(3,5-dichlorophenyl)-1,3-benzoxazole-6-carbonyl]amino]ethyl]-N-methyl-carbamate (200 mg, 0.43 mmol) in HCl\dioxane (4 M, 5 mL) was stirred for 16 h at 25° C. The reaction mixture was concentrated under reduced pressure. The residue was triturated in pet. ether (10 mL), filtered and the solid was collected and dried under reduced pressure to afford the title compound (155.67 mg, 89% yield, 99.1% purity, HCl salt) as white solid.

LCMS: m/z 363.9 [M+H]⁺

¹H NMR (400 MHz, CD₃OD) δ=8.25 (d, J=1.2 Hz, 1H), 8.20 (d, J=2.0 Hz, 2H), 7.98 (dd, J=1.6, 8.4 Hz, 1H), 7.86 (d, J=8.4 Hz, 1H), 7.73 (t, J=2.0 Hz, 1H), 3.74 (t, J=5.6 Hz, 2H), 3.27-3.26 (m, 2H), 2.77 (s, 3H).

### Example 10

### Compound 10: 2-(3,5-dichlorophenyl)-N-(1-hydroxypropan-2-yl)benzo[d]oxazole-6-carboxamide

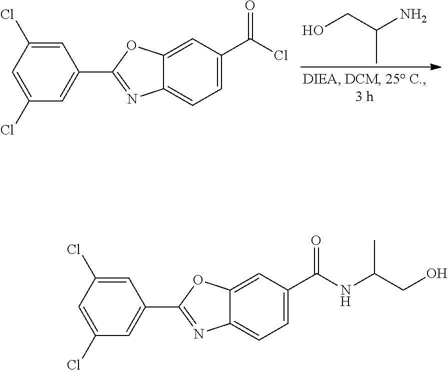

To a solution of 2-aminopropan-1-ol (52 mg, 0.69 mmol) in DCM (10 mL) was added DIEA (178 mg, 1.38 mmol) and 2-(3,5-dichlorophenyl)benzo[d]oxazole-6-carbonyl chloride (150 mg, 0.46 mmol) at 25° C. The reaction mixture was stirred for 16 h at 25° C. LCMS showed the starting material was consumed, and the desired mass was detected. The suspension was filtered and the filter cake was washed with DCM:PE=5:1 (10 mL×3). The solid was concentrated under reduced pressure. The crude product was purified by prep-HPLC (column: Welch Ultimate XB—CN 250×50×10 μm; mobile phase: [Hexane-EtOH, neutral]; B %: 0%-25%, 13 min) to afford the title compound (63.97 mg, 38% yield, 99.7% purity) as a white solid.

LCMS: m/z 365.1 [M+H]⁺

¹H NMR (400 MHz, CD₃OD) δ=8.22-8.21 (m, 3H), 7.95 (dd, J=1.6, 8.4 Hz, 1H), 7.83 (d, J=8.0 Hz, 1H), 7.73 (t, J=2.0 Hz, 1H), 4.23 (qd, J=6.4, 12.4 Hz, 1H), 3.68-3.59 (m, 2H), 1.28 (d, J=6.8 Hz, 3H).

### Example 11

### Compound 11: 2-(3,5-dichlorophenyl)-N-(2-hydroxypropyl)benzo[d]oxazole-6-carboxamide

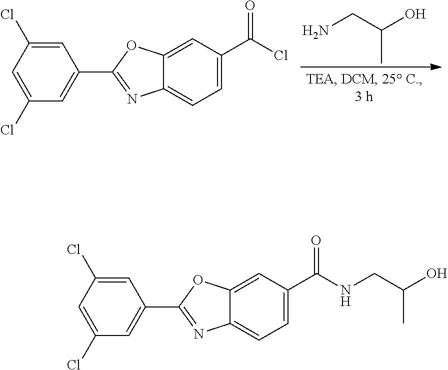

2-(3,5-dichlorophenyl)benzo[d]oxazole-6-carbonyl chloride (150 mg, 0.46 mmol) was added to a solution of 1-aminopropan-2-ol (38 mg, 0.51 mmol) and TEA (140 mg, 1.38 mmol) in DCM (2 mL), and then the mixture was stirred for 3 h at 25° C. The mixture was quenched with water (30 mL) and extracted with EtOAc (30 mL×3). The organic layer was washed with brine (50 mL), concentrated under reduced pressure. The crude product was purified by column chromatography (SiO₂, Petroleum ether:EtOAc=10/1 to 1/1) to afford the title compound (68 mg, 55% yield, 96% purity) as a white solid.

LCMS: m/z 365.0 [M+H]⁺

¹H NMR (400 MHz, DMSO-d₆) δ=8.60 (t, J=5.6 Hz, 1H), 8.28 (d, J=0.8 Hz, 1H), 8.17 (d, J=2.0 Hz, 2H), 8.02-7.85 (m, 3H), 4.77 (d, J=4.8 Hz, 1H), 3.89-3.76 (m, 1H), 3.29-3.19 (m, 2H), 1.09 (d, J=6.4 Hz, 3H).

### Example 12

### Compound 12: 2-(3,5-dichlorophenyl)-N-(2,2-difluorocyclopropyl)benzo[d]-oxazole-6-carboxamide

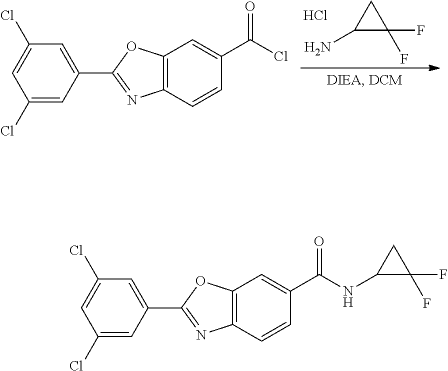 [SMILES: O=C(Cl)c1ccc2nc(-c3cc(Cl)cc(Cl)c3)oc2c1 ; Cl ; NC1CC1(F)F ; O=C(NC1CC1(F)F)c1ccc2nc(-c3cc(Cl)cc(Cl)c3)oc2c1]

To a solution of 2,2-difluorocyclopropanamine (24 mg, 0.18 mmol, HCl salt) in DCM (2 mL) was added DIEA (111 mg, 0.86 mmol) and 2-(3,5-dichlorophenyl)benzo[d]oxazole-6-carbonyl chloride (50 mg, 0.15 mmol). The reaction mixture was stirred for 12 h at 25° C. The reaction mixture was concentrated under reduced pressure. The residue was recrystallized in DCM (5 mL) to afford the title compound (33.15 mg, 55% yield, 97.4% purity) as white solid.

LCMS: m/z 382.8 [M+H]⁺

¹H NMR (400 MHz, DMSO-d₆) δ=8.98 (bs, 1H), 8.27 (s, 1H), 8.16 (d, J=1.6 Hz, 2H), 7.99-7.92 (m, 3H), 3.54-3.48 (m, 1H), 2.05-1.95 (m, 1H), 1.77-1.68 (m, 1H).

### Example 13

### Compound 13: 2-(3,5-dichlorophenyl)-N-(2,2-difluorocyclohexyl)benzo[d]oxazole-6-carboxamide

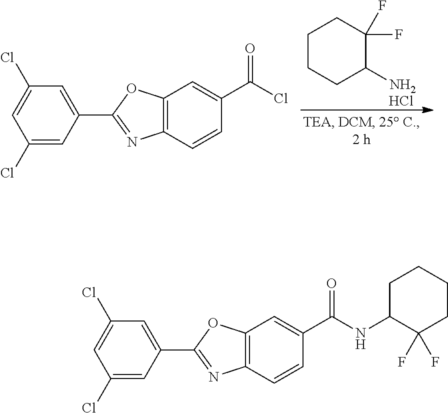

2-(3,5-dichlorophenyl)benzo[d]oxazole-6-carbonyl chloride (100 mg, 0.31 mmol) was added to the mixture of 2,2-difluorocyclohexanamine hydrochloride (52.55 mg, 0.31 mmol) and TEA (55 mg, 1.53 mmol) in DCM (3 mL) and stirred for 2 hrs at 25° C. The mixture was filtered and the cake was washed with DCM (50 mL) to afford the title compound (54.0 mg, 41.05% yield, 99% purity) as a white solid.

LCMS: m/z 425.0 [M+H]⁺

¹H NMR (400 MHz, CDCl₃) δ=8.17 (d, J=1.6 Hz, 2H), 8.12 (s, 1H), 7.90-7.72 (m, 2H), 7.56 (t, J=1.6 Hz, 1H), 6.36 (d, J=9.2 Hz, 1H), 4.58-4.34 (m, 1H), 2.37-2.23 (m, 1H), 1.95-1.72 (m, 3H), 1.60 (s, 1H), 1.55-1.42 (m, 2H).

### Example 14

### Compound 14: 2-(3,5-dichlorophenyl)-N-(3,3-difluorocyclohexyl)benzo[d]oxazole-6-carboxamide

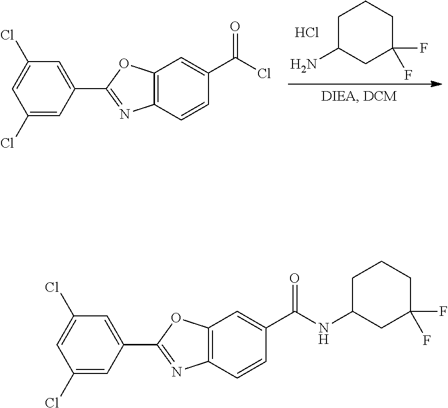 [SMILES: O=C(Cl)c1ccc2nc(-c3cc(Cl)cc(Cl)c3)oc2c1 ; Cl ; NC1CCCC(F)(F)C1 ; O=C(NC1CCCC(F)(F)C1)c1ccc2nc(-c3cc(Cl)cc(Cl)c3)oc2c1]

To a solution of 3,3-difluorocyclohexanamine (95 mg, 0.55 mmol, HCl salt) in DCM (5 mL) was added DIEA (297 mg, 2.30 mmol) and 2-(3,5-dichlorophenyl)benzo[d]oxazole-6-carbonyl chloride (150 mg, 0.46 mmol). The reaction mixture was stirred for 12 hrs at 25° C. The reaction mixture was filtered and washed with DCM (5 mL×3). The cake was collected and dried over under reduced pressure without purification to afford the title compound (158.94 mg, 81% yield, 99.6% purity) as white solid.

LCMS: m/z 424.9 [M+H]⁺.

¹H NMR (400 MHz, CDCl₃) δ=8.16 (d, J=1.6 Hz, 2H), 8.08 (s, 1H), 7.83-7.81 (m, 1H), 7.75-7.73 (m, 1H), 7.55 (s, 1H), 6.41-6.39 (m, 1H), 4.51-4.49 (m, 1H), 2.43-2.31 (m, 1H), 2.02-1.88 (m, 4H), 1.85-1.74 (m, 2H), 1.72-1.65 (m, 1H).

### Example 15

### Compound 15: 2-(3,5-dichlorophenyl)-N-(4,4-difluorocyclohexyl)benzo[d]oxazole-6-carboxamide

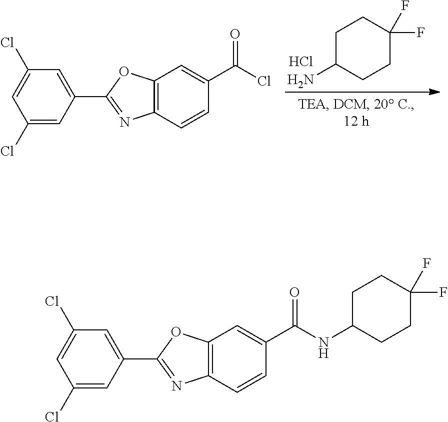 [SMILES: O=C(Cl)c1ccc2nc(-c3cc(Cl)cc(Cl)c3)oc2c1 ; Cl ; NC1CCC(F)(F)CC1 ; O=C(NC1CCC(F)(F)CC1)c1ccc2nc(-c3cc(Cl)cc(Cl)c3)oc2c1]

4,4-difluorocyclohexanamine hydrochloride (57.81 mg, 336.84 umol) was added to the mixture of 2-(3,5-dichlorophenyl)benzo[d]oxazole-6-carbonyl chloride (100 mg, 0.31 mmol) and TEA (154.93 mg, 1.53 mmol) in DCM (2 mL). The mixture was stirred for 12 hrs at 20° C. The mixture was treated with water (50 mL) and filtered to give the cake and the cake was triturated with DMF:DCM (v:v=1:10, 100 mL) to afford the title compound (93.14 mg, 71.02% yield, 99.3% purity) as a white solid.

LCMS: m/z 425.0 [M+H]⁺

¹H NMR (400 MHz, DMSO-d₆) δ=8.48 (d, J=7.6 Hz, 1H), 8.28 (s, 1H), 8.18 (d, J=2.0 Hz, 2H), 8.01-7.96 (m, 2H), 7.95-7.90 (m, 1H), 4.22-3.88 (m, 1H), 2.16-2.01 (m, 3H), 1.99-1.88 (m, 3H), 1.77-1.57 (m, 2H).

### Example 16

### Compound 16: 2-(3,5-dichlorophenyl)-N-(oxetan-3-yl)benzo[d]oxazole-6-carboxamide

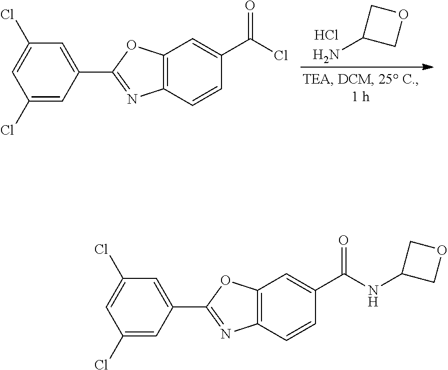 [SMILES: O=C(Cl)c1ccc2nc(-c3cc(Cl)cc(Cl)c3)oc2c1 ; Cl ; NC1COC1 ; O=C(NC1COC1)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of oxetan-3-amine (34 mg, 0.31 mmol, HCl salt) and TEA (155 mg, 1.53 mmol) in DCM (3 mL) and stirred for 1 h at 25° C. The mixture was quenched with water (30 mL) and extracted with EtOAc (30 mL×3). The organic layer was washed with brine (50 mL) and concentrated under reduced pressure. The crude product was purified by column chromatography (SiO₂, Petroleum ether:EtOAc=1:1 to 1:5) to afford the title compound (50.52 mg, 43% yield, 94.9% purity) as a white solid.

LCMS: m/z 362.8 [M+H]⁺.

¹H NMR (400 MHz, DMSO-d₆) δ=9.27 (d, J=6.4 Hz, 1H), 8.30 (s, 1H), 8.18 (d, J=1.6 Hz, 2H), 8.05-7.89 (m, 3H), 5.13-4.95 (m, 1H), 4.80 (t, J=6.8 Hz, 2H), 4.63 (t, J=6.8 Hz, 2H).

### Example 17

Compound 17: 2-(3,5-dichlorophenyl)-N-(tetrahydrofuran-3-yl)benzo[d]oxazole-6-carboxamide

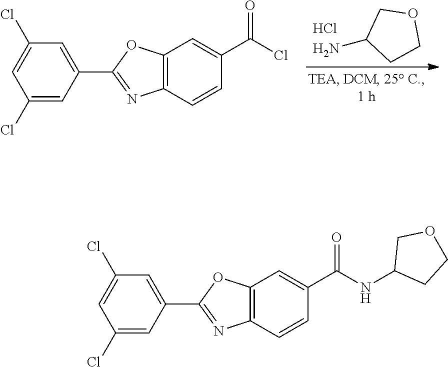 [SMILES: O=C(Cl)c1ccc2nc(-c3cc(Cl)cc(Cl)c3)oc2c1 ; Cl ; NC1CCOC1 ; O=C(NC1CCOC1)c1ccc2nc(-c3cc(Cl)cc(Cl)c3)oc2c1]

2-(3,5-dichlorophenyl)benzo[d]oxazole-6-carbonyl chloride (100 mg, 0.31 mmol was added to the mixture of tetrahydrofuran-3-amine (38 mg, 0.31 mmol, HCl salt) and TEA (155 mg, 1.53 mmol) in DCM (2 mL) and stirred for 1 h at 25° C. The mixture was quenched with water (30 mL) and extracted with EtOAc (30 mL×3). The organic layer was washed with brine (50 mL) and concentrated under reduced pressure. The crude product was purified by column chromatography (SiO₂, Petroleum ether:EtOAc=1:1 to 1:5) to afford the title compound (37.49 mg, 31% yield, 96.7% purity) as a white solid.

LCMS: m/z 376.8 [M+H]⁺.

¹H NMR (400 MHz, DMSO-d₆) δ=8.72 (d, J=6.4 Hz, 1H), 8.30 (s, 1H), 8.18 (d, J=2.0 Hz, 2H), 7.99 (dd, J=2.0, 8.8 Hz 1H), 7.97 (t, J=2.0 Hz, 1H), 7.92 (d, J=8.4 Hz, 1H), 4.58-4.43 (m, 1H), 3.94-3.84 (m, 2H), 3.74 (dt, J=6.0, 8.0 Hz, 1H), 3.64 (dd, J=4.0, 8.8 Hz, 1H), 2.26-2.12 (m, 1H), 2.04-1.89 (m, 1H).

### Example 18

### Compound 18: N-cyclobutyl-2-(3,5-dichlorophenyl)benzo[d]oxazole-6-carboxamide

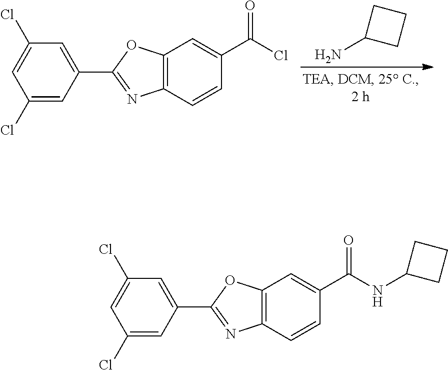 [SMILES: O=C(Cl)c1ccc2nc(-c3cc(Cl)cc(Cl)c3)oc2c1 ; NC1CCC1 ; O=C(NC1CCC1)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of cyclobutanamine (22 mg, 0.31 mmol) and TEA (93 mg, 0.92 mmol) in DCM (2 mL) and stirred for 2 h at 25° C. The reaction mixture was filtered and washed with DCM (5 mL×3). The cake was collected and dried over under reduced pressure without purification to afford the title compound (101.39 mg, 88% yield, 95.6% purity) as a white solid.

LCMS: m/z 361.0 [M+H]⁺.

¹H NMR (400 MHz, CDCl₃) δ=8.16 (d, J=2.0 Hz, 2H), 8.09 (s, 1H), 7.83-7.78 (m, 1H), 7.78-7.72 (m, 1H), 7.56 (t, J=2.0 Hz, 1H), 6.31 (d, J=7.2 Hz, 1H), 4.69-4.59 (m, 1H), 2.58-2.39 (m, 2H), 2.13-1.92 (m, 2H), 1.88-1.75 (m, 2H).

### Example 19

### Compound 19: N-cyclopentyl-2-(3,5-dichlorophenyl)benzo[d]oxazole-6-carboxamide

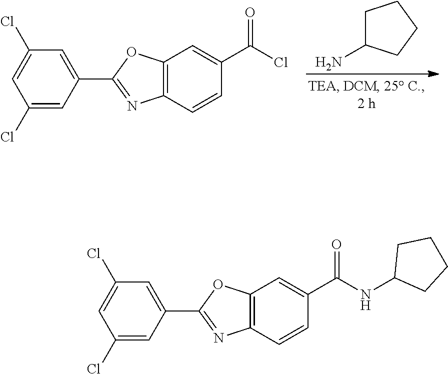 [SMILES: O=C(Cl)c1ccc2nc(-c3cc(Cl)cc(Cl)c3)oc2c1 ; NC1CCCC1 ; O=C(NC1CCCC1)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of cyclopentanamine (26.1 mg, 0.31 mmol) and TEA (93 mg, 0.93 mmol) in DCM (2 mL) and stirred for 2 h at 25° C. The mixture was quenched with water (30 mL) and filtered. The cake was washed with DCM (5 mL×3). The cake was collected and dried under reduced pressure without purification to afford the title compound (83.26 mg, 70.36% yield, 97.1% purity) as a white solid.

LCMS: m/z 374.9 [M+H]⁺.

¹H NMR (400 MHz, DMSO-d₆) δ=8.45 (d, J=7.2 Hz, 1H), 8.27 (d, J=0.8 Hz, 1H), 8.17 (d, J=2.0 Hz, 2H), 7.99-7.94 (m, 2H), 7.92-7.88 (m, 1H), 4.33-4.16 (m, 1H), 2.00-1.85 (m, 2H), 1.80-1.65 (m, 2H), 1.63-1.51 (m, 4H).

### Example 20

### Compound 20: N-cyclopropyl-2-(3,5-dichlorophenyl)benzo[d]oxazole-6-carboxamide

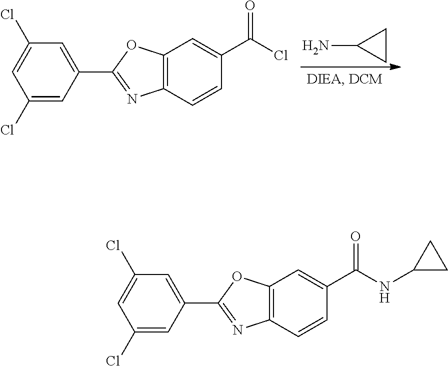

To a solution of cyclopropanamine (31 mg, 0.55 mmol) in DCM (6 mL) was added DIEA (178 mg, 1.38 mmol) and 2-(3,5-dichlorophenyl)benzo[d]oxazole-6-carbonyl chloride (150 mg, 0.46 mmol). The reaction mixture was stirred for 12 h at 25° C. The reaction mixture was concentrated under reduced pressure. The residue was recrystallized from DCM (5 mL) to afford the title compound (133.56 mg, 82% yield, 97.9% purity) as white solid.

LCMS: m/z 346.8 [M+H]⁺

¹H NMR (400 MHz, DMSO-d₆) δ=8.61 (d, J=3.6 Hz, 1H), 8.23 (s, 1H), 8.16 (d, J=1.6 Hz, 2H), 7.96-7.89 (m, 3H), 2.92-2.86 (m, 1H), 0.75-0.71 (m, 2H), 0.63-0.59 (m, 2H).

### Example 21

### Compound 21: 2-(3,5-dichlorophenyl)-N-(1-methylpyrrolidin-3-yl)benzo[d]oxazole-6-carboxamide

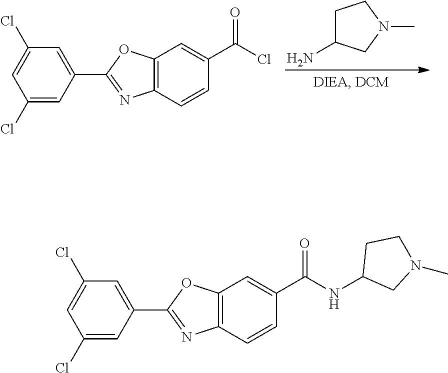

To a solution of 1-methylpyrrolidin-3-amine (55 mg, 0.55 mmol) in DCM (6 mL) was added DIEA (178 mg, 1.38 mmol) and 2-(3,5-dichlorophenyl)benzo[d]oxazole-6-carbonyl chloride (150 mg, 0.46 mmol). The reaction mixture was stirred for 12 h at 25° C. The reaction mixture was concentrated under reduced pressure. The residue was purified by prep-HPLC (column: Welch Ultimate XB-Diol 250×50×10 μm; mobile phase: (Hexane-EtOH); B %: 10%-65%, 17 min) to give the title compound (92.01 mg, 51% yield, 98.8% purity) as white solid.

LCMS: m/z 377.8 [M+H]⁺.

¹H NMR (400 MHz, CDCl₃) δ=8.16-8.14 (m, 3H), 7.85-7.78 (m, 2H), 7.55 (t, J=2.0 Hz, 1H), 7.00-6.96 (m, 1H), 4.78-4.76 (m, 1H), 3.15-3.10 (m, 1H), 2.94-2.92 (m, 1H), 2.64-2.60 (m, 1H), 2.52-2.44 (m, 4H), 2.31 (q, J=8.4 Hz, 1H), 1.91-1.83 (m, 2H).

### Example 22

### Compound 22: 2-(3,5-dichlorophenyl)-N-(1-(2-fluoroethyl)pyrrolidin-3-yl)benzo[d]oxazole-6-carboxamide

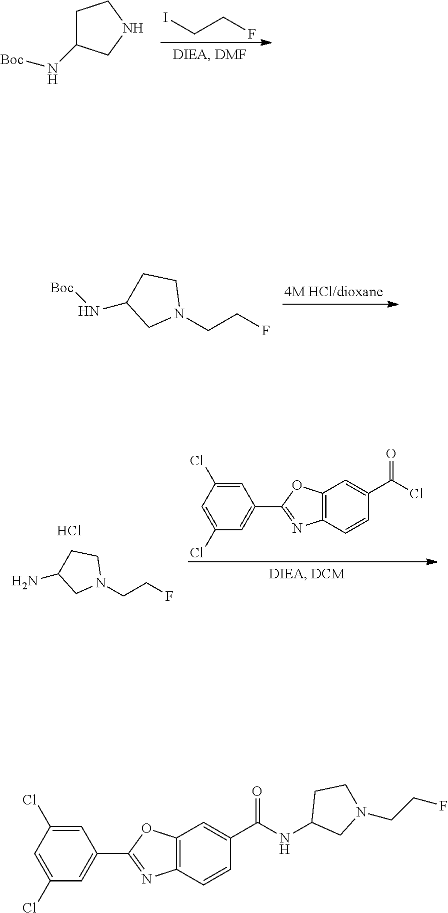

### Step 1: tert-butyl (1-(2-fluoroethyl)pyrrolidin-3-yl)carbamate

To a solution of tert-butyl pyrrolidin-3-ylcarbamate (500 mg, 2.68 mmol) in DMF (10 mL) was added DIEA (1.73 g, 13.42 mmol) and 1-fluoro-2-iodo-ethane (934 mg, 5.37 mmol). The reaction mixture was heated at 60° C. and stirred for 12 h. The reaction mixture was quenched with water (30 mL), extracted with ethyl acetate (30 mL×3). The combined organic phase was washed with brine (10 mL), dried over Na₂SO₄, filtered and then the filtrate was concentrated under reduced pressure. The residue was purified by silica gel column (DCM:MeOH=200:1 to 60:1) to give the title compound (240 mg, 38% yield) as yellow solid.

¹H NMR (400 MHz, CDCl₃) δ=4.86 (bs, 1H), 4.60 (t, J=4.8 Hz, 1H), 4.48 (t, J=4.8 Hz, 1H), 4.17 (bs, 1H), 2.89 (bs, 1H), 2.80 (t, J=4.8 Hz, 1H), 2.73 (t, J=4.8 Hz, 1H), 2.64 (bs, 2H), 2.38-2.36 (m, 1H), 2.30-2.21 (m, 1H), 1.64-1.56 (m, 1H), 1.43 (s, 9H).

### Step 2: 1-(2-fluoroethyl)pyrrolidin-3-amine hydrochloride

A solution of tert-butyl (1-(2-fluoroethyl)pyrrolidin-3-yl)carbamate (240 mg, 1.03 mmol) in HCl/dioxane (4 M, 2 mL) was stirred for 12 h at 25° C. The reaction mixture was concentrated under reduced pressure to afford the title compound (200 mg, crude, HCl salt) as yellow solid, which was used for next step without purification.

### Step 3: 2-(3,5-dichlorophenyl)-N-(1-(2-fluoroethyl)pyrrolidin-3-yl)benzo[d]oxazole-6-carboxamide

To a suspension of 1-(2-fluoroethyl)pyrrolidin-3-amine (155 mg, 0.92 mmol, HCl salt) in DCM (6 mL) was added DIEA (297 mg, 2.30 mmol, 5 eq) and then 2-(3,5-dichlorophenyl)benzo[d]oxazole-6-carbonyl chloride (150 mg, 0.46 mmol) at 25° C. The reaction mixture was stirred for 12 h at 25° C. The reaction mixture was concentrated under reduced pressure. The residue was purified by prep-HPLC (column: Welch Ultimate XB-Diol 250×50×10 μm; mobile phase: (Hexane-EtOH, neutral); B %: 5%-30%, 15 min) to afford the title compound (132.67 mg, 66% yield, 96.5% purity) as white solid.

LCMS: m/z 421.9 [M+H]⁺

¹H NMR (400 MHz, CDCl₃) δ=8.17-8.16 (m, 3H), 7.87-7.85 (m, 1H), 7.82-7.80 (m, 1H), 7.55 (t, J=2.0 Hz, 1H), 7.12-7.0 (m, 1H), 4.83 (bs, 1H), 4.73 (t, J=4.4 Hz, 1H), 4.61 (t, J=4.4 Hz, 1H), 3.31-3.28 (m, 1H), 3.12-3.10 (m, 1H), 3.00 (t, J=4.4 Hz, 1H), 2.93 (t, J=4.4 1H), 2.81-2.77 (m, 1H), 2.54-2.45 (m, 2H), 1.97-1.89 (m, 1H).

### Example 23

### Compound 23: 2-(3,5-dichlorophenyl)-N-(1-(2,2-difluoroethyl)pyrrolidin-3-yl)benzo[d]oxazole-6-carboxamide

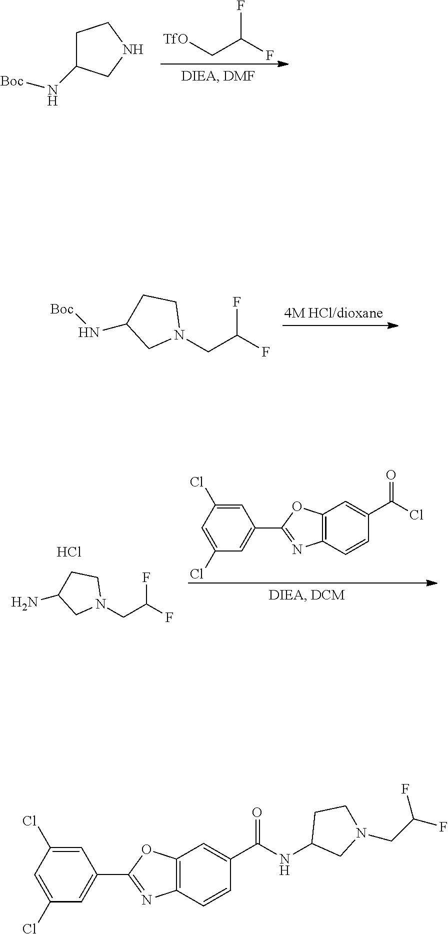

### Step 1: tert-butyl (1-(2,2-difluoroethyl)pyrrolidin-3-yl)carbamate

To a solution of tert-butyl pyrrolidin-3-ylcarbamate (500 mg, 2.68 mmol) in DMF (10 mL) was added DIEA (1.73 g, 13.40 mmol) and 2,2-difluoroethyl trifluoromethanesulfonate (1.15 g, 5.36 mmol, 2 eq). The reaction mixture was quenched with water (30 mL), extracted with ethyl acetate (30 mL×3). The organic layer was separated and washed with brine (10 mL), dried over Na₂SO₄, filtered and then the filtrate was concentrated under reduced pressure. The residue was purified by silica gel column (DCM:MeOH=200:1 to 60:1) to afford the title compound (1.0 g, crude) as yellow oil.

¹H NMR (400 MHz, CDCl₃) δ=5.99-5.69 (m, 1H), 4.84 (bs, 1H), 4.16 (bs, 1H), 2.93-2.90 (m, 1H), 2.86-2.78 (m, 2H), 2.73-2.65 (m, 2H), 2.44-2.42 (m, 1H), 2.28-2.19 (m, 1H), 1.64-1.56 (m, 1H), 1.42 (s, 9H).

### Step 2: 1-(2,2-difluoroethyl)pyrrolidin-3-amine hydrochloride

A solution of tert-butyl (1-(2,2-difluoroethyl)pyrrolidin-3-yl)carbamate (1 g, 4.00 mmol, 1 eq) in HCl/dioxane (4 M, 8 mL) was stirred for 12 h at 25° C. The reaction mixture was concentrated under reduced pressure to afford the title compound (820 mg, crude, HCl salt) as yellow solid, which was used for next step without purification.

### Step 3: 2-(3,5-dichlorophenyl)-N-(1-(2,2-difluoroethyl)pyrrolidin-3-yl)benzo[d]oxazole-6-carboxamide

To a suspension of 1-(2,2-difluoroethyl)pyrrolidin-3-amine (154 mg, 0.83 mmol) in DCM (6 mL) was added DIEA (297 mg, 2.30 mmol) and then 2-(3,5-dichlorophenyl)benzo[d]oxazole-6-carbonyl chloride (150 mg, 0.46 mmol) at 25° C. The reaction mixture was stirred for 12 h at 25° C. The reaction mixture was concentrated under reduced pressure. The residue was purified by prep-HPLC (column: Welch Ultimate XB—SiOH 250×50×10 μm; mobile phase: (Hexane-EtOH, neutral); B %: 0%-30%, 13 min) to afford the title compound (36.09 mg, 18% yield, 99.3% purity) as white solid.

LCMS: m/z 439.9 [M+H]⁺

¹H NMR (400 MHz, CDCl₃) δ=8.17-8.15 (m, 3H), 7.85-7.80 (m, 2H), 7.55 (t, J=2.0 Hz, 1H), 7.00-6.93 (m, 1H), 6.18-5.90 (m, 1H), 4.82-4.78 (m, 1H), 3.30 (bs, 1H), 3.11-2.98 (m, 3H), 2.88-2.84 (m, 1H), 2.60-2.45 (m, 2H), 1.94-1.92 (m, 1H).

### Example 24

### Compound 24: 2-(3,5-dichlorophenyl)-N-(1-(2,2,2-trifluoroethyl)pyrrolidin-3-yl)benzo[d]oxazole-6-carboxamide

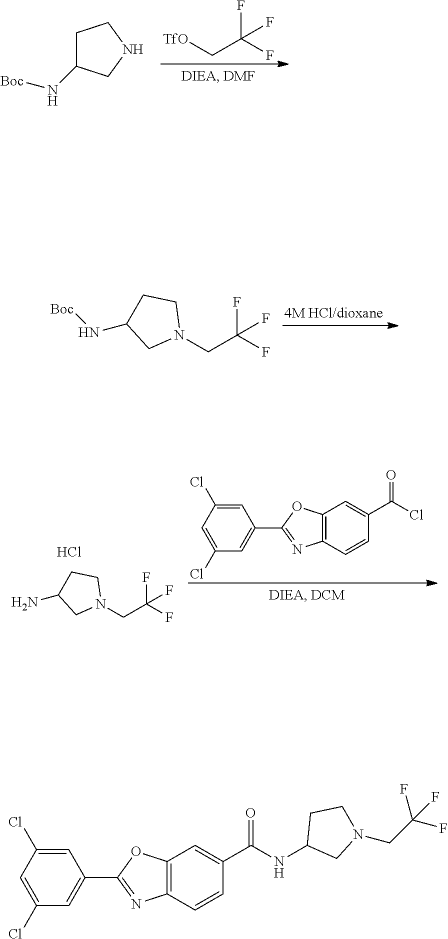

### Step 1: tert-butyl (1-(2,2,2-trifluoroethyl)pyrrolidin-3-yl)carbamate

To a solution of tert-butyl pyrrolidin-3-ylcarbamate (500 mg, 2.68 mmol) in DMF (10 mL) was added DIEA (1.73 g, 13.40 mmol, 2.33 mL) and 2,2,2-trifluoroethyl trifluoromethanesulfonate (1.24 g, 5.36 mmol). The reaction mixture was stirred at 25° C. for 12 h. The reaction mixture was quenched with water (30 mL), extracted with ethyl acetate (30 mL×3). The organic layer was separated and washed with brine (10 mL), dried over Na₂SO₄, filtered and then the filtrate was concentrated under reduced pressure. The residue was purified by silica gel column (DCM:MeOH=200:1 to 60:1) to afford the title compound (450 mg, 63% yield) as white solid.

¹H NMR (400 MHz, CDCl₃) δ=4.82 (bs, 1H), 4.19 (br s, 1H), 3.06 (q, J=9.5 Hz, 3H), 2.90-2.64 (m, 2H), 2.52 (br d, J=7.6 Hz, 1H), 2.31-2.18 (m, 1H), 1.70-1.57 (m, 1H), 1.44 (s, 9H).

### Step 2: 1-(2,2,2-trifluoroethyl)pyrrolidin-3-amine hydrochloride

A solution of tert-butyl (1-(2,2,2-trifluoroethyl)pyrrolidin-3-yl)carbamate (200 mg, 0.75 mmol) in HCl\dioxane (4 M, 2 mL) was stirred for 12 h at 25° C. The mixture was concentrated under reduced pressure to afford the title compound (159 mg, crude, HCl salt) as white solid, which was used for next step without purification.

¹H NMR (400 MHz, DMSO-d₆) δ=8.54 (bs, 3H), 3.94-3.84 (m, 3H), 3.38-3.37 (m, 2H), 3.20-3.10 (m, 2H), 2.34-2.25 (m, 1H), 2.01-1.91 (m, 1H).

### Step 3: 2-(3,5-dichlorophenyl)-N-(1-(2,2,2-trifluoroethyl)pyrrolidin-3-yl)benzo[d]oxazole-6-carboxamide

To a solution of 1-(2,2,2-trifluoroethyl)pyrrolidin-3-amine (150 mg, 0.73 mmol, HCl salt) in DCM (6 mL) was added DIEA (297 mg, 2.30 mmol) and 2-(3,5-dichlorophenyl)benzo[d]oxazole-6-carbonyl chloride (150 mg, 0.46 mmol) at 25° C. The reaction mixture was stirred for 12 h at 25° C. The reaction mixture was concentrated under reduced pressure. The residue was purified by prep-HPLC (column: Welch Ultimate XB—CN 250×50×10 μm; mobile phase: (Hexane-EtOH, neutral); B %: 2%-20%, 12 min) to afford the title compound (121.93 mg, 57% yield, 99.2% purity) as white solid.

LCMS: m/z 357.8 [M+H]⁺

¹H NMR (400 MHz, CDCl₃) δ=8.15 (d, J=2.0 Hz, 2H), 8.09 (s, 1H), 7.81-7.76 (m, 2H), 7.54 (t, J=2.0 Hz, 1H), 6.68 (bd, J=8.0 Hz, 1H), 4.78-4.71 (m, 1H), 3.23-3.12 (m, 3H), 3.01 (bd, J=8.4 Hz, 1H), 2.87 (dd, J=6.4, 9.6 Hz, 1H), 2.57-2.53 (m, 1H), 2.43 (dtd, J=4.0, 8.8, 13.2 Hz, 1H), 1.90-1.85 (m, 1H).

### Example 25

### Compound 25: (2-(3,5-dichlorophenyl)benzo[d]oxazol-6-yl)(4-methylpiperazin-1-yl)methanone

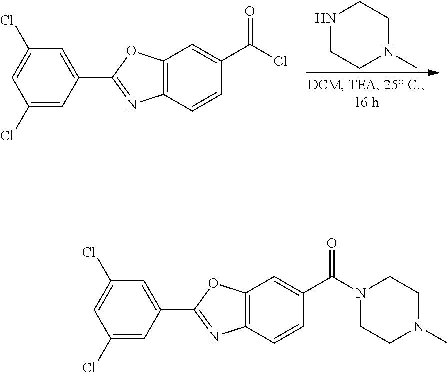

2-(3,5-dichlorophenyl)benzo[d]oxazole-6-carbonyl chloride (100 mg, 0.31 mmol) was added to the solution of 1-methylpiperazine (30.67 mg, 0.31 mmol) and TEA (100 mg, 0.93 mmol) in DCM (2 mL) and stirred for 2 h at 25° C. The mixture was quenched with water (10 mL) and extracted with EtOAc (10 mL×3). The organic phase was washed with brine (20 mL), dried over Na₂SO₄, filtered and concentrated. The residue was purified by column chromatography (SiO₂, DCM:MeOH=100:1 to 5:1) to afford the title compound (95.41 mg, 79.84% yield, 100% purity) as a white solid.

LCMS: m/z 390.1 [M+H]⁺

¹H NMR (400 MHz, CD₃OD) δ=8.19 (d, J=1.6 Hz, 2H), 7.85 (d, J=8.0 Hz, 1H), 7.82 (s, 1H), 7.71 (t, J=2.0 Hz, 1H), 7.50 (dd, J=1.2, 8.0 Hz, 1H), 3.91-3.42 (m, 4H), 2.64-2.38 (m, 4H), 2.34 (s, 3H)

### Example 26

### Compound 26: 2-(3,5-dichlorophenyl)-N-((trans)-3-hydroxycyclobutyl)-benzo[d]oxazole-6-carboxamide

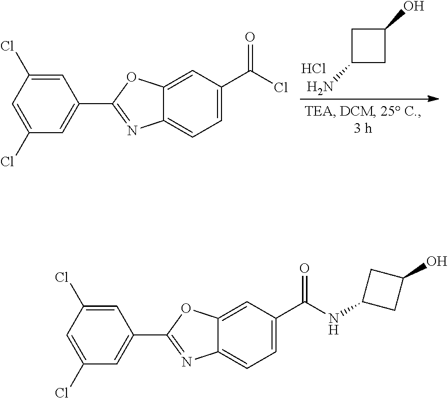 [SMILES: O=C(Cl)c1ccc2nc(-c3cc(Cl)cc(Cl)c3)oc2c1 ; Cl ; N[C@H]1C[C@H](O)C1 ; O=C(N[C@H]1C[C@H](O)C1)c1ccc2nc(-c3cc(Cl)cc(Cl)c3)oc2c1]

2-(3,5-dichlorophenyl)benzo[d]oxazole-6-carbonyl chloride (150 mg, 0.46 mmol) was added to the mixture of trans-3-aminocyclobutanol (63 mg, 0.51 mmol, HCl salt) and TEA (233 mg, 2.30 mmol) in DCM (2 mL) and stirred for 2 h at 25° C. The mixture was filtered and the cake was washed with DCM:methanol=10:1 (50 mL) to afford the title compound (50.66 mg, 29.0% yield, 97.8% purity) as a white solid.

LCMS: m/z 377.0 [M+H]⁺

¹H NMR (400 MHz, DMSO-d₆) δ=8.75 (d, J=6.4 Hz, 1H), 8.27 (d, J=1.2 Hz, 1H), 8.17 (d, J=1.6 Hz, 2H), 8.00-7.94 (m, 2H), 7.94-7.88 (m, 1H), 5.04 (d, J=6.4 Hz, 1H), 4.52-4.40 (m, 1H), 4.39-4.30 (m, 1H), 2.34-2.28 (m, 2H), 2.21-2.15 (m, 2H).

### Example 27

### Compound 27: 2-(3,5-dichlorophenyl)-N-((Cis)-3-hydroxycyclobutyl)benzo-[d]oxazole-6-carboxamide

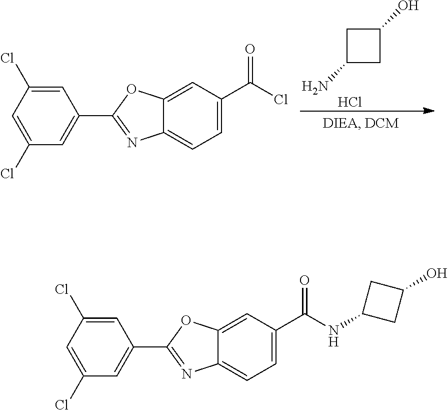

To a solution of cis-3-aminocyclobutanol (85.15 mg, 0.69 mmol, HCl salt) in DCM (10 mL) was added DIEA (208 mg, 1.61 mmol) and (150 mg, 0.46 mmol) at 25° C. The reaction mixture was stirred for 16 h at 25° C. The suspension was filtered and the filter cake was washed with DCM:Petroleum ether=5:1 (30 mL). The solid was concentrated under reduced pressure. The crude product was purified by flash silica gel chromatography (20 g Silica Flash Column, Eluent of 0-30% Dichloromethane/Methanol @ 20 mL/min) to afford the title compound (52.71 mg, 30% yield, 99.3% purity) as a white solid.

LCMS: m/z 377.0 [M+H]⁺

¹H NMR (400 MHz, CD₃OD) δ=8.22 (d, J=2.0 Hz, 2H), 8.19 (d, J=1.2 Hz, 1H), 7.94 (dd, J=1.6, 8.4 Hz, 1H), 7.84 (d, J=8.4 Hz, 1H), 7.74 (t, J=2.0 Hz, 1H), 4.09-4.01 (m, 2H), 2.81-2.74 (m, 2H), 2.06-1.99 (m, 2H).

### Example 28

### Compound 28: 2-(3,5-dichlorophenyl)-N-((cis)-2-hydroxycyclopentyl)benzo-[d]oxazole-6-carboxamide

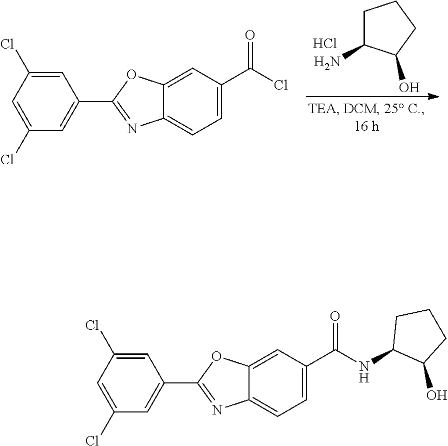 [SMILES: O=C(Cl)c1ccc2nc(-c3cc(Cl)cc(Cl)c3)oc2c1 ; Cl ; N[C@H]1CCC[C@H]1O ; O=C(N[C@H]1CCC[C@H]1O)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of (cis)-2-aminocyclopentanol (42 mg, 0.31 mmol, HCl salt) and TEA (155 mg, 1.53 mmol) in DCM (2 mL) and stirred for 16 h at 25° C. The mixture was filtered and the cake was triturated with DCM (10 mL) to afford the title compound (72.45 mg, 60% yield, 99.4% purity) as a white solid

LCMS: m/z 391.1 [M+H]⁺

¹H NMR (400 MHz, DMSO-d₆) δ=8.32 (d, J=0.8 Hz, 1H), 8.17 (d, J=2.0 Hz, 2H), 8.11 (d, J=7.2 Hz, 1H), 8.00 (dd, J=1.6, 8.4 Hz, 1H), 7.96 (t, J=1.6 Hz, 1H), 7.90 (d, J=8.4 Hz, 1H), 4.74 (d, J=3.6 Hz, 1H), 4.15-4.01 (m, 2H), 1.89-1.71 (m, 4H), 1.67-1.45 (m, 2H).

### Example 29

### Compound 29: 2-(3,5-dichlorophenyl)-N-((trans)-2-hydroxycyclopentyl)benzo-[d]oxazole-6-carboxamide

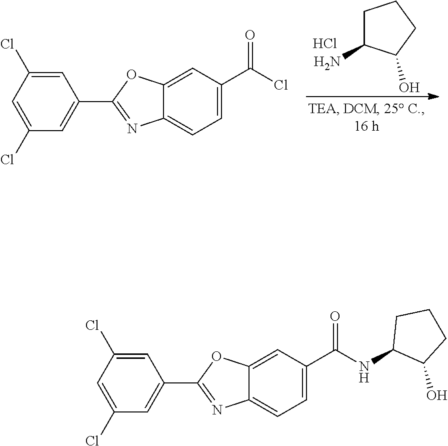 [SMILES: O=C(Cl)c1ccc2nc(-c3cc(Cl)cc(Cl)c3)oc2c1 ; Cl ; N[C@H]1CCC[C@@H]1O ; O=C(N[C@H]1CCC[C@@H]1O)c1ccc2nc(-c3cc(Cl)cc(Cl)c3)oc2c1]

2-(3,5-dichlorophenyl)-1,3-benzoxazole-6-carbonyl chloride (0.1 g, 0.31 mmol) was added to the mixture of (trans)-2-aminocyclopentanol (42 mg, 0.31 mmol, HCl salt) and TEA (155 mg, 1.53 mmol) in DCM (2 mL) and stirred for 16 h at 25° C. The mixture was concentrated and the crude product was purified by column chromatography (SiO₂, Petroleum ether/Ethyl acetate=0/1) to afford the title compound (32.55 mg, 27% yield, 100% purity) as a white solid.

LCMS: m/z 391.1 [M+H]⁺

¹H NMR (400 MHz, DMSO-d₆) δ=8.46 (d, J=7.6 Hz, 1H), 8.25 (s, 1H), 8.16 (d, J=2.0 Hz, 2H), 7.99-7.93 (m, 2H), 7.92-7.86 (m, 1H), 4.53 (d, J=3.6 Hz, 1H), 4.52-4.42 (m, 1H), 4.28-4.20 (m, 1H), 2.16-2.01 (m, 1H), 2.00-1.83 (m, 2H), 1.78-1.66 (m, 1H), 1.57-1.45 (m, 2H).

### Example 30

### Compound 30: 2-(3,5-dichlorophenyl)-N-((cis)-3-hydroxycyclopentyl)benzo-[d]oxazole-6-carboxamide

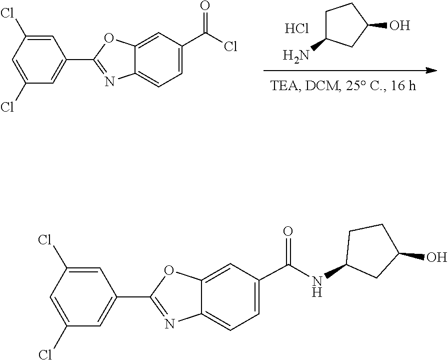 [SMILES: O=C(Cl)c1ccc2nc(-c3cc(Cl)cc(Cl)c3)oc2c1 ; Cl ; N[C@H]1CC[C@@H](O)C1 ; O=C(N[C@H]1CC[C@@H](O)C1)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of (cis)-3-aminocyclopentanol (42 mg, 0.31 mmol, HCl salt) and TEA (155 mg, 1.53 mmol) in DCM (2 mL) and stirred for 16 h at 25° C. The mixture was concentrated purified by column chromatography (SiO₂, Dichloromethane:Methanol=10/1) to afford the title compound (71.69 mg, 58% yield, 96.7% purity) as a white solid.

LCMS: m/z 391.0 [M+H]⁺

¹H NMR (400 MHz, DMSO-d₆) δ=8.49 (d, J=7.2 Hz, 1H), 8.26 (s, 1H), 8.16 (d, J=2.0 Hz, 2H), 7.99-7.93 (m, 2H), 7.93-7.86 (m, 1H), 4.69 (d, J=4.0 Hz, 1H), 4.29-4.17 (m, 1H), 4.17-4.07 (m, 1H), 2.25-2.14 (m, 1H), 1.96-1.84 (m, 1H), 1.81-1.69 (m, 2H), 1.68-1.50 (m, 2H).

### Example 31

### Compound 31: 2-(3,5-dichlorophenyl)-N-((trans)-3-hydroxycyclopentyl)benzo-[d]oxazole-6-carboxamide

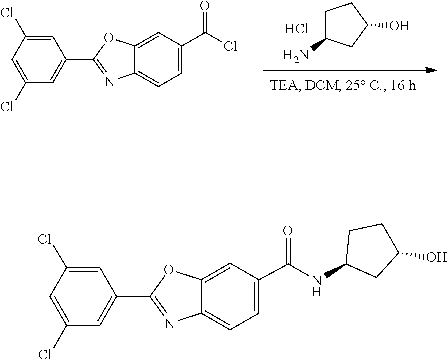 [SMILES: O=C(Cl)c1ccc2nc(-c3cc(Cl)cc(Cl)c3)oc2c1 ; Cl ; N[C@H]1CC[C@H](O)C1 ; O=C(N[C@H]1CC[C@H](O)C1)c1ccc2nc(-c3cc(Cl)cc(Cl)c3)oc2c1]

2-(3,5-dichlorophenyl)benzo[d]oxazole-6-carbonyl chloride (100 mg, 0.31 mmol) 0.31 mmol) was added to the mixture of (trans)-3-aminocyclopentanol (42 mg, 0.31 mmol, HCl) and TEA (155 mg, 1.53 mmol) in DCM (2 mL) and stirred for 16 h at 25° C. The mixture was concentrated purified by column chromatography (SiO₂, Dichloromethane:Methanol=10/1) to afford the title compound (102.02 mg, 84% yield, 98.6% purity) as a white solid

LCMS: m/z 391.0 [M+H]⁺

¹H NMR (400 MHz, DMSO-d₆) δ=8.40 (d, J=6.8 Hz, 1H), 8.27 (d, J=0.8 Hz, 1H), 8.17 (d, J=1.6 Hz, 2H), 8.00-7.94 (m, 2H), 7.93-7.88 (m, 1H), 4.79 (d, J=4.0 Hz, 1H), 4.10-3.97 (m, 2H), 2.11-1.97 (m, 1H), 1.94-1.80 (m, 1H), 1.76-1.61 (m, 2H), 1.58-1.42 (m, 2H).

### Example 32

### Compound 32: 2-(3,5-dichlorophenyl)-N-((cis)-2-hydroxycyclohexyl)benzo-[d]oxazole-6-carboxamide

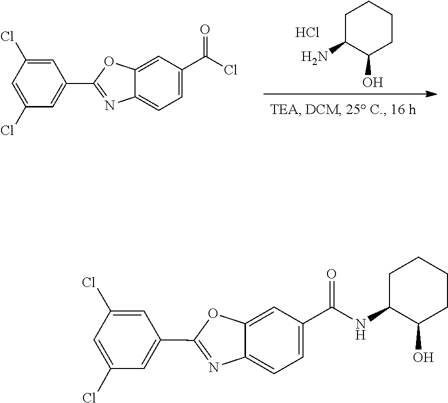 [SMILES: O=C(Cl)c1ccc2nc(-c3cc(Cl)cc(Cl)c3)oc2c1 ; Cl ; N[C@H]1CCCC[C@H]1O ; O=C(N[C@H]1CCCC[C@H]1O)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of (cis)-2-aminocyclohexanol (46 mg, 0.31 mmol, HCl) and TEA (155 mg, 1.53 mmol) in DCM (2 mL) and stirred for 16 h at 25° C. The mixture was filtered and the cake was triturated with DCM (10 mL) to afford the title compound (58.52 mg, 46% yield, 98.4% purity) as a white solid.

LCMS: m/z 405.1 [M+H]⁺

¹H NMR (400 MHz, DMSO-d₆) δ=8.30 (d, J=0.8 Hz, 1H), 8.16 (d, J=1.6 Hz, 2H), 8.03 (d, J=7.6 Hz, 1H), 8.00-7.97 (m, 1H), 7.96 (t, J=2.0 Hz, 1H), 7.93-7.86 (m, 1H), 4.70 (d, J=3.6 Hz, 1H), 3.89 (s, 2H), 1.85-1.63 (m, 3H), 1.62-1.42 (m, 3H), 1.40-1.21 (m, 2H).

### Example 33

### Compound 33: 2-(3,5-dichlorophenyl)-N-((trans)-2-hydroxycyclohexyl)benzo-[d]oxazole-6-carboxamide

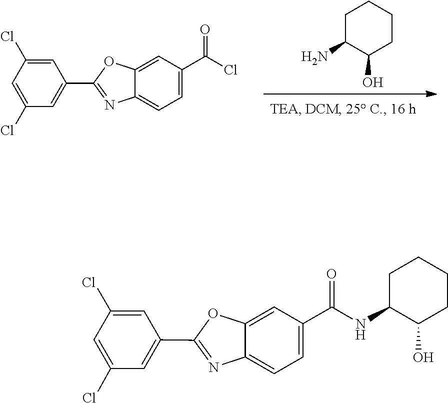 [SMILES: O=C(Cl)c1ccc2nc(-c3cc(Cl)cc(Cl)c3)oc2c1 ; N[C@H]1CCCC[C@H]1O ; O=C(N[C@H]1CCCC[C@@H]1O)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the mixture of (trans)-2-aminocyclohexanol (35 mg, 0.31 mmol) and TEA (155 mg, 1.53 mmol) in DCM (2 mL) and stirred for 16 h at 25° C. The mixture was filtered and the cake was triturated with DCM (10 mL) to afford the title compound (90.47 mg, 73% yield, 99.6% purity) as a white solid.

LCMS: m/z 405.1 [M+H]⁺

¹H NMR (400 MHz, DMSO-d₆) δ=8.33-8.24 (m, 2H), 8.17 (d, J=2.0 Hz, 2H), 8.02-7.97 (m, 1H), 7.96 (t, J=2.0 Hz, 1H), 7.94-7.87 (m, 1H), 4.66 (d, J=5.2 Hz, 1H), 3.74-3.57 (m, 1H), 3.54-3.38 (m, 1H), 1.98-1.80 (m, 2H), 1.66 (d, J=9.2 Hz, 2H), 1.35-1.16 (m, 4H).

### Example 34

### Compound 34: 1-(1H-imidazol-2-yl)propan-2-yl 2-(3,5-dichlorophenyl)benzo-[d]oxazole-6-carboxylate

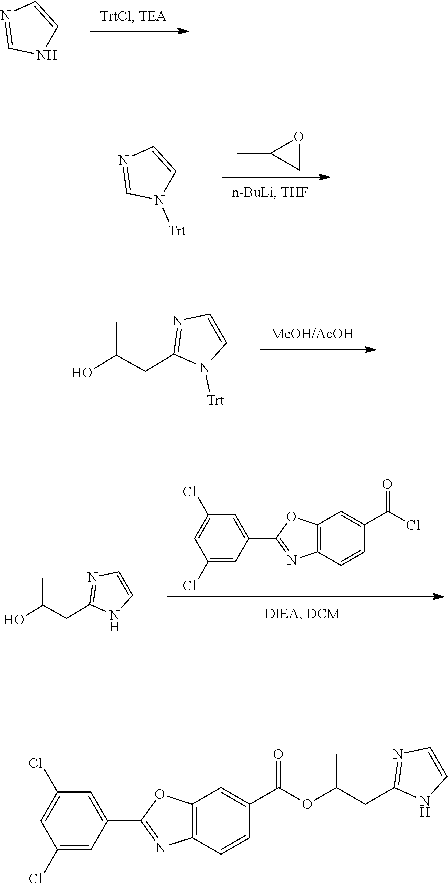

### Step 1: 1-trityl-1H-imidazole

To a solution of imidazole (2.3 g, 33.79 mmol) in DCM (100 mL) was added DIEA (5.24 g, 40.54 mmol) and TrtCl (9.42 g, 33.79 mmol). The reaction mixture was stirred for 12 h at 25° C. The reaction mixture was quenched with water (100 mL) and extracted with DCM (200 mL). The organic layer was separated and washed with brine (100 mL), dried over Na₂SO₄ and filtered. The filtrate was concentrated under reduced pressure. The residue was suspended in EtOAc (100 mL) and heated at 80° C. for 2 h, and then cooled to 25° C. The mixture was filtered and washed with ethyl acetate (20 mL×3). The cake was collected and dried over under reduced pressure to afford the title compound (9.2 g, 88% yield) as white solid.

¹H NMR (400 MHz, DMSO-d₆) δ=7.43-7.35 (m, 10H), 7.10-7.08 (m, 6H), 6.98 (s, 1H), 6.89 (s, 1H).

### Step 2: 1-(1-trityl-1H-imidazol-2-yl)propan-2-ol

To a solution of 1-tritylimidazole (1 g, 3.22 mmol) in THF (20 mL) was n-BuLi (2.5 M, 1.6 mL) at 0° C. under N₂. After 30 min, 2-methyloxirane (281 mg, 4.83 mmol) was added. The reaction mixture was warmed to 25° C. and stirred for 12 h. The reaction mixture was quenched with saturated NH₄Cl solution (30 mL) and extracted with DCM (60 mL). The organic layer was separated and washed with brine (10 mL), dried over Na₂SO₄, and filtered, then the filtrate was concentrated under reduced pressure to afford the title compound (1.0 g, 84% yield) as yellow solid, which was used for next step without purification.

### Step 3: 1-(1H-imidazol-2-yl)propan-2-ol

To a solution of 1-(1-tritylimidazol-2-yl)propan-2-ol (1 g, 2.71 mmol) in MeOH (20 mL) was added AcOH (3.15 g, 52.45 mmol). The reaction mixture was heated at 80° C. and stirred for 12 h. The reaction mixture was concentrated under reduced pressure. The residue was purified by silica gel column (DCM:MeOH=20:1 to 5:1, 0.1% NH₃·H₂O as additive) to afford the title compound (220 mg, 32% yield, 50% purity) as yellow oil.

¹H NMR (400 MHz, CD₃OD) δ=7.04 (s, 1H), 6.91 (s, 1H), 4.12-4.04 (m, 1H), 2.86-2.72 (m, 2H), 1.14 (d, J=6.0 Hz, 3H).

### Step 4: 1-(1H-imidazol-2-yl)propan-2-yl 2-(3,5-dichlorophenyl)benzo[d]oxazole-6-carboxylate

To a solution of 1-(1H-imidazol-2-yl)propan-2-ol (174 mg, 0.69 mmol, 50% purity) in DCM (6 mL) was added DIEA (178 mg, 1.38 mmol) and 2-(3,5-dichlorophenyl)benzo[d]oxazole-6-carbonyl chloride (150 mg, 0.46 mmol). The reaction mixture was stirred for 12 h at 25° C. The reaction mixture was concentrated under reduced pressure. The residue was recrystallized from DCM (5 mL) to afford the title compound (133.6 mg, 69% yield, 98.5% purity) as white solid.

LCMS: m/z 415.9 [M+H]⁺

¹H NMR (400 MHz, DMSO-d₆) δ=11.86 (bs, 1H), 8.26 (s, 1H), 8.14 (d, J=1.6 Hz, 2H), 8.01-7.98 (m, 1H), 7.95-7.91 (m, 2H), 7.0-6.80 (m, 2H), 5.42-5.34 (m, 1H), 3.11-3.0 (m, 2H), 1.36 (d, J=6.0 Hz, 3H).

### Example 35

### Compound 35: 2-(1H-imidazol-5-yl) ethyl 2-(3,5-dichlorophenyl)benzo[d]oxazole-6-carboxylate

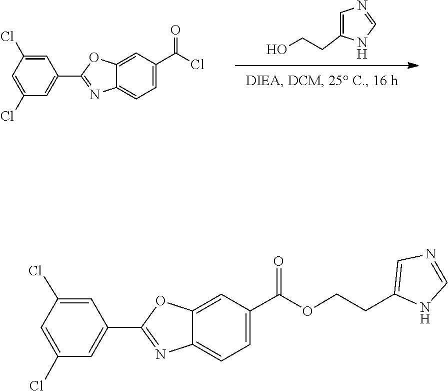 [SMILES: O=C(Cl)c1ccc2nc(-c3cc(Cl)cc(Cl)c3)oc2c1 ; OCCc1cnc[nH]1 ; O=C(OCCc1cnc[nH]1)c1ccc2nc(-c3cc(Cl)cc(Cl)c3)oc2c1]

To a solution of 2-(1H-imidazol-5-yl)ethanol (77 mg, 0.69 mmol) in DCM (10 mL) was added DIEA (178 mg, 1.38 mmol) and 2-(3,5-dichlorophenyl)benzo[d]oxazole-6-carbonyl chloride (150 mg, 0.46 mmol) at 25° C. The reaction mixture was stirred for 16 h at 25° C. The mixture was concentrated under reduced pressure. The residue was purified by prep-HPLC (column: Welch Ultimate XB—CN 250×50×10 μm; mobile phase: (Hexane-EtOH, neutral); B %: 10%-35%, 15 min), followed by prep-TLC (DCM:MeOH=10:1) to afford the title compound (22.47 mg, 12% yield, 99.05% purity) as a white solid.

LCMS: m/z 402.2 [M+H]⁺.

1H NMR (400 MHz, CD₃OD) δ=8.32 (d, J=1.2 Hz, 1H), 8.22 (d, J=2.0 Hz, 2H), 8.10 (dd, J=1.6, 8.4 Hz, 1H), 7.84 (d, J=8.4 Hz, 1H), 7.74 (t, J=2.0 Hz, 1H), 7.64 (bs, 1H), 6.97 (bs, 1H), 4.59 (t, J=6.8 Hz, 2H), 3.10 (t, J=6.8 Hz, 2H).

### Example 36

### Compound 36: 2-(1H-pyrazol-5-yl) ethyl 2-(3,5-dichlorophenyl)benzo[d]oxazole-6-carboxylate

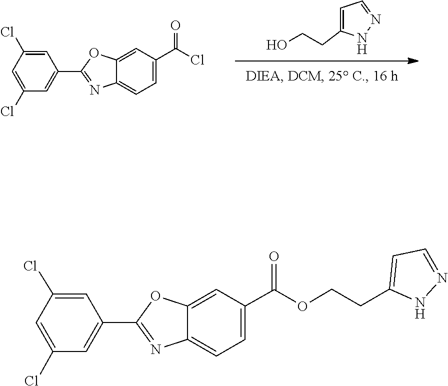 [SMILES: O=C(Cl)c1ccc2nc(-c3cc(Cl)cc(Cl)c3)oc2c1 ; OCCc1ccn[nH]1 ; O=C(OCCc1ccn[nH]1)c1ccc2nc(-c3cc(Cl)cc(Cl)c3)oc2c1]

To a solution of 2-(1H-pyrazol-3-yl)ethanol (77 mg, 0.69 mmol) in DCM (10 mL) was added DIEA (297 mg, 2.30 mmol) and 2-(3,5-dichlorophenyl)-1,3-benzoxazole-6-carbonyl chloride (150 mg, 0.46 mmol) at 25° C. The reaction mixture was stirred for 16 h at 25° C. LCMS showed the starting material was consumed, the desired product mass was detected. The mixture was concentrated under reduced pressure. The residue was purified by prep-HPLC (column: Welch Ultimate XB—SiOH 250×50×10 μm; mobile phase: (Hexane-EtOH, neutral); B %: 0%-20%, 15 min) and prep-TLC (DCM:MeOH=10:1) to afford the title compound (41.1 mg, 21% yield, 96.1% purity) as a white solid.

LCMS: m/z 401.9 [M+H]⁺

1H NMR (400 MHz, CDCl₃) δ=8.52 (d, J=1.2 Hz, 1H), 8.43 (d, J=2.8 Hz, 1H), 8.23-8.20 (m, 3H), 7.88 (d, J=8.4 Hz, 1H), 7.57 (t, J=2.0 Hz, 1H), 6.46 (d, J=2.8 Hz, 1H), 4.01 (t, J=6.0 Hz, 2H), 2.99 (t, J=6.0 Hz, 2H).

### Example 37

### Compound 37: 1-(1H-imidazol-2-yl) butan-2-yl 2-(3,5-dichlorophenyl)benzo-[d]oxazole-6-carboxylate

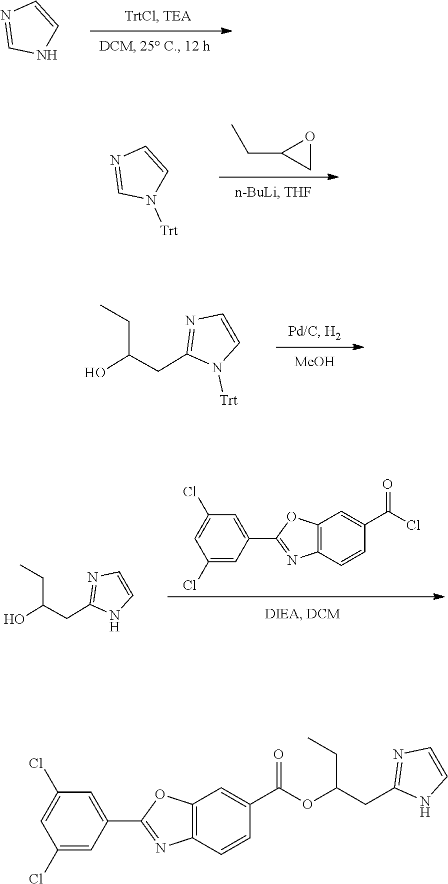

### Step 1: 1-trityl-1H-imidazole

To a solution of imidazole (2.3 g, 33.79 mmol) in DCM (100 mL) was added DIEA (5.24 g, 40.54 mmol) and TrtCl (9.42 g, 33.79 mmol). The reaction mixture was stirred for 12 h at 25° C. A new spot was generated. The reaction mixture was quenched with water (100 mL), extracted with DCM (200 mL). The organic layer was separated and washed with brine (100 mL), dried over Na₂SO₄, filtered and then the filtrate was concentrated under reduced pressure. The residue was suspended in EtOAc (100 mL) and heated at 80° C. for 2 h, and then cooled to 25° C. The mixture was filtered and washed with EtOAc (20 mL×3). The cake was collected and dried over under reduced pressure to afford the title compound (9.2 g, 88% yield) as a white solid.

¹H NMR (400 MHz, DMSO-d₆) δ=7.43-7.35 (m, 10H), 7.10-7.08 (m, 6H), 6.98 (s, 1H), 6.89 (s, 1H).

### Step 2: 1-(1-trityl-1H-imidazol-2-yl) butan-2-ol

To a solution of 1-tritylimidazole (2 g, 6.44 mmol) in THF (40 mL) was added n-BuLi (2.5 M, 3.09 mL) at 0° C. After 30 min, 2-ethyloxirane (929 mg, 12.89 mmol) was added. The reaction mixture was warmed to 25° C. and stirred for 12 h. LCMS showed the starting material remained, the desired mass was detected. The reaction mixture was quenched with saturated NH₄Cl solution (30 mL), washed with EtOAc (30 mL×3). The organic layer was separated and washed with brine (20 mL), dried over Na₂SO₄, filtered and then the filtrate was concentrated under reduced pressure. The residue was purified by silica gel column (DCM:MeOH=100:1 to 30:1) to afford the title compound (590 mg, 24% yield) as yellow solid.

¹H NMR (400 MHz, CDCl₃) δ=7.36-7.33 (m, 9H), 7.13-7.11 (m, 6H), 6.94 (s, 1H), 6.73 (s, 1H), 5.76 (m, 1H), 3.28-3.23 (m, 1H), 2.04-2.0 (m, 1H), 1.90-1.83 (m, 1H), 1.34-1.23 (m, 1H), 1.05-0.95 (m, 1H), 0.56 (t, J=7.2 Hz, 3H).

### Step 3: 1-(1H-imidazol-2-yl) butan-2-ol

To a solution of 1-(1-tritylimidazol-2-yl) butan-2-ol (350 mg, 0.92 mmol) in MeOH (15 mL) was added Pd/C (35 mg). The suspension was degassed under reduced pressure and purged with H₂ for three times. The reaction mixture was stirred under H₂ balloon (15 Psi) at 25° C. for 12 h. LCMS showed the starting material was consumed, and the desired mass was detected. The suspension was filtered and the filter cake was washed with MeOH (10 mL×3). The filtrate was concentrated under reduced pressure affording the crude product as white solid. The crude product was purified by slurry in Petroleum ether (30 mL) at 25° C. for 1 h. The solid was collected by filtration and dried under vacuum to afford the title compound (0.14 g, 84% yield) as a white solid.

¹H NMR (400 MHz, CDCl₃) δ=6.96 (s, 2H), 3.97-3.91 (m, 1H), 2.97-2.93 (m, 1H), 2.78-2.72 (m, 1H), 1.62-1.51 (m, 2H), 0.99 (t, J=7.6 Hz, 3H).

### Step 4: 1-(1H-imidazol-2-yl) butan-2-yl 2-(3,5-dichlorophenyl) benzo[d]oxazole-6-carboxylate

To a solution of 1-(1H-imidazol-2-yl)butan-2-ol (77 mg, 0.55 mmol) in DCM (5 mL) was added DIEA (178 mg, 1.38 mmol) and 2-(3,5-dichlorophenyl)benzo[d]oxazole-6-carbonyl chloride (150 mg, 0.46 mmol) under an atmosphere of nitrogen. The mixture was stirred at 25° C. for 12 h under an atmosphere of nitrogen. The suspension was filtrated and the filter cake was washed with DCM (35 mL). The solid was dried under vacuum to afford the title compound (66.69 mg, 34% yield, 99.9% purity) as a white solid without further purification.

LCMS: m/z 430.1[M+H]⁺.

¹H NMR (400 MHz, DMSO-d₆) δ=11.87 (bs, 1H), 8.29 (d, J=1.2 Hz, 1H), 8.17 (d, J=2 Hz, 2H), 8.03-8.01 (m, 1H), 7.98-7.94 (m, 2H), 7.00-6.77 (m, 2H), 5.34-5.28 (m, 1H), 3.04 (d, J=6 Hz, 2H), 1.80-1.64 (m, 2H), 0.94 (t, J=7.2 Hz, 3H).

### Example 38

### Compound 38: 1-(1H-pyrazol-5-yl)propan-2-yl 2-(3,5-dichlorophenyl) benzo[d]oxazole-6-carboxylate

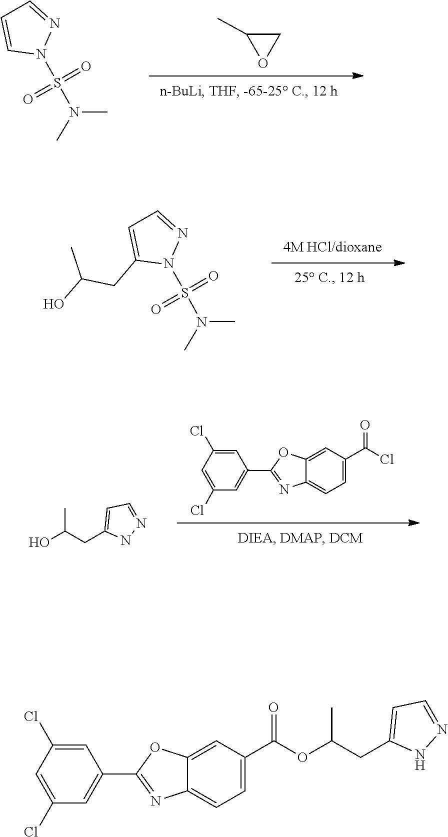

### Step 1: 5-(2-hydroxypropyl)-N,N-dimethyl-1H-pyrazole-1-sulfonamide

To a solution of N,N-dimethylpyrazole-1-sulfonamide (1 g, 5.71 mmol) in THF (10 mL) was added n-BuLi (2.5 M, 2.74 mL) at −65° C. under N₂. The resulting mixture was stirred for 1 h at −65° C. After 1 h, 2-methyloxirane (663 mg, 11.41 mmol) was added. The reaction mixture was warmed to 25° C. slowly and stirred for 12 h. The reaction mixture was quenched with saturated NH₄Cl solution (30 mL) and extracted with ethyl acetate (60 mL). The organic layer was separated and washed with brine (20 mL). It was dried over Na₂SO₄, filtered and then the filtrate was concentrated under reduced pressure. The residue was purified by silica gel column (petroleum ether:EtOAc=10:1 to 1:1) to afford the title compound (530 mg, 39% yield, 97% purity) as colorless oil.

LCMS: m/z 234.0 [M+H]⁺.

### Step 2: 1-(1H-pyrazol-5-yl) propan-2-ol

5-(2-hydroxypropyl)-N,N-dimethyl-pyrazole-1-sulfonamide (500 mg, 2.14 mmol) was dissolved in HCl/dioxane (5 mL) and the reaction mixture was stirred at 25° C. for 12 h. The reaction mixture was concentrated under reduced pressure. The residue was purified by prep-HPLC (column: Phenomenex C18 150×25 mm×10 μm; mobile phase: (water (NH₃·H₂O)-ACN); B %: 0%-24%, 10 min) to afford the title compound (110 mg, 40% yield, 97.9% purity) as a light yellow oil.

LCMS: m/z 127.4 [M+H]⁺.

¹H NMR (400 MHz, DMSO-d₆) δ=12.40-12.25 (m, 1H), 7.43 (bs, 1H), 6.03 (d, J=1.6 Hz, 1H), 4.60 (bs, 1H), 3.86-3.82 (m, 1H), 2.72-2.65 (m, 1H), 2.58-2.53 (m, 1H), 1.03 (d, J=6 Hz, 3H).

### Step 3: 1-(1H-pyrazol-5-yl) propan-2-yl 2-(3,5-dichlorophenyl) benzo[d]oxazole-6-carboxylate

To a solution of 1-(1H-pyrazol-5-yl)propan-2-ol (93 mg, 0.74 mmol) in DCM (2 mL) was added DIEA (237 mg, 1.84 mmol), DMAP (37 mg, 306 mmol) and 2-(3,5-dichlorophenyl)benzo[d]oxazole-6-carbonyl chloride (200 mg, 0.61 mmol). The mixture was stirred at 25° C. for 16 h. The reaction mixture was concentrated under reduced pressure. The residue was purified by flash silica gel chromatography (40 g Silica Flash Column, Eluent of 0-20% R/Petroleum ether @ 80 mL/min, R=(ethyl acetate/ethyl alcohol=3/1)) to afford a crude product. The crude product was purified by prep-HPLC (column: Welch Ultimate XB—SiOH 250 s 50 s 10 μm; mobile phase: [Hexane-EtOH]; B %: 0%-8%, 13 min) to afford the title compound (15.78 mg, 6% yield, 97.8% purity) as a white solid.

LCMS: m/z 416.0 [M+H]⁺.

¹H NMR (400 MHz, DMSO-d₆) δ=8.29 (d, J=0.8 Hz, 1H), 8.18 (d, J=2 Hz, 2H), 8.04-7.95 (m, 3H), 7.58 (d, J=2 Hz, 1H), 6.18 (d, J=2 Hz, 1H), 5.36-5.28 (m, 1H), 3.10-2.99 (m, 2H), 1.36 (d, J=6.4 Hz, 3H).

### Example 39

### Compound 39: 2-hydroxyethyl 2-(3,5-dichlorophenyl)benzo[d]oxazole-6-carboxylate

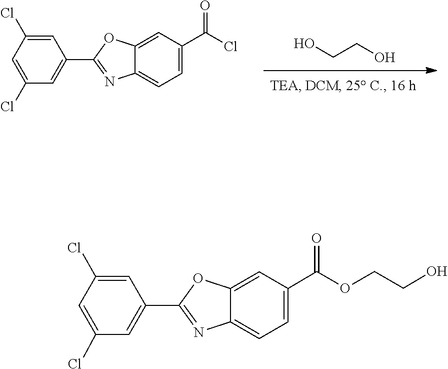

2-(3,5-dichlorophenyl)benzo[d]oxazole-6-carbonyl chloride (100 mg, 0.31 mmol) was added to the solution of ethane-1,2-diol (76 mg, 1.22 mmol) in DCM (2 mL) and TEA (62 mg, 0.61 mmol), then the mixture was stirred for 16 h at 25° C. The mixture was concentrated under reduced pressure. The crude product was purified by column chromatography (SiO₂, Petroleum ether:EtOAc=2:1) to afford the title compound (73.11 mg, 68% yield, 99.6% purity) as a white solid.

LCMS: m/z 352.0 [M+H]⁺

¹H NMR (400 MHz, CDCl₃) δ=8.31 (d, J=1.2 Hz, 1H), 8.22-8.08 (m, 3H), 7.82 (d, J=8.4 Hz, 1H), 7.56 (t, J=2.0 Hz, 1H), 4.59-4.48 (m, 2H), 4.02 (m, 2H).

### Example 40

### Compound 40: 3-hydroxypropyl 2-(3,5-dichlorophenyl)benzo[d]oxazole-6-carboxylate

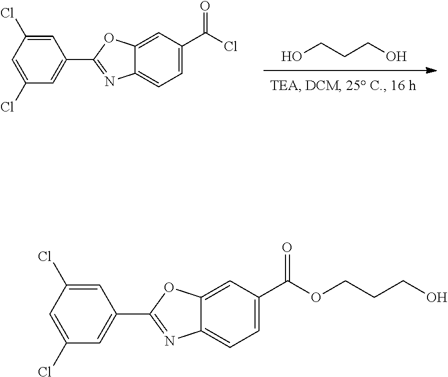 [SMILES: O=C(Cl)c1ccc2nc(-c3cc(Cl)cc(Cl)c3)oc2c1 ; OCCCO ; O=C(OCCCO)c1ccc2nc(-c3cc(Cl)cc(Cl)c3)oc2c1]

2-(3,5-dichlorophenyl)benzo[d]oxazole-6-carbonyl chloride (100 mg, 0.31 mmol) was added to the solution of propane-1,3-diol (70 mg, 0.92 mmol) and TEA (93 mg, 0.92 mmol) in DCM (2 mL) and stirred for 16 h at 25° C. The mixture was concentrated under reduced pressure. The crude product was purified by flash silica gel chromatography (10 g Silica Flash Column, Eluent of 0-30% Ethyl acetate/Petroleum ether gradient @ 60 mL/min) to afford the title compound (80.53 mg, 72% yield, 99.8% purity) as a white solid.

LCMS: m/z 366.0 [M+H]⁺

¹H NMR (400 MHz, CDCl₃) δ=8.28 (d, J=0.8 Hz, 1H), 8.16 (d, J=2.0 Hz, 2H), 8.12 (dd, J=1.6, 8.4 Hz, 1H), 7.81 (d, J=8.4 Hz, 1H), 7.56 (t, J=2.0 Hz, 1H), 4.55 (t, J=6.4 Hz, 2H), 3.82 (t, J=6.0 Hz, 2H), 2.06 (quin, J=6.0 Hz, 2H).

### Example 41

### Compound 41: 4-hydroxybutan-2-yl 2-(3,5-dichlorophenyl) benzo [d] oxazole-6-carboxylate

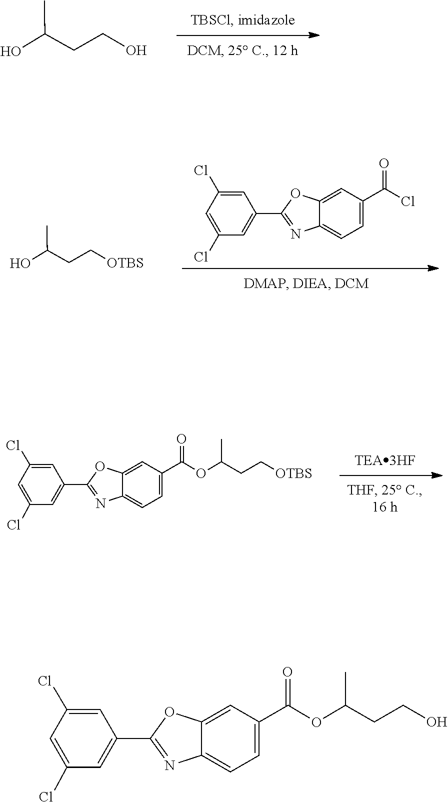

### Step 1: 4-((tert-butyldimethylsilyl) oxy) butan-2-ol

To a solution of butane-1,3-diol (500 mg, 5.55 mmol) in DCM (5 mL) was added imidazole (567 mg, 8.32 mmol) and TBSCl (1.0 g, 6.66 mmol). The mixture was stirred at 25° C. for 12 h. The reaction mixture was quenched by addition water (20 mL) and extracted with dichloromethane 45 mL. The combined organic layers were dried over Na₂SO₄, filtered and concentrated under reduced pressure. The residue was purified by column chromatography (SiO₂, Petroleum ether/Ethyl acetate=1/100 to 10/1) to afford the title compound (811 mg, 72% yield) as a colorless oil.

¹H NMR (400 MHz, CDCl₃) δ=4.06-3.98 (m, 1H), 3.92-3.86 (m, 1H), 3.84-3.78 (m, 1H), 1.70-1.61 (m, 2H), 1.19 (d, J=6.0 Hz, 3H), 0.90 (s, 8H), 0.08 (s, 6H).

### Step 2: 4-((tert-butyldimethylsilyl) oxy) butan-2-yl 2-(3,5-dichlorophenyl) benzo[d]oxazole-6-carboxylate

To a solution of 2-(3,5-dichlorophenyl)benzo[d]oxazole-6-carbonyl chloride (200 mg, 0.61 mmol) in DCM (2 mL) was added DMAP (37 mg, 0.31 mmol), 4-[tert-butyl (dimethyl) silyl] oxybutan-2-ol (188 mg, 0.92 mmol) and DIEA (237 mg, 1.84 mmol). The mixture was stirred at 25° C. for 12 h. The reaction mixture was diluted with water (10 mL) and extracted with dichloromethane (45 mL). The combined organic layers were dried over Na₂SO₄, filtered and concentrated under reduced pressure. The residue was purified by column chromatography (SiO₂, Petroleum ether/Ethyl acetate=1/100 to 10/1) to afford the title compound (288 mg, 95% yield) as a white solid.

LCMS: m/z 494.2 [M+H]⁺.

### Step 3: 4-((tert-butyldimethylsilyl)oxy)butan-2-yl 2-(3,5-dichlorophenyl)benzo-[d]oxazole-6-carboxylate

To a solution of [3-[tert-butyl (dimethyl) silyl] oxy-1-methyl-propyl] 2-(3,5-dichlorophenyl)-1,3-benzoxazole-6-carboxylate (200 mg, 0.40 mmol) in THF (2 mL) was added 3HF.TEA (196 mg, 1.21 mmol). The mixture was stirred at 25° C. for 12 h. The reaction mixture was concentrated under reduced pressure. The residue was purified by prep-HPLC (column: Welch Ultimate XB—NH2 250×50×10 μm; mobile phase: (Hexane-EtOH); B %: 0%-10%, 13 min) to afford the crude product, and the crude product was purified by column chromatography (SiO₂, Petroleum ether/Ethyl acetate=20/1 to 1/1). Then it was purified by prep-TLC (SiO₂, Petroleum ether/Ethyl acetate=1:1) to afford the title compound (28 mg, 18% yield) as a white solid.

LCMS: m/z 380.1 [M+H]⁺.

¹H NMR (400 MHz, CDCl₃) δ=8.31 (s, 1H), 8.19-8.13 (m, 3H), 7.83 (d, J=8.4 Hz, 1H), 7.58 (t, J=2.0 Hz, 1H), 5.47-5.43 (m, 1H), 3.79-3.69 (m, 2H), 2.19-2.16 (m, 1H), 2.06-1.90 (m, 2H), 1.48 (d, J=6.4 Hz, 3H).

### Example 42

### Compound 42: 3-hydroxybutyl 2-(3,5-dichlorophenyl) benzo[d]oxazole-6-carboxylate

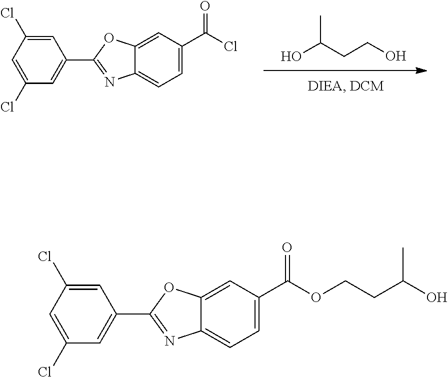

To a solution of butane-1,3-diol (62 mg, 0.69 mmol) in DCM (2 mL) was added DIEA (119 mg, 0.92 mmol) and 2-(3,5-dichlorophenyl)-1,3-benzoxazole-6-carbonyl chloride (150 mg, 0.46 mmol). The mixture was stirred at 25° C. for 16 h. The mixture was concentrated under reduced pressure. The residue was purified by flash silica gel chromatography (20 g Silica Flash Column, Eluent of 0-32% Ethyl acetate/Petroleum ether gradient @ 80 mL/min) to afford the title compound (49.44 mg, 28% yield, 99.8% purity) as a white solid.

LCMS: m/z 380.0 [M+H]⁺

¹H NMR (400 MHz, DMSO-d₆) δ=8.33 (s, 1H), 8.19 (d, J=0.8 Hz, 2H), 8.08-8.05 (m, 1H), 8.00-7.97 (m, 2H), 4.63 (d, J=5.2 Hz, 1H), 4.41-4.38 (m, 2H), 3.87-3.81 (m, 1H), 1.85-1.73 (m, 2H), 1.14 (d, J=6.4 Hz, 3H).

### Example 43 and Example 44

### Compound 43 and Compound 44: 1-hydroxypropan-2-yl 2-(3,5-dichlorophenyl) benzo[d]oxazole-6-carboxylate Both R and S Configuration Isolated

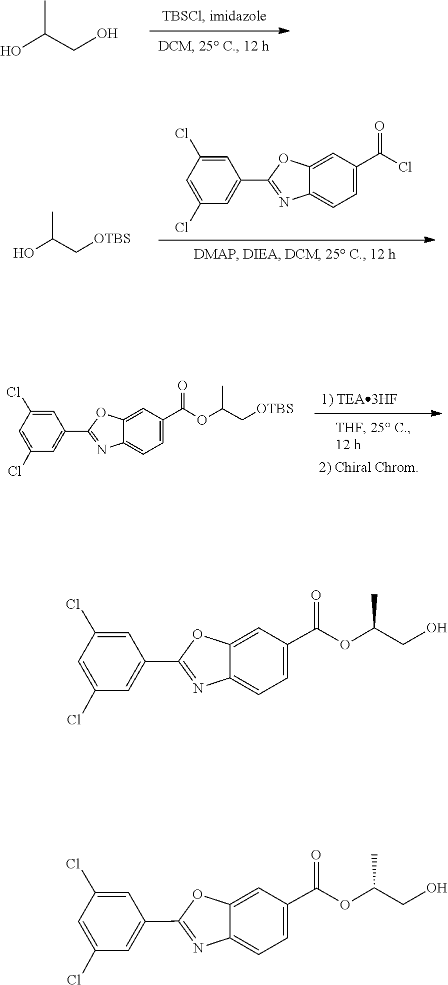

### Step 1: 1-((tert-butyldimethylsilyl) oxy) propan-2-ol

To a solution of propane-1,2-diol (500 mg, 6.57 mmol) in DCM (5 mL) was added imidazole (671 mg, 9.86 mmol) and TBSCl (1.19 g, 7.88 mmol). The mixture was stirred at 25° C. for 12 h. The reaction mixture was quenched by addition water (20 mL) and extracted with dichloromethane (45 mL). The combined organic layers were dried over Na₂SO₄, filtered and concentrated under reduced pressure. The residue was purified by column chromatography (SiO₂, Petroleum ether/Ethyl acetate=I/O to 10/1) to afford the title compound (866 mg, 69% yield) was obtained as a yellow oil.

¹H NMR (400 MHz, CDCl₃) δ=3.78-3.86 (m, 1H), 3.59 (dd, J=3.6, 6.4 Hz, 1H), 3.35 (dd, J=2.0, 8.0 Hz, 1H), 1.13-1.11 (m, 3H), 0.91-0.90 (m, 9H), 0.09-0.08 (m, 1H), 0.07 (s, 5H).

### Step 2: 1-((tert-butyldimethylsilyl) oxy) propan-2-yl 2-(3,5-dichlorophenyl) benzo[d]oxazole-6-carboxylate

To a solution of 2-(3,5-dichlorophenyl)benzo[d]oxazole-6-carbonyl chloride (150 mg, 0.46 mmol) in DCM (6 mL) was added DMAP (56 mg, 0.46 mmol), 1-[tert-butyl(dimethyl)silyl]oxypropan-2-ol (175 mg, 0.92 mmol) and DIEA (178 mg, 1.38 mmol). The mixture was stirred at 25° C. for 12 h. The reaction mixture was quenched by addition water (10 mL) at 25° C. and extracted with dichloromethane (45 mL). The combined organic layers were dried over Na₂SO₄, filtered and concentrated under reduced pressure. The residue was purified by column chromatography (SiO₂, Petroleum ether/Ethyl acetate=100/1 to 20/1) to afford the title compound (110 mg, 26% yield, 52% purity) as a white solid.

LCMS: m/z 481.9 [M+H]⁺.

### Step 3: 1-hydroxypropan-2-yl 2-(3,5-dichlorophenyl) benzo[d]oxazole-6-carboxylate both R and S configuration

To a solution of [2-[tert-butyl (dimethyl) silyl] oxy-1-methyl-ethyl] 2-(3,5-dichlorophenyl)-1,3-benzoxazole-6-carboxylate (100 mg, 0.21 mmol) in THF (1 mL) was added 3HF.TEA (101 mg, 0.62 mmol). The mixture was stirred at 25° C. for 12 h. The reaction mixture was quenched by addition water (10 mL) and extracted with dichloromethane (45 mL). The combined organic layers were dried over Na₂SO₄, filtered and concentrated under reduced pressure. The residue was purified by column chromatography (SiO₂, Petroleum ether/Ethyl acetate=100/1 to 2/1) to give a crude product.

The crude product was further separated by SFC (column: DAICEL CHIRALPAK AD (250 mm×30 mm, 10 μm); mobile phase: (0.1% NH₃H₂O ETOH); B %: 60%-60%, 3.6; 40 min) to afford 1-hydroxypropan-2-yl 2-(3,5-dichlorophenyl) benzo[d]oxazole-6-carboxylate resolved but chirality not determined (33 mg, Ret. Time=0.382 min, 43% yield, 99.6% purity) as a white solid and 1-hydroxypropan-2-yl 2-(3,5-dichlorophenyl) benzo[d]oxazole-6-carboxylate resolved but chirality not determined (34 mg, Ret. Time=0.533 min, 45% yield, 99.8% purity) as a yellow solid.

### Compound 43

LCMS: m/z 366.0 [M+H]⁺.

¹H NMR (400 MHz, CDCl₃) δ=8.30 (d, J=0.8 Hz, 1H), 8.16-8.12 (m, 3H), 7.81 (d, J=8.4 Hz, 1H), 7.56 (t, J=2.0 Hz, 1H), 5.31 (dquin, J=3.6, 6.4 Hz, 1H), 3.88-3.78 (m, 2H), 1.42 (d, J=6.4 Hz, 3H).

### Compound 44

LCMS: m/z 366.0 [M+H]⁺.

¹H NMR (400 MHz, CDCl₃) δ=8.29 (d, J=0.8 Hz, 1H), 8.16-8.12 (m, 3H), 7.81 (d, J=8.4 Hz, 1H), 7.55 (t, J=2.0 Hz, 1H), 5.30 (dquin, J=3.6, 6.4 Hz, 1H), 3.88-3.78 (m, 2H), 1.42 (d, J=6.4 Hz, 3H).

### Example 45

### Compound 45 1-(1,1,1-trifluoropropan-2-yl)pyrrolidin-3-yl 2-(3,5-dichlorophenyl)benzo[d]oxazole-6-carboxylate

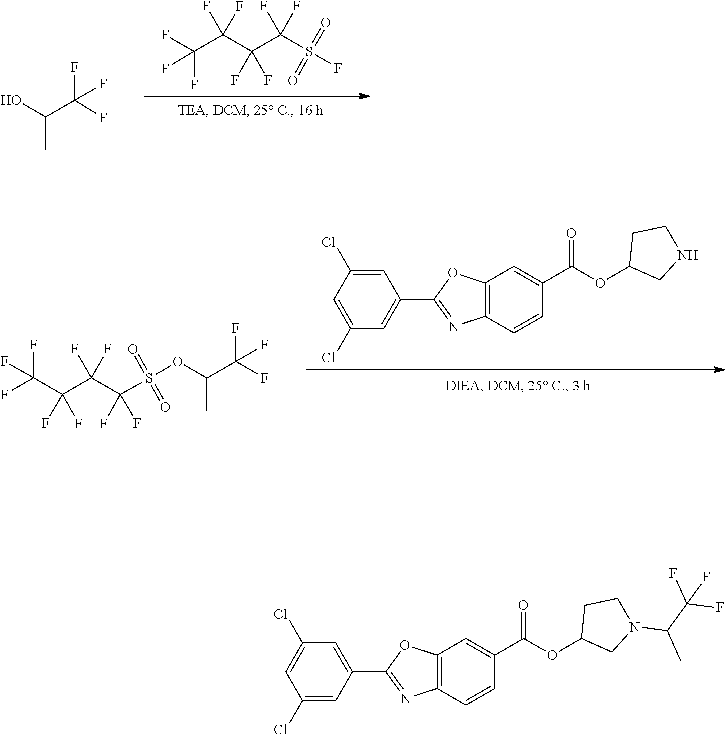

### Step 1: 1,1,1-trifluoropropan-2-yl 1,1,2,2,3,3,4,4,4-nonafluorobutane-1-sulfonate

To a solution of 1,1,1-trifluoropropan-2-ol (680 mg, 5.96 mmol) and TEA (664 mg, 6.56 mmol) in DCM (10 mL) was added 1,1,2,2,3,3,4,4,4-nonafluorobutane-1-sulfonyl fluoride (1.98 g, 6.56 mmol) at 0° C. The mixture was stirred at 30° C. for 16 h. The reaction mixture was concentrated under reduced pressure to afford the title compound (900 mg, crude) as colorless oil, which was used for next step without purification.

### Step 2: 1-(1,1,1-trifluoropropan-2-yl)pyrrolidin-3-yl 2-(3,5-dichlorophenyl)benzo-[d]oxazole-6-carboxylate

Pyrrolidin-3-yl 2-(3,5-dichlorophenyl)-1,3-benzoxazole-6-carboxylate (100 mg, 0.27 mmol), (2,2,2-trifluoro-1-methyl-ethyl) 1,1,2,2,3,3,4,4,4-nonafluorobutane-1-sulfonate (525 mg, 1.33 mmol, 5 eq) and DIEA (343 mg, 2.65 mmol) in DCM (10 mL) was stirred for 3 h at 25° C. The mixture was quenched with water (50 mL) and extracted with EtOAc (30 mL×3). The organic layer was washed with brine (50 mL), dried over Na₂SO₄, filtered and concentrated under reduced pressure. The residue was purified by column chromatography (SiO₂, Petroleum ether:EtOAc=10:1) to afford the title compound (15.5 mg, 95% purity) as a white solid.

LCMS: m/z 472.9 [M+H]⁺.

¹H NMR (400 MHz, CDCl₃) δ=8.27 (s, 1H), 8.16 (s, 2H), 8.12 (d, J=8.4 Hz, 1H), 7.81 (d, J=8.4 Hz, 1H), 7.55 (s, 1H), 5.46 (s, 1H), 3.43-3.20 (m, 2H), 3.12-2.83 (m, 3H), 2.39-2.28 (m, 1H), 2.12-2.02 (m, 1H), 1.31 (d, J=6.8 Hz, 3H).

### Example 46

### Compound 46: Cis-3-(dimethylamino)cyclopentyl 2-(3,5-dichlorophenyl)benzo-[d]oxazole-6-carboxylate—HCl salt

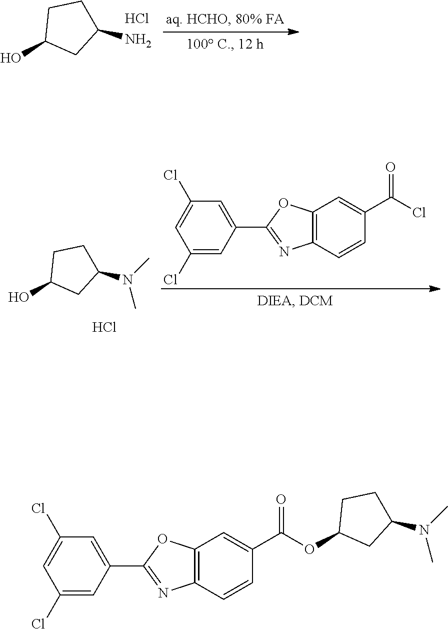

### Step 1: Cis-3-(dimethylamino)cyclopentanol hydrochloride

A solution of cis-3-aminocyclopentanol hydrochloride (200 mg, 1.45 mmol) in HCOOH (2 mL, 80% purity) and HCHO (2.18 g, 72.60 mmol, 2 mL) was heated 100° C. and stirred 12 hrs. The reaction mixture was added 6M HCl (3 mL) and concentrated under reduced pressure. The residue was added 3 mL of EtOAc:EtOH (v:v=3:1) and heated at 80° C. until the mixture turned clear completely, and then the solution was stood for 2 hrs. Solid was precipitated slowly. The solid was collected by filtration and dried over under reduced pressure to afford the title compound (130 mg, 54% yield, HCl salt) as a white solid.

¹H NMR (400 MHz, DMSO-d₆) δ=10.71 (bs, 1H), 4.92 (bs, 1H), 4.06 (quin, J=5.6 Hz, 1H), 3.53-3.43 (m, 1H), 2.68 (d, J=3.6 Hz, 6H), 2.21-2.14 (m, 1H), 1.93-1.87 (m, 2H), 1.71-1.58 (m, 3H).

### Step 2: Cis-3-(dimethylamino)cyclopentyl 2-(3,5-dichlorophenyl)benzo[d]oxazole-6-carboxylate

To a solution of cis-3-(dimethylamino)cyclopentanol hydrochloride (103 mg, 0.62 mmol) in DCM (6 mL) was added DIEA (237.46 mg, 1.84 mmol) and 2-(3,5-dichlorophenyl)benzo[d]oxazole-6-carbonyl chloride (200 mg, 0.61 mmol). The reaction mixture was stirred for 12 hrs at 25° C. The reaction mixture was concentrated under reduced pressure. The residue was purified by prep-HPLC (column: Welch Xtimate C18 150×25 mm×5 μm; mobile phase: (water (0.05% HCl)-ACN); B %: 29%-59%, 10 min) to afford the title compound (50.55 mg, 17% yield, 97.9% purity, HCl) as gray solid.

LCMS: m/z 418.9 [M+H]⁺

¹H NMR (400 MHz, CD₃OD) δ=8.38 (s, 1H), 8.20 (d, J=2.0 Hz, 2H), 8.14 (dd, J=1.2, 8.4 Hz, 1H), 7.85 (d, J=8.4 Hz, 1H), 7.74 (t, J=1.6 Hz, 1H), 5.47-5.42 (m, 1H), 3.74 (quin, J=8.4 Hz, 1H), 2.95 (d, J=2.4 Hz, 6H), 2.84-2.76 (m, 1H), 2.31-2.25 (m, 1H), 2.21-2.15 (m, 1H), 2.13-2.03 (m, 3H).

### Example 47

### Compound 47: Trans-3-(dimethylamino)cyclopentyl 2-(3,5-dichlorophenyl)benzo-[d]oxazole-6-carboxylate—HCl salt

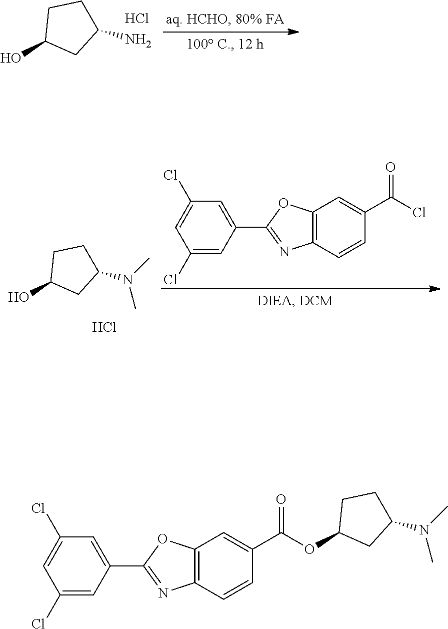

### Step 1: trans-3-(dimethylamino)cyclopentanol hydrochloride

A solution of trans-3-aminocyclopentanol (250 mg, 1.82 mmol, HCl salt) in HCOOH (2 mL, 80% purity) and HCHO (2.73 g, 91 mmol, 2.50 mL) was heated 100° C. and stirred 12 h. The reaction mixture was added 6 M HCl (3 mL) and concentrated under reduced pressure to give the title compound (330 mg, HCl salt) as yellow solid, which was used for next step without purification.

¹H NMR (400 MHz, DMSO-d₆) δ=10.63 (bs, 1H), 4.78 (bs, 1H), 4.21-4.18 (m, 1H), 3.73-3.61 (m, 1H), 2.70 (t, J=4.2 Hz, 6H), 1.96-1.72 (m, 4H), 1.55-1.48 (m, 1H).

### Step 2: trans-3-(dimethylamino)cyclopentyl 2-(3,5-dichlorophenyl)benzo-[d]oxazole-6-carboxylate

To a solution of trans-3-(dimethylamino)cyclopentanol (203 mg, 1.22 mmol, HCl salt) in DCM (6 mL) was added DIEA (237 mg, 1.84 mmol) and 2-(3,5-dichlorophenyl)benzo[d]oxazole-6-carbonyl chloride (200 mg, 0.61 mmol). The reaction mixture was stirred for 12 h at 25° C. The reaction mixture was concentrated under reduced pressure. The residue was purified by prep-HPLC (column: Phenomenex Luna C18 150×25 mm×10 μm; mobile phase: (water (0.05% HCl)-ACN); B %: 30%-60%, 10 min) to afford the title compound HCl salt (29.19 mg, 11% yield, 93% purity) as white solid.

LCMS: m/z 418.9 [M+H]⁺.

¹H NMR (400 MHz, CD₃OD) δ=8.36-8.31 (m, 1H), 8.18 (d, J=1.9 Hz, 2H), 8.14-8.08 (m, 1H), 7.83 (d, J=8.4 Hz, 1H), 7.72 (t, J=2.0 Hz, 1H), 5.50-5.47 (m, 1H), 3.41-3.35 (m, 1H), 2.58 (s, 6H), 2.41-2.22 (m, 3H), 2.05-1.94 (m, 2H), 1.76-1.67 (m, 1H).

### Example 48

### Compound 48: 3,3-dimethylcyclopentyl 2-(3,5-dichlorophenyl)benzo[d]oxazole-6-carboxylate

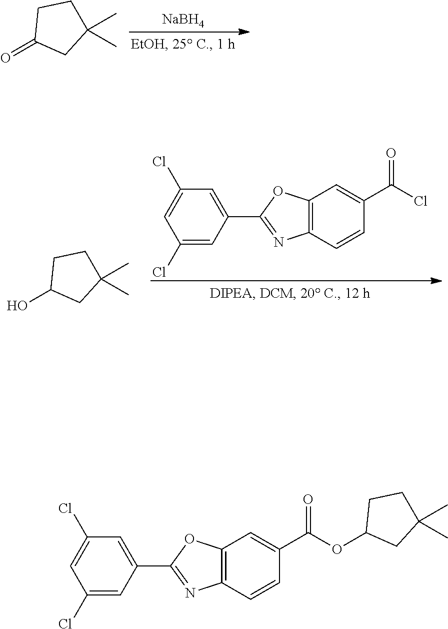

### Step 1: 3,3-dimethylcyclopentanol

To a solution of 3,3-dimethylcyclopentanol (300 mg, 2.67 mmol) in THF (5 mL) was added NaBH₄ (202 mg, 5.35 mmol), following by EtOH (0.5 mL). The mixture was stirred for 2 hrs at 20° C. The mixture was quenched with water (10 mL) and extracted with EtOAc (20 mL×3). The organic layer was washed with brine (50 mL), dried over Na₂SO₄, filtered and concentrated under reduced pressure to afford the title compound (300 mg, 98% yield) as colorless oil, which was used for next step without purification.

¹H NMR (400 MHz, CDCl₃) δ=4.45-4.32 (m, 1H), 2.04-1.96 (m, 1H), 1.84-1.74 (m, 1H), 1.69-1.58 (m, 2H), 1.47-1.33 (m, 2H), 1.13-1.11 (m, 3H), 1.00-0.94 (m, 3H)

### Step 2: 3,3-dimethylcyclopentyl 2-(3,5-dichlorophenyl)benzo[d]oxazole-6-carboxylate

2-(3,5-dichlorophenyl)benzo[d]oxazole-6-carbonyl chloride (100 mg, 0.31 mmol) was added to the solution of 3,3-dimethylcyclopentanol (42 mg, 0.38 umol) and DIPEA (198 mg, 1.53 mmol) in DCM (2 mL) and stirred for 12 hrs at 20° C. The mixture was quenched with water (30 mL) and extracted with EtOAc (30 mL×3). The combined organic layer was washed with brine (50 mL), dried over Na₂SO₄, filtered and concentrated under reduced pressure. The residue was purified by prep-HPLC (column: Welch Ultimate XB-Diol 250×50×10 μm; mobile phase: (Hexane-EtOH, neutral); B %: 0%-10%, 13 min), following by prep-TLC (SiO₂, EtOAc:petroleum ether=1:10) to afford the title compound (13.45 mg, 10.33% yield, 95.1% purity) as a white solid.

LCMS: m/z 404.0 [M+H]⁺

¹H NMR (400 MHz, CDCl₃) δ=8.28 (d, J=0.8 Hz, 1H), 8.18 (d, J=2.0 Hz, 2H), 8.12 (dd, J=1.2, 8.4 Hz, 1H), 7.82 (d, J=8.4 Hz, 1H), 7.57 (t, J=2.0 Hz, 1H), 5.52-5.47 (m, 1H), 2.30-2.21 (m, 1H), 2.02-1.91 (m, 2H), 1.78-1.68 (m, 2H), 1.53-1.46 (m, 1H), 1.20 (s, 3H), 1.08 (s, 3H)

### Example 49

### Compound 49: 1-(pyridin-3-yl)pyrrolidin-3-yl 2-(3,5-dichlorophenyl)benzo-[d]oxazole-6-carboxylate

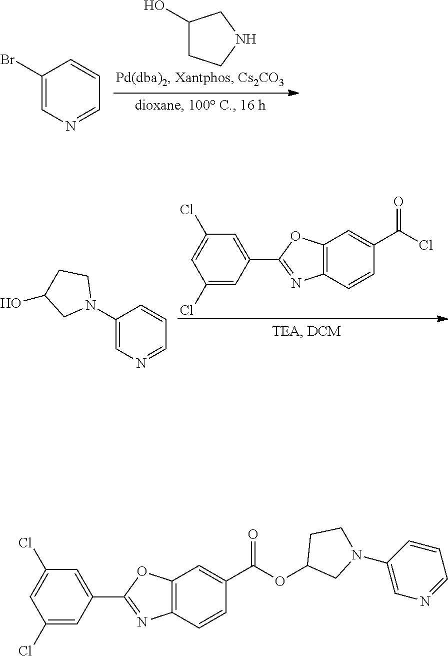

### Step 1: 1-(pyridin-3-yl)pyrrolidin-3-ol

To a solution of 3-bromopyridine (2.72 g, 17.22 mmol, 3 eq) in dioxane (10 mL) was added pyrrolidin-3-ol (500 mg, 5.74 mmol), Pd(dba)₂ (330 mg, 0.57 mmol), Cs₂CO₃ (3.74 g, 11.48 mmol) and Xantphos (664 mg, 1.15 mmol). The reaction mixture was stirred at 90° C. for 12 h. The reaction mixture was concentrated under reduced pressure to give a residue. The residue was purified by silica gel column (DCM:MeOH=100:1 to 30:1) to give 1-(3-pyridyl)pyrrolidin-3-ol (210 mg, 1.28 mmol, 22.28% yield) as yellow oil.

¹H NMR (400 MHz, CD₃OD) δ=7.82 (d, J=2.8 Hz, 1H), 7.78-7.77 (m, 1H), 7.19 (dd, J=4.8, 8.4 Hz, 1H), 6.98-6.96 (m, 1H), 4.55-4.52 (m, 1H), 3.50-3.41 (m, 2H), 3.37-3.32 (m, 2H), 3.22-3.19 (m, 1H), 2.19-2.11 (m, 1H), 2.07-2.01 (m, 1H).

### Step 2: 1-(pyridin-3-yl)pyrrolidin-3-yl 2-(3,5-dichlorophenyl)benzo[d]oxazole-6-carboxylate

To a solution of 1-(3-pyridyl)pyrrolidin-3-ol (150 mg, 0.92 mmol) in DCM (6 mL) was added DIEA (178 mg, 1.38 mmol), DMAP (56 mg, 0.46 mmol) and 2-(3,5-dichlorophenyl)benzo[d] oxazole-6-carbonyl chloride (150 mg, 0.46 mmol). The reaction mixture was stirred for 12 h at 25° C. The reaction mixture was concentrated under reduced pressure. The residue was purified by silica gel column (DCM:MeOH=100:1 to 20:1) to afford the title compound (33.87 mg, 16% yield, 97% purity) as yellow solid.

LCMS: m/z 453.9 [M+H]⁺

¹H NMR (400 MHz, CDCl₃) δ=8.26 (d, J=0.8 Hz, 1H), 8.16 (d, J=2.0 Hz, 2H), 8.10 (dd, J=1.6, 8.4 Hz, 1H), 8.05 (d, J=3.2 Hz, 1H), 8.00 (d, J=4.0 Hz, 1H), 7.81 (d, J=8.4 Hz, 1H), 7.56 (t, J=2.0 Hz, 1H), 7.25-7.23 (m, 1H), 6.98-6.96 (m, 1H), 5.79-5.75 (m, 1H), 3.81 (dd, J=5.2, 11.2 Hz, 1H), 3.65-3.54 (m, 3H), 2.47-2.42 (m, 2H).

### Example 50

### Compound 50: 1-(pyridin-4-yl)pyrrolidin-3-yl 2-(3,5-dichlorophenyl)benzo-[d]oxazole-6-carboxylate

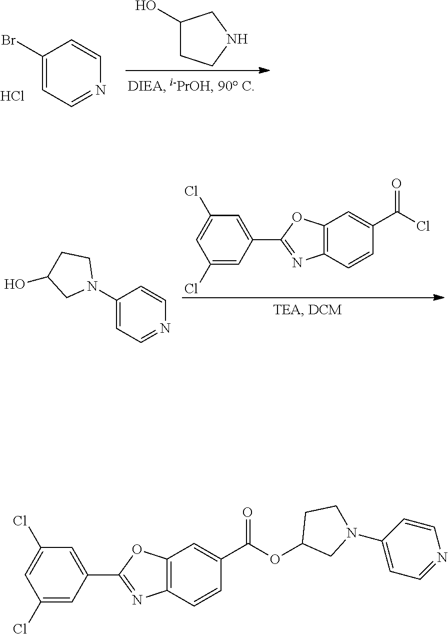

### Step 1: 1-(pyridin-4-yl)pyrrolidin-3-ol

To a solution of pyrrolidin-3-ol (500 mg, 5.74 mmol) and 4-chloropyridine (1.30 g, 11.48 mmol) in i-PrOH (10 mL) was added DIEA (2.97 g, 22.96 mmol). The reaction mixture was heated at 90° C. and stirred for 12 h. The desired mass was detected. The reaction mixture was concentrated under reduced pressure to give a residue. The residue was purified by reverse phase flash (column: Welch Ultimate XB C18 20-40 μm; 120 A; mobile phase: (water (0.1% NH₃·H₂O)-ACN); B %: 5-30%, 30 min) to afford the title compound (71 mg, 7% yield, 97% purity) as yellow solid.

¹H NMR (400 MHz, CD₃OD) δ=8.03 (d, J=6.8 Hz, 2H), 6.53-6.51 (m, 2H), 4.56-4.52 (m, 1H), 3.52-3.40 (m, 3H), 3.26 (bs, 1H), 2.19-2.12 (m, 1H), 2.09-2.03 (m, 1H).

### Step 2: 1-(pyridin-4-yl)pyrrolidin-3-yl 2-(3,5-dichlorophenyl)benzo[d]oxazole-6-carboxylate

To a solution of 1-(pyridin-4-yl)pyrrolidin-3-ol (71 mg, 0.43 mmol) in DCM (6 mL) was added DIEA (178 mg, 1.38 mmol), DMAP (56 mg, 0.46 mmol) and 2-(3,5-dichlorophenyl)benzo[d] oxazole-6-carbonyl chloride (150 mg, 0.46 mmol). The reaction mixture was stirred for 12 h at 25° C. The reaction mixture was concentrated under reduced pressure to give a residue. The residue was purified by prep-HPLC (column: Welch Ultimate XB-Diol 250×50×10 μm; mobile phase: (Hexane-EtOH, neutral); B %: 5%-55%, 20 min) to afford the title compound (38.73 mg, 18% yield, 99% purity) as off-white solid.

LCMS: m/z 453.9 [M+H]⁺

¹H NMR (400 MHz, CDCl₃) δ=8.26-8.25 (m, 3H), 8.15 (d, J=1.6 Hz, 2H), 8.09 (dd, J=1.6, 8.4 Hz, 1H), 7.81 (d, J=8.4 Hz, 1H), 7.56 (t, J=2.0 Hz, 1H), 6.47 (br d, J=6.4 Hz, 2H), 5.78-5.74 (m, 1H), 3.82-3.78 (m, 1H), 3.64-3.60 (m, 3H), 2.46-2.41 (m, 2H).

### Example 51

### Compound 51: 1-(pyrimidin-2-yl)pyrrolidin-3-yl 2-(3,5-dichlorophenyl)benzo-[d]oxazole-6-carboxylate

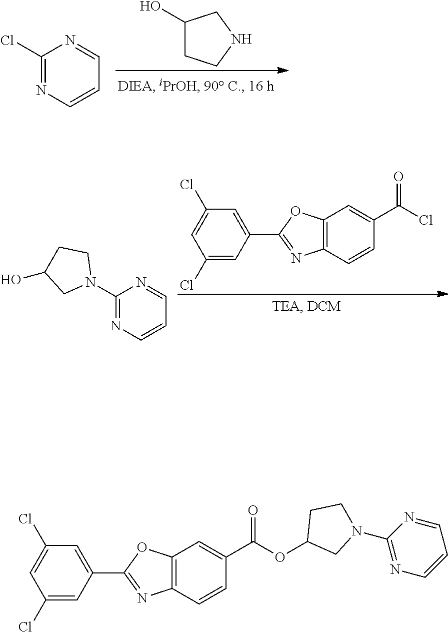

### Step 1: 1-(pyrimidin-2-yl)pyrrolidin-3-ol

To a solution of 2-chloropyrimidine (500 mg, 4.37 mmol) and pyrrolidin-3-ol (456 mg, 5.24 mmol) in i-PrOH (10 mL) was added DIEA (1.13 g, 8.73 mmol) at 25° C. The reaction mixture was heated at 90° C. and stirred for 16 h. The mixture was concentrated under reduced pressure. The residue was purified by silica gel column chromatography (DCM:MeOH=100:1 to 60:1) to afford the title compound (280 mg, 39% yield) as white solid.

¹H NMR (400 MHz, DMSO-d₆) δ=8.31 (d, J=4.8 Hz, 2H), 6.56 (t, J=4.8 Hz, 1H), 4.94 (bs, 1H), 4.36 (bs, 1H), 3.56-3.52 (m, 4H), 3.51-3.50 (m, 1H), 1.99 (dtd, J=4.5, 8.7, 13.0 Hz, 1H), 1.90-1.87 (m, 1H).

### Step 2:1-(pyrimidin-2-yl)pyrrolidin-3-yl 2-(3,5-dichlorophenyl)benzo[d]oxazole-6-carboxylate

To a solution of 2-(3,5-dichlorophenyl)benzo[d]oxazole-6-carbonyl chloride (150 mg, 0.46 mmol) and 1-pyrimidin-2-ylpyrrolidin-3-ol (113.82 mg, 0.69 mmol) in DCM (6 mL) was added DIEA (297 mg, 2.30 mmol) and DMAP (5.61 mg, 0.05 mmol) at 25° C. The reaction mixture was stirred for 12 h at 25° C. The reaction mixture was concentrated under reduced pressure. The crude product was purified by silica gel column chromatography (DCM:MeOH=100:1) to afford the title compound (34.39 mg, 16% yield, 98.2% purity) as a white solid.

LCMS: m/z 454.9 [M+H]⁺.

¹H NMR (400 MHz, CDCl₃) δ=8.35 (d, J=4.8 Hz, 2H), 8.27 (d, J=0.8 Hz, 1H), 8.15 (d, J=2.0 Hz, 2H), 8.10 (dd, J=1.6, 8.4 Hz, 1H), 7.79 (d, J=8.4 Hz, 1H), 7.55 (t, J=2.0 Hz, 1H), 6.54 (t, J=4.8 Hz, 1H), 5.77-5.73 (m, 1H), 3.98-3.89 (m, 3H), 3.78-3.76 (m, 1H), 2.41-2.36 (m, 2H), 1.58 (s, 2H).

### Example 52

### Compound 52: 1-(pyrazin-2-yl)pyrrolidin-3-yl 2-(3,5-dichlorophenyl)benzo-[d]oxazole-6-carboxylate

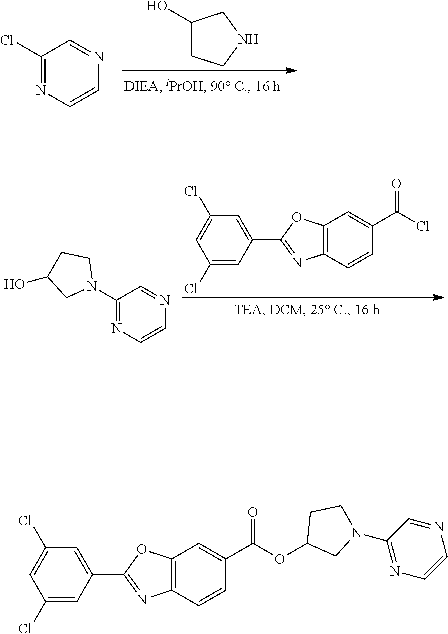

### Step 1: 1-(pyrazin-2-yl)pyrrolidin-3-ol

To a solution of 2-chloropyrazine (1 g, 8.73 mmol, 0.78 mL) and pyrrolidin-3-ol (913 mg, 10.48 mmol) in ⁱPrOH (20 mL) was added DIEA (2.26 g, 17.46 mmol) at 25° C. The reaction mixture was heated at 90° C. and stirred for 16 h. The mixture was concentrated under reduced pressure. The residue was purified by flash silica gel chromatography (20 g SepaFlash® Silica Flash Column, Eluent of 0-10% MeOH/DCM @ 20 mL/min) to afford the title compound (800 mg, 55% yield, 99.0% purity) as yellow solid.

1H NMR (400 MHz, DMSO-d₆) δ=8.01 (dd, J=1.6, 2.8 Hz, 1H), 7.92 (d, J=1.6 Hz, 1H), 7.73 (d, J=2.8 Hz, 1H), 4.99 (d, J=4.0 Hz, 1H), 4.42-4.38 (m, 1H), 3.51-3.35 (m, 4H), 2.04-1.88 (m, 2H).

### Step 2: 1-(pyrazin-2-yl)pyrrolidin-3-yl 2-(3,5-dichlorophenyl)benzo[d]oxazole-6-carboxylate

To a solution of 2-(3,5-dichlorophenyl)benzo[d]oxazole-6-carbonyl chloride (150 mg, 0.46 mmol) and 1-pyrazin-2-ylpyrrolidin-3-ol (190 mg, 1.15 mmol) in DCM (10 mL) was added DIEA (296 mg, 2.30 mmol) and DMAP (56 mg, 0.46 mmol) at 25° C. The reaction mixture was stirred for 16 h at 25° C. The mixture was concentrated under reduced pressure. The residue was purified by flash silica gel chromatography (20 g SepaFlash® Silica Flash Column, Eluent of 0-10% MeOH/DCM @ 20 mL/min) to afford the title compound (91.19 mg, 43% yield, 98.0% purity) as white solid.

LCMS: m/z 454.9 [M+H]⁺.

¹H NMR (400 MHz, CDCl₃) δ=8.26 (d, J=1.2 Hz, 1H), 8.16 (d, J=2.0 Hz, 2H), 8.10 (dd, J=1.6, 8.4 Hz, 2H), 7.97 (bs, 1H), 7.86 (bs, 1H), 7.81 (d, J=8.4 Hz, 1H), 7.56 (t, J=2.0 Hz, 1H), 5.79-5.78 (m, 1H), 3.91-3.73 (m, 4H), 2.46-2.41 (m, 2H).

### Example 53

### Compound 53: 1-(pyrimidin-4-yl)pyrrolidin-3-yl 2-(3,5-dichlorophenyl)benzo-[d]oxazole-6-carboxylate

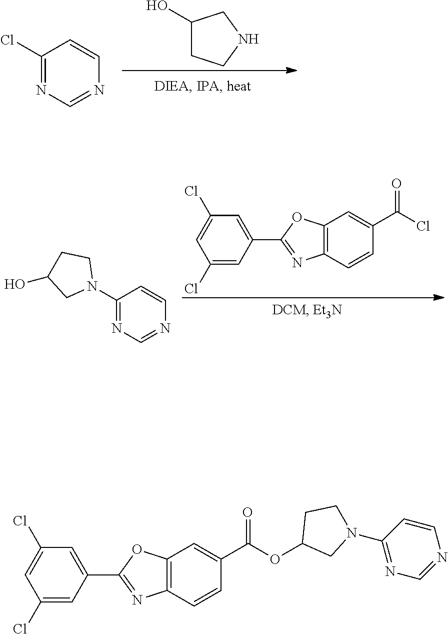

### Step 1: 1-(pyrimidin-4-yl)pyrrolidin-3-ol

To a solution of 4-chloropyrimidine (480 mg, 4.19 mmol) and pyrrolidin-3-ol (438 mg, 5.03 mmol) in iPrOH (10 mL) was added DIEA (1.08 g, 8.38 mmol) at 25° C. The reaction mixture was heated at 90° C. and stirred for 16 h. The mixture was concentrated under reduced pressure. The residue was purified by flash silica gel chromatography (20 g SepaFlash® Silica Flash Column, Eluent of 0-10% Methyl alcohol/Dichloromethane @ 20 mL/min) to afford the title compound (469 mg, 66% yield, 97.6% purity) as brown solid.

¹H NMR (400 MHz, CD₃OD) δ=8.42 (s, 1H), 8.09 (d, J=6.4 Hz, 1H), 6.54 (bs, 1H), 4.54 (bs, 1H), 3.76-3.35 (m, 5H), 2.13-2.08 (m, 2H).

### Step 2: 1-(pyrimidin-4-yl)pyrrolidin-3-yl 2-(3,5-dichlorophenyl)benzo[d]oxazole-6-carboxylate

To a solution of 2-(3,5-dichlorophenyl)benzo[d]oxazole-6-carbonyl chloride (200 mg, 0.61 mmol) and 1-pyrimidin-4-ylpyrrolidin-3-ol (202 mg, 1.22 mmol) in DCM (10 mL) was added DIEA (396 mg, 3.06 mmol) and DMAP (7.48 mg, 0.06 mmol) at 25° C. The reaction mixture was stirred for 16 h at 25° C. LCMS showed the starting material was consumed, the desired mass was detected. The suspension was filtrated and the filter cake was washed with DCM (20 mL×3), and concentrated under reduced pressure. The residue was purified by prep-HPLC (column: Welch Ultimate XB—NH2 250×50×10 μm; mobile phase: (Hexane-EtOH, neutral); B %: 0%-30%, 13 min) to afford the title compound (29.60 mg, 11% yield, 99.2% purity) as white solid.

LCMS: m/z 455.2 [M+H]⁺.

¹H NMR (400 MHz, CDCl₃) δ=8.66 (s, 1H), 8.26 (s, 1H), 8.21 (bd, J=5.6 Hz, 1H), 8.16 (d, J=2.0 Hz, 2H), 8.09 (dd, J=1.2, 8.4 Hz, 1H), 7.81 (d, J=8.4 Hz, 1H), 7.57-7.56 (m, 1H), 6.42 (bd, J=2.8 Hz, 1H), 5.77 (bs, 1H), 4.15-3.74 (m, 4H), 2.51-2.44 (m, 2H).

### Example 54

### Compound 54: 1-(pyridazin-3-yl)pyrrolidin-3-yl 2-(3,5-dichlorophenyl)benzo-[d]oxazole-6-carboxylate

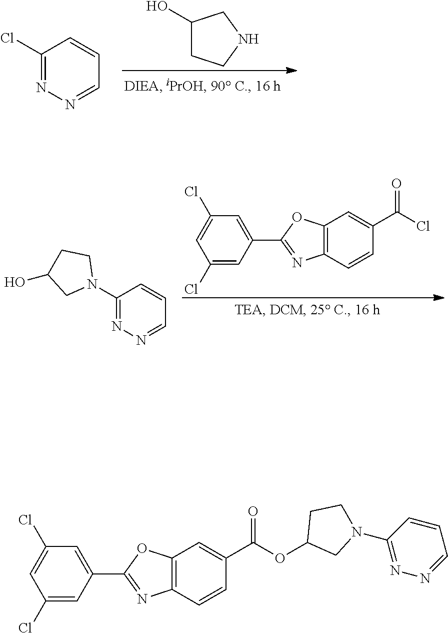

### Step 1: 1-(pyridazin-3-yl)pyrrolidin-3-ol

To the mixture of 3-chloropyridazine (300 mg, 2.62 umol HCl) and DIPEA (770 mg, 1.53 mmol) in ⁱPrOH (2 mL) was added pyrrolidin-3-ol (260 mg, 3.93 mmol) and stirred for 16 h at 80° C. The mixture was concentrated under reduced pressure. The residue was purified by column chromatography (SiO₂, DCM:MeOH=100:1 to 10:1) to afford the title compound (250 mg, 76% yield) as a white solid.

LCMS: m/z 166.2 [M+H]⁺

¹H NMR (400 MHz, CDCl₃) δ=8.40 (s, 1H), 7.23-6.91 (m, 1H), 6.60 (s, 1H), 4.60 (d, J=3.2 Hz, 1H), 3.70-3.49 (m, 4H), 1.44 (d, J=6.4 Hz, 1H), 1.36 (d, J=6.4 Hz, 1H)

### Step 2: 1-(pyridazin-3-yl)pyrrolidin-3-yl 2-(3,5-dichlorophenyl)benzo[d]oxazole-6-carboxylate

2-(3,5-dichlorophenyl)benzo[d]oxazole-6-carbonyl chloride (150 mg, 0.45 mmol) was added to the solution of 1-(pyridazin-3-yl)pyrrolidin-3-ol (150 mg, 0.90 mmol) and TEA (140 mg, 1.36 mmol) in DCM (6 mL) and stirred for 16 h at 25° C. The mixture was quenched with water (10 mL) and extracted with EtOAc (10 mL×3), the organic was washed with brine (20 mL), dried over Na₂SO₄, filtered and concentrated. The residue was purified by column chromatography (SiO₂, DCM:MeOH=5:1) to afford the title compound (81.16 mg, 37.7% yield, 96.7% purity) as a white solid.

LCMS: m/z 455.1 [M+H]⁺

¹H NMR (400 MHz, CDCl₃) δ=8.63 (d, J=4.4 Hz, 1H), 8.27 (s, 1H), 8.17 (d, J=1.6 Hz, 2H), 8.10 (d, J=8.0 Hz, 1H), 7.82 (d, J=8.4 Hz, 1H), 7.61-7.47 (m, 2H), 7.02 (d, J=8.4 Hz, 1H), 5.82 (s, 1H), 4.04-3.87 (m, 4H), 2.59-2.45 (m, 2H)

### Example 55

### Compound 55: Octahydroindolizin-1-yl 2-(3,5-dichlorophenyl)benzo[d]oxazole-6-carboxylate

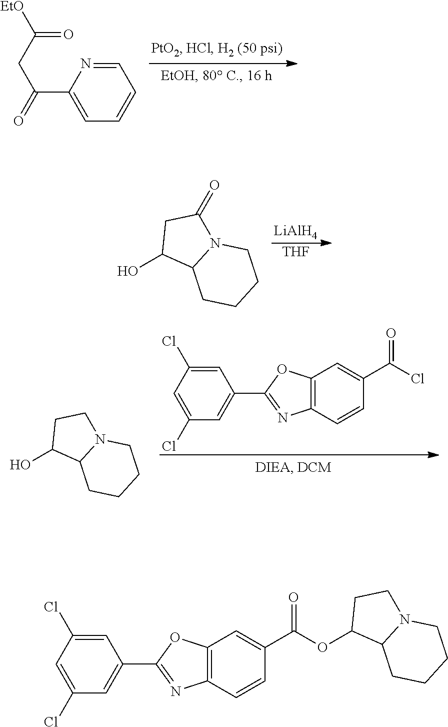

### Step 1: 1-hydroxyhexahydroindolizin-3(2H)-one

To a solution of ethyl 3-oxo-3-(2-pyridyl)propanoate (2 g, 10.35 mmol) in EtOH (40 mL) was added HCl (104.85 mg, 1.04 mmol, 36% purity) and PtO₂ (470.14 mg, 2.07 mmol) under N₂. The suspension was degassed under reduced pressure and purged with H₂ for three times. The resulting mixture was stirred under H₂ (50 Psi) for 12 h at 80° C. The reaction mixture was filtered through a pad of celite and washed with EtOH (10 mL×3). The filtrate was concentrated under reduced pressure. The residue was purified by silica gel column (DCM:MeOH=100:1 to 30:1) to afford the title compound (1.1 g, 68% yield) as yellow oil.

¹H NMR (400 MHz, CDCl₃) δ=4.40-4.05 (m, 2H), 3.44-3.26 (m, 1H), 2.84-2.61 (m, 3H), 2.39-2.33 (m, 1H), 2.05-1.65 (m, 3H), 1.45-1.05 (m, 3H).

### Step 2: octahydroindolizin-1-ol

To a solution of 1-hydroxyhexahydroindolizin-3(2H)-one (500 mg, 3.22 mmol) in THF (10 mL) was added LiAlH₄ (306 mg, 8.05 mmol) at 0° C. The reaction mixture was warmed to 25° C. and stirred for 12 h. The reaction mixture was quenched with Na₂SO₄·10H₂O (200 mg) at 0° C. The mixture was filtered and the filtrated was concentrated under reduced pressure to the title compound (345 mg, 76% yield) as yellow oil, which was used for next step without purification.

### Step 3: octahydroindolizin-1-yl 2-(3,5-dichlorophenyl)benzo[d]oxazole-6-carboxylate

To a solution of octahydroindolizin-1-ol (130 mg, 0.92 mol) in DCM (6 mL) was added DIEA (178 mg) and 2-(3,5-dichlorophenyl)benzo[d]oxazole-6-carbonyl chloride (150 mg, 0.46 mmol). The reaction mixture was stirred for 12 h at 25° C. The reaction mixture was concentrated under reduced pressure. The residue was purified by prep-HPLC (column: Welch Ultimate XB-Diol 250×50×10 μm; mobile phase: (Hexane-EtOH); B %: 0%-25%, 20 min) to afford the title compound (13.37 mg, 6% yield, 96.0% purity) as white solid.

LCMS: m/z 431.0 [M+H]⁺

¹H NMR (400 MHz, CD₃OD) δ=8.32 (d, J=0.8 Hz, 1H), 8.19 (d, J=2.0 Hz, 2H), 8.10 (dd, J=1.2, 8.4 Hz, 1H), 7.83 (d, J=8.4 Hz, 1H), 7.71 (t, J=2.0 Hz, 1H), 5.06-5.01 (m, 1H), 3.12-3.02 (m, 2H), 2.52-2.41 (m, 2H), 2.29-2.17 (m, 2H), 2.03-2.00 (m, 1H), 1.88-1.83 (m, 1H), 1.82-1.75 (m, 1H), 1.71-1.68 (m, 1H), 1.63-1.56 (m, 1H), 1.41-1.33 (m, 2H).

### Example 56

### Compound 56: 3-(3-phenylpropyl)-8-(pyrimidin-2-yl)-1,3,8-triazaspiro [4.5]decane-2,4-dione

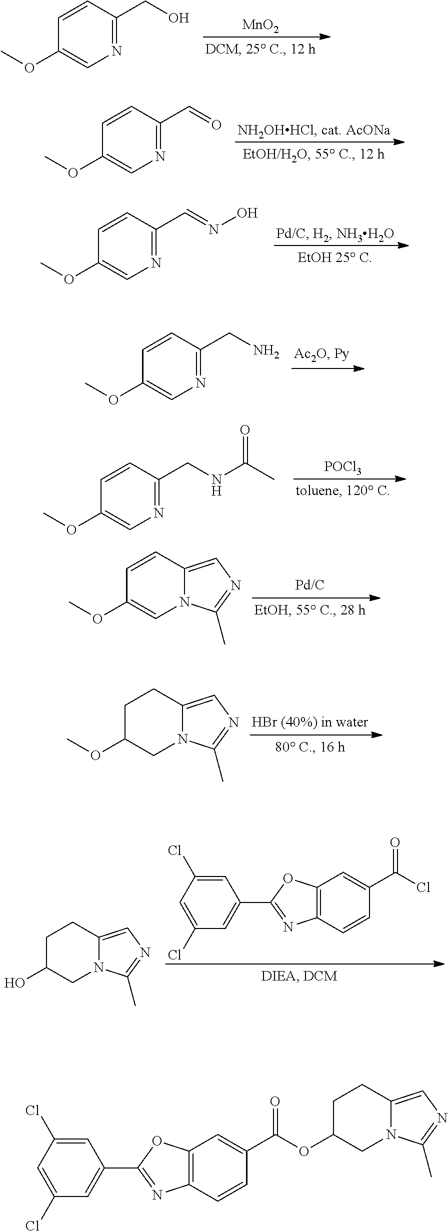

### Step 1: 5-methoxypicolinaldehyde

To a solution of (5-methoxy-2-pyridyl) methanol (9.3 g, 66.83 mmol) in DCM (100 mL) was added MnO₂ (29.05 g, 334.17 mmol) at 25° C. The reaction mixture was stirred for 16 h at 25° C. The suspension was filtrated through a pad of celite and the filter cake was washed with EtOAc (50 mL×3). The filtrate was concentrated under reduced pressure. The residue was purified by flash silica gel chromatography (40 g Silica Flash Column, Eluent of 0-100% EtOAc/Petroleum ether gradient @ 80 mL/min) to afford the title compound (4.6 g, 50% yield, 100% purity) as a white solid.

LCMS: m/z 138.0 [M+H]⁺.

### Step 2: 5-methoxypicolinaldehyde oxime

To a solution of 5-methoxypyridine-2-carbaldehyde (4.6 g, 33.54 mmol) in ethanol (44 mL) and water (22 mL) was added hydroxylamine hydrochloride (2.80 g, 40.25 mmol) and sodium acetate (275 mg, 3.35 mmol) at 25° C. The reaction mixture was heated at 55° C. and stirred for 12 h. The mixture was concentrated under reduced pressure to afford the title compound (6.8 g, 96% yield) as a white solid.

LCMS: m/z 153.1 [M+H]⁺.

### Step 3: (5-methoxypyridin-2-yl) methanamine

To a solution of 5-methoxypyridine-2-carbaldehyde oxime (10 g, 65.72 mmol) in EtOH (100 mL) was added Pd/C (1 g) and NH₃·H₂O (45.50 g, 428.44 mmol, 50 mL) under N₂. The reaction mixture was degassed under reduced pressure and purged with H₂ for three times. The reaction mixture was stirred for 36 h under H₂ (50 Psi) at 25° C. The reaction mixture was filtered through a pad of celite and washed with EtOH (10 mL×3). The filtrate was concentrated under reduced pressure to afford the title compound (10 g, crude) as a white solid.

LCMS: m/z 139.2 [M+H]⁺.

### Step 4: N-((5-methoxypyridin-2-yl) methyl) acetamide

To a solution of (5-methoxy-2-pyridyl) methanamine (10 g, 72.38 mmol) in Py (100 mL) was added Ac₂O (14.78 g, 144.75 mmol, 13.56 mL) at 0° C. The reaction mixture was warmed to 25° C. and stirred for 6 h. The reaction mixture was concentrated under reduced pressure. The residue was purified by silica gel column (DCM:MeOH=50:1 to 20:1) to afford the title compound (2.0 g, 15% yield) as yellow oil.

LCMS: m/z 181.2 [M+H]⁺.

### Step 5: 6-methoxy-3-methylimidazo [1,5-a] pyridine

To a solution of N-[(5-methoxy-2-pyridyl) methyl] acetamide (2.0 g, 11.10 mmol) in toluene (40 mL) was added POCl₃ (8.51 g, 55.49 mmol). The reaction mixture was heated at 120° C. and stirred for 2 h. The reaction mixture was quenched with saturated NaHCO₃ solution (60 mL), extracted with ethyl acetate (100 mL). The organic layer was separated and washed with brine (30 mL), and dried over Na₂SO₄, filtered and then the filtrate was concentrated under reduced pressure. The residue was purified by silica gel column (DCM MeOH=10:1 to 50:1) to afford the title compound (860 mg, 48% yield) as black brown oil.

LCMS: m/z 163.1 [M+H]⁺.

### Step 6: 6-methoxy-3-methyl-5,6,7,8-tetrahydroimidazo [1,5-a] pyridine

To a solution of 6-methoxy-3-methyl-imidazo [1,5-a]pyridine (398 mg, 2.45 mmol) in EtOH (5 mL) was added Pd/C (50 mg, 0.18 mmol) under N₂. The reaction mixture was degassed under reduced pressure and purged with H₂ for three times. The reaction mixture was stirred under H₂ (50 Psi) at 25° C. for 28 h. The suspension was filtrated and the filter cake was washed with MeOH (40 mL). The filtrate was concentrated under reduced pressure. The residue was purified by silica gel column chromatography (DCM:MeOH=100:1 to 6:1) to afford the title compound (400 mg, 90% yield, 92.2% purity) as brown oil.

LCMS: m/z 167.3 [M+H]⁺.

### Step 7: 3-methyl-5,6,7,8-tetrahydroimidazo [1,5-a] pyridin-6-ol

A solution of 6-methoxy-3-methyl-5,6,7,8-tetrahydroimidazo [1,5-a] pyridine (200 mg, 1.20 mmol) in HBr (14.90 g, 73.66 mmol) was heated at 80° C. The reaction mixture was stirred for 16 h. The pH of mixture was adjusted to 7 by using ammonium hydroxide and concentrated under reduced pressure. The residue was purified by prep-HPLC (column: Phenomenex C18 150×25 mm×10 μm; mobile phase: (water (NH₃·H₂O)-ACN); B %: 0%-23%, 10 min) to afford the title compound (70 mg, 38% yield, 100% purity) as yellow solid.

LCMS: m/z 153.3 [M+H]⁺.

¹H NMR (400 MHz, CDCl₃) δ=6.65 (s, 1H), 4.37 (bs, 1H), 3.94-3.86 (m, 2H), 3.01-2.93 (m, 1H), 2.76 (td, J=6.4, 16.4 Hz, 1H), 2.32 (s, 3H), 2.01-1.91 (m, 2H).

### Step 8: 3-methyl-5,6,7,8-tetrahydroimidazo [1,5-a] pyridin-6-yl 2-(3,5-dichlorophenyl) benzo[d]oxazole-6-carboxylate

To a solution of 3-methyl-5,6,7,8-tetrahydroimidazo[1,5-a]pyridin-6-ol (70 mg, 459 mmol) and DIEA (178 mg, 1.38 mmol) in DCM (5 mL) was added DMAP (56 mg, 0.46 mmol) and 2-(3,5-dichlorophenyl)-1,3-benzoxazole-6-carbonyl chloride (150 mg, 0.46 mmol) at 25° C. The reaction mixture was stirred for 16 h at 25° C. The mixture was concentrated under reduced pressure. The residue was purified by prep-HPLC (column: Welch Ultimate XB-Diol 150×40 mm×10 μm; mobile phase: (Hexane-EtOH, neutral); B %: 0%-20%, 11 min) to afford the title compound (29.97 mg, 15% yield, 100% purity) as a yellow solid.

LCMS: m/z 442.0 [M+H]⁺.

¹H NMR (400 MHz, CD₃OD) δ=8.29 (d, J=1.2 Hz, 1H), 8.19 (d, J=2.0 Hz, 2H), 8.08 (dd, J=1.6, 8.4 Hz, 1H), 7.83 (d, J=8.4 Hz, 1H), 7.72 (t, J=2.0 Hz, 1H), 6.66 (s, 1H), 5.69 (dd, J=2.0, 6.0 Hz, 1H), 4.27-4.18 (m, 2H), 3.07-2.91 (m, 2H), 2.35-2.30 (m, 4H), 2.15-2.07 (m, 1H).

### Example 57

### Compound 57: 1-(1,1,1-trifluoro-2-methylpropan-2-yl)pyrrolidin-3-yl 2-(3,5-dichlorophenyl)benzo[d]oxazole-6-carboxylate

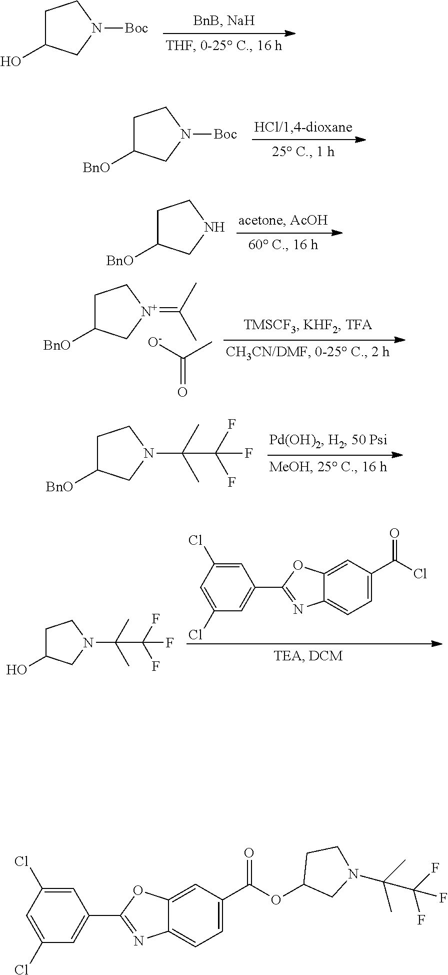

### Step 1: tert-butyl 3-(benzyloxy)pyrrolidine-1-carboxylate

NaH (1.92 g, 48.1 mmol, 60% purity) was added to a solution of tert-butyl 3-hydroxypyrrolidine-1-carboxylate (6 g, 32.1 mmol) in THF (60 mL) at 0° C. and stirred for 1 h, then BnBr (6.58 g, 38.5 mmol) was added at 0° C. The mixture was stirred for another 16 h at 25° C. The mixture was quenched with water (50 mL), then extracted with EtOAc (50 mL×3). The organic layer was washed with brine (80 mL), dried over Na₂SO₄, filtered and concentrated under reduced pressure. The crude product was purified by column chromatography (SiO₂, Petroleum ether:EtOAc=10:1 to 1:1) to afford the title compound (5.6 g, 63% yield) as colorless oil.

¹H NMR (400 MHz, CDCl₃) δ=7.40-7.23 (m, 5H), 4.52 (s, 2H), 4.16-4.07 (m, 1H), 3.55-3.37 (m, 4H), 2.07-2.01 (m, 1H), 1.93 (m, 1H), 1.46 (s, 9H).

### Step 2: 3-(benzyloxy)pyrrolidine

A solution of tert-butyl 3-benzyloxypyrrolidine-1-carboxylate (1.7 g, 6.13 mmol) in 4 M HCl/dioxane (10 mL) was stirred for 1 h at 25° C. The mixture was concentrated under reduced pressure to afford the title compound (1.3 g, crude) as a red oil, which was used for next step without purification.

### Step 3: 3-(benzyloxy)-1-(propan-2-ylidene)pyrrolidin-1-ium acetate

A solution of 3-benzyloxypyrrolidine (1.3 g, 7.33 mmol) and AcOH (660 mg, 11.0 mmol) in acetone (50 mL) was stirred for 16 h at 60° C. The mixture was concentrated under reduced pressure to afford the title compound (2 g, crude) as a brown oil, which was used for next step without purification.

### Step 4: 3-(benzyloxy)-1-(1,1,1-trifluoro-2-methylpropan-2-yl)pyrrolidine

TMSCF₃ (1.54 g, 10.82 mmol) was added to a mixture of 3-(benzyloxy)-1-(propan-2-ylidene) pyrrolidin-1-ium acetate (2 g, 7.21 mmol, crude), KHF₂ (563 mg, 7.21 mmol) and TFA (822 mg, 7.21 mmol) in CH₃CN (30 mL) and DMF (10 mL) at 0° C., then the mixture was stirred for 2 h at 25° C. The mixture was quenched with water (30 mL) and extracted with EtOAc (30 mL×3). The organic layer was washed with brine (50 mL) and concentrated under reduced pressure. The crude product was purified by column chromatography (SiO₂, Petroleum ether:EtOAc=10:1) to afford the title compound (150 mg, (0.15 g, 5% yield, 70% purity) as a yellow oil.

¹H NMR (400 MHz, CDCl₃) δ=7.26 (m, 5H), 4.57-4.43 (m, 2H), 4.14-4.02 (m, 1H), 3.14 (dd, J=6.4, 9.6 Hz, 1H), 2.96 (q, J=8.0 Hz, 1H), 2.92-2.81 (m, 2H), 2.08-1.96 (m, 1H), 1.87 (m, 1H), 1.29 (s, 6H)

### Step 5: 1-(1,1,1-trifluoro-2-methylpropan-2-yl)pyrrolidin-3-ol

Pd(OH)₂ (73 mg, 20% purity) was added to the solution of 3-benzyloxy-1-(2,2,2-trifluoro-1,1-dimethyl-ethyl)pyrrolidine (0.15 g, 0.52 mmol) in MeOH (3 mL) under N₂ atmosphere. The suspension was degassed and purged with H₂ for 3 times. The mixture was stirred under H₂ (50 Psi) at 25° C. for 16 h. The mixture was filtered through diatomite and the filtrate was concentrated under reduced pressure to afford the title compound (100 mg, crude) as a yellow oil, which was used for next step without purification.

¹H NMR (400 MHz, DMSO-d₆) δ=4.39 (s, 1H), 3.66-3.32 (m, 4H), 2.08-1.98 (m, 1H), 1.89 (s, 1H), 1.53 (s, 6H).

### Step 6: 1-(1,1,1-trifluoro-2-methylpropan-2-yl)pyrrolidin-3-yl 2-(3,5-dichlorophenyl)benzo[d]oxazole-6-carboxylate

2-(3,5-dichlorophenyl)benzo[d]oxazole-6-carbonyl chloride (0.1 g, 0.31 mmol) was added to a solution of 1-(2,2,2-trifluoro-1,1-dimethyl-ethyl)pyrrolidin-3-ol (61 mg, 0.31 mmol) and DMAP (37.41 mg, 0.31 mmol) in DCM (1 mL) and stirred for 16 h at 25° C. The mixture was filtered and the filtrate was purified by normal phase prep-HPLC (column: Welch Ultimate XB-Diol 250×50×10 μm; mobile phase: (Hexane-EtOH); B %: 5%-5%, 10 min), following by prep-TLC (petroleum ether:EtOAc=10:1) to afford the title compound (6.4 mg, 4.0% yield, 95% purity) as a white solid.

LCMS: m/z 487.0 [M+H]⁺

¹H NMR (400 MHz, CDCl₃) δ=8.28 (d, J=0.8 Hz, 1H), 8.17 (d, J=2.0 Hz, 2H), 8.12 (dd, J=1.6, 7.6 Hz, 1H), 7.81 (d, J=8.4 Hz, 1H), 7.56 (t, J=2.0 Hz, 1H), 5.51-5.37 (m, 1H), 3.37 (m, 1H), 3.10 (m, 2H), 2.98 (m, 1H), 2.34-2.21 (m, 1H), 2.09-2.01 (m, 1H), 1.25 (s, 6H).

### Example 58 and Example 59

### Compound 58: (cis)-hexahydro-1H-pyrrolizin-1-yl 2-(3,5-dichlorophenyl)benzo[d]oxazole-6-carboxylate; Compound 59: (trans)-hexahydro-1H-pyrrolizin-1-yl 2-(3,5-dichlorophenyl)benzo[d]oxazole-6-carboxylate

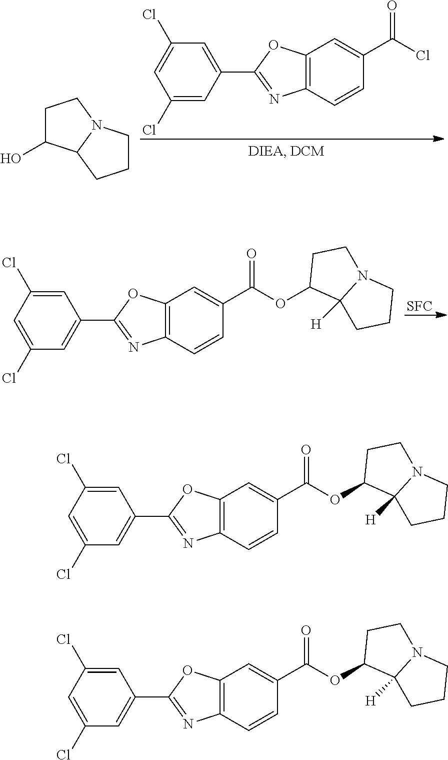

To a solution of hexahydro-1H-pyrrolizin-1-ol (312 mg, 2.45 mmol) in DCM (10 mL) was added DIEA (475 mg, 3.67 mmol) and DMAP (150 mg, 1.22 mmol), following by 2-(3,5-dichlorophenyl)benzo[d]oxazole-6-carbonyl chloride (400 mg, 1.22 mmol) at 25° C. The reaction mixture was stirred for 12 h at 25° C. The reaction mixture was concentrated under reduced pressure. The residue was purified by prep-HPLC (column: Welch Ultimate XB—NH2 250×50×10 μm; mobile phase: (Hexane-EtOH); B %: 0%-20%, 15 min) to give the title compound (96 mg) as yellow solid, which was separated by SFC (column: DAICEL CHIRALPAK AD (250 mm×30 mm, 10 μm); mobile phase: (0.1% NH₃·H₂O; MeOH); B %: 50%-50%; 40 min).

Compound 58 (13.37 mg, 3% yield, 97.1% purity) (P1, Ret. time=0.802 min) as off-white solid.

LCMS: m/z 417.2 [M+H]⁺

1H NMR (400 MHz, CDCl₃) δ=8.32 (d, J=1.2 Hz, 1H), 8.17 (d, J=2.0 Hz, 2H), 8.13 (dd, J=1.6, 8.4 Hz, 1H), 7.82 (d, J=8.4 Hz, 1H), 7.56 (t, J=2.0 Hz, 1H), 5.32-5.31 (m, 1H), 4.05-3.93 (m, 1H), 3.68-3.48 (m, 2H), 3.03-2.97 (m, 1H), 2.76-2.69 (m, 1H), 2.36-2.31 (m, 2H), 2.26-2.22 (m, 1H), 2.01-1.95 (m, 2H), 1.76-1.67 (m, 1H).

Compound 59 (37.47 mg, 7% yield, 98.4% purity) (P2, Ret. time=1.956 min) as off-white solid.

LCMS: m/z 376.3 [M+H]⁺

¹H NMR (400 MHz, CDCl₃) δ=8.30 (d, J=0.8 Hz, 1H), 8.17 (d, J=2.0 Hz, 2H), 8.12 (dd, J=1.2, 8.4 Hz, 1H), 7.81 (d, J=8.4 Hz, 1H), 7.55 (t, J=2.0 Hz, 1H), 5.26-5.23 (m, 1H), 3.76 (t, J=7.2 Hz, 1H), 3.45-3.39 (m, 1H), 3.28 (bs, 1H), 2.92-2.87 (m, 1H), 2.67-2.60 (m, 1H), 2.32-2.23 (m, 2H), 2.13-2.06 (m, 1H), 1.95-1.81 (m, 2H), 1.68-1.58 (m, 1H).

### Example 60

### Compound 60: 4,5,6,7-tetrahydro-1H-benzo[d]imidazol-6-yl 2-(3,5-dichlorophenyl) benzo[d]oxazole-6-carboxylate

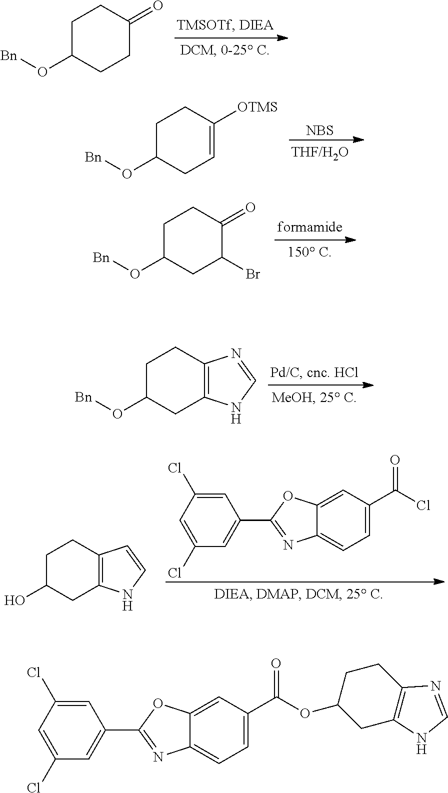

### Step 1: ((4-(benzyloxy) cyclohex-1-en-1-yl) oxy) trimethylsilane

To a solution of 4-benzyloxycyclohexanone (5 g, 24.48 mmol) in DCM (50 mL) was added DIEA (9.49 g, 73.43 mmol) and TMSOTf (8.16 g, 36.72 mmol) at 0° C. The reaction mixture was stirred for 1 h at 0° C. The reaction mixture was stirred for 16 h at 25° C. The mixture was quenched by H₂O (100 mL). The resulting mixture was transferred to a funnel and extracted with DCM (40 mL×2). The combined organic layers were concentrated under reduced pressure to afford the title compound (6.76 g, crude) as a yellow solid.

### Step 2: 4-(benzyloxy)-2-bromocyclohexanone

To a solution of (4-benzyloxycyclohexen-1-yl) oxy-trimethyl-silane (6.76 g, 24.45 mmol) in THF (30 mL) and H₂O (30 mL) was added NBS (5.22 g, 29.34 mmol) at 0° C. The mixture was transferred to a funnel and extracted with EtOAc (50 mL×2). The combined organic layers were concentrated under reduced pressure. The residue was purified by silica gel column chromatography (Petroleum ether:EtOAc=20:1 to 6:1) to afford the title compound (4.92 g, 71% yield) as colorless oil.

¹H NMR (400 MHz, DMSO-d₆) δ=7.41-7.34 (m, 5H), 5.03 (dd, J=5.6, 11.2 Hz, 1H), 4.59-4.58 (m, 2H), 3.91-3.86 (m, 1H), 2.72-2.64 (m, 2H), 2.54-2.52 (m, 1H), 2.34-2.27 (m, 1H), 2.17-2.08 (m, 1H), 2.00-1.96 (m, 1H).

### Step 3: 6-(benzyloxy)-4,5,6,7-tetrahydro-1H-benzo[d]imidazole

A solution of 4-benzyloxy-2-bromo-cyclohexanone (4.92 g, 17.38 mmol) in formamide (111.19 g, 2.47 mol) was heated at 150° C. and stirred for 6 h. The mixture was quenched by NaOH (10 mL). The resulting mixture was transferred to a funnel. The aqueous layer mixture was extracted with ethyl acetate (40 mL×3) and concentrated under reduced pressure. The residue was purified by silica gel column chromatography (DCM:MeOH=100:1 to 30:1) to give the crude product. Then it was purified by MPLC (Column 120g Flash silica gel Column Welch Ultimate XB C18 20-40 μm; 120 A; Solvent for sample dissolution about 2.00 grams of sample dissolved in 10 ml of MeOH; Flow rate 70 ml/min; Mobile phase H₂O+ACN; Gradient B % 10-55% 18 min; 55% 5 min 6; Instrument Biotage Prime) to afford the title compound (400 mg, 10% yield, 100% purity) as brown gum.

LCMS: m/z 229.2 [M+H]⁺

### Step 4: 4,5,6,7-tetrahydro-1H-benzo[d]imidazol-6-ol

To a solution of 6-benzyloxy-4,5,6,7-tetrahydro-1H-benzimidazole (390 mg, 1.71 mmol) in MeOH (2 mL) was added H₂ under N₂. The reaction mixture was degassed under reduced pressure and purged with Pd/C (40 mg) for three times. The reaction mixture was stirred under H₂ (50 Psi) at 25° C. for 12 h. LCMS showed the reaction didn't react, so HCl (0.5 mL) was added to the reaction mixture and continued to stirred for 16 h at 25° C. The suspension was filtered and the filter cake was washed with MeOH (20 mL). The filtrate was concentrated under reduced pressure to afford the title compound (180 mg, 1.25 mmol, 73.36% yield, 96.2% purity) as yellow gum

LCMS: m/z 139.3 [M+H]⁺

### Step 5: 4,5,6,7-tetrahydro-1H-benzo[d]imidazol-6-yl 2-(3,5-dichlorophenyl) benzo[d]oxazole-6-carboxylate

To a solution of 4,5,6,7-tetrahydro-3H-benzimidazol-5-ol (95.20 mg, 0.69 mmol) and DIEA (178 mg, 1.38 mmol) in DCM (10 mL) was added DMAP (56 mg, 0.46 mmol) and 2-(3,5-dichlorophenyl)-1,3-benzoxazole-6-carbonyl chloride (150 mg, 0.46 mmol) at 25° C. The reaction mixture was stirred for 16 h at 25° C. The mixture was concentrated under reduced pressure. The residue was purified by flash silica gel chromatography (20 g Silica Flash Column, Eluent of 0-10% Dichloromethane/Methanol @ 40 mL/min) to give the crude product and it was further purified by prep-HPLC (column: Welch Ultimate XB-Diol 250×50×10 μm; mobile phase: (Hexane-EtOH, neutral); B %: 15%-45%, 13 min) to afford the title compound (4.11 mg, 2.0% yield, 95.6% purity) as a white solid.

LCMS: m/z 428.0 [M+H]⁺

¹H NMR (400 MHz, CD₃OD) δ=8.29 (d, J=1.2 Hz, 1H), 8.20 (d, J=2.0 Hz, 2H), 8.08 (dd, J=1.2, 8.4 Hz, 1H), 7.83 (d, J=8.4 Hz, 1H), 7.72 (t, J=2.0 Hz, 1H), 7.55 (s, 1H), 5.56-5.50 (m, 1H), 3.12 (dd, J=4.8, 15.6 Hz, 1H), 2.91-2.72 (m, 3H), 2.30-2.14 (m, 2H).

### Example 61

### Compound 61: (cis)-3-hydroxycyclopentyl 2-(3,5-dichlorophenyl)benzo[d]oxazole-6-carboxylate

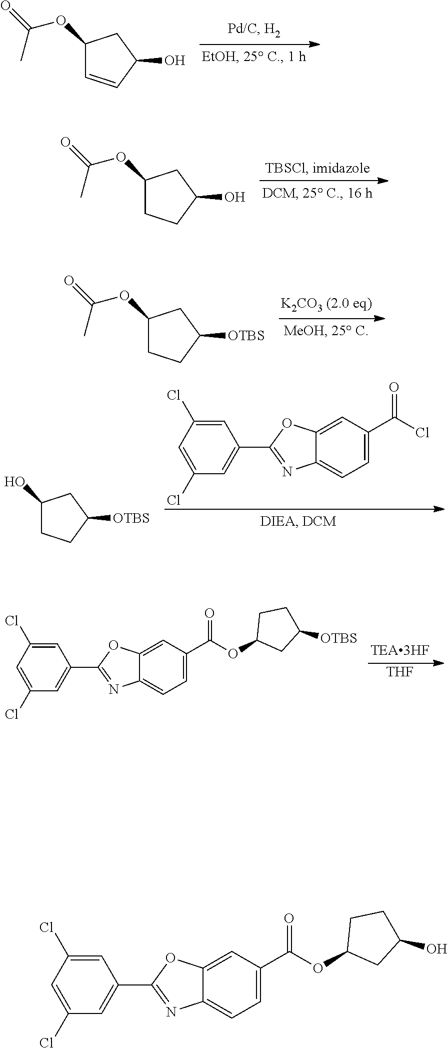

### Step 1: (cis)-3-hydroxycyclopentyl acetate

To a solution of (cis)-4-hydroxycyclopent-2-en-1-yl acetate (650 mg, 4.57 mmol) in EtOH (25 mL) was added Pd/C (50 mg). The suspension was degassed under reduced pressure and purged with H₂ for three times. The reaction mixture was stirred under H₂ balloon (15 Psi) at 25° C. for 2 h. The mixture was filtered and concentrated under reduced pressure to afford the title compound (500 mg, crude) as a white solid, which was used into the next step without further purification.

### Step 2: (cis)-3-((tert-butyldimethylsilyl) oxy) cyclopentyl acetate

To a suspension of (cis)-3-hydroxycyclopentyl acetate (500 mg, 3.47 mmol) in DCM (10 mL) was added TBSCl (784 mg, 5.20 mmol) and imidazole (472 mg, 6.94 mmol) and the mixture was stirred at 25° C. for 16 h. The reaction mixture was partitioned between H₂O (50 mL) and EtOAc (100 mL). The organic phase was separated, washed with brine (40 mL), dried over Na₂SO₄, filtered and concentrated under reduced pressure. The residue was purified by column chromatography (SiO₂, Petroleum ether:EtOAc=20:1 to 10:1) to afford the title compound (530 mg, 59% yield) as a colorless oil.

¹H NMR (400 MHz, DMSO-d₆) δ=4.96-4.91 (m, 1H), 4.24-4.19 (m, 1H), 2.24-2.17 (m, 1H), 1.96 (s, 3H), 1.90-1.82 (m, 1H), 1.77-1.67 (m, 2H), 1.63-1.55 (m, 1H), 1.50-1.45 (m, 1H), 0.85 (s, 9H), 0.03 (d, J=1.6 Hz, 6H).

### Step 3: (cis)-3-((tert-butyldimethylsilyl) oxy) cyclopentanol

To a solution of (cis)-3-((tert-butyldimethylsilyl)oxy)cyclopentyl acetate (530 mg, 2.05 mmol) in MeOH (10 mL) was added K₂CO₃ (567 mg, 4.10 mmol) and stirred at 25° C. for 16 h. The mixture was filtered and concentrated under reduced pressure. The residue was purified by column chromatography (SiO₂, Petroleum ether/EtOAc=20/1 to 10/1) to afford the title compound (390 mg, 88% yield) as a colorless oil.

### Step 4: (cis)-3-((tert-butyldimethylsilyl) oxy) cyclopentyl 2-(3,5-dichlorophenyl) benzo [d] oxazole-6-carboxylate

To a solution of (cis)-3-((tert-butyldimethylsilyl)oxy)cyclopentyl 2-(3,5-dichlorophenyl)benzo[d]oxazole-6-carboxylate (199 mg, 0.92 mmol) in DCM (5 mL) was added DIEA (237 mg, 1.84 mmol), DMAP (75 mg, 0.61 mmol) and 2-(3,5-dichlorophenyl)-1,3-benzoxazole-6-carbonyl chloride (200 mg, 0.61 mmol) and the mixture was stirred 25° C. for 12 h. mixture was concentrated under reduced pressure. The residue was purified by column chromatography (SiO₂, Petroleum ether:EtOAc=50:1 to 20:1) to afford the title compound (210 mg, 68% yield) as a white oil.

LCMS: m/z 506.1[M+H]⁺.

### Step 5: (cis)-3-hydroxycyclopentyl 2-(3,5-dichlorophenyl)benzo[d]oxazole-6-carboxylate

To a solution of (cis)-3-((tert-butyldimethylsilyl)oxy)cyclopentyl 2-(3,5-dichlorophenyl)benzo[d]oxazole-6-carboxylate (210 mg, 0.41 mmol) in THF (5 mL) was added 3HF9.TEA (201 mg, 1.24 mmol) and the mixture was stirred at 25° C. for 16 h. The reaction mixture was adjusted pH=7-8 by TEA and then diluted with H₂O (35 mL) and extracted with EtOAc (50 mL). The combined organic layers were washed with brine (25 mL), dried over Na₂SO₄, filtered and concentrated under reduced pressure. The residue was purified by flash silica gel chromatography (20 g Silica Flash Column, Eluent of 0-35% Ethyl acetate/Petroleum ether gradient @ 80 mL/min) to afford the title compound (68.54 mg, 41% yield, 98% purity) as a white solid.

LCMS: m/z 392.0[M+H]⁺.

¹H NMR (400 MHz, CDCl₃) δ=8.28 (s, 1H), 8.16-8.10 (m, 3H), 7.80 (bd, J=8.4 Hz, 1H), 7.55 (d, J=1.6 Hz, 1H), 5.47-5.45 (m, 1H), 4.44 (bd, J=2.4 Hz, 1H), 2.38-2.31 (m, 1H), 2.16-2.11 (m, 2H), 2.04-1.96 (m, 3H).

### Example 62

### Compound 62: (trans)-3-hydroxycyclopentyl 2-(3,5-dichlorophenyl)benzo-[d]oxazole-6-carboxylate

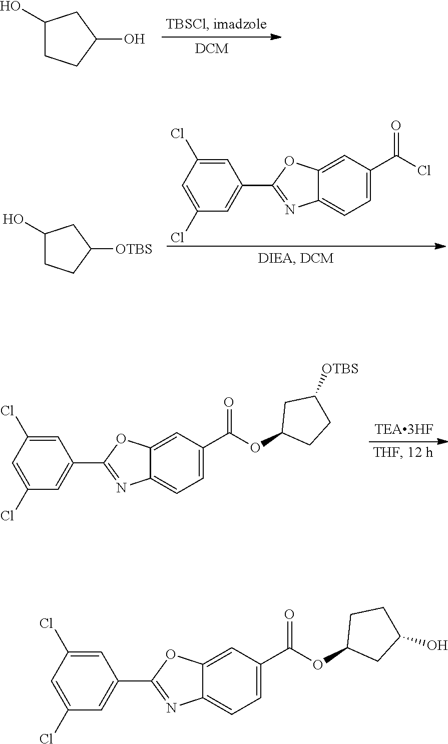 [SMILES: OC1CCC(O)C1 ; CC(C)(C)[Si](C)(C)OC1CCC(O)C1 ; O=C(Cl)c1ccc2nc(-c3cc(Cl)cc(Cl)c3)oc2c1 ; CC(C)(C)[Si](C)(C)O[C@@H]1CC[C@@H](OC(=O)c2ccc3nc(-c4cc(Cl)cc(Cl)c4)oc3c2)C1 ; O=C(O[C@H]1CC[C@H](O)C1)c1ccc2nc(-c3cc(Cl)cc(Cl)c3)oc2c1]

### Step 1: 3-((tert-butyldimethylsilyl) oxy) cyclopentanol

To a solution of cyclopentane-1,3-diol (2 g, 19.58 mmol) in DCM (20 mL) was added TBSCl (2.95 g, 19.58 mmol) and imidazole (1.33 g, 19.58 mmol) at 0° C. and the mixture was stirred at 25° C. for 12 h. The reaction mixture was diluted with H₂O (40 mL) and extracted with DCM (50 mL). The combined organic layers were washed with brine (30 mL), dried over Na₂SO₄, filtered and concentrated under reduced pressure. The residue was purified by column chromatography (SiO₂, Petroleum ether/Ethyl acetate=20/1 to 10/1) to afford the title compound (0.66 g, 16% yield) as a colorless oil.

¹H NMR (400 MHz, CD₃OD) δ=4.47-4.42 (m, 1H), 4.37-4.32 (m, 1H), 2.09-1.94 (m, 2H), 1.79 (t, J=5.2 Hz, 2H), 1.56-1.46 (m, 2H), 0.89 (s, 9H), 0.06 (s, 6H).

### Step 2: (trans)-3-((tert-butyldimethylsilyl) oxy) cyclopentyl 2-(3,5-dichlorophenyl) benzo[d] oxazole-6-carboxylate

To a solution of 3-[tert-butyl(dimethyl)silyl]oxycyclopentanol (100 mg, 0.46 mmol) in DCM (5 mL) was added DIEA (119 mg, 0.92 mmol), DMAP (19 mg, 0.15 mmol) and 2-(3,5-dichlorophenyl)benzo[d]oxazole-6-carbonyl chloride (100 mg, 0.31 mmol) and the mixture was stirred at 25° C. for 12 h. The reaction mixture was concentrated under reduced pressure. The residue was purified by column chromatography (SiO₂, Petroleum ether/Ethyl acetate=50/1) to afford the title compound (99 mg, 60% yield, 94% purity) as a white solid.

LCMS: m/z 506.4[M+H]⁺.

¹H NMR (400 MHz, CDCl₃) δ=8.25 (s, 1H), 8.17 (d, J=1.6 Hz, 2H), 8.10-8.08 (m, 1H), 7.80 (d, J=8.4 Hz, 1H), 7.57-7.55 (m, 1H), 5.56-5.52 (m, 1H), 4.49-4.46 (m, 1H), 2.37-2.28 (m, 1H), 2.12-1.98 (m, 3H), 1.87-1.80 (m, 1H), 1.72-1.64 (m, 1H), 0.90 (s, 9H), 0.08 (s, 6H).

### Step 3: (trans)-3-hydroxycyclopentyl 2-(3,5-dichlorophenyl) benzo[d]oxazole-6-carboxylate

To a solution of [(trans)-3-[tert-butyl(dimethyl)silyl]oxycyclopentyl] 2-(3,5-dichlorophenyl)-1,3-benzoxazole-6-carboxylate (99 mg, 0.18 mmol) in THF (2 mL) was added 3HF.TEA (89 mg, 0.55 mmol) and stirred at 25° C. for 16 h. The reaction mixture was adjusted pH7-8 by TEA and then diluted with H₂O (20 mL) and extracted with Ethyl acetate (35 mL). The combined organic layers were washed with brine (15 mL), dried over Na₂SO₄, filtered and concentrated under reduced pressure. The residue was purified by flash silica gel chromatography (20 g Silica Flash Column, Eluent of 0-35% Ethyl acetate/Petroleum ether gradient @ 60 mL/min) to afford the title compound (40.26 mg, 55% yield, 98% purity) as a white solid.

LCMS: m/z 392.0[M+H]⁺.

¹H NMR (400 MHz, CDCl₃) δ=8.26 (s, 1H), 8.17 (d, J=2.0 Hz, 2H), 8.10-8.07 (m, 1H), 7.80 (d, J=8.4 Hz, 1H), 7.56-7.55 (m, 1H), 5.61-5.56 (m, 1H), 4.62-4.57 (m, 1H), 2.40-2.30 (m, 1H), 2.19-2.09 (m, 3H), 1.95-1.88 (m, 1H), 1.78-1.71 (m, 1H), 1.44 (bd, J=1.6 Hz, 1H).

### Example 63

### Compound 63: (cis)-3-hydroxycyclobutyl 2-(3,5-dichlorophenyl) benzo[d]oxazole-6-carboxylate

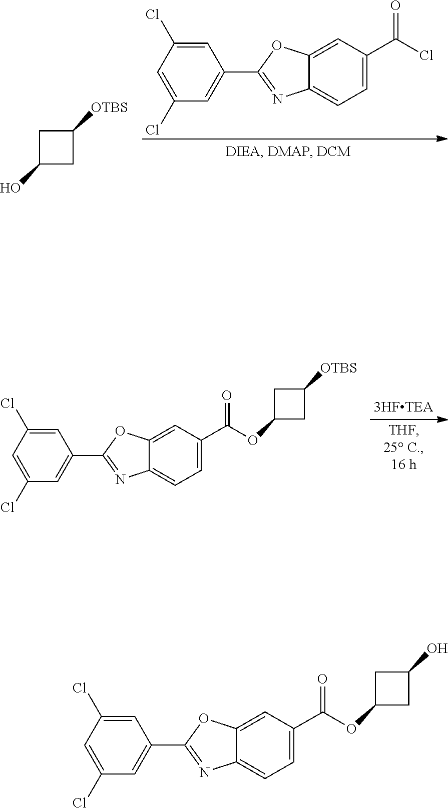 [SMILES: CC(C)(C)[Si](C)(C)O[C@H]1C[C@@H](O)C1 ; O=C(Cl)c1ccc2nc(-c3cc(Cl)cc(Cl)c3)oc2c1 ; CC(C)(C)[Si](C)(C)O[C@H]1C[C@@H](OC(=O)c2ccc3nc(-c4cc(Cl)cc(Cl)c4)oc3c2)C1 ; O=C(O[C@H]1C[C@@H](O)C1)c1ccc2nc(-c3cc(Cl)cc(Cl)c3)oc2c1]

### Step 1: (cis)-3-((tert-butyldimethylsilyl)oxy)cyclobutyl 2-(3,5-dichlorophenyl) benzo[d]oxazole-6-carboxylate

To a solution of 3-((tert-butyldimethylsilyl)oxy)cyclobutan-1-ol (139 mg, 0.69 mmol) in DCM (2 mL) was added DMAP (28 mg, 0.23 mmol), DIEA (178 mg, 1.38 mmol) and 2-(3,5-dichlorophenyl)benzo[d]oxazole-6-carbonyl chloride (150 mg, 0.46 mmol). The mixture was stirred at 25° C. for 12 h. The reaction mixture was quenched by addition water (10 mL) at 25° C. and extracted with dichloromethane (15 mL×3). The combined organic layers were dried over Na₂SO₄, filtered and concentrated under reduced pressure. The residue was purified by column chromatography (SiO₂, Petroleum ether/Ethyl acetate=100/1 to 10/1) to afford the title compound (124 mg, 55% yield) as white solid.

LCMS: m/z 493.9 [M+H]⁺

¹H NMR (400 MHz, CDCl₃) δ=8.28 (d, J=0.4 Hz, 1H), 8.17-8.11 (m, 3H), 7.81 (d, J=8.4 Hz, 1H), 7.55 (t, J=2.0 Hz, 1H), 4.84 (quin, J=7.2 Hz, 1H), 4.06 (quin, J=7.2 Hz, 1H), 2.95-2.88 (m, 2H), 2.28-2.20 (m, 2H), 0.91 (s, 9H), 0.08 (s, 6H).

### Step 2: (cis)-3-hydroxycyclobutyl 2-(3,5-dichlorophenyl) benzo[d]oxazole-6-carboxylate

A solution of (cis)-3-((tert-butyldimethylsilyl)oxy)cyclobutyl 2-(3,5-dichlorophenyl)benzo[d]oxazole-6-carboxylate (124 mg, 0.25 mmol) in THF (2 mL) and 3HF.TEA (122 mg, 0.76 mmol) was stirred for 16 h at 25° C. The reaction mixture was quenched by water (3 mL). The aqueous layer mixture was extracted with ethyl acetate (20 mL×3) and concentrated under reduced pressure. The residue was purified by flash silica gel chromatography (20 g Silica Flash Column, Eluent of 0-30% Ethyl acetate/Petroleum ether gradient @ 40 mL/min) to afford the title compound (59.62 mg, 62% yield, 99.8% purity) as white solid.

LCMS: m/z 377.9 [M+H]⁺

¹H NMR (400 MHz, CDCl₃) δ=8.29 (s, 1H), 8.17 (d, J=2.0 Hz, 2H), 8.12 (dd, J=1.2, 8.4 Hz, 1H), 7.81 (d, J=8.4 Hz, 1H), 7.56 (t, J=2.0 Hz, 1H), 4.88 (q, J=7.2 Hz, 1H), 4.14 (qd, J=6.8, 13.6 Hz, 1H), 3.04-2.97 (m, 2H), 2.27-2.20 (m, 2H), 1.88 (d, J=5.6 Hz, 1H).

### Example 64

### Compound 64: (trans)-3-hydroxycyclobutyl 2-(3,5-dichlorophenyl) benzo[d]oxazole-6-carboxylate

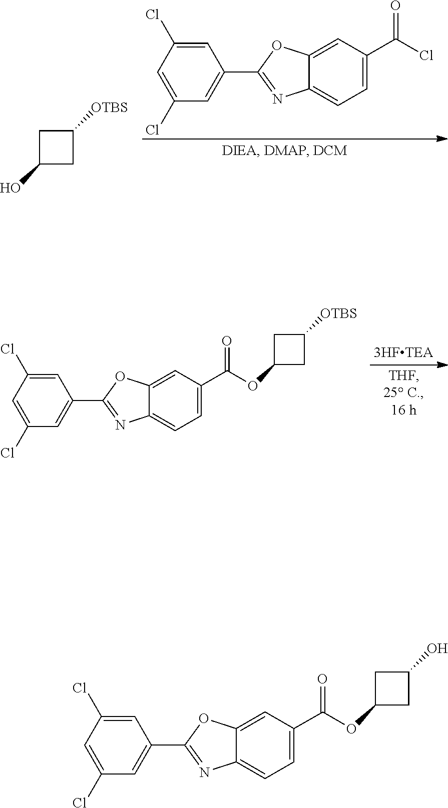 [SMILES: CC(C)(C)[Si](C)(C)O[C@H]1C[C@H](O)C1 ; O=C(Cl)c1ccc2nc(-c3cc(Cl)cc(Cl)c3)oc2c1 ; CC(C)(C)[Si](C)(C)O[C@H]1C[C@H](OC(=O)c2ccc3nc(-c4cc(Cl)cc(Cl)c4)oc3c2)C1 ; O=C(O[C@H]1C[C@H](O)C1)c1ccc2nc(-c3cc(Cl)cc(Cl)c3)oc2c1]

### Step 1: (trans)-3-((tert-butyldimethylsilyl) oxy) cyclobutyl 2-(3,5-dichlorophenyl) benzo[d]oxazole-6-carboxylate

To a solution of (trans)-3-((tert-butyldimethylsilyl)oxy)cyclobutanol (139 mg, 0.69 mmol) and 2-(3,5-dichlorophenyl)-1,3-benzoxazole-6-carbonyl chloride (150 mg, 0.46 mmol) in DCM (2 mL) was added DIEA (178 mg, 1.38 mmol), DMAP (28 mg, 0.23 mmol) and 2-(3,5-dichlorophenyl)benzo[d]oxazole-6-carbonyl chloride (150 mg, 459.33 umol). The mixture was stirred at 25° C. for 12 h. The reaction mixture was quenched by addition water 10 mL at 25° C. and extracted with dichloromethane (15 mL×3). The combined organic layers were dried over Na₂SO₄, filtered and concentrated under reduced pressure. The residue was purified by column chromatography (SiO₂, Petroleum ether/Ethyl acetate=100/1 to 10/1) to afford the title compound (159 mg, 70% yield) as white solid.

LCMS: m/z 492.2 [M+H]⁺

¹H NMR (400 MHz, CDCl₃) δ=8.29 (d, J=0.8 Hz, 1H), 8.17-8.12 (m, 3H), 7.82 (d, J=8.4 Hz, 1H), 7.56 (t, J=1.6 Hz, 1H), 5.38 (tt, J=3.2, 6.8 Hz, 1H), 4.64 (quin, J=6.8 Hz, 1H), 2.57-2.46 (m, 4H), 0.91 (s, 8H), 0.08-0.06 (m, 6H).

### Step 2: (trans)-3-hydroxycyclobutyl 2-(3,5-dichlorophenyl)benzo[d]oxazole-6-carboxylate

A solution of (trans)-3-((tert-butyldimethylsilyl)oxy)cyclobutyl 2-(3,5-dichlorophenyl) benzo[d]oxazole-6-carboxylate (159 mg, 0.32 mmol) in THF (2 mL) and 3HF.TEA (156 mg, 0.97 mmol) was stirred for 16 h at 25° C. The pH of mixture was adjusted to 8 by using TEA (3 mL). The aqueous layer mixture was extracted with dichloromethane (20 mL×3) and concentrated under reduced pressure. The residue was purified by flash silica gel chromatography (20 g Silica Flash Column, Eluent of 0-40% Ethyl acetate/Petroleum ether gradient @ 40 mL/min) to afford the title compound (72.67 mg, 60% yield, 100% purity) as a white solid.

LCMS: m/z 378.0 [M+H]⁺.

¹H NMR (400 MHz, CDCl₃) δ=8.27 (s, 1H), 8.16 (s, 2H), 8.12 (d, J=8.4 Hz, 1H), 7.81 (d, J=8.4 Hz, 1H), 7.55 (s, 1H), 5.46 (s, 1H), 3.43-3.20 (m, 2H), 3.12-2.83 (m, 3H), 2.39-2.28 (m, 1H), 2.12-2.02 (m, 1H), 1.31 (d, J=6.8 Hz, 3H).

### Example 65

### Compound 65: 3-oxooctahydroindolizin-1-yl 2-(3,5-dichlorophenyl)benzo-[d]oxazole-6-carboxylate

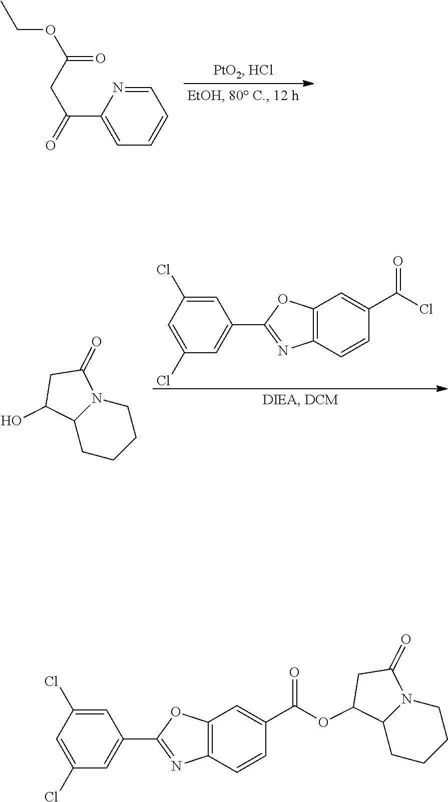 [SMILES: CCOC(=O)CC(=O)c1ccccn1 ; O=C1CC(O)C2CCCCN12 ; O=C(Cl)c1ccc2nc(-c3cc(Cl)cc(Cl)c3)oc2c1 ; O=C(OC1CC(=O)N2CCCCC12)c1ccc2nc(-c3cc(Cl)cc(Cl)c3)oc2c1]

### Step 1: 1-hydroxyhexahydroindolizin-3(2H)-one

To a solution of ethyl 3-oxo-3-(2-pyridyl) propanoate (2 g, 10.35 mmol) in EtOH (40 mL) was added cnc. HCl (105 mg, 1.04 mmol) and PtO₂ (470 mg, 2.07 mmol) under N₂. The suspension was degassed under reduced pressure and purged with H₂ for three times. The resulting mixture was stirred under H₂ (50 Psi) for 12 h at 80° C. The reaction mixture was filtered through a pad of celite and washed with EtOH (10 mL×3). The filtrate was concentrated under reduced pressure. The residue was purified by silica gel column (DCM:MeOH=100:1 to 30:1) to afford the title compound (1.1 g, 68% yield) as yellow oil.

¹H NMR (400 MHz, CDCl₃) δ=4.40-4.05 (m, 2H), 3.44-3.26 (m, 1H), 2.84-2.61 (m, 3H), 2.39-2.33 (m, 1H), 2.05-1.65 (m, 3H), 1.45-1.05 (m, 3H).

### Step 2: 3-oxooctahydroindolizin-1-yl 2-(3,5-dichlorophenyl)benzo[d]oxazole-6-carboxylate

To a solution of 1-hydroxyhexahydroindolizin-3(2H)-one (107 mg, 0.69 mmol) and DIEA (178 mg, 1.38 mmol) in DCM (5 mL) was added DMAP (56 mg, 0.46 mmol) and 2-(3,5-dichlorophenyl)benzo[d]oxazole-6-carbonyl chloride (150 mg, 0.46 mmol) at 25° C. The reaction mixture was stirred for 16 h at 25° C. The mixture was concentrated under reduced pressure. The residue was purified by silica gel column chromatography (Petroleum ether:EtOAc=10:1 to 1:1) to afford the title compound (45.43 mg, 22% yield, 97.1% purity) as a white solid.

LCMS: m/z 445.1 [M+H]⁺

¹H NMR (400 MHz, CD₃OD) δ=8.41 (s, 1H), 8.25 (t, J=2.4 Hz, 2H), 8.18-8.15 (m, 1H), 7.89 (dd, J=2.0, 8.4 Hz, 1H), 7.77-7.76 (m, 1H), 5.28-5.25 (m, 1H), 4.17-4.13 (m, 1H), 3.76 (bd, J=2.8, 12.0 Hz, 1H), 3.09-3.03 (m, 1H), 2.85 (bt, J=12.8 Hz, 1H), 2.67 (bd, J=18.0 Hz, 1H), 2.19-2.16 (m, 1H), 1.99-1.96 (m, 1H), 1.76-1.72 (m, 1H), 1.67-1.57 (m, 1H), 1.46-1.34 (m, 2H).

### Example 66

### Compound 66: 1-(2-methoxyethyl) pyrrolidin-3-yl 2-(3,5-dichlorophenyl) benzo[d]oxazole-6-carboxylate

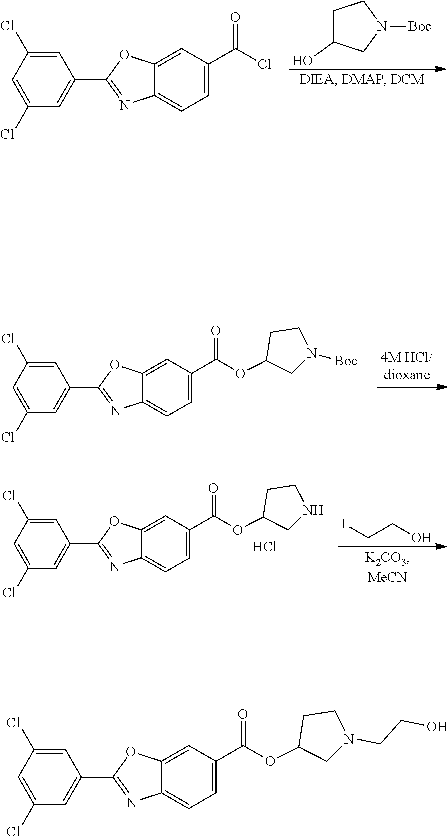

### Step 1: 1-(tert-butoxycarbonyl) pyrrolidin-3-yl 2-(3,5-dichlorophenyl) benzo[d]oxazole-6-carboxylate

To a solution of tert-butyl 3-hydroxypyrrolidine-1-carboxylate (430 mg, 2.30 mmol) in DCM (5 mL) was added DIEA (396 mg, 3.06 mmol) and DMAP (187 mg, 1.53 mmol) and 2-(3,5-dichlorophenyl)-1,3-benzoxazole-6-carbonyl chloride (0.5 g, 1.53 mmol). The mixture was stirred at 25° C. for 16 h. The reaction mixture was concentrated under reduced pressure.

The residue was purified by column chromatography (SiO₂, Petroleum ether:EtOAc=20:1 to 10:1) to afford the title compound (800 mg, crude) as a white solid.

¹H NMR (400 MHz, CDCl₃) δ=7.38 (s, 1H), 7.26-7.20 (m, 3H), 6.93 (d, J=8.4 Hz, 1H), 6.69-6.65 (m, 1H), 4.68 (bs, 1H), 2.84-2.68 (m, 4H), 1.35 (bs, 2H), 0.59 (bs, 9H).

### Step 2: pyrrolidin-3-yl 2-(3,5-dichlorophenyl) benzo[d]oxazole-6-carboxylate

(1-tert-butoxycarbonylpyrrolidin-3-yl)2-(3,5-dichlorophenyl)-1,3-benzoxazole-6-carboxylate (800 mg, 1.68 mmol) was dissolved in HCl/dioxane (50 mL) and the mixture was stirred at 25° C. for 12 h. The suspension was filtered and the filter cake was washed with Petroleum ether (60 mL). The solid was concentrated under vacuum without purification to afford the title compound (438 mg, 1.06 mmol, 63% yield, 99.8% purity, HCl salt) was obtained as a white solid.

¹H NMR (400 MHz, CD₃OD) δ=8.41 (d, J=0.8 Hz, 1H), 8.20 (d, J=2.0 Hz, 2H), 8.18-8.16 (m, 1H), 7.87 (d, J=8.4 Hz, 1H), 7.75 (t, J=1.6 Hz, 1H), 5.74-5.70 (m, 1H), 3.66-3.65 (m, 2H), 3.60-3.55 (m, 2H), 2.47-2.42 (m, 2H).

### Step 3: 1-(2-methoxyethyl) pyrrolidin-3-yl 2-(3,5-dichlorophenyl) benzo[d]oxazole-6-carboxylate

To a solution of pyrrolidin-3-yl 2-(3,5-dichlorophenyl)-1,3-benzoxazole-6-carboxylate (100 mg, 242 mmol, HCl salt) and K₂CO₃ (100 mg, 0.73 mmol) in MeCN (1 mL) was added 2-iodoethanol (46 mg, 0.27 mmol). The reaction mixture was stirred at 25° C. for 16 h. The suspension was quenched by addition H₂O (2 mL), filtrated and the filter cake was concentrated under reduced pressure. The residue was purified by prep-HPLC (column: Welch Ultimate XB—SiOH 250×50×10 μm; mobile phase: (Hexane-EtOH); B %: 0%-32%, 13 min) to afford the title compound (10.98 mg, 10% yield, 96.8% purity) was obtained as a white solid.

LCMS: m/z 421.1 [M+H]⁺.

¹H NMR (400 MHz, CD₃OD) δ=8.40 (s, 1H), 8.22 (d, J=1.6 Hz, 2H), 8.15 (d, J=8.4 Hz, 1H), 7.85 (d, J=8.4 Hz, 1H), 7.74 (s, 1H), 5.50-5.45 (m, 1H), 3.73 (t, J=6.0 Hz, 2H), 3.04-2.94 (m, 3H), 2.78-2.67 (m, 2H), 2.63-2.57 (m, 1H), 2.47-2.38 (m, 1H), 2.10-2.03 (m, 1H).

### Example 67

### Compound 67: 1-(2-methoxyethyl) pyrrolidin-3-yl 2-(3,5-dichlorophenyl) benzo[d]oxazole-6-carboxylate

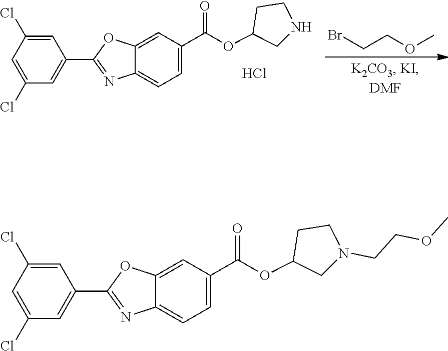

To a solution of pyrrolidin-3-yl 2-(3,5-dichlorophenyl)-1,3-benzoxazole-6-carboxylate (150 mg, 0.40 mmol) and 1-bromo-2-methoxy-ethane (55 mg, 0.40 mmol, 37.34 uL) in DMF (2 mL) was added K₂CO₃ (137.39 mg, 0.99 mmol) and KI (66 mg, 0.40 mmol). The reaction mixture was stirred at 25° C. for 16 h, and then the mixture was stirred at 40° C. for 12 h. The reaction mixture was partitioned between ethyl acetate (30 mL) and H₂O (25 mL). The organic phase was separated and washed with brine (30 mL). It was dried over Na₂SO₄, filtered and concentrated under reduced pressure. The residue was purified by column chromatography (SiO₂, Petroleum ether/R=20/1 to 1/1, R=(ethyl acetate/ethyl alcohol=3/1)) to afford the title compound (26.63 mg, 15% yield, 97% purity) as a white solid.

LCMS: m/z 435.0 [M+H]⁺.

¹H NMR (400 MHz, CD₃OD) δ=8.38 (d, J=1.2 Hz, 1H), 8.20 (d, J=2 Hz, 2H), 8.16-8.13 (m, 1H), 7.84 (d, J=8.4 Hz, 1H), 7.73 (t, J=2 Hz, 1H), 5.49-5.44 (m, 1H), 3.58 (t, J=5.6 Hz, 2H), 3.37 (s, 3H), 3.04-2.96 (m, 3H), 2.84-2.73 (m, 2H), 2.65-2.59 (m, 1H), 2.46-2.37 (m, 1H), 2.10-2.02 (m, 1H).

### Example 68

Compound 68: Octahydroindolizin-2-ol (available from Sigma-Aldrich) is reacted with 2-(3,5-dichlorophenyl)-1,3-benzoxazole-6-carbonyl chloride under standard conditions (DIEA, DMAP) to afford Compound 68.

### Example 69

### Compound 69 and Compound 70: Octahydroindolizin-8-yl 2-(3,5-dichlorophenyl)benzo[d]oxazole-6-carboxylate

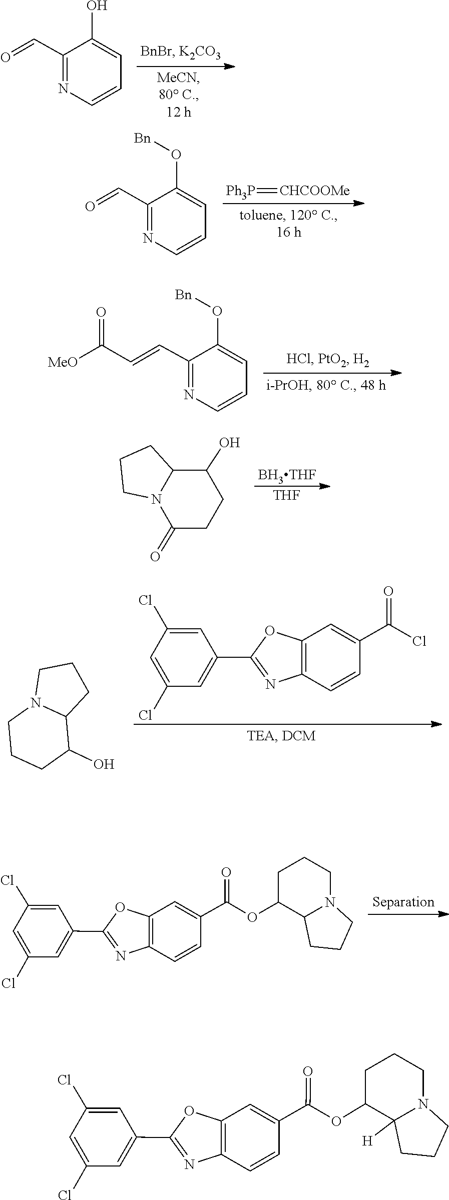

### Step 1: 3-(Benzyloxy)picolinaldehyde

BnBr (5.67 g, 33.14 mmol) was added to the mixture of 3-hydroxypicolinaldehyde (4 g, 32.49 mmol) and K₂CO₃ (5.39 g, 38.99 mmol) in CH₃CN (40 mL) and stirred at 80° C. for 16 hrs. The mixture was quenched with water (20 mL) and extracted with DCM (20 mL×3), the organic was washed with brine (30 mL), dried over Na₂SO₄, filtered and concentrated under reduced pressure. The crude product was purified by chromatography (SiO₂, Pet. ether/EtOAc=3/1) to afford the title compound (3 g, 43% yield) as a yellow oil.

### Step 2: (E)-Methyl 3-(3-(benzyloxy)pyridin-2-yl)acrylate

A solution of 3-(benzyloxy)picolinaldehyde (3 g, 14.07 mmol) and methyl 2-(triphenylphosphoranylidene)acetate (5.64 g, 16.88 mmol) in toluene (30 mL) was stirred at 120° C. for 3 hrs. The mixture was concentrated and the residue dissolved with EtOAc (30 mL), washed with water (50 mL), extracted with EtOAc (20 mL×2), the organic was washed with brine (40 mL), dried over Na₂SO₄, filtered and concentrated under reduced pressure. The crude product was purified by chromatography (SiO₂, Pet. ether/EtOAc=3/1) to afford the title compound (2.9 g, 76% yield) as a light yellow oil.

### Step 3: 8-Hydroxyhexahydroindolizin-5(1H)-one

To a solution of (E)-methyl 3-(3-(benzyloxy)pyridin-2-yl)acrylate (2.9 g, 10.77 mmol) in i-PrOH (30 mL) was added PtO₂ (489 mg, 2.15 mmol) and HCl (12 M, 0.09 mL) under N₂ atmosphere. The suspension was degassed and purged with H₂ for 3 times. The mixture was stirred under H₂ (50 Psi) at 80° C. for 48 hrs. The mixture was filtered through a pad of celite and the filtrate concentrated under reduced pressure. The crude product was purified by column chromatography (SiO₂, CH₂Cl₂:CH₃OH=100:1 to 10:1) to afford the title compound (1.2 g, 72% yield) as a light yellow oil.

¹H NMR (400 MHz, CDCl₃) δ=4.15-4.09 (m, 1H), 3.83 (s, 1H), 3.57-3.53 (m, 1H), 2.69-2.66 (m, 1H), 2.42-2.32 (m, 2H), 2.05-1.98 (m, 3H), 1.77-1.66 (m, 4H)

### Step 4: Octahydroindolizin-8-ol

BH₃.THF (1 M, 10.00 mL) was added to the mixture of 8-hydroxyhexahydroindolizin-5(1H)-one (1 g, 6.44 mmol) in THF (2 mL) at 0° C. under N₂, then the mixture was stirred at 25° C. for 6 hrs. The mixture was quenched with CH₃OH (10 mL) at 0° C., then concentrated to give the crude product. The crude product was purified by column chromatography (SiO₂, CH₂Cl₂:CH₃OH=10:1) to afford the title compound (500 mg, 55% yield) as a colorless oil.

¹H NMR (400 MHz, CDCl₃) δ=4.48-4.42 (m, 1H), 3.37-3.33 (m, 1H), 3.25-3.20 (m, 1H), 3.14-3.09 (m, 1H), 2.82-2.77 (m, 1H), 2.63-2.56 (m, 1H), 1.99-1.94 (m, 3H), 1.87-1.80 (m, 2H), 1.71-1.63 (m, 1H), 1.58-1.48 (m, 2H).

### Step 5: Octahydroindolizin-8-yl 2-(3,5-dichlorophenyl)benzo[d]oxazole-6-carboxylate

3-Hydroxypicolinaldehyde (500 mg, 1.53 mmol) was added to the mixture of octahydroindolizin-8-ol (324.31 mg, 2.30 mmol), DMAP (187 mg, 1.53 mmol) and TEA (465 mg, 4.59 mmol) in DCM (10 mL) and stirred at 25° C. for 16 hrs. The mixture was concentrated under reduced pressure, then the crude product was purified by column chromatography (SiO₂, Pet. ether/EtOAc=3/1 to 1/1), followed by SFC (DAICEL CHIRALPAK IG (250 mm×30 mm, 10 μm); (0.1% NH₃H₂O/MeOH); B %: 60%) to afford two isomers.

Isomer 1 (22.59 mg, 99.7% purity, 0.937 min) as a white solid.

LCMS: m/z 431.0 [M+H]⁺

¹H NMR (400 MHz, CD₃OD) δ=8.60 (d, J=1.2 Hz, 1H), 8.29-8.27 (m, 1H), 8.20 (d, J=2.0 Hz, 2H), 7.85 (d, J=8.4 Hz, 1H), 7.73 (t, J=1.6 Hz, 1H), 5.45 (s, 1H), 3.23 (s, 1H), 2.50-2.33 (m, 3H), 2.13 (d, J=12.4 Hz, 1H), 2.04-1.81 (m, 5H), 1.77-1.66 (m, 3H).

Isomer 2 (15.55 mg, 99.8% purity, 1.258 min) as a white solid.

LCMS: m/z 431.0 [M+H]⁺

¹H NMR (400 MHz, CD₃OD) δ=8.61 (d, J=0.8 Hz, 1H), 8.29-8.26 (m, 1H), 8.20 (d, J=2.0 Hz, 2H), 7.85 (d, J=8.4 Hz, 1H), 7.73 (t, J=1.6 Hz, 1H), 5.43 (s, 1H), 3.19 (t, J=6.4 Hz, 1H), 2.42-2.37 (m, 1H), 2.27-2.25 (m, 2H), 2.14-2.11 (m, 1H), 2.00-1.96 (m, 1H), 1.87-1.81 (m, 3H), 1.75-1.64 (m, 4H).

### Example 70

### Compound 71 and Compound 72: Octahydro-1H-quinolizin-1-yl 2-(3,5-dichlorophenyl)benzo[d]oxazole-6-carboxylate

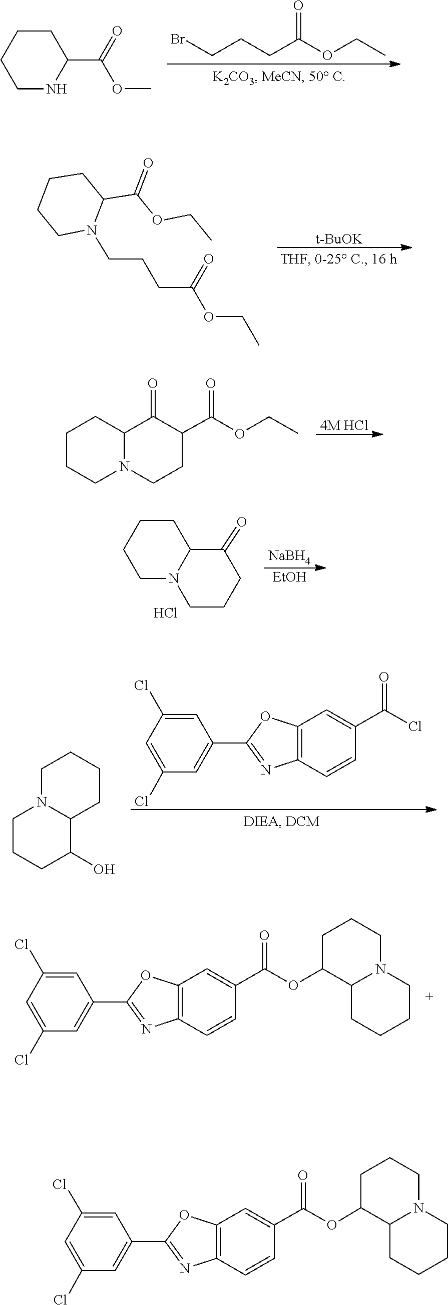

### Step 1: Ethyl 1-(4-ethoxy-4-oxobutyl)piperidine-2-carboxylate

To a solution of methyl piperidine-2-carboxylate (5 g, 34.92 mmol) and ethyl 4-bromobutanoate (6.81 g, 34.92 mmol) in MeCN (100 mL) was added K₂CO₃ (4.83 g, 34.92 mmol) stirred for 16 hrs at 50° C. The reaction mixture was partitioned between EtOAc (50 mL) and water (30 mL). The organic phase was separated, washed with water 30 mL (10 mL×3), dried over Na₂SO₄, filtered and concentrated under reduced pressure. The residue was purified by column chromatography (SiO₂, Pet. ether:EtOAc=2:1 to 1:1) to afford the title compound (4.5 g, 50% yield) as a yellow oil.

¹H NMR (400 MHz, CDCl₃) δ=4.10-4.02 (m, 2H), 3.67-3.61 (m, 3H), 3.10-2.95 (m, 2H), 2.51-2.41 (m, 1H), 2.34-2.20 (m, 3H), 2.18-2.06 (m, 1H), 1.81-1.64 (m, 4H), 1.54 (br s, 3H), 1.36-1.27 (m, 1H), 1.23-1.17 (m, 3H).

### Step 2: Ethyl 1-oxooctahydro-1H-quinolizine-2-carboxylate

To a solution of methyl 1-(4-ethoxy-4-oxo-butyl)piperidine-2-carboxylate (3 g, 11.66 mmol) in THF (30 mL) was added t-BuOK (1 M, 29.15 mL) under N₂ at 0° C. The reaction mixture was stirred for 16 hrs at 25° C. The mixture was quenched by saturated ammonium chloride solution (20 mL). The resulting mixture was transferred to a separated funnel, and the aqueous layer mixture was extracted with EtOAc (30 mL×3), dried over anhydrous sodium sulfate, filtered, concentrated under reduced pressure. The title compound (2.21 g, 81% yield) as a yellow oil which was used without purification.

LCMS: m/z 226.1 [M+H]⁺

### Step 3: Octahydro-1H-quinolizin-1-one hydrochloride

A solution of ethyl 1-oxooctahydro-1H-quinolizine-2-carboxylate (1 g, 4.44 mmol) in HCl (6 M, 10 mL) was heated to 80° C. and stirred for 16 hrs. The mixture was concentrated under reduced pressure. The title compound (1.1 g, crude, HCl salt) as a brown oil, which was used without purification.

LCMS: m/z 154.1 [M+H]⁺

### Step 4: Octahydro-1H-quinolizin-1-ol

To a solution of octahydro-1H-quinolizin-1-one hydrochloride (1.1 g, 5.80 mmol, HCl) in EtOH (15 mL) was added NaBH₄ (280 mg, 7.40 mmol) under N₂ at 0° C. The reaction mixture was stirred for 16 hrs at 25° C. The mixture was quenched by NH₄Cl (5 mL), the resulting mixture was concentrated under reduced pressure. The title compound (1 g) was isolated as a brown solid and used without purification.

### Step 5: Octahydro-1H-quinolizin-1-yl 2-(3,5-dichlorophenyl)benzo[d]oxazole-6-carboxylate

To a solution of octahydro-1H-quinolizin-1-ol (951 mg, 6.12 mmol) in DCM (10 mL) was added DIEA (792 mg, 6.12 mmol) and DMAP (150 mg, 1.22 mmol), and then was added 2-(3,5-dichlorophenyl)benzo[d]oxazole-6-carbonyl chloride (400 mg, 1.22 mmol) at 25° C. The reaction mixture was stirred for 16 hrs at 25° C. The mixture was concentrated under reduced pressure. The residue was purified by silica gel column chromatography (Pet. ether:EtOAc=10:1 to 1:1), followed by prep-HPLC (Welch Ultimate XB—NH₂ 250×50×10 μm; (Hexane-EtOH); B %: 0%-10%) to give the racemate, which was further separated by SFC (DAICEL CHIRALPAK IG (250×30 mm, 10 μm); (0.1% NH₃H₂O MeOH); B %: 60-70%) to afford two isomers.

Isomer 1 (6.63 mg, 96.3% purity as a white solid; Ret. Time: 0.877, 1.169)

LCMS: m/z 445.1 [M+H]⁺

¹H NMR (400 MHz, CDCl₃) δ=8.28 (s, 1H), 8.17 (d, J=1.6 Hz, 2H), 8.12 (d, J=8.4 Hz, 1H), 7.81 (d, J=8.4 Hz, 1H), 7.56 (t, J=1.6 Hz, 1H), 4.87-4.81 (m, 1H), 2.95-2.92 (m, 1H), 2.85-2.83 (m, 1H), 2.28-2.24 (m, 1H), 2.13-2.09 (m, 2H), 2.05-2.01 (m, 1H), 1.94 (br d, J=9.2 Hz, 1H), 1.84-1.74 (m, 3H), 1.53-1.38 (m, 3H), 1.31 (br s, 2H).

Isomer 2 (7.78 mg, 93.6% purity as a white solid; Ret. Time: 1.681)

LCMS: m/z 445.0 [M+H]⁺

¹H NMR (400 MHz, CDCl₃) δ=8.29 (s, 1H), 8.18 (d, J=2.0 Hz, 2H), 8.13 (dd, J=1.2, 8.4 Hz, 1H), 7.82 (d, J=8.4 Hz, 1H), 7.57 (t, J=2.0 Hz, 1H), 4.88-4.81 (m, 1H), 2.94 (br d, J=8.8 Hz, 1H), 2.88-2.83 (m, 1H), 2.29-2.26 (m, 1H), 2.16-2.09 (m, 2H), 2.05-2.01 (m, 1H), 1.95 (br d, J=8.0 Hz, 1H), 1.85-1.75 (m, 3H), 1.46 (dq, J=4.4, 12.0 Hz, 3H), 1.34-1.29 (m, 2H).

### Example 71

Compound 73 is prepared as follows:

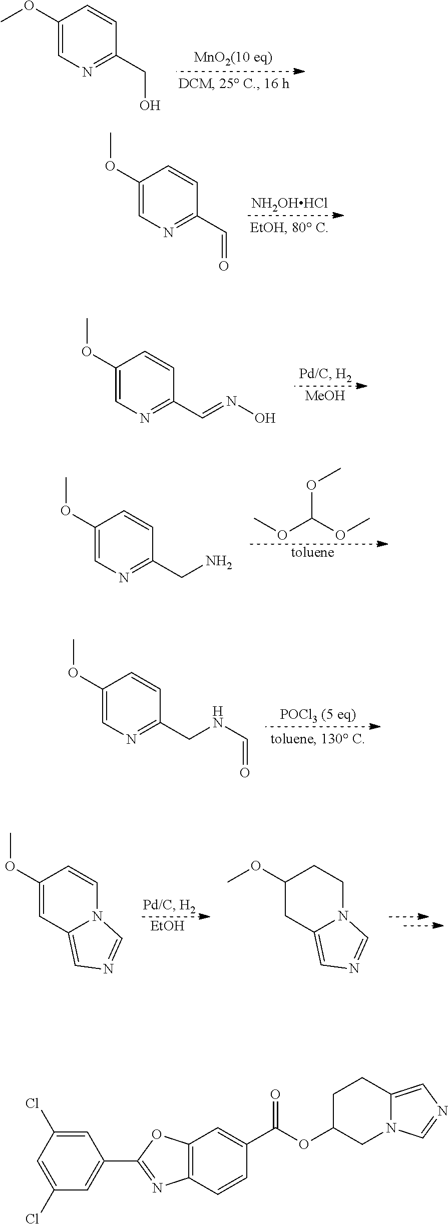

### Example 72

Compound 74 and Compound 75 are prepared as follows:

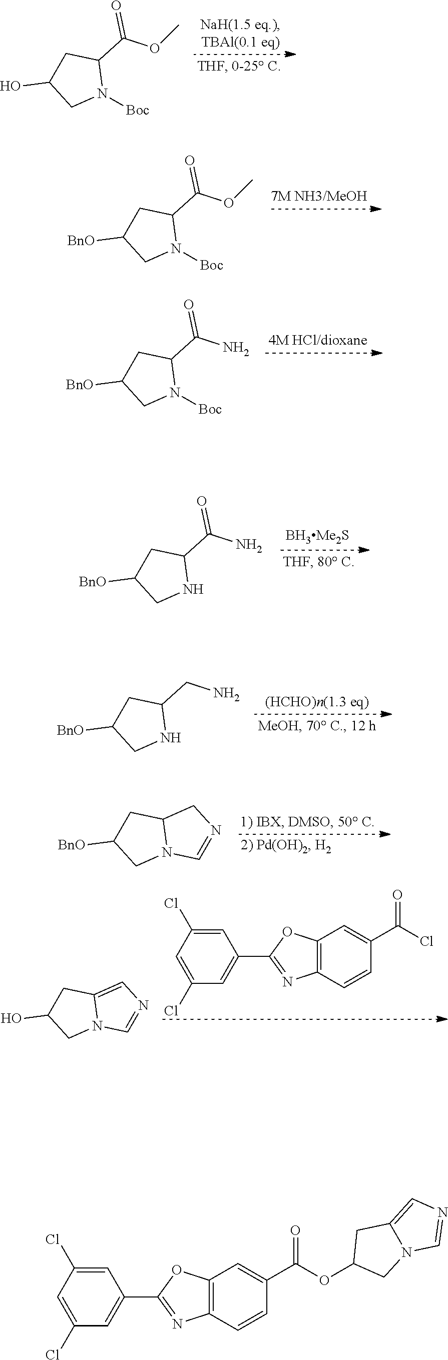

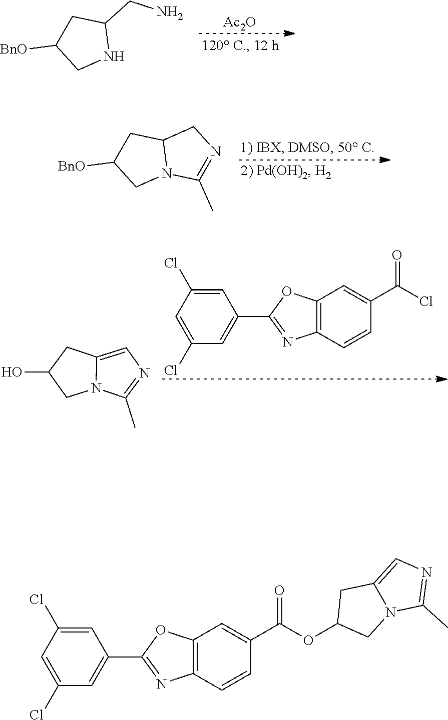

### Example 73

Compound 76 is prepared as follows:

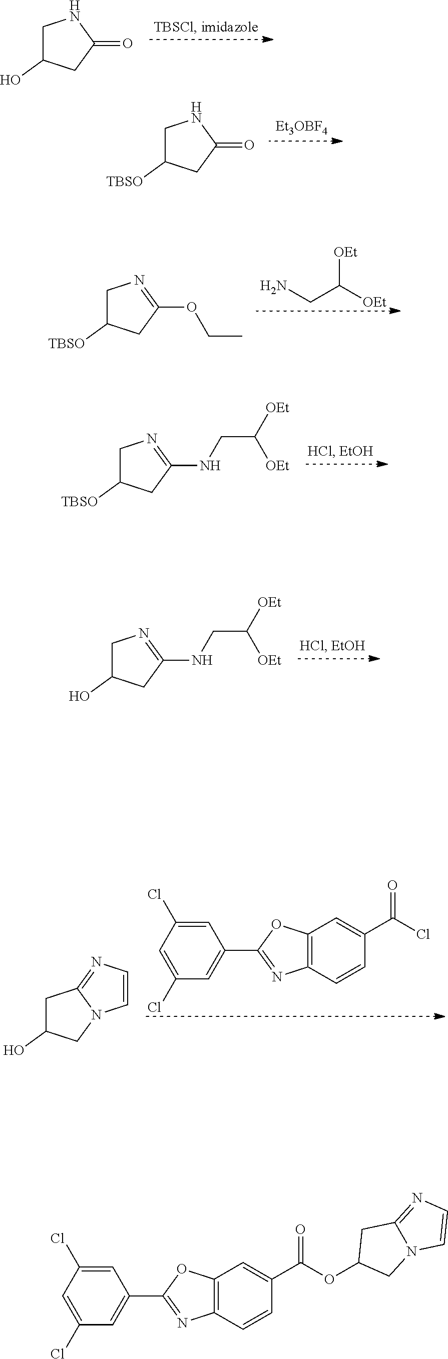

### Example 74

Compound 77 is prepared as follows:

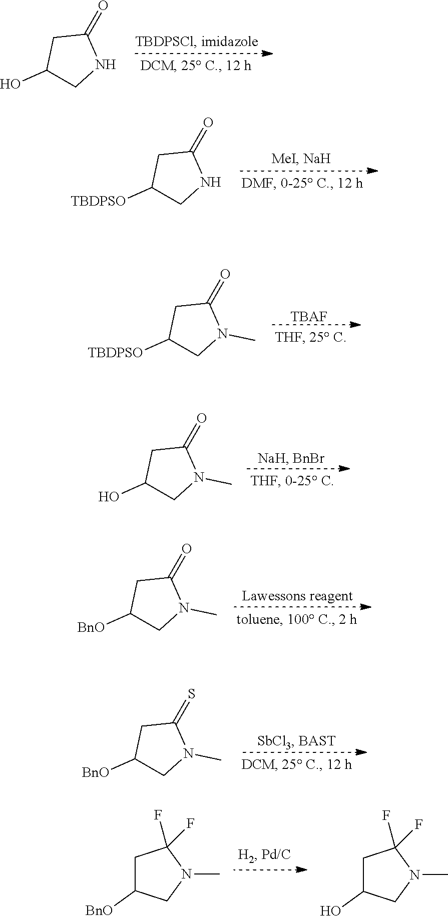

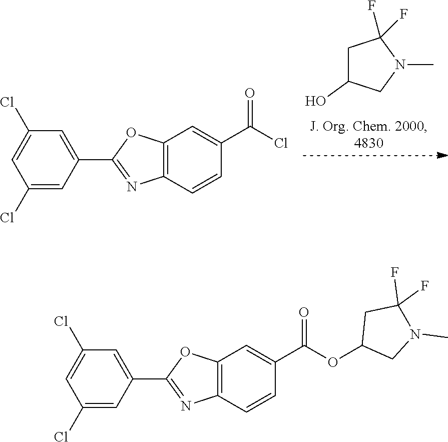

### Example 75

Compound 78 is prepared as follows:

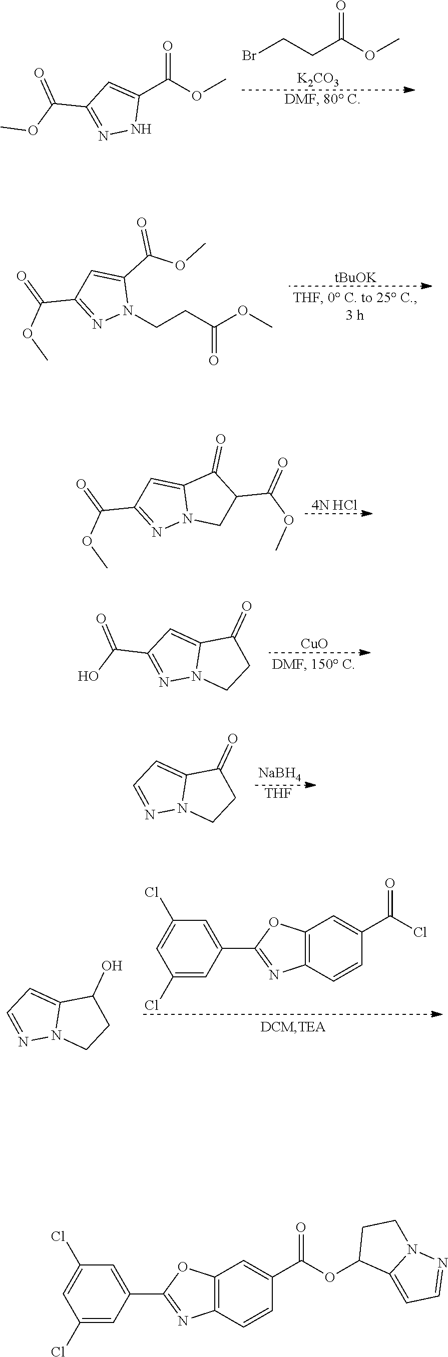

### Example 76

Compound 79 is prepared as follows:

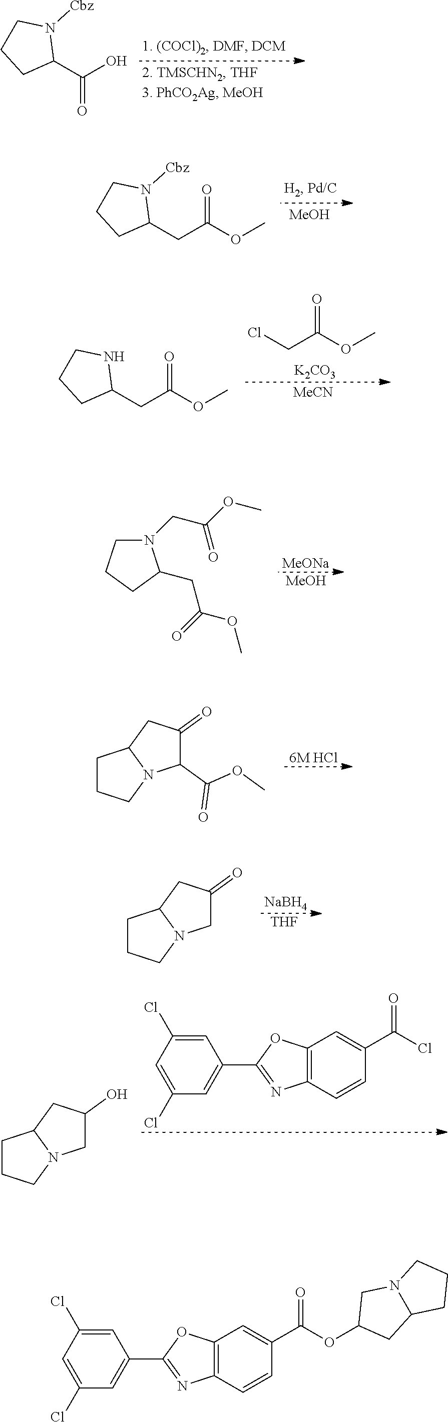

### Example 77

### Compound 87: 2-(3,5-Dichlorophenyl)-N,N-dimethylbenzo[d]oxazole-6-carboxamide

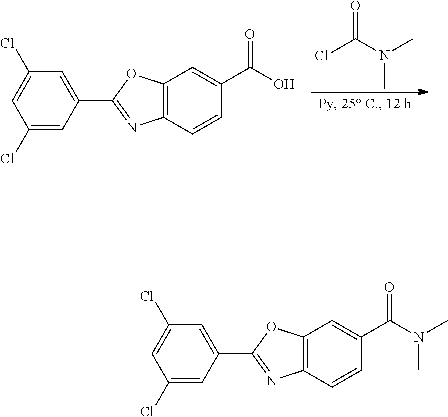

To a solution of 2-(3,5-dichlorophenyl)benzo[d]oxazole-6-carboxylic acid (100 mg, 0.32 mmol) in pyridine (2 mL) was added N,N-dimethylcarbamoyl chloride (38 mg, 0.36 mmol) at 25° C. The mixture was stirred for 16 h at 25° C. The mixture was quenched by HCl (0.1 M, 10 mL), the resulting mixture was transferred to a separated funnel, and the aqueous layer mixture was extracted with ethyl acetate (10 mL×2). The combined organic layers were washed with brine (10 mL), dried over anhydrous sodium sulfate, filtered, and concentrated under reduced pressure. The residue was purified by flash silica gel chromatography (20 g Silica Flash Column, Eluent of 0-40% Ethyl acetate/Petroleum ether @ 40 mL/min) to afford the title compound (18.53 mg, 17% yield) as white solid.

LCMS: m/z 334.9 [M+H]⁺

¹H NMR (400 MHz, CDCl₃) δ=8.16 (d, J=2.0 Hz, 2H), 7.80 (d, J=8.4 Hz, 1H), 7.70 (s, 1H), 7.54 (t, J=2.0 Hz, 1H), 7.46 (dd, J=1.2, 8.4 Hz, 1H), 3.16 (s, 3H), 3.04 (s, 3H).

### Example 78

Further compounds provided herein were prepared as shown in Table 2:

TABLE 2

| | | Coupling with acid |
|---|---|---|
| | | chloride and appropriate |
| Compound | | alcohol or amine similar |
| Number | Structure | to Example |
| 80 | 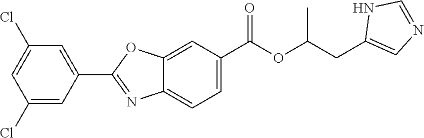 [SMILES: CC(Cc1cnc[nH]1)OC(=O)c1ccc2nc(-c3cc(Cl)cc(Cl)c3)oc2c1] | 34 |
| 81 | 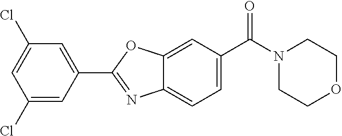 [SMILES: O=C(c1ccc2nc(-c3cc(Cl)cc(Cl)c3)oc2c1)N1CCOCC1] | 25 |
| 82 | 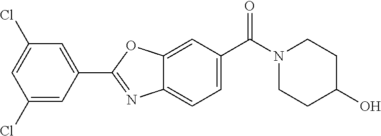 [SMILES: O=C(c1ccc2nc(-c3cc(Cl)cc(Cl)c3)oc2c1)N1CCC(O)CC1] | 25 |
| 83 | 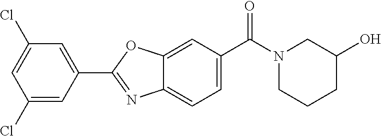 [SMILES: O=C(c1ccc2nc(-c3cc(Cl)cc(Cl)c3)oc2c1)N1CCCC(O)C1] | 25 |
| 84 | 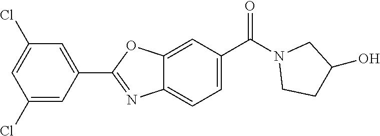 [SMILES: O=C(c1ccc2nc(-c3cc(Cl)cc(Cl)c3)oc2c1)N1CCC(O)C1] | 25 |
| 85 | 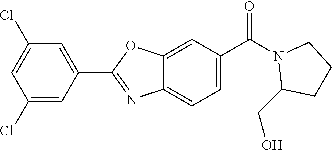 [SMILES: O=C(c1ccc2nc(-c3cc(Cl)cc(Cl)c3)oc2c1)N1CCCC1CO] | 25 |
| 86 | 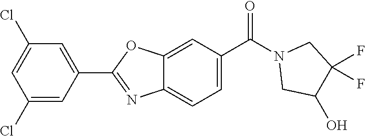 [SMILES: O=C(c1ccc2nc(-c3cc(Cl)cc(Cl)c3)oc2c1)N1CC(O)C(F)(F)C1] | 25 |
| 87 | 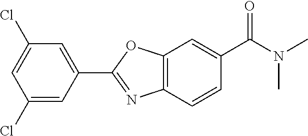 [SMILES: CN(C)C(=O)c1ccc2nc(-c3cc(Cl)cc(Cl)c3)oc2c1] | 77 |
| 88 | 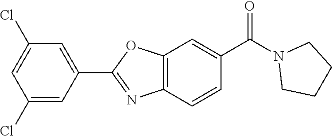 [SMILES: O=C(c1ccc2nc(-c3cc(Cl)cc(Cl)c3)oc2c1)N1CCCC1] | 77 |
| 89 | 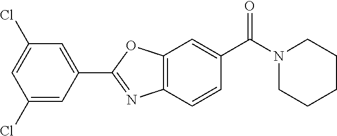 [SMILES: O=C(c1ccc2nc(-c3cc(Cl)cc(Cl)c3)oc2c1)N1CCCCC1] | 77 |
| 90 | 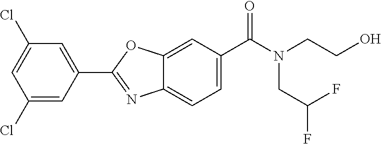 [SMILES: O=C(c1ccc2nc(-c3cc(Cl)cc(Cl)c3)oc2c1)N(CCO)CC(F)F] | 25 |
| 91 | 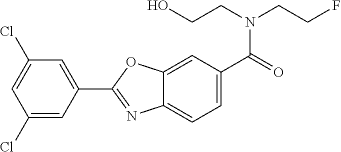 [SMILES: O=C(c1ccc2nc(-c3cc(Cl)cc(Cl)c3)oc2c1)N(CCO)CCF] | 25 |
| 92 | 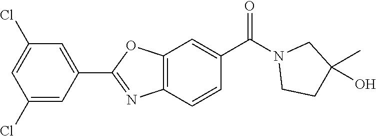 [SMILES: CC1(O)CCN(C(=O)c2ccc3nc(-c4cc(Cl)cc(Cl)c4)oc3c2)C1] | 25 |
| 93 | 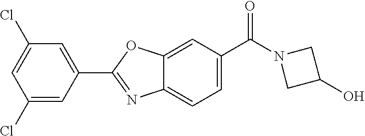 [SMILES: O=C(c1ccc2nc(-c3cc(Cl)cc(Cl)c3)oc2c1)N1CC(O)C1] | 25 |
| 94 | 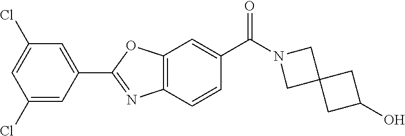 [SMILES: O=C(c1ccc2nc(-c3cc(Cl)cc(Cl)c3)oc2c1)N1CC2(CC(O)C2)C1] | 25 |
| 95 | 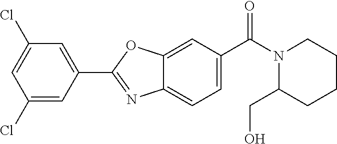 [SMILES: O=C(c1ccc2nc(-c3cc(Cl)cc(Cl)c3)oc2c1)N1CCCCC1CO] | 25 |
| 96 | 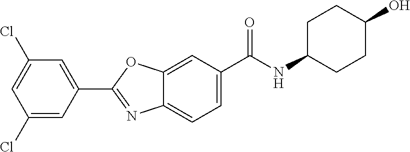 [SMILES: O=C(N[C@H]1CC[C@@H](O)CC1)c1ccc2nc(-c3cc(Cl)cc(Cl)c3)oc2c1] | 25 |
| 97 | 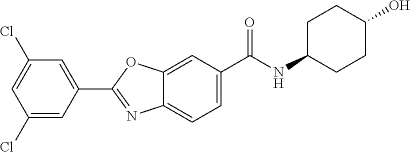 [SMILES: O=C(N[C@H]1CC[C@H](O)CC1)c1ccc2nc(-c3cc(Cl)cc(Cl)c3)oc2c1] | 25 |
| 98 | 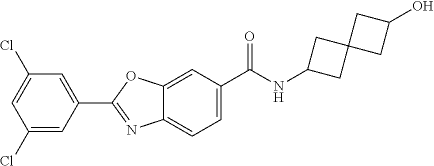 [SMILES: O=C(NC1CC2(CC(O)C2)C1)c1ccc2nc(-c3cc(Cl)cc(Cl)c3)oc2c1] | 25 |
| 99 | 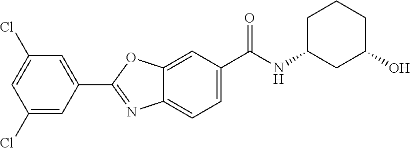 [SMILES: O=C(N[C@@H]1CCC[C@H](O)C1)c1ccc2nc(-c3cc(Cl)cc(Cl)c3)oc2c1] | 25 |
| 100 | 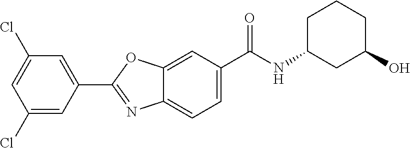 [SMILES: O=C(N[C@@H]1CCC[C@@H](O)C1)c1ccc2nc(-c3cc(Cl)cc(Cl)c3)oc2c1] | 25 |
| 101 | 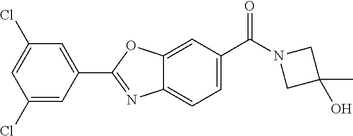 [SMILES: CC1(O)CN(C(=O)c2ccc3nc(-c4cc(Cl)cc(Cl)c4)oc3c2)C1] | 25 |
| 102 | 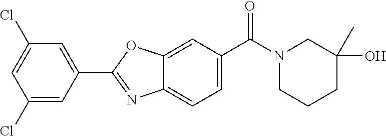 [SMILES: CC1(O)CCCN(C(=O)c2ccc3nc(-c4cc(Cl)cc(Cl)c4)oc3c2)C1] | 25 |
| 103 | 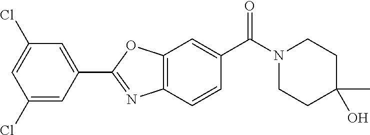 [SMILES: CC1(O)CCN(C(=O)c2ccc3nc(-c4cc(Cl)cc(Cl)c4)oc3c2)CC1] | 25 |
| 104 | 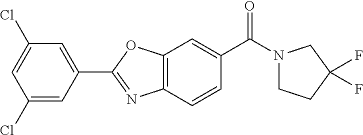 [SMILES: O=C(c1ccc2nc(-c3cc(Cl)cc(Cl)c3)oc2c1)N1CCC(F)(F)C1] | 25 |
| 105 | 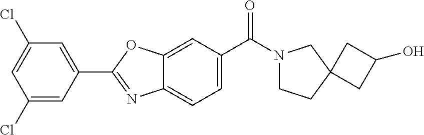 [SMILES: O=C(c1ccc2nc(-c3cc(Cl)cc(Cl)c3)oc2c1)N1CCC2(CC(O)C2)C1] | 25 |
| 106 | 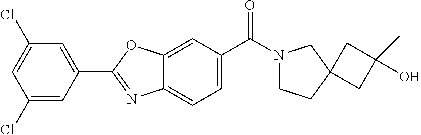 [SMILES: CC1(O)CC2(CCN(C(=O)c3ccc4nc(-c5cc(Cl)cc(Cl)c5)oc4c3)C2)C1] | 25 |
| 107 | 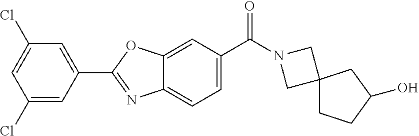 [SMILES: O=C(c1ccc2nc(-c3cc(Cl)cc(Cl)c3)oc2c1)N1CC2(CCC(O)C2)C1] | 25 |
| 108 | 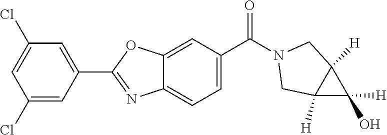 [SMILES: O=C(c1ccc2nc(-c3cc(Cl)cc(Cl)c3)oc2c1)N1C[C@@H]2[C@H](O)[C@@H]2C1] | 25 |
| 109 | 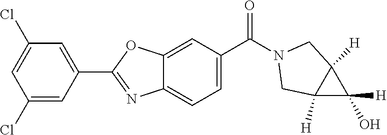 [SMILES: O=C(c1ccc2nc(-c3cc(Cl)cc(Cl)c3)oc2c1)N1C[C@@H]2[C@@H](O)[C@@H]2C1] | 25 |
| 110 | 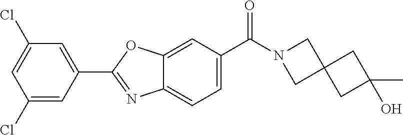 [SMILES: CC1(O)CC2(CN(C(=O)c3ccc4nc(-c5cc(Cl)cc(Cl)c5)oc4c3)C2)C1] | 25 |
| 111 | 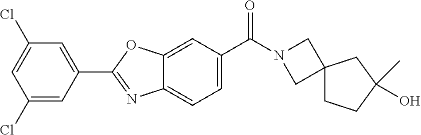 [SMILES: CC1(O)CCC2(CN(C(=O)c3ccc4nc(-c5cc(Cl)cc(Cl)c5)oc4c3)C2)C1] | 25 |
| 112 | 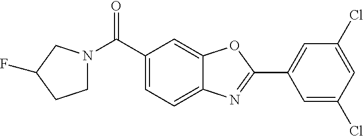 [SMILES: O=C(c1ccc2nc(-c3cc(Cl)cc(Cl)c3)oc2c1)N1CCC(F)C1] | 25 |
| 113 | 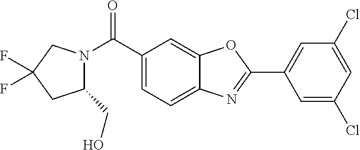 [SMILES: O=C(c1ccc2nc(-c3cc(Cl)cc(Cl)c3)oc2c1)N1CC(F)(F)C[C@H]1CO] | 25 |
| 114 | 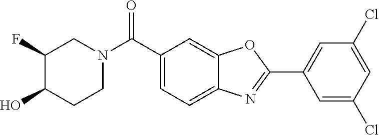 [SMILES: O=C(c1ccc2nc(-c3cc(Cl)cc(Cl)c3)oc2c1)N1CC[C@@H](O)[C@@H](F)C1] | 25 |
| 115 | 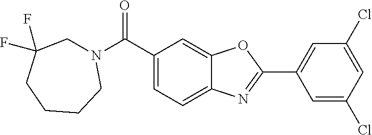 [SMILES: O=C(c1ccc2nc(-c3cc(Cl)cc(Cl)c3)oc2c1)N1CCCCC(F)(F)C1] | 25 |
| 116 | 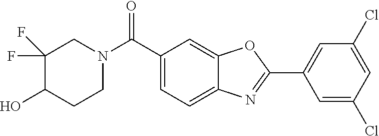 [SMILES: O=C(c1ccc2nc(-c3cc(Cl)cc(Cl)c3)oc2c1)N1CCC(O)C(F)(F)C1] | 25 |
| 117 | 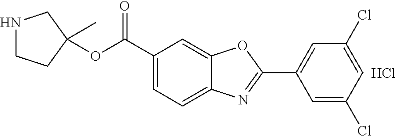 [SMILES: CC1(OC(=O)c2ccc3nc(-c4cc(Cl)cc(Cl)c4)oc3c2)CCNC1 ; Cl] | BocPyrrolidineMeOH, DMAP, DIEA, DCM, then de-Boc 4N HCl/Dioxane |
| 118 | 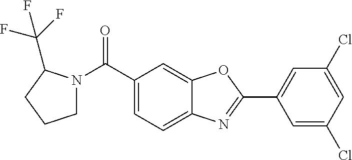 [SMILES: O=C(c1ccc2nc(-c3cc(Cl)cc(Cl)c3)oc2c1)N1CCCC1C(F)(F)F] | 25 |
| 119 | 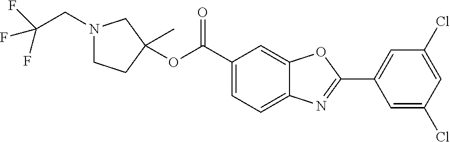 [SMILES: CC1(OC(=O)c2ccc3nc(-c4cc(Cl)cc(Cl)c4)oc3c2)CCN(CC(F)(F)F)C1] | Alkylate cmpd 117 TfOCH₂CF₃, DIEA, DMF |
| 120 | 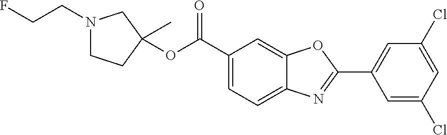 [SMILES: CC1(OC(=O)c2ccc3nc(-c4cc(Cl)cc(Cl)c4)oc3c2)CCN(CCF)C1] | Alkylate cmpd 117 ICH₂CH₂F, DIEA, DMF |
| 121 | 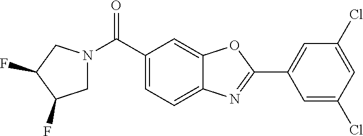 [SMILES: O=C(c1ccc2nc(-c3cc(Cl)cc(Cl)c3)oc2c1)N1C[C@@H](F)[C@@H](F)C1] | 25 |
| 122 | 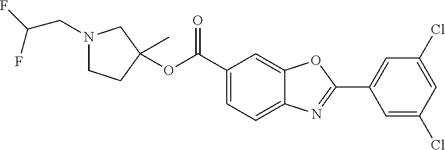 [SMILES: CC1(OC(=O)c2ccc3nc(-c4cc(Cl)cc(Cl)c4)oc3c2)CCN(CC(F)F)C1] | Alkylate cmpd 117 TfOCH₂CHF₂, DIEA, DMF |
| 123 | 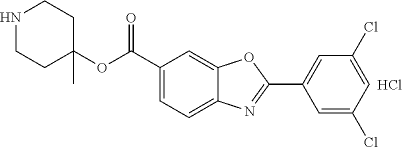 [SMILES: CC1(OC(=O)c2ccc3nc(-c4cc(Cl)cc(Cl)c4)oc3c2)CCNCC1 ; Cl] | BocPiperidineMeOH, DMAP, DIEA, DCM, then de-Boc 4N HCl/Dioxane |
| 124 | 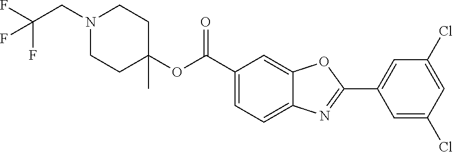 [SMILES: CC1(OC(=O)c2ccc3nc(-c4cc(Cl)cc(Cl)c4)oc3c2)CCN(CC(F)(F)F)CC1] | Alkylate cmpd 123 with TfOCH₂CF₃, DIEA, DMF |
| 125 | 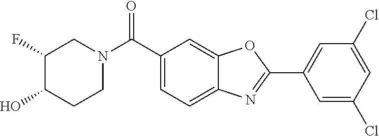 [SMILES: O=C(c1ccc2nc(-c3cc(Cl)cc(Cl)c3)oc2c1)N1CC[C@H](O)[C@H](F)C1] | 25 |
| 126 | 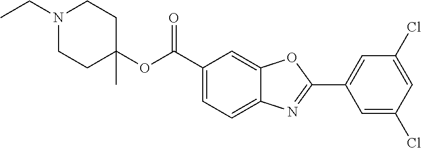 [SMILES: CCN1CCC(C)(OC(=O)c2ccc3nc(-c4cc(Cl)cc(Cl)c4)oc3c2)CC1] | DMAP, DIEA, DCM |

Analytical data for these compounds is provided in Table 3 below:

TABLE 3

| Compound | | LC/MS |
|---|---|---|
| Number | ¹H-NMR (400 MHz) | m/z M + 1 |
| 80 | (CDCl₃) δ = 8.27 (d, J = 0.8 Hz, 1H), 8.16 (d, J = 2.0 Hz, 2H), | 416.1 |
| | 8.11-8.09 (m, 1H), 7.80 (d, J = 8.4 Hz, 1H), 7.64 (s, 1H), 7.56 | |
| | (t, J = 2.0 Hz, 1H), 6.91 (s, 1H), 5.47-5.40 (m, 1H), 3.14-3.08 | |
| | (m, 1H), 3.05-3.00 (m, 1H), 1.45 (d, J = 6.0 Hz, 3H) | |
| 81 | (CDCl₃) δ = 8.15 (s, 2H), 7.81 (d, J = 8.0 Hz, 1H), 7.70 (s, 1H), | 377.0 |
| | 7.54 (s, 1H), 7.45 (br d, J = 8.0 Hz, 1H), 3.90-3.49 (m, 8H) | |
| 82 | (CDCl₃) δ = 8.16 (d, J = 2.0 Hz, 2H), 7.80 (d, J = 8.0 Hz, 1H), | 391.0 |
| | 7.68 (d, J = 0.8 Hz, 1H), 7.54 (t, J = 2.0 Hz, 1H), 7.41 (dd, | |
| | J = 1.6 Hz, J = 8.4 Hz 1H), 4.26-4.14 (m, 1H), 4.06-3.99 (m, | |
| | 1H), 3.80-3.69 (m, 1H), 3.53-3.20 (m, 2H), 2.04-1.82 (m, | |
| | 2H), 1.68-1.58 (m, 2H) | |
| 83 | (CDCl₃) δ = 8.14 (s, 2H), 7.79 (d, J = 8.0 Hz, 1H), 7.71 (s, 1H), | 391.0 |
| | 7.53 (t, J = 1.8 Hz, 1H), 7.46 (br d, J = 8.0 Hz, 1H), 4.05-3.32 | |
| | (m, 6H), 1.94 (br s, 2H), 1.81-1.64 (m, 2H) | |
| 84 | (DMSO-d₆) δ = 8.15 (d, J = 1.2 Hz, 2H), 8.00-7.92 (m, 2H), | 377.0 |
| | 7.89 (d, J = 8.2 Hz, 1H), 7.60 (d, J = 8.4 Hz, 1H), 5.09-4.94 (m, | |
| | 1H), 4.43-4.16 (m, 1H), 3.65-3.55 (m, 2H), 3.46-3.40 (m, | |
| | 1H), 3.30-3.18 (m, 1H), 2.01-1.78 (m, 2H). | |
| 85 | (CDCl₃) δ = 8.15 (d, J = 2.0 Hz, 2H), 7.81-7.79 (m, 2H), | 391.0 |
| | 7.57-7.54 (m, 2H), 4.69 (bd, J = 5.6 Hz, 1H), 4.48-4.43 (m, 1H), | |
| | 3.88-3.76 (m, 2H), 3.57-3.53 (m, 2H), 2.25-2.18 (m, 1H), 1.93-1.90 | |
| | (m, 1H), 1.84-1.69 (m, 2H) | |
| 86 | (CDCl₃) δ = 8.16 (d, J = 1.6 Hz, 2H), 7.83-7.80 (m, 2H), | 412.9 |
| | 7.57-7.55 (m, 2H), 4.39-4.29 (m, 1H), 4.15-3.99 (m, 2H), | |
| | 3.94-3.64 (m, 2H), 2.56-2.53 (m, 1H) | |
| 87 | (CDCl₃) δ = 8.16 (d, J = 2.0 Hz, 2H), 7.80 (d, J = 8.4 Hz, 1H), | 334.9 |
| | 7.70 (s, 1H), 7.54 (t, J = 2.0 Hz, 1H), 7.46 (dd, J = 1.2, 8.4 Hz, | |
| | 1H), 3.16 (s, 3H), 3.04 (s, 3H) | |
| 88 | (CDCl₃) δ = 8.18 (s, 2H), 7.83-7.81 (m, 2H), 7.61-7.56 (m, | 360.9 |
| | 2H), 3.72 (t, J = 6.8 Hz, 2H), 3.51 (t, J = 6.4 Hz, 2H), 2.03 (td, | |
| | J = 7.2, 13.6 Hz, 2H), 1.97-1.91 (m, 2H) | |
| 89 | (CDCl₃) δ = 8.16 (d, J = 2.0 Hz, 2H), 7.79 (d, J = 8.0 Hz, 1H), | 375.0 |
| | 7.67 (s, 1H), 7.54 (t, J = 1.6 Hz, 1H), 7.45-7.42 (m, 1H), 3.76-3.69 | |
| | (m, 2H), 3.46-3.45 (m, 2H), 1.71 (s, 6H) | |
| 90 | (DMSO-d₆) δ = 8.17 (d, J = 2.0 Hz, 2H), 7.97-7.92 (m, 3H), | 414.9 |
| | 7.49 (bd, J = 8.0 Hz, 1H), 6.44-6.12 (m, 1H), 4.91-4.88 (m, | |
| | 1H), 3.95-3.41 (m, 6H) | |
| 91 | (DMSO-d₆) δ = 8.16 (d, J = 1.6 Hz, 2H), 7.95-7.89 (m, 3H), | 397.1 |
| | 7.46 (bd, J = 6.8 Hz, 1H), 4.83 (bs, 1H), 4.74-4.47 (m, 2H), | |
| | 3.86-3.37 (m, 6H) | |
| 92 | (CDCl₃) δ = 8.15 (d, J = 2.0 Hz, 2H), 7.79 (d, J = 8.4 Hz, 2H), | 391.0 |
| | 7.61-7.54 (m, 2H), 3.91-3.46 (m, 4H), 2.04-1.91 (m, 2H), | |
| | 1.53-1.41 (m, 3H) | |
| 93 | (DMSO-d₆) δ = 8.16 (d, J = 2.0 Hz, 2H), 8.03 (d, J = 0.8 Hz, | 363.0 |
| | 1H), 7.96 (t, J = 1.6 Hz, 1H), 7.90 (d, J = 8.4 Hz, 1H), 7.73-7.70 | |
| | (m, 1H), 5.78 (d, J = 5.6 Hz, 1H), 4.54-4.52 (m, 2H), 4.31-4.27 | |
| | (m, 1H), 4.11 (bs, 1H), 3.83 (bd, J = 10.0 Hz, 1H) | |
| 94 | (CDCl₃) δ = 8.15 (d, J = 2.0 Hz, 2H), 7.91 (d, J = 0.8 Hz, 1H), | 403.0 |
| | 7.78 (d, J = 8.4 Hz, 1H), 7.67-7.65 (m, 1H), 7.55 (t, J = 1.6 Hz, | |
| | 1H), 4.31-4.22 (m, 5H), 2.63-2.62 (m, 2H), 2.19-2.14 (m, | |
| | 2H), 1.86 (s, 1H) | |
| 95 | (CDCl₃) δ = 8.15 (d, J = 1.6 Hz, 2H), 7.79 (d, J = 8.4 Hz, 1H), | 405.1 |
| | 7.71 (s, 1H), 7.54-7.53 (m, 1H), 7.48-7.46 (dd, J = 8.4 Hz, 1.6 | |
| | Hz, 1H), 4.75-4.45 (br s, 1H), 4.05-3.97 (m, 2H), 3.80-3.65 | |
| | (m, 1H), 3.14-3.05 (m, 1H), 1.76-1.55 (m, 6H) | |
| 96 | (DMSO-d₆) δ = 8.36 (d, J = 8.0 Hz, 1H), 8.27 (s, 1H), 8.14 (d, | 405.1 |
| | J = 2.0 Hz, 2H), 7.98 (d, J = 8.4 Hz, 1H), 7.94 (d, J = 1.6 Hz, 1H), | |
| | 7.87 (d, J = 8.4 Hz, 1H), 4.38 (d, J = 2.8 Hz, 1H), 3.90-3.70 (m, | |
| | 2H), 1.81-1.65 (m, 4H), 1.60-1.45 (m, 4H) | |
| 97 | (DMSO-d₆) δ = 8.34 (d, J = 8.0 Hz, 1H), 8.25 (s, 1H), 8.16 (d, | 405.0 |
| | J = 2.0 Hz, 2H), 7.96-7.90 (m, 2H), 7.89 (d, J = 8.4 Hz, 1H), 4.57 | |
| | (d, J = 4.4 Hz, 1H), 3.80-3.65 (m, 1H), 3.45-3.33 (m, 1H), | |
| | 1.95-1.75 (m, 4H), 1.45-1.20 (m, 4H) | |
| 98 | (CD₃OD) δ = 8.25-8.10 (br m, 3H), 7.90 (br d, J = 8.0 Hz, 1H), | 417.2 |
| | 7.80 (br d, J = 8.4 Hz, 1H), 7.71 (br s, 1H), 4.45-4.35 (br m, | |
| | 1H), 4.20-4.10 (br m, 1H), 2.55-2.30 (br m, 4H), 2.20-2.10 | |
| | (br m, 2H), 2.00-1.90 (br m, 2H) | |
| 99 | (DMSO-d₆) δ = 8.44 (d, J = 7.6 Hz, 1H), 8.25 (s, 1H), 8.15 (d, | 405.0 |
| | J = 1.6 Hz, 2H), 8.00-7.85 (m, 3H), 4.69 (d, J = 4.8 Hz, 1H), | |
| | 3.90-3.75 (br m, 1H), 3.55-3.40 (br m, 1H), 2.10-2.00 (br m, 1H), | |
| | 1.85-1.65 (br m, 3H), 1.35-1.20 (br m, 3H), 1.15-1.00 (br m, 1H) | |
| 100 | (DMSO-d₆) δ = 8.29 (d, J = 7.6 Hz, 1H), 8.26 (s, 1H), 8.16 (d, | 405.1 |
| | J = 1.6 Hz, 2H), 8.00-7.88 (m, 3H), 4.46 (d, J = 2.8 Hz, 1H), | |
| | 4.60-4.35 (m, 1H), 4.05-3.95 (m, 1H), 1.85-1.65 (m, 3H), 1.63-1.45 | |
| | (m, 3H), 1.43-1.25 (m, 2H) | |
| 101 | (CDCl₃) δ = 8.09 (s, 2H), 7.84 (s, 1H), 7.74 (d, J = 8.4 Hz, 1H), | 376.9 |
| | 7.63 (d, J = 8.0 Hz, 1H), 7.52 (s, 1H), 4.46 (d, J = 2.8 Hz, 1H), | |
| | 4.35-4.10 (m, 4H), 1.59 (s, 3H) | |
| 102 | (CDCl₃) δ = 8.15 (d, J = 2.0 Hz, 2H), 7.79 (d, J = 8.4 Hz, 1H), | 405.0 |
| | 7.75 (s, 1H), 7.54 (t, J = 2.0 Hz, 1H), 7.49 (d, J = 8.4 Hz, 1H), | |
| | 4.55-3.35 (br m, 2H), 3.15-3.00 (br m, 2H), 2.00-1.50 (br m, | |
| | 3H), 1.45-1.10 (br m, 4H) | |
| 103 | (CDCl₃) δ = 8.15 (d, J = 1.6 Hz, 2H), 7.79 (d, J = 8.0 Hz, 1H), | 405.1 |
| | 7.68 (s, 1H), 7.54 (d, J = 2.0 Hz, 1H), 7.44 (d, J = 8.4 Hz, 1H), | |
| | 4.45-4.25 (br m, 1H), 3.70-3.20 (br m, 4H), 1.80-1.60 (br m, | |
| | 4H), 1.33 (s, 3H) | |
| 104 | (CDCl₃) δ = 8.16 (d, J = 2.0 Hz, 2H), 7.82 (d, J = 8.0 Hz, 1H), | 396.9 |
| | 7.69 (s, 1H), 7.60-7.45 (m, 2H), 4.15-3.70 (br m, 4H), 2.50-2.35 | |
| | (br m, 2H) | |
| 105 | (DMSO-d₆) δ = 8.17-8.16 (m, 2H), 7.99-7.95 (m, 2H), 7.91-7.85 | 417.2 |
| | (m, 1H), 7.59 (d, J = 8.4 Hz, 1H), 5.10-4.90 (m, 1H), 4.25-3.90 | |
| | (br m, 1H), 3.60-3.30 (m, 4H), 2.30-2.05 (br m, 2H), | |
| | 1.95-1.65 (br m, 4H) | |
| 106 | (DMSO-d₆) δ = 8.17-8.16 (m, 2H), 7.98-7.95 (m, 2H), 7.92-7.85 | 431.2 |
| | (m, 1H), 7.59 (d, J = 8.4 Hz, 1H), 4.90-4.90 (m, 1H), 3.55-3.35 | |
| | (br m, 4H), 2.15-1.80 (br m, 6H), 1.95-1.24, 1.10 (s, 3H) | |
| 107 | (CD₃OD) δ = 8.20 (d, J = 2.0 Hz, 2H), 8.02 (d, J = 1.6 Hz, 1H), | 416.9 |
| | 7.85-7.80 (m, 1H), 7.76-7.72 (m, 2H), 4.45-4.05 (m, 5H), | |
| | 2.20-2.10 (br m, 2H), 2.00-1.90 (br m, 3H), 1.70-1.60 (br m, 1H) | |
| 108 | (DMSO-d₆) δ = 8.15 (d, J = 2.0 Hz, 2H), 7.95 (t, J = 1.6 Hz, 1H), | 389.0 |
| | 7.85-7.75 (m, 2H), 7.49 (d, J = 8.0 Hz, 1H), 5.33 (s, 1H), 3.85-3.75 | |
| | (m, 2H), 3.70-3.60 (m, 1H), 3.40-3.30 (m, 2H), 1.65-1.55 (m, 2H) | |
| 109 | (DMSO-d₆) δ = 8.15 (d, J = 2.0 Hz, 2H), 8.05-7.75 (m, 3H), | 389.1 |
| | 7.51 (d, J = 8.0 Hz, 1H), 5.43 (d, J = 1.2 Hz, 1H), 3.88-3.85 (br d, | |
| | J = 4.0 Hz, 1H), 3.70-3.60 (br m, 1H), 3.55-3.45 (br m, 1H), | |
| | 3.35-3.30 (m, 1H), 2.98 (br s, 1H), 1.70-1.55 (br m, 2H) | |
| 110 | (DMSO-d₆) δ = 8.16 (s, 2H), 8.04 (br s, 1H), 7.97 (br s, 1H), | 417.0 |
| | 7.89 (d, J = 8.4 Hz, 1H), 7.72 (d, J = 8.0 Hz, 1H), 4.91 (d, J = | |
| | 6.0 Hz, 1H), 4.40 (br s, 1H), 4.32 (br s, 1H), 4.10 (br s, 1H), 4.03 | |
| | (br s, 1H), 2.21 (br s, 4H), 1.19, 1.13 (br s, 3H) | |
| 111 | (CD₃OD) δ = 8.20 (d, J = 1.6 Hz, 2H), 8.02 (s, 1H), 7.84 (d, J = | 431.0 |
| | 8.4 Hz, 1H), 7.74 (d, J = 8.4 Hz, 2H), 4.55-4.00 (m, 4H), 2.25-2.15 | |
| | (br m, 1H), 2.10-2.00 (br m, 2H), 1.95-1.85 (m, 1H), 1.80-1.70 | |
| | (br m, 2H), 1.30 (br s, 3H) | |
| 112 | (CD₃OD) δ = 8.21 (d, J = 1.6 Hz, 2H), 7.95 (d, J = 9.2 Hz, 1H), | 379.0 |
| | 7.86 (d, J = 8.0 Hz, 1H), 7.44 (t, J = 2.0 Hz, 1H), 7.65 (d, J = 8.8 | |
| | Hz, 1H), 5.32 (br t, J = 48 Hz, 1H), 3.95-3.60 (br m, 4H), 2.40-2.05 | |
| | (br m, 2H) | |
| 113 | (CD₃OD) δ = 8.22 (d, J = 1.6 Hz, 2H), 7.96 (s, 1H), 7.87 (d, J = | 427.0 |
| | 8.4 Hz, 1H), 7.44 (t, J = 2.0 Hz, 1H), 7.64 (d, J = 8.0 Hz, 1H), | |
| | 4.70-4.50 (br m, 1H), 4.10-3.95 (br m, 2H), 3.90-3.65 (br m, | |
| | 2H), 2.65-2.50 (br m, 2H) | |
| 114 | (CDCl₃) δ = 8.15 (d, J = 2.0 Hz, 2H), 7.81 (d, J = 4.0 Hz, 1H), | 408.9 |
| | 7.70 (s, 1H), 7.54 (t, J = 2.0 Hz, 1H), 7.46 (d, J = 8.0 Hz, 1H), | |
| | 4.95-3.10 (br m, 7H), 2.17 (br m, 1H), 2.00-1.80 (br m, 1H) | |
| 115 | (CDCl₃) δ = 8.15 (d, J = 1.6 Hz, 2H), 7.81 (d, J = 8.4 Hz, 1H), | 425.1 |
| | 7.67 (s, 1H), 7.40 (t, J = 2.0 Hz, 1H), 7.42 (d, J = 8.0 Hz, 1H), | |
| | 4.25-3.40 (br m, 4H), 2.50-2.05 (br m, 2H), 2.00-1.65 (br m, 4H) | |
| 116 | (CDCl₃) δ = 8.15 (d, J = 2.0 Hz, 2H), 7.82 (d, J = 8.4 Hz, 1H), | 426.4 |
| | 7.70 (s, 1H), 7.55 (s, 1H), 7.45 (d, J = 8.0 Hz, 1H), 4.15-3.45 | |
| | (br m, 5H), 2.42 (br s, 1H), 2.20-1.80 (br m, 2H) | |
| 117 | (CDCl₃) δ = 8.33 (s, 1H), 8.20-7.85 (m, 3H), 7.72 (d, J = 8.4 | 391.1 |
| | Hz, 1H), 7.52 (d, J = 2.0 Hz, 1H), 4.00 (d, J = 12.4 Hz, 1H), | |
| | 3.70-3.45 (br m, 2H), 4.39 (d, J = 12.4 Hz, 1H), 2.95-2.75 (br m, | |
| | 1H), 2.25-2.10 (br m, 2H), 1.88 (s, 3H) | |
| 118 | (CDCl₃) δ = 8.15 (d, J = 2.0 Hz, 2H), 7.85 (s, 1H), 7.81 (d, J = | 429.1 |
| | 8.0 Hz, 1H), 7.62 (d, J = 8.4 Hz, 1H), 7.55 (t, J = 2.0 Hz, 1H), | |
| | 5.30-5.00 (br m, 1H), 3.80-3.50 (br m, 2H), 2.30-2.00 (br m, | |
| | 3H), 2.00-1.80 (br m, 1H) | |
| 119 | (CDCl₃) δ = 8.23 (s, 1H), 8.16 (d, J = 1.6 Hz, 2H), 8.07 (d, J = | 473.1 |
| | 8.4 Hz, 1H), 7.79 (d, J = 8.4 Hz, 1H), 7.55 (t, J = 2.0 Hz, 1H), | |
| | 3.30-3.05 (m, 4H), 3.00-2.85 (m, 2H), 2.60-2.40 (m, 1H), | |
| | 2.20-2.10 (m, 1H), 1.77 (s, 3H) | |
| 120 | (CDCl₃) δ = 8.24 (s, 1H), 8.16 (d, J = 2.0 Hz, 2H), 8.08 (d, J = | 437.1 |
| | 8.4 Hz, 1H), 7.79 (d, J = 8.4 Hz, 1H), 7.55 (t, J = 2.0 Hz, 1H), | |
| | 4.64 (t, J = 5.2 Hz, 1H), 4.52 (t, J = 5.2 Hz, 1H), 3.17 (d, J = | |
| | 10.8 Hz, 1H), 3.00 (d, J = 10.8 Hz, 1H), 2.95-2.65 (m, 4H), | |
| | 2.60-2.40 (m, 1H), 2.20-2.10 (m, 1H), 1.76 (s, 3H) | |
| 121 | (CDCl₃) δ = 8.16 (d, J = 2.0 Hz, 2H), 7.95-7.75 (m, 2H), | 397.1 |
| | 7.70-7.45 (m, 2H), 5.40-4.90 (br m, 2H), 4.15-3.75 (br m, 4H) | |
| 122 | (CDCl₃) δ = 8.40-8.00 (br m, 4H), 7.90-7.75 (bm, 1H), 7.65-7.50 | 455.2 |
| | (br m, 1H), 7.50-7.25 (br m, 1H), 6.15-5.65 (br t, 1H), | |
| | 3.35-3.15 (br m, 1H), 3.15-3.00 (br m, 1H), 3.00-2.80 (br m, | |
| | 4H), 2.65-2.40 (br m, 1H), 2.30-2.05 (br m, 1H), 1.78 (br s, 3H) | |
| 123 | (CD₃OD) δ = 8.39 (s, 1H), 8.21 (d, J = 1.6 Hz, 2H), 8.15 (d, J = | 405.0 |
| | 8.4 Hz, 1H), 7.86 (d, J = 8.0 Hz, 1H), 7.75 (t, J = 2.0 Hz, 1H), | |
| | 3.40-3.20 (m, 4H), 2.80-2.65 (br d, 2H), 2.05-1.95 (br m, | |
| | 2H), 1.76 (s, 3H) | |
| 124 | (CDCl₃) δ = 8.25 (s, 1H), 8.17 (d, J = 2.0 Hz, 2H), 8.09 (d, J = | 487.0 |
| | 8.4 Hz, 1H), 7.81 (d, J = 8.4 Hz, 1H), 7.56 (t, J = 2.0 Hz, 1H), | |
| | 3.05-2.95 (m, 2H), 2.90-2.75 (br m, 2H), 2.75-2.65 (br t, 2H), | |
| | 2.45-2.35 (br d, 2H), 1.90-1.75 (br m, 2H), 1.68 (s, 3H) | |
| 125 | (CDCl₃) δ = 8.16 (d, J = 1.6 Hz, 2H), 7.81 (d, J = 8.0 Hz, 1H), | 409.3 |
| | 7.71 (s, 1H), 7.55 (t, J = 1.6 Hz, 1H), 7.46 (d, J = 8.0 Hz, 1H), | |
| | 5.00-3.05 (br m, 7H), 2.10-1.90 (br m, 2H) | |
| 126 | (CDCl₃) δ = 8.25 (s, 1H), 8.18 (d, J = 2.0 Hz, 2H), 8.08 (d, J = | 433.1 |
| | 8.4 Hz, 1H), 7.81 (d, J = 8.4 Hz, 1H), 7.56 (t, J = 2.0 Hz, 1H), | |
| | 3.00-2.85 (br m, 1H), 2.60-2.40 (br m, 5H), 2.05-1.90 (br m, | |
| | 2H), 1.70-1.60 (br m, 5H), 1.26 (s, 3H) | |

### Example 79

A stability assay in liver S9 (rat or human) was used to evaluate the ability of a compound provided herein to convert to an active TTR stabilizer. The test compound was added to liver S9 and incubated at 37° C. in a water bath at a concentration of 1 μM. At each time point (0, 5, 10, 20, 30 and 60 min), stop solution (tolbutamide plus labetalol) was added to precipitate protein and mixed thoroughly. After centrifugation, an aliquot of supernatant was analyzed by LC-MS/MS. The percentage of formation of active agent was calculated at each time point.

### Example 80

For a compound provided herein to be an effective TTR stabilizer drug to halt and/or prevent the ocular and cerebral TTR amyloid deposition TTR amyloidosis, it has to be able to penetrate into the brain and CSF (surrogate for eye penetration) and deliver a sufficient amount of TTR stabilizer to stop TTR dissociation. A pharmacokinetic study in rat was used to evaluate the compounds. Male Sprague-Dawley (SD) rats (200-220 g weight) were acclimated for at least 2 to 3 days before being placed on study. All animals had access to certified rodent diet and water at libitum. Appropriate amount of the test compound was accurately weighed and mixed with appropriate volume of vehicle (such as DMSO/sterile water for iv dosing or 0.5% methylcellulose homogenous suspension or solution for oral administration or as a solution in a mixture NMP/PEG400/solutol/water) to administer a dose of 2, 5 or 10 mg/kg. For IV dosing the test compound was administered via tail vein or indwelling cannula. For oral dosing, the test compound was administered by oral gavage. Blood and CSF samples were collected at selected timepoints. Blood collection was performed from saphenous vein or tail vein of each animal into polypropylene tubes at each timepoint. All blood samples were transferred into EDTA-K2 tubes and centrifuged for 15 minutes at 4° C. for plasma collection. Plasma samples were kept at −80° C. until LC/MSMS analysis. CSF was collected from cisterna magna at each timepoint and quick frozen over dry ice and kept at −80° C. until LC/MSMS analysis. Brains were harvested immediately after the terminal bleeding (˜24 hrs post dosing). The blood of the brain was perfused with normal saline. The brain was quickly picked and placed into centrifuge tube. The weight of brain samples was recorded. 4-Fold homogenization solution (MeOH/15 mM PBS (1:2)) was added into the tube according to the weighed samples. The brain was homogenized using a Polytron (3 strokes or more until homogenous, each 30 seconds) on wet ice. The samples were quick frozen over dry ice and kept at −80° C. until LC/MSMS analysis. Using a LC-MSMS method for the quantitative determination of test compound in biological matrixes, amount of test compound and active agent were measured in plasma and CSF at selected timepoints post-dosing and in brain at 24 hrs post dosing. Plasma concentration versus time data was analyzed by non-compartmental approaches using the Phoenix WinNonlin 6.3 software program. As reference, an oral dose of 2 mg/kg of tafamidis gave a CSF to plasma ratio over 24 hrs around 0.01 and a brain to plasma ratio at 24 hrs around 0.02.

Results

Results from the rat liver S9 stability assays are shown in Table 4A.

Results from rat PK (CSF/plasma ratio and brain/plasma ratio at 24 hrs) are shown in Table 4B and Table 4C, respectively.

TABLE 4A
In vitro Rat Liver S9 Stability Assay -
Formation of tafamidis at 60 min

| Cmpd # | Formation* |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 26 | A |
| 27 | A |
| 28 | A |
| 29 | A |
| 30 | A |
| 31 | A |
| 32 | A |
| 33 | A |
| 34 | A |
| 36 | D |
| 37 | A |
| 38 | B |
| 40 | B |
| 41 | C |
| 43 | C |
| 44 | C |
| 45 | B |
| 46 | A |
| 47 | A |
| 48 | B |
| 49 | B |
| 50 | A |
| 51 | B |
| 52 | A |
| 53 | A |
| 54 | A |
| 55 | B |
| 56 | A |
| 57 | A |
| 58 | A |
| 59 | A |
| 60 | A |
| 63 | A |
| 64 | B |
| 65 | C |
| 66 | A |
| 67 | A |
| 69 | A |
| 70 | A |
| 71 | B |
| 72 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | A |
| 91 | A |
| 92 | A |
| 93 | A |
| 94 | A |
| 95 | A |
| 96 | A |
| 97 | A |
| 98 | A |
| 99 | A |
| 100 | A |
| 101 | A |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | A |
| 114 | A |
| 115 | A |
| 116 | A |
| 117 | A |
| 118 | A |
| 119 | A |
| 120 | A |
| 121 | A |
| 122 | C |
| 123 | A |
| 124 | A |
| 125 | A |
| 126 | A |

*Formation: A is <25%, B is ≥25 to <50%, C is ≥50 to <75% and D is ≥75%

TABLE 4B
In vivo Rat PK - Tafamidis CSF/plasma Ratio over 24 hrs (po)

| Cmpd # | CSF/Plasma Ratio* |
|---|---|
| 7 | C |
| 34 | B |
| 83 | A |
| 84 | A |
| 93 | A |
| 101 | B |
| 104 | A |

*CSF/Plasma Ratio: A is <0.015, B is ≥0.015 to <0.02, and C is ≥0.02

TABLE 4C
In vivo Rat PK - Tafamidis Brain/plasma Ratio at 24 hrs (po)

| Cmpd # | CSF/Plasma Ratio* |
|---|---|
| 7 | A |
| 34 | B |
| 83 | A |
| 84 | B |
| 93 | B |
| 101 | B |
| 104 | C |

*Brain/Plasma Ratio at 24 hrs: A is <0.04, B is ≥0.04 to <0.08, and C is ≥0.08

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound of Formula I: 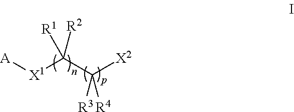
or a pharmaceutically acceptable salt or solvate thereof, wherein:
A [symbol] is 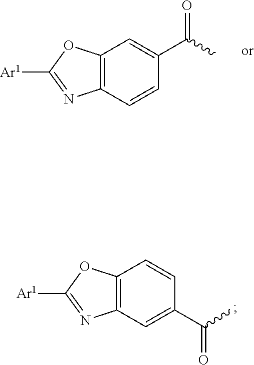
X¹ is O or NR⁵;
X² is H, halo, heteroaryl, CN, OR⁶ or NR⁷R⁸;
n is an integer from 1-2;
p is an integer from 0-3;
Ar¹ is aryl or heteroaryl, optionally substituted with halo, OR⁹, CN, COOH, CONR⁷R⁸, alkyl, haloalkyl, —(CR¹⁰R¹¹)_{q}OR⁹, —(CR¹⁰R¹¹)_{q}NR⁷R⁸ or —(CR¹⁰R¹¹)_{q}SH;
q is an integer from 0-6;
R¹-R⁸ are selected from (i)-(viii):
(i) R¹, R², R³ and R⁴ are each independently H, halo, haloalkyl, alkyl, aryl, heteroaryl, aralkyl or heteroaralkyl (all optionally substituted); and R⁵, R⁶, R⁷ and R⁸ are each independently H, haloalkyl, alkyl, aryl, heteroaryl, aralkyl, heteroaralkyl (all optionally substituted) or —(CH₂)ₘOR⁹; where m is an integer from 1-3; or
(ii) R¹ and R³ together with the atoms to which they are attached form a 3-6 membered ring, and R² and R⁴-R⁸ are selected as above; or
(iii) R¹ and R⁵ together with the atoms to which they are attached form a 3-6 membered ring, and R²-R⁴ and R⁶-R⁸ are selected as above; or
(iv) R¹ and R⁷ together with the atoms to which they are attached form a 3-6 membered ring, and R²-R⁶ and R⁸ are selected as above; or
(v) R¹ and R⁶ together with the atoms to which they are attached form a 3-6 membered ring, and R²-R⁵ and R⁷-R⁸ are selected as above; or
(vi) R³ and R⁵ together with the atoms to which they are attached form a 3-6 membered ring, and R¹-R², R⁴ and R⁶-R⁸ are selected as above; or
(vii) R³ and R⁸ together with the atoms to which they are attached form a 3-6 membered ring, and R¹-R² and R⁴-R⁷ are selected as above; or
(viii) R⁵ and R⁷ together with the atoms to which they are attached form a 3-6 membered ring, and R¹-R⁴, R⁶ and R⁸ are selected as above;
R⁹ is H, haloalkyl, alkyl, aryl, heteroaryl, aralkyl or heteroaralkyl (all optionally substituted); and
R¹⁰ and R¹¹ are each independently H, halogen, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted) or OR⁹.

2. The compound of claim 1, wherein:
A [symbol] is 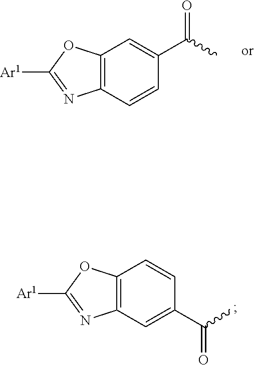
X¹ is O or NR⁵;
X² is H, halo, heteroaryl, CN, OR⁶ or NR⁷R⁸;
n is 1;
p is an integer from 0-3;
Ar¹ is aryl or heteroaryl, optionally substituted with halo, OR⁹, CN, COOH, CONR⁷R⁸, alkyl, haloalkyl, —(CR¹⁰R¹¹)_{q}OR⁹, —(CR¹⁰R¹¹)_{q}NR⁷R⁸ or —(CR¹⁰R¹¹)_{q}SH;
q is an integer from 0-6;
R¹-R⁸ are selected from (i)-(vi):
(i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
(ii) R¹ and R³ together with the atoms to which they are attached form a 3-6 membered ring, and R² and R⁴-R⁸ are selected as above; or
(iii) R¹ and R⁷ together with the atoms to which they are attached form a 3-6 membered ring, and R²-R⁶ and R⁸ are selected as above; or
(iv) R¹ and R⁶ together with the atoms to which they are attached form a 3-6 membered ring, and R²-R⁵ and R⁷-R⁸ are selected as above; or
(v) R³ and R⁸ together with the atoms to which they are attached form a 3-6 membered ring, and R¹-R² and R⁴-R⁷ are selected as above; or
(vi) R⁵ and R⁷ together with the atoms to which they are attached form a 3-6 membered ring, and R¹-R⁴, R⁶ and R⁸ are selected as above;
R⁹ is H or alkyl; and
R¹⁰ and R¹¹ are each independently H, halogen, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted) or OR⁹.

3. The compound of claim 1 or claim 2, wherein:
A [symbol] is 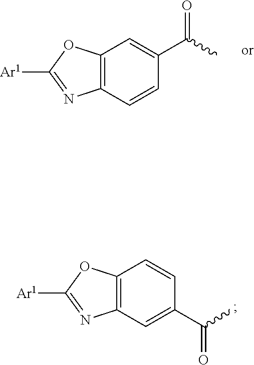
X¹ is O or NR⁵;
X² is H, halo, heteroaryl, CN, OR⁶ or NR⁷R⁸;
n is 1;
p is an integer from 0-3;
Ar¹ is aryl, optionally substituted with halo, OR⁹, CN, COOH, CONR⁷R⁸ or haloalkyl;
R¹-R⁸ are selected from (i)-(vi):
(i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
(ii) R¹ and R³ together with the atoms to which they are attached form a 3-6 membered ring, and R² and R⁴-R⁸ are selected as above; or
(iii) R¹ and R⁷ together with the atoms to which they are attached form a 3-6 membered ring, and R²-R⁶ and R⁸ are selected as above; or
(iv) R¹ and R⁶ together with the atoms to which they are attached form a 3-6 membered ring, and R²-R⁵ and R⁷-R⁸ are selected as above; or
(v) R³ and R¹ together with the atoms to which they are attached form a 3-6 membered ring, and R¹-R² and R⁴-R⁷ are selected as above; or
(vi) R⁵ and R⁷ together with the atoms to which they are attached form a 3-6 membered ring, and R¹-R⁴, R⁶ and R⁸ are selected as above; and
R⁹ is H or alkyl.

4. The compound of any one of claims 1-3, wherein:
A [symbol] is 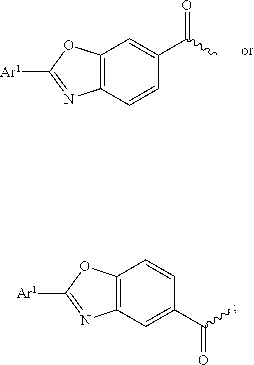
X¹ is O or NR⁵;
X² is H, halo, heteroaryl, CN, OR⁶ or NR⁷R⁸;
n is 1;
p is an integer from 0-3;
Ar¹ is aryl, optionally substituted with halo;
R¹-R⁸ are selected from (i)-(vi):
(i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
(ii) R¹ and R³ together with the atoms to which they are attached form a 3-6 membered ring, and R² and R⁴-R⁸ are selected as above; or
(iii) R¹ and R⁷ together with the atoms to which they are attached form a 3-6 membered ring, and R²-R⁶ and R⁸ are selected as above; or
(iv) R¹ and R⁶ together with the atoms to which they are attached form a 3-6 membered ring, and R²-R⁵ and R⁷-R⁸ are selected as above; or
(v) R³ and R⁸ together with the atoms to which they are attached form a 3-6 membered ring, and R¹-R² and R⁴-R⁷ are selected as above; or
(vi) R⁵ and R⁷ together with the atoms to which they are attached form a 3-6 membered ring, and R¹-R⁴, R⁶ and R⁸ are selected as above; and
R⁹ is H or alkyl.

5. The compound of any one of claims 1-4, wherein:
A [symbol] is 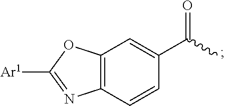
X¹ is O or NR⁵;
X² is H, halo, heteroaryl, CN, OR⁶ or NR⁷R⁸;
n is 1;
p is an integer from 0-3;
Ar¹ is aryl, optionally substituted with halo;
R¹-R⁸ are selected from (i)-(vi):
(i) R¹, R², R³ and R⁴ are each independently H, halo or optionally substituted alkyl; and R⁵, R⁶, R⁷ and R⁸ are each independently H, haloalkyl, optionally substituted alkyl or —(CH₂)ₘOR⁹; where m is an integer from 2-3; or
(ii) R¹ and R³ together with the atoms to which they are attached form a 3-6 membered carbocyclic ring, and R² and R⁴-R⁸ are selected as above; or
(iii) R¹ and R⁷ together with the atoms to which they are attached form a 3-6 membered heterocyclic ring, and R²-R⁶ and R⁸ are selected as above; or
(iv) R¹ and R⁶ together with the atoms to which they are attached form a 3-6 membered heterocyclic ring, and R²-R⁵ and R⁷-R⁸ are selected as above; or
(v) R³ and R⁸ together with the atoms to which they are attached form a 3-6 membered heterocyclic ring, and R¹-R² and R⁴-R⁷ are selected as above; or
(vi) R⁵ and R⁷ together with the atoms to which they are attached form a 3-6 membered heterocyclic ring, and R¹-R⁴, R⁶ and R⁸ are selected as above; and
R⁹ is H or alkyl.

6. The compound of any one of claims 1-5, wherein X¹ is O.

7. The compound of any one of claims 1-5, wherein X¹ NR⁵.

8. The compound of any one of claims 1-7, wherein X² is H.

9. The compound of any one of claims 1-7, wherein X² is halo.

10. The compound of any one of claims 1-7, wherein X² is F.

11. The compound of any one of claims 1-7, wherein X² is heteroaryl.

12. The compound of any one of claims 1-7, wherein X² is imidazolyl, pyridinyl, pyrimidinyl, pyrazinyl or pyridazinyl.

13. The compound of any one of claims 1-7, wherein X² is 2- or 4-imidazolyl, 3- or 4-pyridinyl, 2- or 4-pyrimidinyl, 2-pyrazinyl or 3-pyridazinyl.

14. The compound of any one of claims 1-7, wherein X² is CN.

15. The compound of any one of claims 1-7, wherein X² is OR⁶.

16. The compound of any one of claims 1-7, wherein X² is OH or OCH₃.

17. The compound of any one of claims 1-7, wherein X² is NR⁷R⁸.

18. The compound of any one of claims 1-7, wherein X² is NHMe or NMe₂.

19. The compound of any one of claims 1-18, wherein p is 0.

20. The compound of any one of claims 1-18, wherein p is 1.

21. The compound of any one of claims 1-18, wherein p is 2.

22. The compound of any one of claims 1-18, wherein p is 3.

23. The compound of any one of claims 1-22, wherein Ar¹ is phenyl optionally substituted with halo.

24. The compound of any one of claims 1-22, wherein Ar¹ is phenyl substituted with 1-2 halo.

25. The compound of any one of claims 1-22, wherein Ar¹ is dichlorophenyl.

26. The compound of any one of claims 1-22, wherein Ar¹ is 3,5-dichlorophenyl.

27. The compound of any one of claims 1-26, wherein R¹ is H or alkyl.

28. The compound of any one of claims 1-26, wherein R¹ is H, methyl or ethyl.

29. The compound of any one of claims 1-26, wherein R¹ is H.

30. The compound of any one of claims 1-29, wherein R² is H.

31. The compound of any one of claims 1-30, wherein R³ is H, halo or alkyl.

32. The compound of any one of claims 1-30, wherein R³ is H, F or methyl.

33. The compound of any one of claims 1-30, wherein R³ is H.

34. The compound of any one of claims 1-33, wherein R⁴ is H, halo or alkyl.

35. The compound of any one of claims 1-33, wherein R⁴ is H, F or methyl.

36. The compound of any one of claims 1-33, wherein R⁴ is H.

37. The compound of any one of claims 1-36, wherein R⁵ is H or alkyl.

38. The compound of any one of claims 1-36, wherein R⁵ is H or methyl.

39. The compound of any one of claims 1-36, wherein R⁵ is H.

40. The compound of any one of claims 1-39, wherein R⁶ is H or alkyl.

41. The compound of any one of claims 1-39, wherein R⁶ is H or methyl.

42. The compound of any one of claims 1-39, wherein R⁶ is H.

43. The compound of any one of claims 1-42, wherein R⁷ is H or alkyl.

44. The compound of any one of claims 1-42, wherein R⁷ is H or methyl.

45. The compound of any one of claims 1-42, wherein R⁷ is H.

46. The compound of any one of claims 1-42, wherein R⁷ is methyl.

47. The compound of any one of claims 1-46, wherein R⁸ is H or alkyl.

48. The compound of any one of claims 1-46, wherein R⁸ is H or methyl.

49. The compound of any one of claims 1-46, wherein R⁸ is H.

50. The compound of any one of claims 1-46, wherein R⁸ is methyl.

51. The compound of any one of claims 1-50, wherein R⁹ is H or alkyl.

52. The compound of any one of claims 1-50, wherein R⁹ is H or methyl.

53. The compound of any one of claims 1-50, wherein R⁹ is H.

54. The compound of any one of claims 1-50, wherein R⁹ is methyl.

55. The compound of any one of claims 1-54, wherein R¹⁰ and R¹¹ are each independently H, alkyl or OR⁹.

56. The compound of any one of claims 1-54, wherein R¹⁰ and R¹¹ are each independently H, methyl or OH.

57. The compound of any one of claims 1-54, wherein R¹⁰ and R¹¹ are each independently H or methyl.

58. The compound of any one of claims 1-26, 30 and 34-57, wherein R¹ and R³ together with the atoms to which they are attached form a 3-6 membered carbocyclic ring.

59. The compound of any one of claims 1-26, 30 and 34-57, wherein R¹ and R³ together form methylene, ethylene or propylene.

60. The compound of any one of claims 1, 6-26, 30-36 and 40-57, wherein R¹ and R⁵ in Formula I together with the atoms to which they are attached form a 3-6 membered heterocyclic ring.

61. The compound of any one of claims 1, 6-26, 30-36 and 40-57, wherein R¹ and R⁵ in Formula I together form optionally substituted ethylene, propylene, butylene or pentylene.

62. The compound of any one of claims 1, 6-26, 30-36 and 40-57, wherein R¹ and R⁵ in Formula I together form ethylene, propylene, butylene or pentylene, each optionally substituted with OH, halo, hydroxyalkyl, alkyl, perfluoroalkyl, spirocycloalkyl or fused cycloalkyl.

63. The compound of any one of claims 1, 6-26, 30-36 and 40-57, wherein R¹ and R⁵ in Formula I together form propylene or butylene, each optionally substituted with OH, F, CH₂OH, methyl, CF₃, spirocyclopentyl, spirocyclobutyl or fused cyclopropyl.

64. The compound of any one of claims 1, 6-26, 30-36 and 40-57, wherein R¹ and R⁵ in Formula I together form propylene or butylene.

65. The compound of any one of claims 1, 6-26, 30-36 and 40-57, wherein R¹ and R⁵ in Formula I together form butylene.

66. The compound of any one of claims 1-26, 30-42 and 47-57, wherein R¹ and R⁷ together with the atoms to which they are attached form a 3-6 membered heterocyclic ring.

67. The compound of any one of claims 1-26, 30-42 and 47-57, wherein R¹ and R⁷ together form optionally substituted methylene, ethylene or propylene.

68. The compound of any one of claims 1-26, 30-42 and 47-57, wherein R¹ and R⁷ together form methylene, ethylene, propylene, —CH₂C(O)— or —CH₂CF₂—.

69. The compound of any one of claims 1-26, 30-42 and 47-57, wherein R¹ and R⁷ together form methylene, ethylene or propylene.

70. The compound of any one of claims 1-26, 30-42 and 47-57, wherein R¹ and R⁷ together form ethylene or propylene.

71. The compound of any one of claims 1-26, 30-42 and 47-57, wherein R¹ and R⁷ together form ethylene.

72. The compound of any one of claims 1-26, 30-39 and 43-57, wherein R¹ and R⁶ together with the atoms to which they are attached form a 3-6 membered heterocyclic ring.

73. The compound of any one of claims 1-26, 30-39 and 43-57, wherein R¹ and R⁶ together form methylene or ethylene.

74. The compound of any one of claims 1-30, 34-46 and 51-57, wherein R³ and R⁸ together with the atoms to which they are attached form a 3-6 membered heterocyclic ring.

75. The compound of any one of claims 1-30, 34-46 and 51-57, wherein R³ and R⁸ together form propylene or butylene.

76. The compound of any one of claims 1-36, 40-42 and 47-57, wherein R⁵ and R⁷ together with the atoms to which they are attached form a 3-6 membered heterocyclic ring.

77. The compound of any one of claims 1-36, 40-42 and 47-57, wherein R⁵ and R⁷ together form ethylene.

78. The compound of claim 1, wherein X¹ is O; X² is NR⁷R⁸; R¹ and R⁷ together with the atoms to which they are attached form a 3-6 membered heterocyclic ring; R², R³ and R⁴ are each independently H, halo, haloalkyl, alkyl, aryl, heteroaryl, aralkyl or heteroaralkyl (all optionally substituted); R⁸ is independently H, haloalkyl, alkyl, aryl, heteroaryl, aralkyl, heteroaralkyl (all optionally substituted) or —(CH₂)ₘOR⁹ where m is an integer from 1-3; n is 1 and p is 2.

79. The compound of claim 1, wherein X¹ is O, X² is NR⁷R⁸; R¹ and R⁷ together form optionally substituted ethylene or propylene; R², R³ and R⁴ are each independently H, halo or alkyl; R⁸ is independently H or alkyl; n is 1 and p is 2.

80. The compound of claim 1, wherein X¹ is O, X² is NR⁷R⁸; R¹ and R⁷ together form ethylene or propylene; R² is H or methyl; R³ and R⁴ are each independently H, halo or alkyl; R⁸ is H, alkyl or haloalkyl; n is 1 and p is 2.

81. The compound of claim 1, wherein X¹ is O, X² is NR⁷R⁸; R¹ and R⁷ together form ethylene or propylene; R² is methyl; R³ and R⁴ are each independently H; R⁸ is ethyl, 2,2,2-trifluoroethyl, 2-fluoro-1-ethyl or 2,2-difluoro-1-ethyl; n is 1 and p is 2.

82. The compound of claim 1, wherein X¹ is NR⁵; X² is H; R¹ and R⁵ together with the atoms to which they are attached form a 3-6 membered heterocyclic ring; R² is independently H, halo, haloalkyl, alkyl, aryl, heteroaryl, aralkyl or heteroaralkyl (all optionally substituted); n is 1 and p is 0.

83. The compound of claim 1, wherein X¹ is NR⁵; X² is H; R¹ and R⁵ together form optionally substituted ethylene, propylene, butylene or pentylene; R² is independently H, halo or alkyl; n is 1 and p is 0.

84. The compound of claim 1, wherein X¹ is NR⁵; X² is H; R¹ and R⁵ together form propylene or butylene, each optionally substituted with OH, F, CH₂OH, methyl, CF₃, spirocyclopentyl, spirocyclobutyl or fused cyclopropyl; R² is H; n is 1 and p is 0.

85. The compound of claim 1, wherein X¹ is NR⁵; X² is H; R¹ and R³ together with the atoms to which they are attached form a 3-6 membered heterocyclic ring; R² and R⁴ are each independently H, halo, haloalkyl, alkyl, aryl, heteroaryl, aralkyl or heteroaralkyl (all optionally substituted); R⁵ is H or alkyl; n is 1 and p is 1.

86. The compound of claim 1, wherein X¹ is NR⁵; X² is H; R¹ and R³ together form methylene, ethylene or propylene; R² and R⁴ are each independently H, halo or alkyl; R⁵ is H or alkyl; n is 1 and p is 1.

87. The compound of claim 1, wherein X¹ is NR⁵; X² is H; R¹ and R³ together form propylene optionally substituted with OH; R² and R⁴ are each independently H or methyl; R⁵ is H; n is 1 and p is 1.

88. The compound of claim 1, wherein X¹ is NR⁵; X² is H; R¹ and R³ together form propylene optionally substituted with OH; R², R⁴ and R⁵ are each H; n is 1 and p is 1.

89. A compound of Formula II: 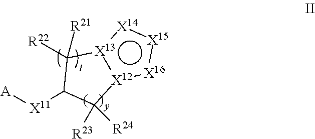
or a pharmaceutically acceptable salt or solvate thereof, wherein:
A [symbol] is 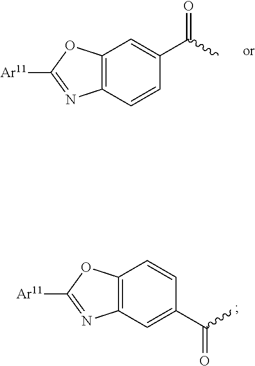
X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl or heteroaryl, optionally substituted with halo, OR³⁰, CN, COOH, CONR³¹R³², alkyl, haloalkyl, —(CR³³R³⁴)_{q}OR³⁰, —(CR³³R³⁴)_{q}NR³¹R³² or —(CR³³R³⁴)_{q}SH or CF₃;
q is an integer from 0-6;
R²¹, R²², R²³, R²⁴, R²⁵, R²⁶, R²⁷, R²⁸, R²⁹, R³⁰, R³¹, R³², R³⁵ and R³⁶ are each independently H, alkyl, aryl, aralkyl, heteroaryl or heteroaralkyl (all optionally substituted); and
R³³ and R³⁴ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR³⁰.

90. The compound of claim 89, wherein:
A [symbol] is 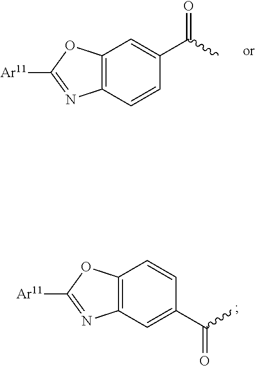
X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo, OR³⁰, CN, COOH, CONR³¹R³² or haloalkyl;
R²¹, R²², R²³, R²⁴, R²⁵, R²⁶, R²⁷, R²⁸, R²⁹, R³⁰, R³¹, R³², R³⁵ and R³⁶ are each independently H, alkyl, aryl, aralkyl, heteroaryl or heteroaralkyl (all optionally substituted).

91. The compound of claim 89 or claim 90, wherein:
A [symbol] is 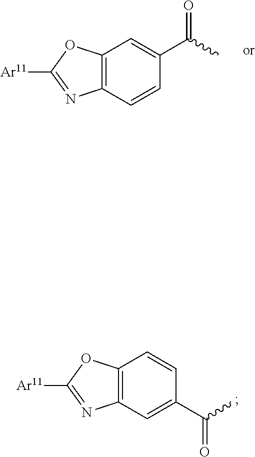
X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo; and
R²¹, R²², R²³, R²⁴, R²⁵, R²⁶, R²⁷, R²⁸, R²⁹, R³⁵ and R³⁶ are each independently H, alkyl, aryl, aralkyl, heteroaryl or heteroaralkyl (all optionally substituted).

92. The compound of any one of claims 89-91, wherein:
A [symbol] is 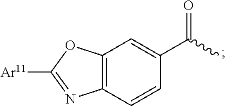
X¹¹ is O or NR²⁶;
X¹² is N or C;
X¹³ is N or C;
X¹⁴ is CR²⁹, N, ═CR²⁹—CR³⁵R³⁶—, ═N—CR³⁵R³⁶— or ═CR²⁹—NR²⁷—;
X¹⁵ is N or CR²⁸;
X¹⁶ is NR²⁶ or CR²⁵;
t is an integer from 1-3 and y is an integer from 0-2, wherein t+y≥2;
Ar¹¹ is aryl, optionally substituted with halo; and
R²¹, R²², R²³, R²⁴, R²⁵, R²⁶, R²⁷, R²⁸, R²⁹, R³⁵ and R³⁶ are each independently H, alkyl, aryl, aralkyl, heteroaryl or heteroaralkyl (all optionally substituted).

93. The compound of any one of claims 89-92, wherein X¹¹ is O.

94. The compound of any one of claims 89-93, wherein X¹¹ is NR²⁶.

95. The compound of any one of claims 89-94, wherein X¹¹ is NH.

96. The compound of any one of claims 89-95, wherein the ring formed by X¹²—X¹⁶ is one of the following:

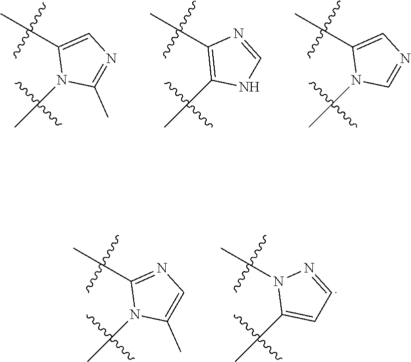

97. The compound of any one of claims 89-96, wherein X¹² is N.

98. The compound of any one of claims 89-96, wherein X¹² is C.

99. The compound of any one of claims 89-98, wherein X¹³ is C or N.

100. The compound of any one of claims 89-98, wherein X¹³ is C.

101. The compound of any one of claims 89-98, wherein X¹³ is N.

102. The compound of any one of claims 89-101, wherein X¹⁴ is CR²⁹ or N.

103. The compound of any one of claims 89-101, wherein X¹⁴ is CH or N.

104. The compound of any one of claims 89-101, wherein X¹⁴ is CR²⁹.

105. The compound of any one of claims 89-101, wherein X¹⁴ is CH.

106. The compound of any one of claims 89-101, wherein X¹⁴ is N.

107. The compound of any one of claims 89-106, wherein X¹⁵ is CR²⁸.

108. The compound of any one of claims 89-106, wherein X¹⁵ is CH or N.

109. The compound of any one of claims 89-106, wherein X¹⁵ is CH.

110. The compound of any one of claims 89-106, wherein X¹⁵ is N.

111. The compound of any one of claims 89-110, wherein X¹⁶ is CR²⁵.

112. The compound of any one of claims 89-110, wherein X¹⁶ is CH, CMe or NH.

113. The compound of any one of claims 89-110, wherein X¹⁶ is CH.

114. The compound of any one of claims 89-110, wherein X¹⁶ is CMe.

115. The compound of any one of claims 89-110, wherein X¹⁶ is NR²⁶.

116. The compound of any one of claims 89-110, wherein X¹⁶ is NH.

117. The compound of any one of claims 89-116, wherein R³⁵ and R³⁶ are independently H or optionally substituted alkyl.

118. The compound of any one of claims 89-116, wherein R³⁵ and R³⁶ are independently H or unsubstituted alkyl.

119. The compound of any one of claims 89-116, wherein R³⁵ and R³⁶ are H.

120. The compound of any one of claims 89-116, wherein R³⁵ and R³⁶ are independently H or methyl.

121. The compound of any one of claims 89-120, wherein t is 2 or 3 and y is 0 or 1.

122. The compound of any one of claims 89-120, wherein t is 1 and y is 1.

123. The compound of any one of claims 89-120, wherein t is 2 and y is 0 or 1.

124. The compound of any one of claims 89-120, wherein t is 2 and y is 1.

125. The compound of any one of claims 89-120, wherein t is 2 and y is 0.

126. The compound of any one of claims 89-125, wherein Ar¹¹ is phenyl, optionally substituted with halo.

127. The compound of any one of claims 89-125, wherein Ar¹¹ is phenyl, optionally substituted with chloro.

128. The compound of any one of claims 89-125, wherein Ar¹¹ is dichlorophenyl.

129. The compound of any one of claims 89-125, wherein Ar¹¹ is 3,5-dichlorophenyl.

130. The compound of any one of claims 89-129, wherein R²¹, R²², R²³, R²⁴, R²⁵, R²⁶, R²⁷, R²⁸, R²⁹, R³⁰, R³¹, R³², R³⁵ and R³⁶ are each independently H or optionally substituted alkyl.

131. The compound of any one of claims 89-129, wherein R²¹, R²², R²³, R²⁴, R²⁵, R²⁶, R²⁷ and R²⁸ are each independently H or unsubstituted alkyl.

132. The compound of any one of claims 89-129, wherein R²¹, R²², R²³, R²⁴, R²⁵, R²⁶, R²⁷ and R²⁸ are each independently H or methyl.

133. The compound of any one of claims 89-129, wherein R²¹, R²², R²³ and R²⁴ are each independently H or unsubstituted alkyl.

134. The compound of any one of claims 89-129, wherein R²¹, R²², R²³ and R²⁴ are each H.

135. The compound of any one of claims 89-129, wherein R²⁵, R²⁸, R²⁹, R³⁵ and R³⁶ are independently H or unsubstituted alkyl.

136. The compound of any one of claims 89-129, wherein R²⁵, R²⁸, R²⁹, R³⁵ and R³⁶ are independently H or methyl.

137. The compound of any one of claims 89-136, wherein R²⁶ and R²⁷ are each H.

138. The compound of any one of claims 72-137, wherein R³⁰ is H or unsubstituted alkyl.

139. The compound of any one of claims 72-137, wherein R³⁰ is H or methyl.

140. The compound of any one of claims 72-139, wherein R³¹ and R³² are independently H or unsubstituted alkyl.

141. The compound of any one of claims 72-139, wherein R³¹ and R³² are independently H or methyl.

142. The compound of any one of claims 1-88, wherein the compound of Formula I has the structure:

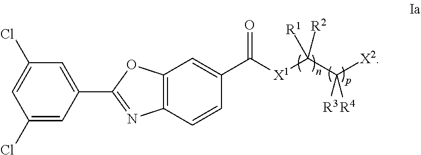

143. The compound of any one of claims 89-141, wherein the compound of Formula II has the structure:

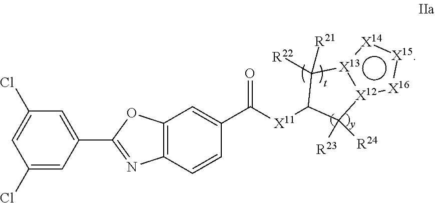

144. A compound selected from:

| Compound # | Structure |
|---|---|
| 1 | 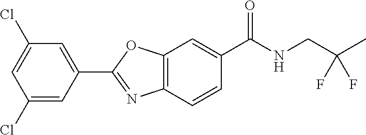 [SMILES: CC(F)(F)CNC(=O)c1ccc2nc(-c3cc(Cl)cc(Cl)c3)oc2c1] |
| 2 | 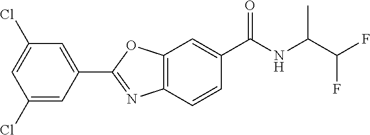 [SMILES: CC(NC(=O)c1ccc2nc(-c3cc(Cl)cc(Cl)c3)oc2c1)C(F)F] |
| 3 | 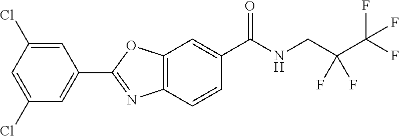 [SMILES: O=C(NCC(F)(F)C(F)(F)F)c1ccc2nc(-c3cc(Cl)cc(Cl)c3)oc2c1] |
| 4 | 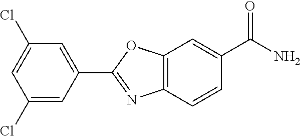 [SMILES: NC(=O)c1ccc2nc(-c3cc(Cl)cc(Cl)c3)oc2c1] |
| 5 | 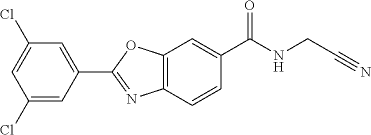 [SMILES: N#CCNC(=O)c1ccc2nc(-c3cc(Cl)cc(Cl)c3)oc2c1] |
| 6 | 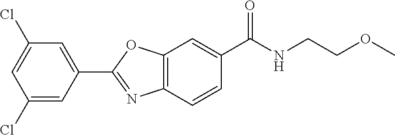 [SMILES: COCCNC(=O)c1ccc2nc(-c3cc(Cl)cc(Cl)c3)oc2c1] |
| 7 | 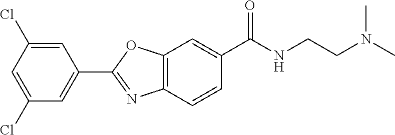 [SMILES: CN(C)CCNC(=O)c1ccc2nc(-c3cc(Cl)cc(Cl)c3)oc2c1] |
| 8 | 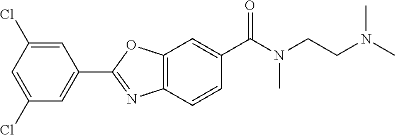 [SMILES: CN(C)CCN(C)C(=O)c1ccc2nc(-c3cc(Cl)cc(Cl)c3)oc2c1] |
| 9 | 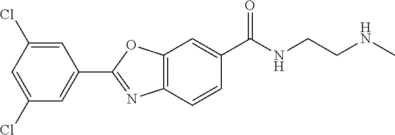 [SMILES: CNCCNC(=O)c1ccc2nc(-c3cc(Cl)cc(Cl)c3)oc2c1] |
| 10 | 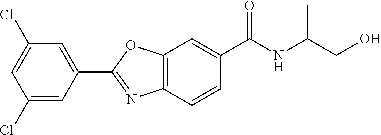 [SMILES: CC(CO)NC(=O)c1ccc2nc(-c3cc(Cl)cc(Cl)c3)oc2c1] |
| 11 | 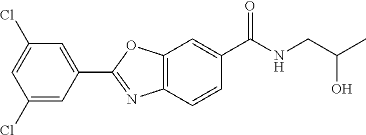 [SMILES: CC(O)CNC(=O)c1ccc2nc(-c3cc(Cl)cc(Cl)c3)oc2c1] |
| 12 | 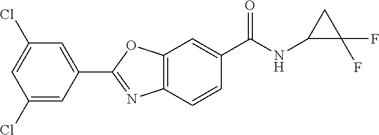 [SMILES: O=C(NC1CC1(F)F)c1ccc2nc(-c3cc(Cl)cc(Cl)c3)oc2c1] |
| 13 | 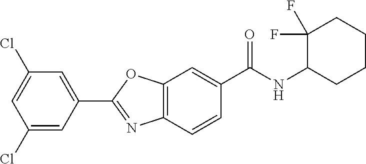 [SMILES: O=C(NC1CCCCC1(F)F)c1ccc2nc(-c3cc(Cl)cc(Cl)c3)oc2c1] |
| 14 | 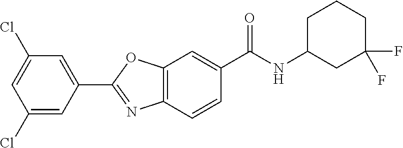 [SMILES: O=C(NC1CCCC(F)(F)C1)c1ccc2nc(-c3cc(Cl)cc(Cl)c3)oc2c1] |
| 15 | 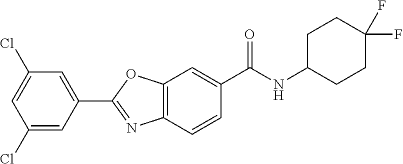 [SMILES: O=C(NC1CCC(F)(F)CC1)c1ccc2nc(-c3cc(Cl)cc(Cl)c3)oc2c1] |
| 16 | 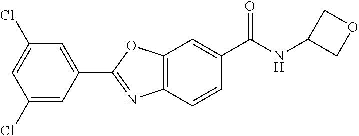 [SMILES: O=C(NC1COC1)c1ccc2nc(-c3cc(Cl)cc(Cl)c3)oc2c1] |
| 17 | 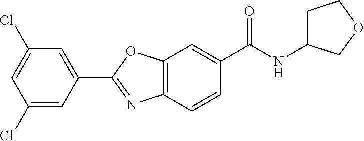 [SMILES: O=C(NC1CCOC1)c1ccc2nc(-c3cc(Cl)cc(Cl)c3)oc2c1] |
| 18 | 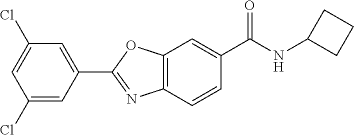 [SMILES: O=C(NC1CCC1)c1ccc2nc(-c3cc(Cl)cc(Cl)c3)oc2c1] |
| 19 | 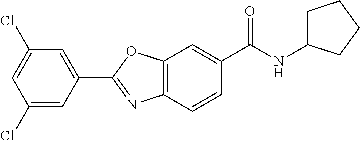 [SMILES: O=C(NC1CCCC1)c1ccc2nc(-c3cc(Cl)cc(Cl)c3)oc2c1] |
| 20 | 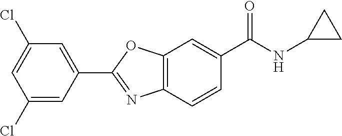 [SMILES: O=C(NC1CC1)c1ccc2nc(-c3cc(Cl)cc(Cl)c3)oc2c1] |
| 21 | 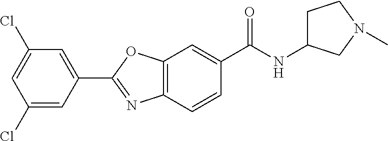 [SMILES: CN1CCC(NC(=O)c2ccc3nc(-c4cc(Cl)cc(Cl)c4)oc3c2)C1] |
| 22 | 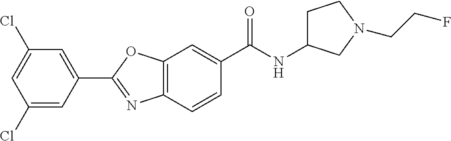 [SMILES: O=C(NC1CCN(CCF)C1)c1ccc2nc(-c3cc(Cl)cc(Cl)c3)oc2c1] |
| 23 | 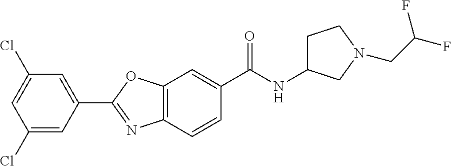 [SMILES: O=C(NC1CCN(CC(F)F)C1)c1ccc2nc(-c3cc(Cl)cc(Cl)c3)oc2c1] |
| 24 | 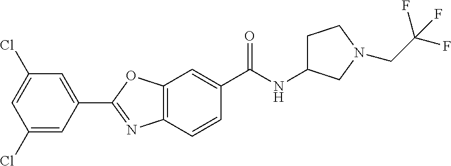 [SMILES: O=C(NC1CCN(CC(F)(F)F)C1)c1ccc2nc(-c3cc(Cl)cc(Cl)c3)oc2c1] |
| 25 | 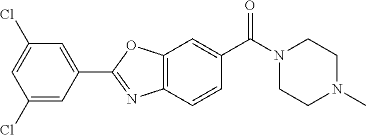 [SMILES: CN1CCN(C(=O)c2ccc3nc(-c4cc(Cl)cc(Cl)c4)oc3c2)CC1] |
| 26 | 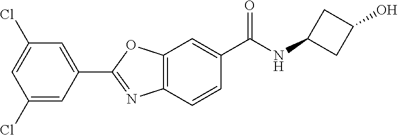 [SMILES: O=C(N[C@H]1C[C@H](O)C1)c1ccc2nc(-c3cc(Cl)cc(Cl)c3)oc2c1] |
| 27 | 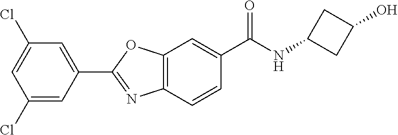 [SMILES: O=C(N[C@H]1C[C@@H](O)C1)c1ccc2nc(-c3cc(Cl)cc(Cl)c3)oc2c1] |
| 28 | 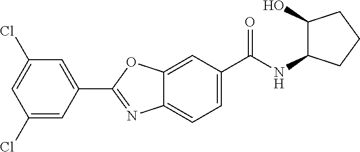 [SMILES: O=C(N[C@@H]1CCC[C@@H]1O)c1ccc2nc(-c3cc(Cl)cc(Cl)c3)oc2c1] |
| 29 | 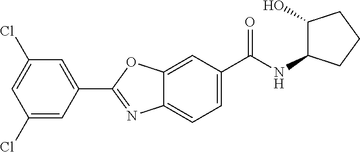 [SMILES: O=C(N[C@@H]1CCC[C@H]1O)c1ccc2nc(-c3cc(Cl)cc(Cl)c3)oc2c1] |
| 30 | 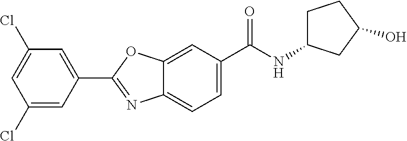 [SMILES: O=C(N[C@@H]1CC[C@H](O)C1)c1ccc2nc(-c3cc(Cl)cc(Cl)c3)oc2c1] |
| 31 | 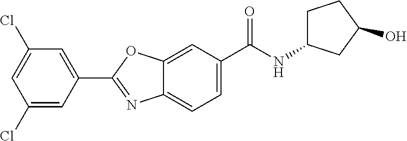 [SMILES: O=C(N[C@@H]1CC[C@@H](O)C1)c1ccc2nc(-c3cc(Cl)cc(Cl)c3)oc2c1] |
| 32 | 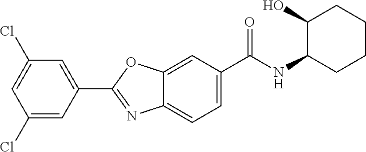 [SMILES: O=C(N[C@@H]1CCCC[C@@H]1O)c1ccc2nc(-c3cc(Cl)cc(Cl)c3)oc2c1] |
| 33 | 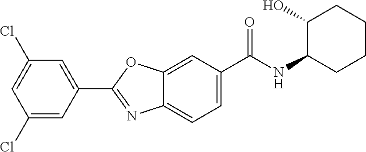 [SMILES: O=C(N[C@@H]1CCCC[C@H]1O)c1ccc2nc(-c3cc(Cl)cc(Cl)c3)oc2c1] |
| 34 | 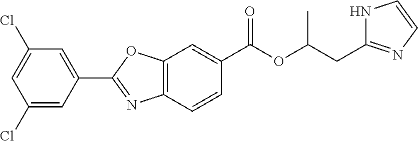 [SMILES: CC(Cc1ncc[nH]1)OC(=O)c1ccc2nc(-c3cc(Cl)cc(Cl)c3)oc2c1] |
| 35 | 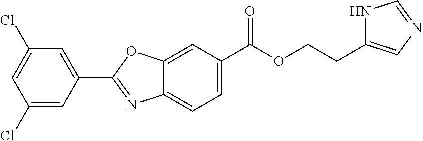 [SMILES: O=C(OCCc1cnc[nH]1)c1ccc2nc(-c3cc(Cl)cc(Cl)c3)oc2c1] |
| 36 | 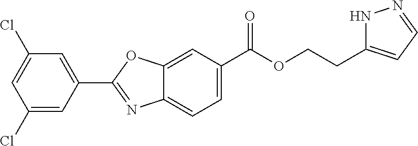 [SMILES: O=C(OCCc1ccn[nH]1)c1ccc2nc(-c3cc(Cl)cc(Cl)c3)oc2c1] |
| 37 | 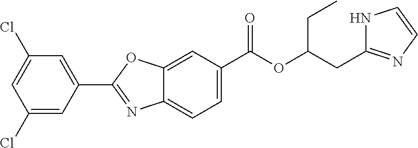 [SMILES: CCC(Cc1ncc[nH]1)OC(=O)c1ccc2nc(-c3cc(Cl)cc(Cl)c3)oc2c1] |
| 38 | 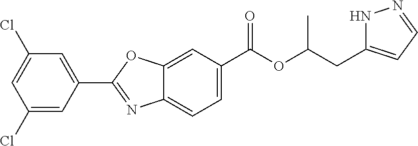 [SMILES: CC(Cc1ccn[nH]1)OC(=O)c1ccc2nc(-c3cc(Cl)cc(Cl)c3)oc2c1] |
| 39 | 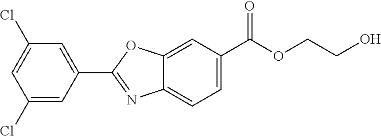 [SMILES: O=C(OCCO)c1ccc2nc(-c3cc(Cl)cc(Cl)c3)oc2c1] |
| 40 | 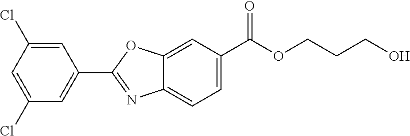 [SMILES: O=C(OCCCO)c1ccc2nc(-c3cc(Cl)cc(Cl)c3)oc2c1] |
| 41 | 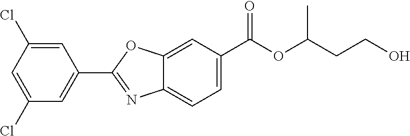 [SMILES: CC(CCO)OC(=O)c1ccc2nc(-c3cc(Cl)cc(Cl)c3)oc2c1] |
| 42 | 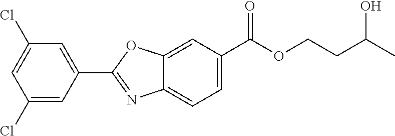 [SMILES: CC(O)CCOC(=O)c1ccc2nc(-c3cc(Cl)cc(Cl)c3)oc2c1] |
| 43 | 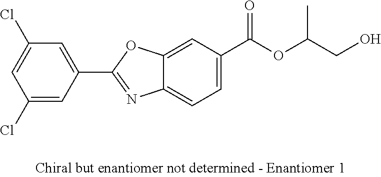 [SMILES: CC(CO)OC(=O)c1ccc2nc(-c3cc(Cl)cc(Cl)c3)oc2c1] |
| 44 | 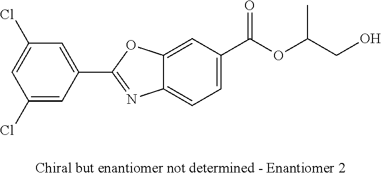 [SMILES: CC(CO)OC(=O)c1ccc2nc(-c3cc(Cl)cc(Cl)c3)oc2c1] |
| 45 | 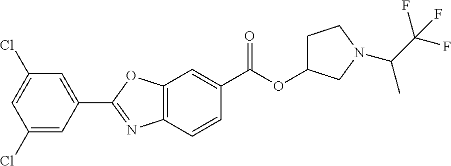 [SMILES: CC(N1CCC(OC(=O)c2ccc3nc(-c4cc(Cl)cc(Cl)c4)oc3c2)C1)C(F)(F)F] |
| 46 | 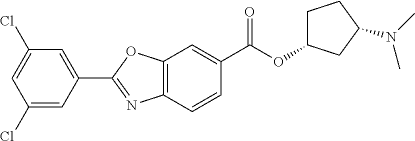 [SMILES: CN(C)[C@H]1CC[C@@H](OC(=O)c2ccc3nc(-c4cc(Cl)cc(Cl)c4)oc3c2)C1] |
| 47 | 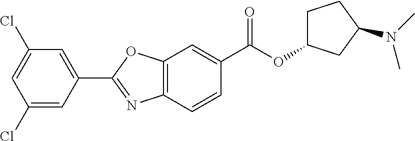 [SMILES: CN(C)[C@@H]1CC[C@@H](OC(=O)c2ccc3nc(-c4cc(Cl)cc(Cl)c4)oc3c2)C1] |
| 48 | 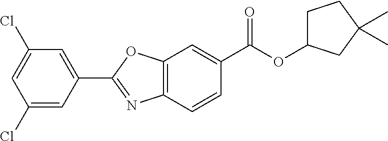 [SMILES: CC1(C)CCC(OC(=O)c2ccc3nc(-c4cc(Cl)cc(Cl)c4)oc3c2)C1] |
| 49 | 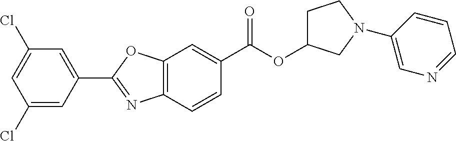 [SMILES: O=C(OC1CCN(c2cccnc2)C1)c1ccc2nc(-c3cc(Cl)cc(Cl)c3)oc2c1] |
| 50 | 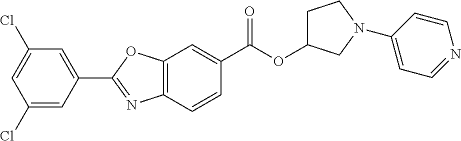 [SMILES: O=C(OC1CCN(c2ccncc2)C1)c1ccc2nc(-c3cc(Cl)cc(Cl)c3)oc2c1] |
| 51 | 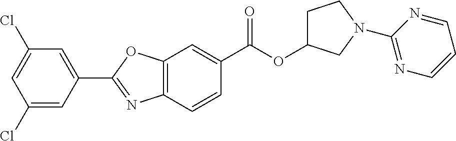 [SMILES: O=C(OC1CCN(c2ncccn2)C1)c1ccc2nc(-c3cc(Cl)cc(Cl)c3)oc2c1] |
| 52 | 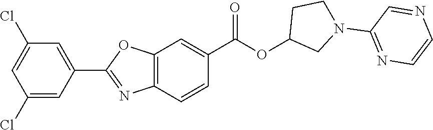 [SMILES: O=C(OC1CCN(c2cnccn2)C1)c1ccc2nc(-c3cc(Cl)cc(Cl)c3)oc2c1] |
| 53 | 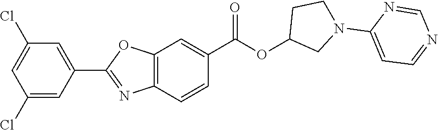 [SMILES: O=C(OC1CCN(c2ccncn2)C1)c1ccc2nc(-c3cc(Cl)cc(Cl)c3)oc2c1] |
| 54 | 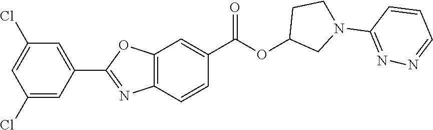 [SMILES: O=C(OC1CCN(c2cccnn2)C1)c1ccc2nc(-c3cc(Cl)cc(Cl)c3)oc2c1] |
| 55 | 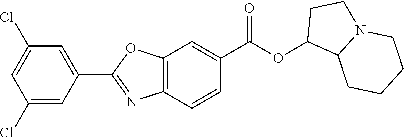 [SMILES: O=C(OC1CCN2CCCCC12)c1ccc2nc(-c3cc(Cl)cc(Cl)c3)oc2c1] |
| 56 | 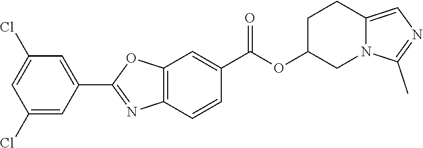 [SMILES: Cc1ncc2n1CC(OC(=O)c1ccc3nc(-c4cc(Cl)cc(Cl)c4)oc3c1)CC2] |
| 57 | 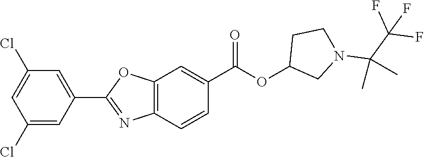 [SMILES: CC(C)(N1CCC(OC(=O)c2ccc3nc(-c4cc(Cl)cc(Cl)c4)oc3c2)C1)C(F)(F)F] |
| 58 | 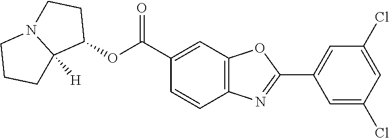 [SMILES: O=C(O[C@H]1CCN2CCC[C@H]12)c1ccc2nc(-c3cc(Cl)cc(Cl)c3)oc2c1] |
| 59 | 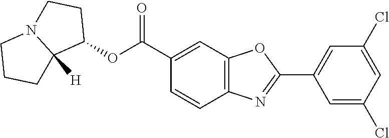 [SMILES: O=C(O[C@H]1CCN2CCC[C@@H]12)c1ccc2nc(-c3cc(Cl)cc(Cl)c3)oc2c1] |
| 60 | 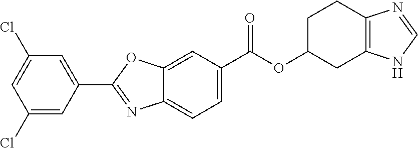 [SMILES: O=C(OC1CCc2nc[nH]c2C1)c1ccc2nc(-c3cc(Cl)cc(Cl)c3)oc2c1] |
| 61 | 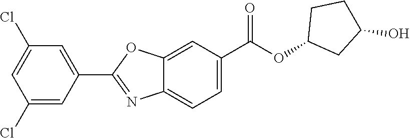 [SMILES: O=C(O[C@@H]1CC[C@H](O)C1)c1ccc2nc(-c3cc(Cl)cc(Cl)c3)oc2c1] |
| 62 | 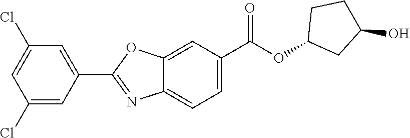 [SMILES: O=C(O[C@@H]1CC[C@@H](O)C1)c1ccc2nc(-c3cc(Cl)cc(Cl)c3)oc2c1] |
| 63 | 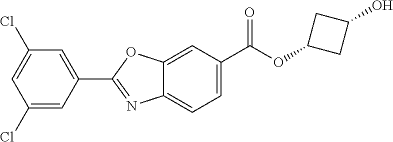 [SMILES: O=C(O[C@H]1C[C@@H](O)C1)c1ccc2nc(-c3cc(Cl)cc(Cl)c3)oc2c1] |
| 64 | 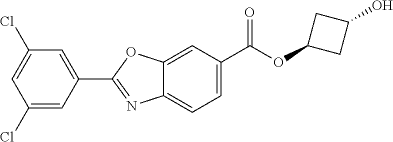 [SMILES: O=C(O[C@H]1C[C@H](O)C1)c1ccc2nc(-c3cc(Cl)cc(Cl)c3)oc2c1] |
| 65 | 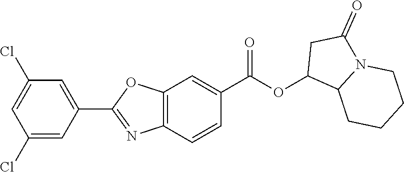 [SMILES: O=C(OC1CC(=O)N2CCCCC12)c1ccc2nc(-c3cc(Cl)cc(Cl)c3)oc2c1] |
| 66 | 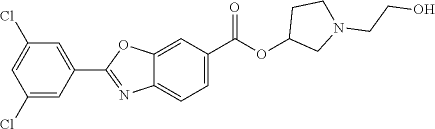 [SMILES: O=C(OC1CCN(CCO)C1)c1ccc2nc(-c3cc(Cl)cc(Cl)c3)oc2c1] |
| 67 | 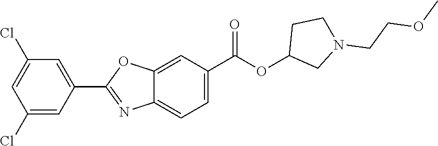 [SMILES: COCCN1CCC(OC(=O)c2ccc3nc(-c4cc(Cl)cc(Cl)c4)oc3c2)C1] |
| 68 | 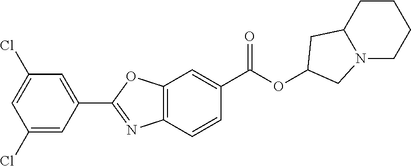 [SMILES: O=C(OC1CC2CCCCN2C1)c1ccc2nc(-c3cc(Cl)cc(Cl)c3)oc2c1] |
| 69 | 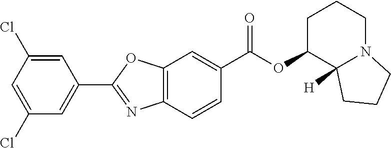 [SMILES: O=C(O[C@H]1CCCN2CCC[C@H]12)c1ccc2nc(-c3cc(Cl)cc(Cl)c3)oc2c1] |
| 70 | 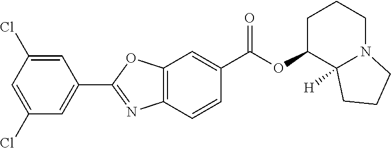 [SMILES: O=C(O[C@H]1CCCN2CCC[C@@H]12)c1ccc2nc(-c3cc(Cl)cc(Cl)c3)oc2c1] |
| 71 | 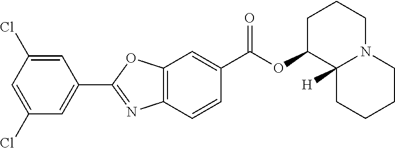 [SMILES: O=C(O[C@H]1CCCN2CCCC[C@H]12)c1ccc2nc(-c3cc(Cl)cc(Cl)c3)oc2c1] |
| 72 | 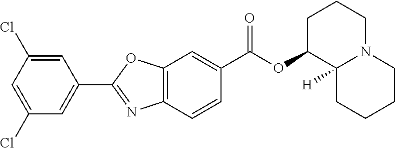 [SMILES: O=C(O[C@H]1CCCN2CCCC[C@@H]12)c1ccc2nc(-c3cc(Cl)cc(Cl)c3)oc2c1] |
| 73 | 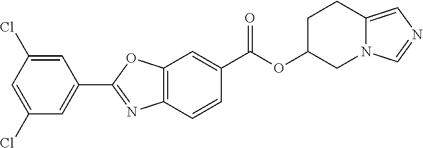 [SMILES: O=C(OC1CCc2cncn2C1)c1ccc2nc(-c3cc(Cl)cc(Cl)c3)oc2c1] |
| 74 | 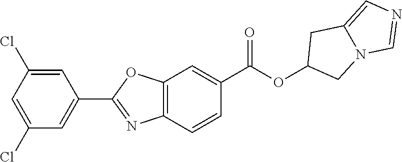 [SMILES: O=C(OC1Cc2cncn2C1)c1ccc2nc(-c3cc(Cl)cc(Cl)c3)oc2c1] |
| 75 | 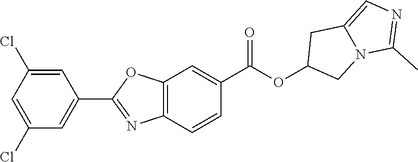 [SMILES: Cc1ncc2n1CC(OC(=O)c1ccc3nc(-c4cc(Cl)cc(Cl)c4)oc3c1)C2] |
| 76 | 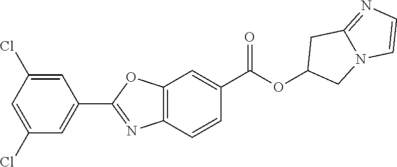 [SMILES: O=C(OC1Cc2nccn2C1)c1ccc2nc(-c3cc(Cl)cc(Cl)c3)oc2c1] |
| 77 | 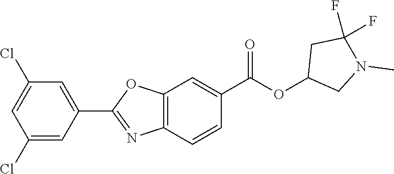 [SMILES: CN1CC(OC(=O)c2ccc3nc(-c4cc(Cl)cc(Cl)c4)oc3c2)CC1(F)F] |
| 78 | 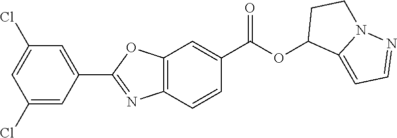 [SMILES: O=C(OC1CCn2nccc21)c1ccc2nc(-c3cc(Cl)cc(Cl)c3)oc2c1] |
| 79 | 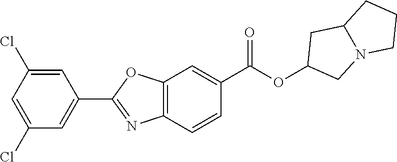 [SMILES: O=C(OC1CC2CCCN2C1)c1ccc2nc(-c3cc(Cl)cc(Cl)c3)oc2c1] |
| 80 | 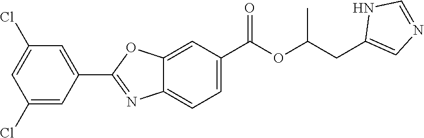 [SMILES: CC(Cc1cnc[nH]1)OC(=O)c1ccc2nc(-c3cc(Cl)cc(Cl)c3)oc2c1] |
| 81 | 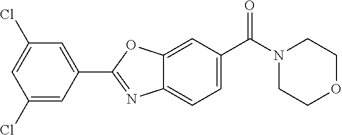 [SMILES: O=C(c1ccc2nc(-c3cc(Cl)cc(Cl)c3)oc2c1)N1CCOCC1] |
| 82 | 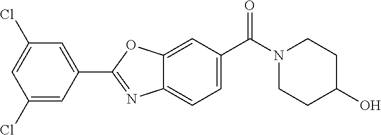 [SMILES: O=C(c1ccc2nc(-c3cc(Cl)cc(Cl)c3)oc2c1)N1CCC(O)CC1] |
| 83 | 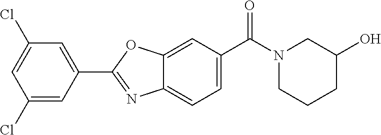 [SMILES: O=C(c1ccc2nc(-c3cc(Cl)cc(Cl)c3)oc2c1)N1CCCC(O)C1] |
| 84 | 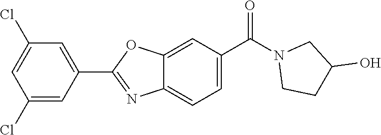 [SMILES: O=C(c1ccc2nc(-c3cc(Cl)cc(Cl)c3)oc2c1)N1CCC(O)C1] |
| 85 | 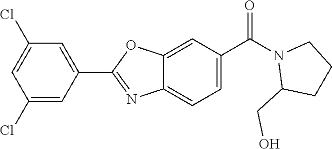 [SMILES: O=C(c1ccc2nc(-c3cc(Cl)cc(Cl)c3)oc2c1)N1CCCC1CO] |
| 86 | 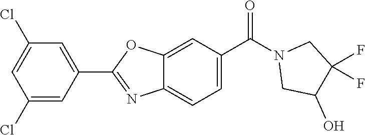 [SMILES: O=C(c1ccc2nc(-c3cc(Cl)cc(Cl)c3)oc2c1)N1CC(O)C(F)(F)C1] |
| 87 | 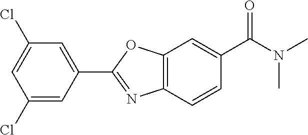 [SMILES: CN(C)C(=O)c1ccc2nc(-c3cc(Cl)cc(Cl)c3)oc2c1] |
| 88 | 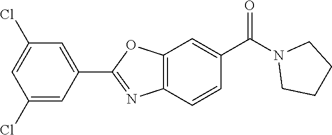 [SMILES: O=C(c1ccc2nc(-c3cc(Cl)cc(Cl)c3)oc2c1)N1CCCC1] |
| 89 | 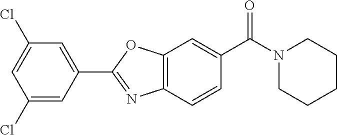 [SMILES: O=C(c1ccc2nc(-c3cc(Cl)cc(Cl)c3)oc2c1)N1CCCCC1] |
| 90 | 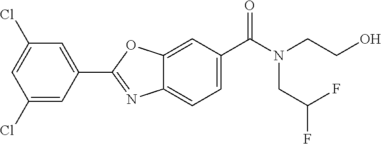 [SMILES: O=C(c1ccc2nc(-c3cc(Cl)cc(Cl)c3)oc2c1)N(CCO)CC(F)F] |
| 91 | 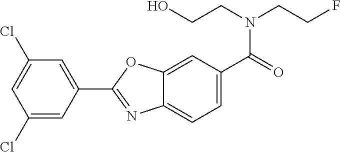 [SMILES: O=C(c1ccc2nc(-c3cc(Cl)cc(Cl)c3)oc2c1)N(CCO)CCF] |
| 92 | 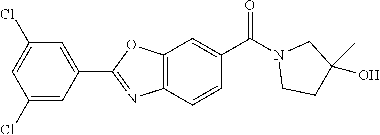 [SMILES: CC1(O)CCN(C(=O)c2ccc3nc(-c4cc(Cl)cc(Cl)c4)oc3c2)C1] |
| 93 | 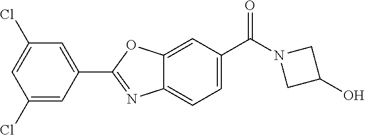 [SMILES: O=C(c1ccc2nc(-c3cc(Cl)cc(Cl)c3)oc2c1)N1CC(O)C1] |
| 94 | 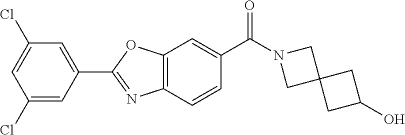 [SMILES: O=C(c1ccc2nc(-c3cc(Cl)cc(Cl)c3)oc2c1)N1CC2(CC(O)C2)C1] |
| 95 | 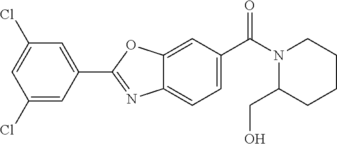 [SMILES: O=C(c1ccc2nc(-c3cc(Cl)cc(Cl)c3)oc2c1)N1CCCCC1CO] |
| 96 | 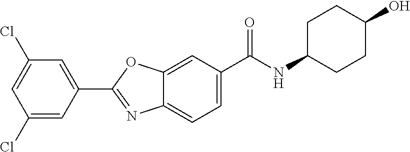 [SMILES: O=C(N[C@H]1CC[C@@H](O)CC1)c1ccc2nc(-c3cc(Cl)cc(Cl)c3)oc2c1] |
| 97 | 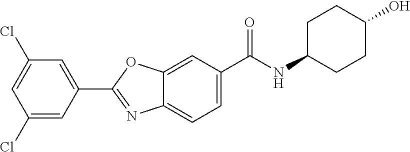 [SMILES: O=C(N[C@H]1CC[C@H](O)CC1)c1ccc2nc(-c3cc(Cl)cc(Cl)c3)oc2c1] |
| 98 | 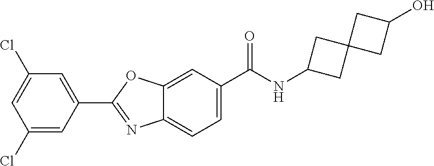 [SMILES: O=C(NC1CC2(CC(O)C2)C1)c1ccc2nc(-c3cc(Cl)cc(Cl)c3)oc2c1] |
| 99 | 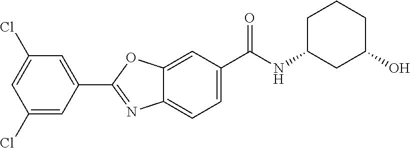 [SMILES: O=C(N[C@@H]1CCC[C@H](O)C1)c1ccc2nc(-c3cc(Cl)cc(Cl)c3)oc2c1] |
| 100 | 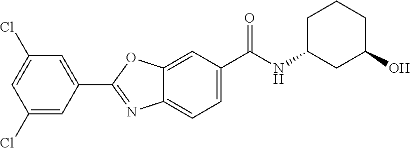 [SMILES: O=C(N[C@@H]1CCC[C@@H](O)C1)c1ccc2nc(-c3cc(Cl)cc(Cl)c3)oc2c1] |
| 101 | 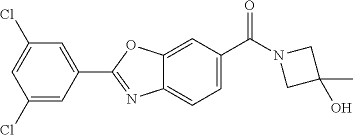 [SMILES: CC1(O)CN(C(=O)c2ccc3nc(-c4cc(Cl)cc(Cl)c4)oc3c2)C1] |
| 102 | 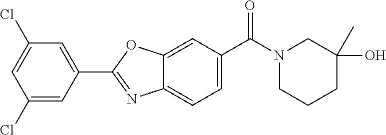 [SMILES: CC1(O)CCCN(C(=O)c2ccc3nc(-c4cc(Cl)cc(Cl)c4)oc3c2)C1] |
| 103 | 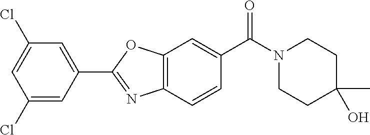 [SMILES: CC1(O)CCN(C(=O)c2ccc3nc(-c4cc(Cl)cc(Cl)c4)oc3c2)CC1] |
| 104 | 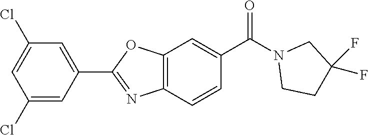 [SMILES: O=C(c1ccc2nc(-c3cc(Cl)cc(Cl)c3)oc2c1)N1CCC(F)(F)C1] |
| 105 | 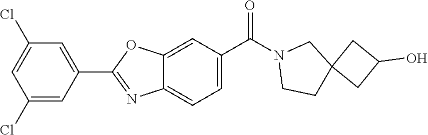 [SMILES: O=C(c1ccc2nc(-c3cc(Cl)cc(Cl)c3)oc2c1)N1CCC2(CC(O)C2)C1] |
| 106 | 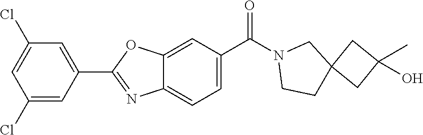 [SMILES: CC1(O)CC2(CCN(C(=O)c3ccc4nc(-c5cc(Cl)cc(Cl)c5)oc4c3)C2)C1] |
| 107 | 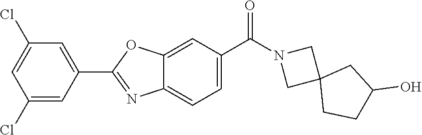 [SMILES: O=C(c1ccc2nc(-c3cc(Cl)cc(Cl)c3)oc2c1)N1CC2(CCC(O)C2)C1] |
| 108 | 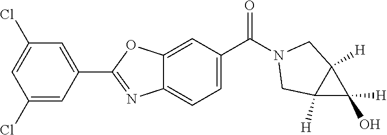 [SMILES: O=C(c1ccc2nc(-c3cc(Cl)cc(Cl)c3)oc2c1)N1C[C@@H]2[C@H](O)[C@@H]2C1] |
| 109 | 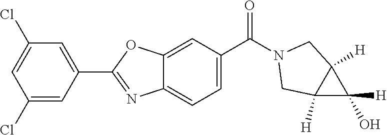 [SMILES: O=C(c1ccc2nc(-c3cc(Cl)cc(Cl)c3)oc2c1)N1C[C@@H]2[C@@H](O)[C@@H]2C1] |
| 110 | 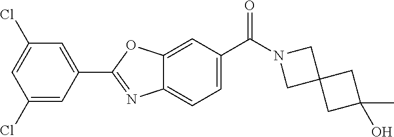 [SMILES: CC1(O)CC2(CN(C(=O)c3ccc4nc(-c5cc(Cl)cc(Cl)c5)oc4c3)C2)C1] |
| 111 | 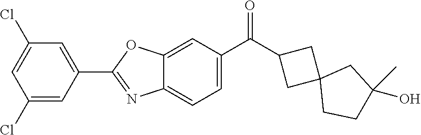 [SMILES: CC1(O)CCC2(CC(C(=O)c3ccc4nc(-c5cc(Cl)cc(Cl)c5)oc4c3)C2)C1] |
| 112 | 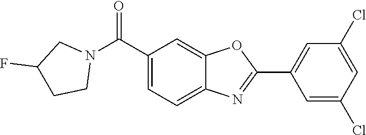 [SMILES: O=C(c1ccc2nc(-c3cc(Cl)cc(Cl)c3)oc2c1)N1CCC(F)C1] |
| 113 | 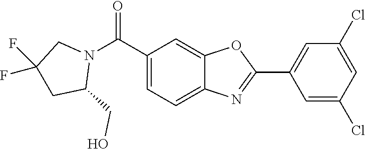 [SMILES: O=C(c1ccc2nc(-c3cc(Cl)cc(Cl)c3)oc2c1)N1CC(F)(F)C[C@H]1CO] |
| 114 | 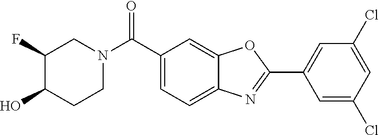 [SMILES: O=C(c1ccc2nc(-c3cc(Cl)cc(Cl)c3)oc2c1)N1CC[C@@H](O)[C@@H](F)C1] |
| 115 | 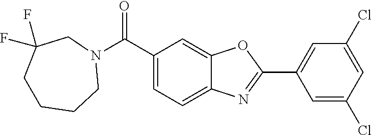 [SMILES: O=C(c1ccc2nc(-c3cc(Cl)cc(Cl)c3)oc2c1)N1CCCCC(F)(F)C1] |
| 116 | 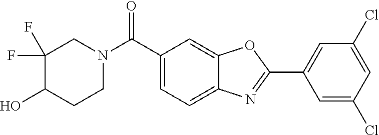 [SMILES: O=C(c1ccc2nc(-c3cc(Cl)cc(Cl)c3)oc2c1)N1CCC(O)C(F)(F)C1] |
| 117 | 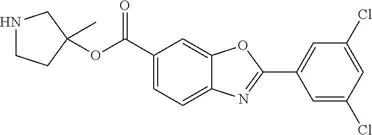 [SMILES: CC1(OC(=O)c2ccc3nc(-c4cc(Cl)cc(Cl)c4)oc3c2)CCNC1] |
| 118 | 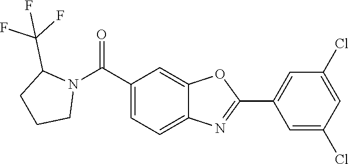 [SMILES: O=C(c1ccc2nc(-c3cc(Cl)cc(Cl)c3)oc2c1)N1CCCC1C(F)(F)F] |
| 119 | 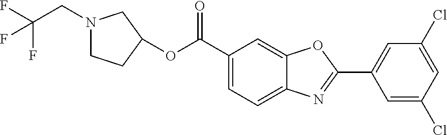 [SMILES: O=C(OC1CCN(CC(F)(F)F)C1)c1ccc2nc(-c3cc(Cl)cc(Cl)c3)oc2c1] |
| 120 | 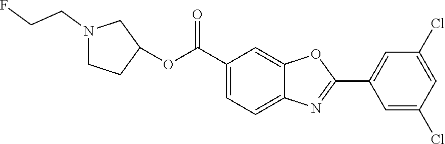 [SMILES: O=C(OC1CCN(CCF)C1)c1ccc2nc(-c3cc(Cl)cc(Cl)c3)oc2c1] |
| 121 | 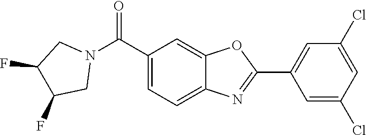 [SMILES: O=C(c1ccc2nc(-c3cc(Cl)cc(Cl)c3)oc2c1)N1C[C@@H](F)[C@@H](F)C1] |
| 122 | 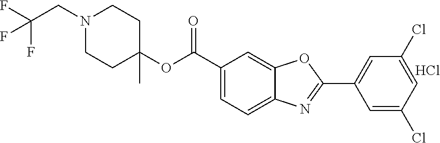 [SMILES: CC1(OC(=O)c2ccc3nc(-c4cc(Cl)cc(Cl)c4)oc3c2)CCN(CC(F)(F)F)CC1 ; Cl] |
| 123 | 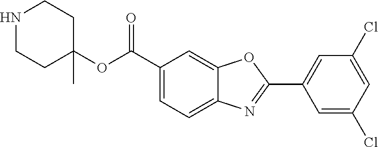 [SMILES: CC1(OC(=O)c2ccc3nc(-c4cc(Cl)cc(Cl)c4)oc3c2)CCNCC1] |
| 124 | 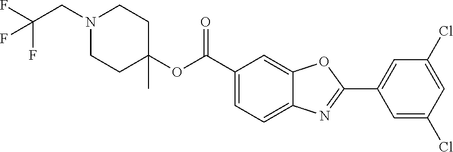 [SMILES: CC1(OC(=O)c2ccc3nc(-c4cc(Cl)cc(Cl)c4)oc3c2)CCN(CC(F)(F)F)CC1] |
| 125 | 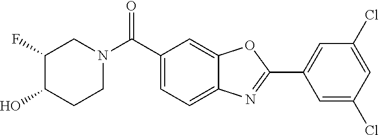 [SMILES: O=C(c1ccc2nc(-c3cc(Cl)cc(Cl)c3)oc2c1)N1CC[C@H](O)[C@H](F)C1] |
| 126 | 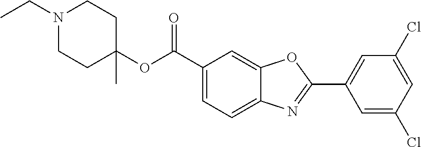 [SMILES: CCN1CCC(C)(OC(=O)c2ccc3nc(-c4cc(Cl)cc(Cl)c4)oc3c2)CC1] |

145. A pharmaceutical composition, comprising the compound of any one of claims 1-144 and a pharmaceutically acceptable carrier.

146. A method of inhibiting or preventing TTR aggregation and/or amyloid formation in the eye or CNS of a subject, comprising administering to the subject the compound of any one of claims 1-144 or the pharmaceutical composition of claim 145.

147. A method of inhibiting or preventing TTR aggregation and/or amyloid formation in peripheral nerves or cardiac tissues of a subject, comprising administering to the subject the compound of any one of claims 1-144 or the pharmaceutical composition of claim 145.

148. A method of treating a subject having peripheral TTR amyloidosis or ocular or cerebral amyloid angiopathy, comprising administering to the subject the compound of any one of claims 1-144 or the pharmaceutical composition of claim 145.

149. A method of treating a subject having familial amyloid polyneuropathy, familial amyloid cardiomyopathy, TTR oculoleptomeningeal amyloidosis or senile systemic amyloidosis, comprising administering to the subject the compound of any one of claims 1-144 or the pharmaceutical composition of claim 145.
